(19) United States
(12) Patent Application Publication
(10) Pub. No.: US 2025/0382640 A1
(43) Pub. Date: Dec. 18, 2025

(54) LIPID NANOPARTICLES FOR DELIVERY OF NUCLEIC ACIDS

(71) Applicant: Juno Therapeutics, Inc., Seattle, WA (US)

(72) Inventors: Leah Marie CAFFREY, Seattle, WA (US); Julie SHI, Seattle, WA (US); Wangzhong SHENG, San Diego, CA (US); Jeffrey O'BRIEN, La Jolla, CA (US); Wei DING, Princeton, NJ (US); Hung Yen LEE, Princeton, NJ (US); Scott MILLER, Princeton, NJ (US)

(73) Assignee: Juno Therapeutics, Inc., Seattle, WA (US)

(21) Appl. No.: 18/879,043

(22) PCT Filed: Jun. 29, 2023
(86) PCT No.: PCT/US2023/069453

§ 371 Date: Dec. 26, 2024

Related U.S. Application Data
(60) Provisional application No. 63/356,953, filed on Jun. 29, 2022.

(51) Int. Cl. C12N 15/88

(52) U.S. Cl. CPC C12N 15/88

(57) ABSTRACT

The present disclosure relates to compositions comprising lipid nanoparticles for delivering nucleic acid molecules into cells. Also included are methods for producing and using such compositions.

### CROSS-REFERENCE TO RELATED APPLICATIONS

This application claims priority to U.S. Provisional Patent Application No. 63/356,953, filed Jun. 29, 2022, entitled “Lipid Nanoparticles for Delivery of Nucleic Acids,” which is herein incorporated by reference in its entirety for all purposes.

### FIELD

The present disclosure relates in some aspects to lipid nanoparticles (LNPs) and compositions thereof for delivery of nucleic acid molecules, e.g., deoxyribonucleic acid (DNA), into lymphocytes, e.g., T cells. Also provided are methods for formulating LNPs, and for delivering nucleic acid molecules into lymphocytes, e.g., T cells, using LNP compositions, including in connection with gene editing and cell therapy.

### BACKGROUND

Viral vector-based delivery of nucleic acid, such as nucleic acid encoding a recombinant receptor (e.g., a CAR), into T cells can be effective in the production of T cells expressing the recombinant receptor, and such recombinant receptor-expressing T cells can be used in adoptive T cell therapies. The engineered expression of recombinant receptors, such as chimeric antigen receptors (CARs), on the surface of T cells enables the redirection of T cell specificity. In clinical studies, CAR-T cells, for example anti-CD19 CAR-T cells, have produced durable, complete responses in both leukemia and lymphoma patients (Porter et al. (2015) Sci Transl Med., 7:303ra139; Kochenderfer (2015) J. Clin. Oncol., 33: 540-9; Lee et al. (2015) Lancet, 385:517-28; Maude et al. (2014) N Engl J Med, 371:1507-17). Similarly, viral-vector based delivery of nucleic acid, such as nucleic acid encoding molecular gene editing components, into cells can be effective in the production of genetically edited cells for use in gene therapy applications.

Various strategies for delivering nucleic acid molecules into cells are available, including transfection- and transduction-based techniques. Among strategies for use in cell therapy are viral-vector based techniques of introducing nucleic acids into cells. Similarly, viral vectors are commonly used to effect gene editing. However, the production of viral vector-based compositions is labor intensive and resource-consuming. Improved non-viral compositions for delivering nucleic acid molecules into cells, and methods of producing and using the same, are therefore needed.

In certain contexts, viral-based methods of engineering cells (e.g., T cells) may not always be entirely satisfactory. Viral vectors, such as lentiviral vectors, are commonly used to genetically engineer T cells to express recombinant receptors (e.g., CARs), as well as to genetically edit cells for use in gene therapy applications. Such viral vectors must be of consistently high quality to ensure predictable genetic engineering of cells. In addition, viral vectors must be produced on a large scale, without compromising their quality, in order to produce therapeutic drug products containing a sufficient number of engineered cells. It is estimated that manufacturing of such viral vectors requires weeks. (Levine et al. (2017) Mol. Ther. Methods Clin. Dev., 4:92-101). Engineering cells with viral vectors is equally time- and labor-consuming. Continuous monitoring is necessary to ensure the safety of the viral vectors and cells engineered therewith. Combined with a limited number of manufacturing facilities, these characteristics of viral vector-based cell engineering make scaled production challenging and expensive. (Eyles et al. (2019) J. Chem. Technol. Biotechnol., 94:1008-16).

Another drawback of viral-vector based methods of cell engineering is their limitation in the size of the cargo (e.g., nucleic acid) they can deliver. For example, retroviral vectors, frequently used for gene delivery and capable of integrating into a host genome, contain approximate 8 kilobases (kb) of capacity for insertion of a transgene. Adenoviruses are able to deliver larger DNA particles, such as up to about 36 or 38 kb, but cannot integrate into a host genome. Adeno-associated vectors (AAV) are capable of integrating into a host genome, but have a packaging capacity of only about 4.7 kb. Thus, commonly used viral vectors can suffer from either an inability to integrate into a host genome, an inability to incorporate large transgenes, or both. (Nayerossadat et al. (2012) Adv. Biomed. Res., 1:27). By contrast, LNPs are capable of delivering larger cargo, including by delivery of nucleic acid molecules by transposon and CRISPR-Cas-mediated systems.

Non-viral methods of gene delivery and engineering have been investigated, including the use of DNA guns, electroporation, and ultrasound. However, these methods tend to suffer from low efficiency. Id. To date, a number of cationic lipid polymers have been developed for gene delivery, but in vivo studies have revealed substantial toxicity and low transfection efficiency. Id.

In addition, many methods of introducing nucleic acid into a cell, such as for genetic engineering purposes, rely on the introduction of ribonucleic acid (RNA) into cells. For example, it has been shown that lipid nanoparticles can be used to introduce RNA into cells. In some aspects, RNA encoding machinery of the CRISPR-Cas9 system is introduced into cells, such as for gene editing (Finn et al., Cell Reports (2018) 22(9):2227-35; Miller et al. Angew Chem Int Ed Engl (2017) 56(4):1059-63). In other aspects, RNA such as short interfering RNA (siRNA) or short hairpin RNA (shRNA) is introduced into cells, for the purpose of suppressing or disrupting a gene and/or its expression. (Cullis and Hope, Mol Ther (2017) 25(7):1467-75). By contrast, delivery of DNA into T cells, particular primary T cells, remains challenging. Non-viral methods of gene delivery to primary T cells often suffer from low efficiency, toxicity, or both (Rahimmanesh et al., Res Pharm Sci (2020) 15(5):437-46).

### SUMMARY

Provided herein are lipid nanoparticles (LNPs) that deliver deoxyribonucleic acid (DNA) and/or ribonucleic acid (RNA) payloads to cells. Also provide herein are fused nanoparticles that deliver multiple payloads to cells, in particular, nucleic acid molecules.

In one aspect, provided herein is a lipid nanoparticle (LNP) comprising: (1) an ionizable lipid comprising a diketopiperazine ring core, wherein the ionizable lipid is an ionizable amino lipid; and (2) a deoxyribonucleic acid (DNA) molecule. In another aspect, provided herein is a lipid nanoparticle (LNP) comprising: (1) an ionizable lipid comprising a dioxolane ring, wherein the ionizable lipid is an ionizable amino lipid; and (2) a deoxyribonucleic acid (DNA) molecule.

In some embodiments, the ionizable lipid is an ionizable amino lipidoid.

In some embodiments, the mass fraction of the ionizable lipid in the LNP is between about 30% and about 65%. In some embodiments, the mass fraction of the ionizable lipid in the LNP is between about 40% and about 60%. In some embodiments, the mass fraction of the ionizable lipid in the LNP is between about 30% and about 50%. In some embodiments, the mass fraction of the ionizable lipid in the LNP is between about 35% and about 45%. In some embodiments, the mass fraction of the ionizable lipid in the LNP is between about 40% and about 45%. In some embodiments, the mass fraction of the ionizable lipid in the LNP is between about 50% and about 60%. In some embodiments, the mass fraction of the ionizable lipid in the LNP is between about 40% and about 50%. In some embodiments, the mass fraction of the ionizable lipid in the LNP is about 40%. In some embodiments, the mass fraction of the ionizable lipid in the LNP is about 45%. In some embodiments, the mass fraction of the ionizable lipid in the LNP is about 50%. In some embodiments, the mass fraction of the ionizable lipid in the LNP is about 55%.

In some embodiments, the ionizable lipid comprises an unsaturated tail. In some embodiments, the unsaturated tail is an unsaturated linoleic tail. In some embodiments, the ionizable lipid is: (a) OF-C4-Deg-Lin, or an analog thereof; (b) cKK-E12, or an analog thereof; or (c) DLin-KC2-DMA, or an analog thereof. In some embodiments, the ionizable lipid is OF-C4-Deg-Lin.

In some embodiments, the ionizable lipid is OF-C4-Deg-Lin and the mass fraction of the ionizable lipid in the LNP is between about 30% and about 65%. In some embodiments, the ionizable lipid is OF-C4-Deg-Lin and the mass fraction of the ionizable lipid in the LNP is between about 40% and about 60%. In some embodiments, the ionizable lipid is OF-C4-Deg-Lin and the mass fraction of the ionizable lipid in the LNP is between about 30% and about 50%. In some embodiments, the ionizable lipid is OF-C4-Deg-Lin and the mass fraction of the ionizable lipid in the LNP is between about 40% and about 45%. In some embodiments, the ionizable lipid is OF-C4-Deg-Lin and the mass fraction of the ionizable lipid in the LNP is between about 35% and about 45%. In some embodiments, the ionizable lipid is OF-C4-Deg-Lin and the mass fraction of the ionizable lipid in the LNP is about 40%, about 45%, about 50%, about 55% or about 60%.

In some embodiments, the ionizable lipid is cKK-E12 and the mass fraction of the ionizable lipid in the LNP is between about 30% and about 65%. In some embodiments, the ionizable lipid is cKK-E12 and the mass fraction of the ionizable lipid in the LNP is between about 40% and about 60%. In some embodiments, the ionizable lipid is cKK-E12 and the mass fraction of the ionizable lipid in the LNP is between about 30% and about 50%. In some embodiments, the ionizable lipid is cKK-E12 and the mass fraction of the ionizable lipid in the LNP is between about 40% and about 45%. In some embodiments, the ionizable lipid is cKK-E12 and the mass fraction of the ionizable lipid in the LNP is between about 35% and about 45%. In some embodiments, the ionizable lipid is cKK-E12 and the mass fraction of the ionizable lipid in the LNP is about 40%, about 45%, about 50%, about 55% or about 60%.

In some embodiments, the ionizable lipid is DLin-KC2-DMA and the mass fraction of the ionizable lipid in the LNP is between about 30% and about 65%. In some embodiments, the ionizable lipid is DLin-KC2-DMA and the mass fraction of the ionizable lipid in the LNP is between about 40% and about 60%. In some embodiments, the ionizable lipid is DLin-KC2-DMA and the mass fraction of the ionizable lipid in the LNP is between about 30% and about 50%. In some embodiments, the ionizable lipid is DLin-KC2-DMA and the mass fraction of the ionizable lipid in the LNP is between about 40% and about 45%. In some embodiments, the ionizable lipid is DLin-KC2-DMA and the mass fraction of the ionizable lipid in the LNP is between about 35% and about 45%. In some embodiments, the ionizable lipid is DLin-KC2-DMA and the mass fraction of the ionizable lipid in the LNP is about 40%, about 45%, about 50%, about 55% or about 60%.

In some embodiments, the LNP comprises a helper lipid. In some embodiments, the mass fraction of the helper lipid in the LNP is between about 18% and about 22%. In some embodiments, the mass fraction of the helper lipid in the LNP is about 19%. In some embodiments, the helper lipid is 1-stearoyl-2-oleoyl-sn-glycero-3-phosphocholine (SOPC). In some embodiments, the helper lipid is 1,2-distearoyl-sn-glycero-3-phosphocholine (DSPC).

In some embodiments, the LNP comprises a polyethylene glycol (PEG)-conjugated lipid. In some embodiments, the mass fraction of the PEG-conjugated lipid in the LNP is between about 2% and about 3%. In some embodiments, the mass fraction of the PEG-conjugated lipid in the LNP is about 2.5%. In some embodiments, the PEG-conjugated lipid is DMG-PEG2000.

In some embodiments, the LNP comprises cholesterol. In some embodiments, the mass fraction of the cholesterol in the LNP is between about 30% and about 40%. In some embodiments, the mass fraction of the cholesterol in the LNP is about 35%.

In some embodiments, the LNP comprises more than one cationic lipid. In some embodiments, the LNP comprises two cationic lipids. In some embodiments, the LNP comprises an ionizable cationic lipid and a non-ionizable cationic lipid. The non-ionizable lipid has a higher pKa than the ionizable lipid and would predominantly be charged in both the bloodstream. In some embodiments, the mass fraction of the non-ionizable cationic lipid in the LNP is between about 0.1% and about 40%. In some embodiments, the mass fraction of the non-ionizable cationic lipid in the LNP is between about 0.2% and about 20%. In some embodiments, the mass fraction of the non-ionizable cationic lipid in the LNP is between about 0.2% and about 20%. In some embodiments, the mass fraction of the non-ionizable cationic lipid in the LNP is between about 0.5% and about 10%. In some embodiments, the mass fraction of the non-ionizable cationic lipid in the LNP is between about 1% and about 7%. In some embodiments, the mass fraction of the non-ionizable cationic lipid in the LNP is between about 1% and about 6%. In some embodiments, the mass fraction of the non-ionizable cationic lipid in the LNP is between about 2% and about 5%. In some embodiments, the mass fraction of the non-ionizable cationic lipid in the LNP is between about 0.2% and about 1%.

In some embodiments, the non-ionizable cationic lipid has the following structure:

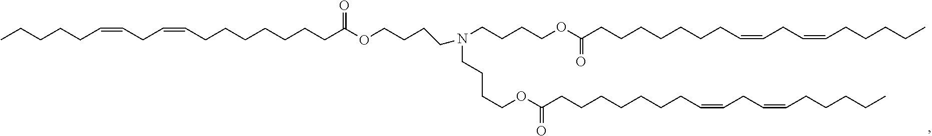 [SMILES: CCCCC/C=C\C/C=C\CCCCCCCC(=O)OCCCCN(CCCCOC(=O)CCCCCCC/C=C\C/C=C\CCCCC)CCCCOC(=O)CCCCCCC/C=C\C/C=C\CCCCC]

which is referred to herein as Lipid T1. In some embodiments, the mass fraction of Lipid T1 in the LNP is between about 0.1% and about 40%. In some embodiments, the mass fraction of Lipid T1 in the LNP is between about 0.2% and about 20%. In some embodiments, the mass fraction of Lipid T1 in the LNP is between about 0.2% and about 20%. In some embodiments, the mass fraction of Lipid T1 in the LNP is between about 0.5% and about 10%. In some embodiments, the mass fraction of Lipid T1 in the LNP is between about 1% and about 7%. In some embodiments, the mass fraction of Lipid T1 in the LNP is between about 1% and about 6%. In some embodiments, the mass fraction of Lipid T1 in the LNP is between about 2% and about 5%. In some embodiments, the mass fraction of Lipid T1 in the LNP is between about 0.2% and about 1%.

In some embodiments, the mass fraction of the DNA molecule in the LNP is between about 2% and about 6%. In some embodiments, the mass fraction of the DNA molecule in the LNP is between about 3% and about 4%. In some embodiments, the mass fraction of the DNA molecule in the LNP is about 3.5%.

Also provided herein is a lipid nanoparticle (LNP) comprising: (1) an ionizable lipid, or an analog thereof, wherein the ionizable lipid is DLin-KC2-DMA; and (2) a deoxyribonucleic acid (DNA) sequence, wherein the mass fraction of the ionizable lipid in the LNP is between about 35% and about 45%. In some embodiments, the mass fraction of the ionizable lipid in the LNP is about 40%.

Also provided herein is a lipid nanoparticle (LNP) comprising: (1) between about 35% and about 45% mass fraction of an ionizable lipid, wherein the ionizable lipid is OF-C4-Deg-Lin; (2) between about 3% and about 4% mass fraction of a deoxyribonucleic acid (DNA) molecule; (3) between about 18% and about 22% mass fraction of a helper lipid; (4) between about 2% and about 3% mass fraction of a polyethylene glycol (PEG)-conjugated lipid; and (5) between about 30% and about 40% mass fraction of cholesterol.

Also provided herein is a lipid nanoparticle (LNP) comprising: (1) between about 35% and about 45% mass fraction of an ionizable lipid, wherein the ionizable lipid is cKK-E12; (2) between about 3% and about 4% mass fraction of a deoxyribonucleic acid (DNA) molecule; (3) between about 18% and about 22% mass fraction of a helper lipid; (4) between about 2% and about 3% mass fraction of a polyethylene glycol (PEG)-conjugated lipid; and (5) between about 30% and about 40% mass fraction of cholesterol.

Also provided herein is a lipid nanoparticle (LNP) comprising: (1) between about 35% and about 45% mass fraction of an ionizable lipid, wherein the ionizable lipid is DLin-KC2-DMA; (2) between about 3% and about 4% mass fraction of a deoxyribonucleic acid (DNA) molecule; (3) between about 18% and about 22% mass fraction of a helper lipid; (4) between about 2% and about 3% mass fraction of a polyethylene glycol (PEG)-conjugated lipid; and (5) between about 30% and about 40% mass fraction of cholesterol.

Also provided herein is a lipid nanoparticle (LNP) comprising: (1) Lipid T1 and (2) a deoxyribonucleic acid (DNA) molecule. In some embodiments, the mass fraction of Lipid T1 in the LNP is between about 0.1% and about 60%. In some embodiments, the mass fraction of Lipid T1 in the LNP is between about 1% and about 40%. In some embodiments, the mass fraction of Lipid T1 in the LNP is between about 0.2% and about 20%. In some embodiments, the mass fraction of Lipid T1 in the LNP is between about 0.2% and about 20%. In some embodiments, the mass fraction of Lipid T1 in the LNP is between about 0.5% and about 10%. In some embodiments, the mass fraction of Lipid T1 in the LNP is between about 1% and about 7%. In some embodiments, the mass fraction of Lipid T1 in the LNP is between about 1% and about 6%. In some embodiments, the mass fraction of Lipid T1 in the LNP is between about 2% and about 5%. In some embodiments, the mass fraction of Lipid T1 in the LNP is between about 0.2% and about 1%. In some embodiments, the mass fraction of Lipid T1 in the LNP is between about 10% and about 55%. In some embodiments, the mass fraction of Lipid T1 in the LNP is between about 20% and about 50%. In some embodiments, the mass fraction of Lipid T1 in the LNP is between about 35% and about 45%. In some embodiments, the LNP does not comprise another cationic or ionizable lipid.

In some embodiments, the DNA molecule comprises a transgene. In some embodiments, the transgene encodes a recombinant receptor. In some embodiments, the DNA molecule is a closed end DNA (ceDNA) vector. In some embodiments, the transgene is positioned between protelomerase binding sequences. In some embodiments, the transgene is operably linked to a promoter and positioned between inverted terminal repeats (ITRs). In some embodiments, the ceDNA vector is between about 2 kilobases and about 10 kilobases. In some embodiments, the ceDNA vector is between about 4 kilobases and about 8 kilobases.

In some embodiments, the recombinant receptor is a chimeric antigen receptor (CAR) or a T cell receptor (TCR). In some embodiments, the recombinant receptor is a TCR. In some embodiments, the recombinant receptor is a CAR. In some embodiments, the CAR comprises an extracellular antigen-binding domain, a transmembrane domain, and an intracellular region. In some embodiments, the extracellular antigen-binding domain is an antibody or an antigen-binding fragment thereof that binds to an antigen that is associated with, or expressed on, a cell or tissue of a disease or condition. In some embodiments, the CAR is a single antigen directed CAR. In some embodiments, the CAR is a bispecific CAR. In some embodiments, the DNA (e.g., ceDNA) molecule encoding the bispecific CAR is at least 6 kilobases, at least 7 kilobases, or at least 8 kilobases. In some embodiments, the bispecific CAR is between about 6 kilobases and about 8 kilobases. In some embodiments, the bispecific CAR is about 8 kilobases.

In some embodiments, the antigen is selected from the group consisting of αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRCSA), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), and Wilms Tumor 1 (WT-1). In some embodiments, the antigen is BCMA. In some embodiments, the antigen is CD19. In some embodiments, the antigen is CD20. In some embodiments, the antigen is CD22. In some embodiments, the antigen is GPRC5D.

In some embodiments, the intracellular region comprises an intracellular signaling domain that is or comprises an intracellular signaling domain of a CD3 chain, or a signaling portion thereof. In some embodiments, the intracellular region comprises one or more costimulatory signaling domain(s) comprising an intracellular signaling domain selected from the group consisting of: a CD28, a 4-1BB, an ICOS, or a signaling portion thereof. In some embodiments, the intracellular region comprises one or more costimulatory signaling domain(s) comprising an intracellular signaling domain of 4-1BB.

In some embodiments, the DNA molecule comprises a single-stranded DNA oligonucleotide (ssODN) or a double-stranded DNA oligonucleotide (dsODN), the ssODN or the dsODN comprising a nucleotide sequence that is homologous to a target genomic locus. In some embodiments, the DNA molecule comprises a single-stranded DNA oligonucleotide (ssODN) comprising a nucleotide sequence that is homologous to a target genomic locus. In some embodiments, the DNA molecule comprises a double-stranded DNA oligonucleotide (dsODN) comprising a nucleotide sequence that is homologous to a target genomic locus.

Also provided herein is a co-formulated lipid nanoparticle (co-LNP) comprising: (1) a deoxyribonucleic acid (DNA) molecule and a ribonucleic acid (RNA) molecule; and (2) a first ionizable lipid and a second ionizable lipid. In some embodiments, the LNP comprises a third ionizable lipid. In some embodiments, (i) the DNA molecule is associated with the first ionizable lipid; and (ii) the RNA molecule is associated with the second ionizable lipid and/or the third ionizable lipid.

Provided herein is a co-formulated lipid nanoparticle (co-LNP) comprising a fusion of a first lipid nanoparticle (LNP) and a second lipid nanoparticle (LNP), wherein, prior to fusion: (1) the first LNP comprises: (i) a deoxyribonucleic acid (DNA) molecule; and (ii) a first ionizable lipid; and (2) the second LNP comprises: (i) a ribonucleic acid (RNA) molecule; and (ii) a second ionizable lipid. In some embodiments, the first LNP and second LNP, prior to fusion, are precursor LNPs that are not fully formed. As set forth herein, precursor LNPs are generated in an acidic environment (e.g., at a pH between about 4 and about 5). In some embodiments, the first ionizable lipid of the first LNP forms an ionic bond with the DNA molecule and the second ionizable lipid of the second LNP forms an ionic bond with the RNA molecule. In some embodiments, following fusion of the first and second precursor LNPs to form the fused co-LNP, the first ionizable lipid remains substantially associated (complexed) with the DNA molecule and the second ionizable lipid remains substantially associated (complexed) with the RNA molecule. For instance, in some embodiments, more than 75% of the first ionizable lipid remains associated with the DNA molecule and more than 75% of the second ionizable lipid remains associated with the RNA molecule in the fused co-LNP. In some embodiments, more than 80% of the first ionizable lipid remains associated with the DNA molecule and more than 80% of the second ionizable lipid remains associated with the RNA molecule in the fused co-LNP. In some embodiments, more than 85% of the first ionizable lipid remains associated with the DNA molecule and more than 85% of the second ionizable lipid remains associated with the RNA molecule in the fused co-LNP. In some embodiments, more than 90% of the first ionizable lipid remains associated with the DNA molecule and more than 90% of the second ionizable lipid remains associated with the RNA molecule in the fused co-LNP. In some embodiments, more than 95% of the first ionizable lipid remains associated with the DNA molecule and more than 95% of the second ionizable lipid remains associated with the RNA molecule in the fused co-LNP. In some embodiments, more than 99% of the first ionizable lipid remains associated with the DNA molecule and more than 99% of the second ionizable lipid remains associated with the RNA molecule in the fused co-LNP. In some embodiments, all of the first ionizable lipid remains associated with the DNA molecule and all of the second ionizable lipid remains associated with the RNA molecule in the fused co-LNP. In some embodiments, between about 75% and about 90% of the first ionizable lipid remains associated with the DNA molecule and between about 75% and about 90% of the second ionizable lipid remains associated with the RNA molecule in the fused co-LNP. In some embodiments, more than 75% of the first ionizable lipid remains associated with the DNA molecule and more than 75% of the second ionizable lipid remains associated with the RNA molecule in the fused co-LNP. In some embodiments, between about 75% and about 99% of the first ionizable lipid remains associated with the DNA molecule and between about 75% and about 99% of the second ionizable lipid remains associated with the RNA molecule in the fused co-LNP.

In some embodiments, the shell of the fused co-LNP comprises a mixture of lipids from each of the precursor LNPs. In some embodiments, the shell of the fused co-LNP is a hybrid of the lipids that comprise the two precursor LNPs.

In some embodiments, the second LNP comprises a first RNA molecule and a second RNA molecule. In some such embodiments the first mRNA molecule is an mRNA and the second RNA molecule is a guide RNA.

In some embodiments, the first ionizable lipid and/or the second ionizable lipid of the co-LNP is an ionizable amino lipid. In some embodiments, the first ionizable lipid is an ionizable amino lipid. In some embodiments, the second ionizable lipid is an ionizable amino lipid. In some embodiments, the first ionizable lipid and the second ionizable lipid is an ionizable amino lipid.

In some embodiments, the co-LNP comprises a volumetric ratio of the first LNP to the second LNP that is between about 3:1 and about 1:3.

In some embodiments, the fused co-LNP comprises a third LNP, wherein the third LNP comprises, prior to fusion: (i) an RNA molecule; and (ii) a third ionizable lipid. In some embodiments, the third ionizable lipid is an ionizable amino lipid. Co-LNPs comprising three nucleic acid molecules are also referred to herein as tri-LNPs.

In some embodiments, the RNA molecule of the second LNP (i.e., first RNA molecule) and the RNA of the third LNP (i.e., second RNA molecule) in the tri-LNP are different. In some embodiments, the first mRNA molecule is an mRNA and the second RNA molecule is a guide RNA. In some embodiments, the third LNP, prior to fusion, is a precursor LNP that is not fully formed. In some embodiments, the second LNP and the third LNP are fused by methods described herein to form an RNA co-LNP comprising the first RNA molecule and the second RNA molecule. The RNA co-LNP can then be fused the first LNP to form the tri-LNP. In some embodiments, following fusion of the second and third precursor LNPs to form the fused RNA co-LNP, the second ionizable lipid remains substantially associated (complexed) with the first RNA molecule (e.g., mRNA) and the third ionizable lipid remains substantially associated (complexed) with the second RNA molecule (e.g., guide RNA). For instance, in some embodiments, more than 75% of the second ionizable lipid remains associated with the first RNA molecule and more than 75% of the third ionizable lipid remains associated with the second RNA molecule in the fused RNA co-LNP. In some embodiments, more than 80% of the second ionizable lipid remains associated with the first RNA molecule and more than 80% of the third ionizable lipid remains associated with the second RNA molecule in the fused RNA co-LNP. In some embodiments, more than 85% of the second ionizable lipid remains associated with the first RNA molecule and more than 85% of the third ionizable lipid remains associated with the second RNA molecule in the fused RNA co-LNP. In some embodiments, more than 90% of the second ionizable lipid remains associated with the first RNA molecule and more than 90% of the third ionizable lipid remains associated with the second RNA molecule in the fused RNA co-LNP. In some embodiments, more than 95% of the second ionizable lipid remains associated with the first RNA molecule and more than 95% of the third ionizable lipid remains associated with the second RNA molecule in the fused RNA co-LNP. In some embodiments, more than 99% of the second ionizable lipid remains associated with the first RNA molecule and more than 99% of the third ionizable lipid remains associated with the second RNA molecule in the fused RNA co-LNP. In some embodiments, all of the second ionizable lipid remains associated with the first RNA molecule and all of the third ionizable lipid remains associated with the second RNA molecule in the fused RNA co-LNP. In some embodiments, between about 75% and about 90% of the second ionizable lipid remains associated with the first RNA molecule and, or between about 80% and about 90% of the third ionizable lipid remains associated with the second RNA molecule in the fused RNA co-LNP. In some embodiments, between about 75% and about 99% of the second ionizable lipid remains associated with the first RNA molecule and, between about 75% and about 99% of the third ionizable lipid remains associated with the second RNA molecule in the fused RNA co-LNP. In some embodiments, the fused RNA co-LNP comprising the first RNA molecule and the second RNA molecule are fused with the first LNP comprising the DNA molecule to generate a co-LNP comprising the DNA molecule, the first RNA molecule and the third RNA molecule. These fused LNPs are also referred to herein as tri-LNPs.

In some embodiments, the tri-LNP comprises a volumetric ratio of the first LNP to the second and third LNPs that is between about 3:1 and about 1:3.

In some embodiments, the co-LNPs and tri-LNPs formed by fusion methods described herein show a fluorescence energy transfer (FRET). In particular embodiments, FRET is demonstrated by attaching individual fluorescent dyes, a donor and acceptor, to each of the precursor LNPs prior to mixing (under acidic conditions) and neutralization. Fluorescence emission from the acceptor dye indicates the level of fusion. In some embodiments, the FRET emission signal of the fused co-LNP is greater that the fluorescence emission signal of a mixture of two individual LNPs that are not fused together. In some embodiments, the normalized FRET signal immediately following neutralization is greater than 0.3. In some embodiments, the normalized FRET signal immediately following neutralization is greater than 0.35. In some embodiments, the normalized FRET signal immediately following neutralization is greater than 0.38. In some embodiments, the normalized FRET signal immediately following neutralization is greater than 0.4. In some embodiments, the normalized FRET signal immediately following neutralization is between about 0.35 and 0.42 In some embodiments, the normalized FRET signal immediately following neutralization is between about 0.38 and 0.42 In any of the foregoing embodiments, the normalized FRET signal may be calculated by the method described in Example 17.

In some embodiments, the co-LNP comprises a first helper lipid and a second helper lipid. In some embodiments, the co-LNP comprises a polyethylene glycol (PEG)-conjugated lipid. In some embodiments, the co-LNP comprises cholesterol.

In some embodiments, the co-LNP or tri-LNP has an average size of between about 50 nm and 150 nm, or between about 75 nm and about 125 nm, as measured by dynamic light scattering (DLS). In some embodiments, the co-LNP has an average size of between about 50 nm and 150 nm, as measured by dynamic light scattering (DLS). In some embodiments, the co-LNP or tri-LNP has an average size of between about 75 nm and about 125 nm, as measured by dynamic light scattering (DLS).

In some embodiments, the first ionizable lipid of the co-LNP or tri-LNP comprises a diketopiperazine ring core. In some embodiments, the first ionizable lipid of the co-LNP or tri-LNP comprises an unsaturated tail. In some embodiments, the unsaturated tail is an unsaturated linoleil tail. In some embodiments, the first ionizable lipid is: (a) OF-C4-Deg-Lin, or an analog thereof; or (b) cKK-E12, or an analog thereof. In some embodiments, the first ionizable lipid is OF-C4-Deg-Lin or an analog thereof. In some embodiments, the first ionizable lipid is OF-C4-Deg-Lin. In some embodiments, the first ionizable lipid is DLin-KC2-DMA or an analog thereof. In some embodiments, the first ionizable lipid is DLin-KC2-DMA.

In some embodiments, the first ionizable lipid and the second ionizable lipid of the co-LNP or tri-LNP are the same. In some embodiments, the first ionizable lipid of the co-LNP or tri-LNP and the second ionizable lipid of the co-LNP or tri-LNP are different. In some embodiments, the second ionizable lipid and the third ionizable lipid of the tri-LNP are the same.

In some embodiments, the second ionizable lipid of the co-LNP or tri-LNP comprises a diketopiperazine ring core. In some embodiments, the second ionizable lipid of the co-LNP or tri-LNP comprises an unsaturated tail. In some embodiments, the unsaturated tail is an unsaturated linoleic tail. In some embodiments, the second ionizable of the co-LNP lipid is: (a) OF-C4-Deg-Lin, or an analog thereof; or (b) cKK-E12, or an analog thereof. In some embodiments, the second ionizable lipid is OF-C4-Deg-Lin, or an analog thereof. In some embodiments, the second ionizable lipid is OF-C4-Deg-Lin. In some embodiments, the second ionizable lipid is co-LNP s cKK-E12, or an analog thereof. In some embodiments, the second ionizable lipid is cKK-E12. In some embodiments, the second ionizable lipid is DLin-MC3-DMA, or an analog thereof. In some embodiments, the second ionizable lipid is DLin-MC3-DMA.

In some embodiments, the first helper lipid of the co-LNP or tri-LNP is 1-stearoyl-2-oleoyl-sn-glycero-3-phosphocholine (SOPC). In some embodiments, the second helper lipid of the co-LNP or tri-LNP is 1,2-distearoyl-sn-glycero-3-phosphocholine (DSPC). In some embodiments, the third LNP of the tri-LNP comprises a third helper lipid. In some embodiments, the second helper lipid and the third helper lipid of the tri-LNP are the same.

In some embodiments, a co-LNP comprises a fusion of two or more LNPs, any of which may comprise a non-ionizable cationic lipid. In some embodiments, the mass fraction of the non-ionizable cationic lipid in the co-LNP is between about 0.5% and about 7%. In some embodiments, the mass fraction of the non-ionizable cationic lipid in the LNP is between about 0.5% and about 5%. In other embodiments, the mass fraction of the non-ionizable cationic lipid in the co-LNP is between about 1% and about 6%. In other embodiments, the mass fraction of the non-ionizable cationic lipid in the co-LNP is between about 2% and about 5%. In some embodiments, the co-LNP is a tri-LNP.

In some embodiments, the co-LNP or tri-LNP comprises a non-ionizable cationic lipid. In some embodiments, the non-ionizable cationic lipid has the following structure (referred to as Lipid T1):

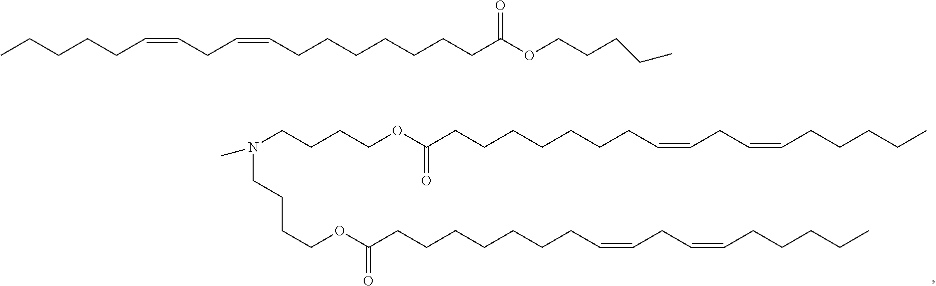

In some embodiments, the mass fraction of Lipid T1 in the co-LNP is between about 0.1% and about 60%. In some embodiments, the mass fraction of Lipid T1 in the co-LNP is between about 0.1% and about 40%. In some embodiments, the mass fraction of Lipid T1 in the co-LNP is between about 0.2% and about 20%. In some embodiments, the mass fraction of Lipid T1 in the co-LNP is between about 0.2% and about 20%. In some embodiments, the mass fraction of Lipid T1 in the co-LNP is between about 0.5% and about 10%. In some embodiments, the mass fraction of Lipid T1 in the co-LNP is between about 1% and about 7%. In some embodiments, the mass fraction of Lipid T1 in the co-LNP is between about 1% and about 6%. In some embodiments, the mass fraction of Lipid T1 in the co-LNP is between about 2% and about 5%. In some embodiments, the mass fraction of Lipid T1 in the co-LNP is between about 0.2% and about 1%.

In some embodiments, the DNA molecule of the co-LNP or tri-LNP comprises a transgene. In some embodiments, the transgene encodes a recombinant receptor. In some embodiments, the DNA molecule is a closed end DNA vector. In some embodiments, the transgene is positioned between protelomerase binding sequences. In some embodiments, the transgene is operably linked to a promoter and positioned between inverted terminal repeats (ITRs).

In some embodiments, the RNA molecule encodes a transposase. In some embodiments, the transposase is a piggyBac transposase or a Sleeping Beauty transposase.

In some embodiments, the DNA molecule of the co-LNP or tri-LNP comprises a single-stranded DNA oligonucleotide (ssODN) or a double-stranded DNA oligonucleotide (dsODN), the ssODN or the dsODN comprising a nucleotide sequence that is homologous to a target genomic locus. In some embodiments, the DNA molecule comprises a single-stranded DNA oligonucleotide (ssODN) comprising a nucleotide sequence that is homologous to a target genomic locus. In some embodiments, the DNA molecule comprises double-stranded DNA oligonucleotide (dsODN) comprising a nucleotide sequence that is homologous to a target genomic locus.

In some embodiments, the RNA molecule of the co-LNP or tri-LNP is or comprises a guide RNA (gRNA). In some embodiments, the RNA molecule is a guide RNA (gRNA). In some embodiments, the RNA molecule comprises a guide RNA (gRNA). In some embodiments, the gRNA is a single guide RNA (sgRNA). In some embodiments, the gRNA is complexed with a recombinant nuclease capable of inducing a DNA break. In some embodiments, the RNA molecule is or comprises a nucleotide sequence encoding a recombinant nuclease capable of inducing a DNA break. In some embodiments, the RNA molecule is: (1) a gRNA; and (2) a nucleotide sequence encoding a recombinant nuclease capable of inducing a DNA break. In some embodiments, the RNA molecule of the second LNP is a gRNA, and the RNA molecule of the third LNP is a nucleotide sequence encoding a recombinant nuclease capable of inducing a DNA break. In some embodiments, the recombinant nuclease is a zinc finger nuclease (ZFN), a transcription activator-like effector nuclease (TALEN), or a CRISPR-associated nuclease (Cas). In some embodiments, the recombinant nuclease is a Cas nuclease. In some embodiments, the Cas is Cas9 or Cas12a. In some embodiments, the Cas is Cas9. In some embodiments, the Cas is Cas12a.

In some embodiments, the recombinant receptor is a chimeric antigen receptor (CAR) or a T cell receptor (TCR). In some embodiments, the recombinant receptor is a TCR. In some embodiments, the recombinant receptor is a CAR. In some embodiments, the CAR comprises an extracellular antigen-binding domain, a transmembrane domain, and an intracellular region. In some embodiments, the extracellular antigen-binding domain is an antibody or an antigen-binding fragment thereof that binds to an antigen that is associated with, or expressed on a cell or tissue of a disease or condition. In some embodiments, the CAR is a single antigen directed CAR. In some embodiments, the CAR is a bispecific CAR. In some embodiments, the CAR is a bispecific CAR. In some embodiments, the DNA (e.g., ceDNA) molecule encoding the bispecific CAR is at least 6 kilobases, at least 7 kilobases, or at least 8 kilobases.

In some embodiments, the antigen is selected from the group consisting of αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), and Wilms Tumor 1 (WT-1). In some embodiments, the antigen is BCMA. In some embodiments, the antigen is CD19. In some embodiments, the antigen is CD20. In some embodiments, the antigen is CD22. In some embodiments, the antigen is GPRC5D.

In some embodiments, the intracellular region comprises an intracellular signaling domain that is or comprises an intracellular signaling domain of a CD3 chain, or a signaling portion thereof. In some embodiments, the intracellular region comprises one or more costimulatory signaling domain(s) comprising an intracellular signaling domain selected from the group consisting of: a CD28, a 4-1BB, an ICOS, or a signaling portion thereof. In some embodiments, the intracellular region comprises one or more costimulatory signaling domain(s) comprising an intracellular signaling domain of 4-1BB.

Also provided herein is a method of producing a lipid nanoparticle (LNP), the method comprising: (1) adding to an organic solvent comprising ethanol: (a) an ionizable lipid; (b) a helper lipid; (c) a polyethylene glycol (PEG)-conjugated lipid; and (d) cholesterol, thereby generating an organic phase; (2) adding to an aqueous solvent having an acidic pH, a deoxyribonucleic acid (DNA) molecule, thereby generating an aqueous phase; and (3) combining the organic phase and the aqueous phase by laminar flow mixing in a device, thereby generating an LNP.

In some embodiments, the ionizable lipid is an ionizable amino lipid selected from the group consisting of OF-C4-Deg-Lin, or an analog thereof, cKK-E12, or an analog thereof, and DLin-KC2-DMA, or an analog thereof. In some embodiments, the ionizable lipid is OF-C4-Deg-Lin or an analog thereof. In some embodiments, the ionizable lipid is OF-C4-Deg-Lin. In some embodiments, the ionizable lipid is cKK-E12. In some embodiments, the ionizable lipid is DLin-KC2-DMA.

In some embodiments, a flow rate of the aqueous phase in the device is between about 8 mL/min and about 10 mL/min. In some embodiments, a flow rate of the organic phase in the device is between about 2 mL/min and about 4 mL/min. In some embodiments, the flow rate of the aqueous phase is about 9 mL/min, and the flow rate of the organic phase is about 3 mL/min.

In some embodiments, the aqueous solvent is an acetate buffer. In some embodiments, the pH of the acetate buffer is between about 3.0 and about 4.5. In some embodiments, the pH of the acetate buffer is about 4.0.

In some embodiments, the method comprises collecting the generated LNP from the device in the acetate buffer. In some embodiments, the method comprises washing the collected LNP with an isotonic buffer. In some embodiments, the method comprises filtering the LNP in the isotonic buffer with a filter to remove an LNP greater than 200 nM in diameter. In some embodiments, the isotonic buffer is PBS.

Also provided herein is a LNP produced by any of the methods herein.

Also provided herein is a co-formulated lipid nanoparticle (co-LNP) comprising: (1) a first ribonucleic acid (RNA) molecule and a second ribonucleic acid (RNA) molecule; and (2) a first ionizable lipid and a second ionizable lipid, wherein one of the first and second RNA molecules encodes a recombinant nuclease capable of inducing a DNA break; and the other of the first and second RNA molecules is a guide RNA (gRNA). In some embodiments, the co-LNP is formed by a fusion of two precursor LNPs, thereby generating a fused co-LNP.

Also provided herein is a co-formulated lipid nanoparticle (co-LNP) comprising a fusion of a first lipid nanoparticle (LNP) and a second lipid nanoparticle (LNP), wherein: (1) the first LNP comprises: (i) a ribonucleic acid (RNA) molecule; and (ii) a first ionizable lipid; and (2) the second LNP comprises: (i) a ribonucleic acid (RNA) molecule; and (ii) a second ionizable lipid, wherein one of the first and second RNA molecules encodes a recombinant nuclease capable of inducing a DNA break; and the other of the first and second RNA molecules is a guide RNA (gRNA).

In some embodiments, the gRNA is a single guide RNA (sgRNA). In some embodiments, the recombinant nuclease is a zinc finger nuclease (ZFN), a transcription activator-like effector nuclease (TALEN), or a CRISPR-associated nuclease (Cas). In some embodiments, the recombinant nuclease is a Cas nuclease. In some embodiments, the Cas is Cas9 or Cas12a. In some embodiments, the Cas is Cas9. In some embodiments, the Cas is Cas12a.

In some embodiments, the co-LNP comprises a volumetric ratio of the first LNP to the second LNP that is about 1:1.

In some embodiments, the first ionizable lipid is an amino lipidoid comprising a diketopiperazine ring core. In some embodiments, the first ionizable lipid comprises an unsaturated tail. In some embodiments, the unsaturated tail is an unsaturated linoleic tail. In some embodiments, the first ionizable lipid is: (a) OF-C4-Deg-Lin, or an analog thereof; or (b) cKK-E12, or an analog thereof. In some embodiments, the first ionizable lipid is OF-C4-Deg-Lin, or an analog thereof. In some embodiments, the first ionizable lipid is OF-C4-Deg-Lin. In some embodiments, the first ionizable lipid is cKK-E12, or an analog thereof. In some embodiments, the first ionizable lipid is cKK-E12. In some embodiments, the first ionizable lipid is DLin-MC3-DMA, or an analog thereof. In some embodiments, the first ionizable lipid is DLin-MC3-DMA.

In some embodiments, the first ionizable lipid and the second ionizable lipid are the same.

In some embodiments, the second ionizable lipid is DLin-MC3-DMA, or an analog thereof. In some embodiments, the second ionizable lipid is DLin-MC3-DMA. In some embodiments, the second ionizable lipid is cKK-E12, or an analog thereof. In some embodiments, the second ionizable lipid is cKK-E12.

Also provided herein is a method of producing a co-formulated lipid nanoparticle (co-LNP), comprising: (1) mixing, in an acidic buffer: (a) a first lipid nanoparticle (LNP) comprising a first ionizable lipid and a nucleic acid molecule; and (b) a second LNP comprising a second ionizable lipid and a ribonucleic acid (RNA) molecule, thereby generating a composition comprising the first LNP and the second LNP; and (2) neutralizing the composition comprising the first LNP and the second LNP, thereby generating a co-LNP, which is a fusion of the first LNP and the second LNP, wherein the nucleic acid molecule in (a) is a deoxyribonucleic acid (DNA) molecule or a ribonucleic acid (RNA) molecule. In some embodiments, the first LNP and second LNP, prior to fusion, are precursor LNPs that are not fully formed.

In some embodiments, the nucleic acid molecule in (a) is a DNA molecule. In some embodiments, the nucleic acid molecule in (a) is an RNA molecule.

In some embodiments, the volumetric ratio of the first LNP to the second LNP in the composition is between about 3:1 and about 1:3. In some embodiments, the volumetric ratio of the first LNP to the second LNP in the composition is between about 2:1 and about 1:2.

In some embodiments, the method comprises mixing, in the acidic buffer, (c) a third LNP comprising a third ionizable lipid and an RNA molecule, thereby generating a composition comprising the first, second, and third LNPs. In some embodiments, the volumetric ratio of the first LNP to the second and third LNPs in the composition is between about 3:1 and about 1:3. In some embodiments, the volumetric ratio of the first LNP to the second and third LNPs in the composition is between about 2:1 and about 1:2.

In some embodiments, the acidic buffer is an acetate buffer. In some embodiments, the acidic buffer has a pH of between about 3.0 and about 4.5. In some embodiments, the acidic buffer has a pH of between about 4.0 and about 5.0. In some embodiments, the acidic buffer has a pH of about 4.0. In some embodiments, the acidic buffer is neutralized to a pH of between about 6.0 and about 7.5, or between about 6.5 and about 7.0. In some embodiments, the acidic buffer is neutralized to a pH of between about 6.0 and about 7.5. In some embodiments, the acidic buffer is neutralized to a pH of between about 6.5 and about 7.0. In some embodiments, neutralizing the composition comprising the first LNP and the second LNP comprises adding an isotonic buffer. In some embodiments, the isotonic buffer has a pH of about 7.4. In some embodiments, neutralizing the composition comprising the first LNP and the second LNP comprises adding at least about 6 parts of the isotonic buffer to 1 part of the acidic buffer. In some embodiments, neutralizing the composition comprising the first LNP and the second LNP comprises adding between about 6-7 parts of the isotonic buffer to 1 part of the acidic buffer. In some embodiments, the isotonic buffer is phosphate buffered saline (PBS).

Also provided herein is a co-LNP produced by any of the methods provided herein.

Also provide herein is a lipid nanoparticle (LNP) comprising: (a) an ionizable lipid; (b) a helper lipid that is 1,2-distearoyl-sn-glycero-3-phosphocholine (DSPC); (c) a polyethylene glycol (PEG)-conjugated lipid that is DMG-PEG2000; (d) cholesterol; and (e) a ribonucleic acid (RNA) molecule.

In some embodiments, the ionizable lipid is an ionizable amino lipid selected from the group consisting of OF-C4-Deg-Lin, an analog thereof, cKK-E12, an analog thereof, DLin-MC3-DMA, and an analog thereof. In some embodiments, the ionizable lipid is OF-C4-Deg-Lin. In some embodiments, the ionizable lipid is cKK-E12. In some embodiments, the ionizable lipid is DLin-MC3-DMA. In some embodiments, the ionizable lipid is a lipidoid comprising a diketopiperazine amino core. In some embodiments, the ionizable lipid comprises an unsaturated tail. In some embodiments, the unsaturated tail is an unsaturated linoleic tail.

In some embodiments, the mass fraction of the ionizable lipid in the LNP is between about 50% and about 65%. In some embodiments, the mass fraction of DSPC in the LNP is between about 10% and about 15%. In some embodiments, the mass fraction of DMG-PEG2000 in the LNP is between about 5% and about 7.5%. In some embodiments, the mass fraction of cholesterol in the LNP is between about 15% and about 25%. In some embodiments, the mass fraction of the RNA molecule in the LNP is between about 3% and about 10%.

In some embodiments, the mass fraction of the ionizable lipid in the LNP is between about 32% and about 36%. In some embodiments, the mass fraction of DSPC is between about 15% and about 20%. In some embodiments, the mass fraction of DMG-PEG2000 is between about 3.5% and about 5.5%. In some embodiments, the mass fraction of cholesterol is between about 35% and about 45%. In some embodiments, the mass fraction of the RNA molecule is between about 3% and about 4%.

In some embodiments, the RNA molecule encodes a transposase. In some embodiments, the transposase is a piggyBac transposase or a Sleeping Beauty transposase. In some embodiments, the RNA molecule comprises a guide RNA (gRNA). In some embodiments, the gRNA is a single guide RNA (sgRNA). In some embodiments, the gRNA is complexed. In some embodiments, with a recombinant nuclease capable of inducing a DNA break. In some embodiments, the RNA sequence comprises a nucleotide sequence encoding a recombinant nuclease capable of inducing a DNA break. In some embodiments, the recombinant nuclease is a zinc finger nuclease (ZFN), a transcription activator-like effector nuclease (TALEN), or a CRISPR-associated nuclease (Cas). In some embodiments, the recombinant nuclease is a Cas nuclease. In some embodiments, the recombinant nuclease is Cas9. In some embodiments, the recombinant nuclease is Cas12a.

Also provided herein is a composition comprising: (1) a LNP provided herein comprising a DNA molecule; and (2) a LNP comprising a RNA molecule.

Also provided herein is a composition comprising: (1) a LNP provided herein comprising a DNA molecule; and (b) a LNP provided herein comprising a RNA molecule.

Also provided herein is a composition comprising: (1) a LNP comprising a DNA molecule; and (2) a LNP comprising a RNA molecule, wherein the composition comprises a solution having a pH of about 3.0, about 3.5, about 4.0, about 4.5, or about 5.0, or having a pH between about 3.0 to about 5.0, between about 3.0 to about 4.0, between about 3.5 to about 4.5, or between about 4.0 to about 5.0. In certain embodiments, the LNP comprising the DNA molecule is not fully formed. In certain embodiments, the LNP comprising the RNA molecule is not fully formed. In certain embodiments, the LNP comprising the DNA molecule and the LNP comprising the RNA molecule are not fully formed.

Also provided herein is a composition comprising: (1) a first LNP comprising a DNA molecule; (2) a second LNP comprising a first RNA molecule, and (3) a third LNP comprising a second RNA molecule, wherein the composition comprises a solution having a pH of about 3.0, about 3.5, about 4.0, about 4.5, or about 5.0, or having a pH between about 3.0 to about 5.0, between about 3.0 to about 4.0, between about 3.5 to about 4.5, or between about 4.0 to about 5.0. In certain embodiments, any or all of the first, second, or third LNPs are not fully formed.

Also provided herein is a co-formulated lipid nanoparticle (co-LNP) comprising a fusion of: (1) a LNP comprising a DNA molecule provided herein; and (2) a LNP comprising a RNA molecule.

Also provide herein is a co-formulated lipid nanoparticle (co-LNP) comprising a fusion of (1) a LNP comprising a DNA molecule; and (2) a LNP comprising a RNA molecule provided herein.

Also provide herein is a co-formulated lipid nanoparticle (co-LNP) comprising a fusion of (1) a LNP comprising a DNA molecule provided herein; and (2) a LNP comprising a RNA molecule provided herein.

Also provided herein is a combination of: (1) a LNP comprising a DNA molecule provided herein; and (2) a ribonucleoprotein (RNP) complex.

Also provided herein is a composition comprising any of the LNPs provided herein. Also provided herein is a composition comprising any of the co-LNPs provided herein.

Also provided herein is a method of genetically engineering an immune cell, the method comprising: (1) introducing a ribonucleic acid (RNA) molecule into an immune cell by electroporation; and (2) incubating the immune cell with a RNA LNP provided herein or a composition comprising the cell.

Also provided herein is a method of genetically engineering an immune cell, the method comprising: (1) introducing a ribonucleoprotein complex (RNP) into an immune cell by electroporation; and (2) incubating the immune cell with a RNA LNP provided herein or a composition comprising the same.

Also provided herein is a method of genetically engineering an immune cell, the method comprising incubating the immune cell with (1) a RNA LNP provide herein or a composition thereof; and (2) a DNA LNP provided herein or a composition thereof.

Also provided herein is a method of genetically engineering an immune cell, the method comprising incubating an immune cell with a co-LNP provided herein or a composition thereof.

In some embodiments, the immune cell is a lymphocyte. In some embodiments, the immune cell is a T cell. In some embodiments, the T cell is a primary T cell. In some embodiments, the T cell is a CD4+ T cell or a CD8+ T cell.

In some embodiments, at the time of incubating the immune cell with the LNP, the co-LNP (or tri-LNP), or the composition, the immune cell is activated. In some embodiments, at the time of incubating the immune cell with the LNP, the co-LNP (or tri-LNP), or the composition, the immune cell expresses CD25, CD26, CD27, CD28, CD30, CD71, CD154, CD40L, CD134, or a combination thereof. In some embodiments, the immune cell is incubated under stimulating conditions prior to incubating the immune cell with the LNP, the co-LNP (or tri-LNP), or the composition. In some embodiments, the immune cell is incubated under stimulating conditions for between about 24 hours and about 72 hours, or for about 48 hours. In some embodiments, the stimulating conditions comprise incubation with a stimulatory reagent capable of activating an intracellular signaling domain of a component of a TCR complex and an intracellular signaling domain of a costimulatory molecule. In some embodiments, the stimulatory reagent comprises a primary agent that binds to CD3 and a secondary agent that binds to a T cell costimulatory molecule. In some embodiments, the costimulatory molecule is selected from the group consisting of CD28, 4-1BB, OX40, and ICOS. In some embodiments, the primary agent is an anti-CD3 antibody or antigen-binding fragment, and the secondary agent is an anti-CD28 antibody or antigen-binding fragment. In some embodiments, the immune cell is incubated with apolipoprotein E (ApoE) prior to incubating the immune cell with the LNP, the co-LNP (or tri-LNP), or the composition. In some embodiments, the ApoE is ApoE4.

Also provided herein is an immune cell produced by any of the methods herein. Also provided herein is a composition comprising a plurality of immune cells produced by any of the method herein.

Also provided herein is a method of treatment comprising administering the immune cell produced by any of the methods herein, or a composition thereof, to a subject having a disease or disorder. Also provided herein is use of the immune cell produced by any of the methods herein, or a composition thereof, for treating a disease or disorder. Also provided herein is use of the immune cell produced by any of the methods herein, or a composition thereof, in the manufacture of a medicament for treating a disease or disorder.

### BRIEF DESCRIPTION OF THE DRAWINGS

FIGS. 1 and 2 depict the percent of live cells (horizontal lines) and the percent of live cells expressing GFP (vertical bars), following transfection of primary human T cells from a donor with LNPs produced from various formulations and containing superfolder GFP plasmid DNA. The concentration of plasmid DNA is shown along the x-axis (0.75, 1.5, 3.0, or 6.0 g/mL).

FIG. 3 shows the variability of GFP expression among primary human T cells from four different donors, following transfection with LNPs produced from various formulations and containing superfolder GFP plasmid DNA. The concentration of plasmid DNA is shown along the x-axis (0.75, 1.5, 3.0, or 6.0 g/mL).

FIGS. 4A and 4B show the effects of varying the mass fraction percentage of the ionizable lipid (DLin-KC2-DMA), the helper lipid (SOPC), the polyethylene glycol (PEG) lipid (DMG-PEG2000), or cholesterol, on LNP size.

FIG. 4C shows the relationship between particle size and the mass fraction percentage of the ionizable lipid (DLin-KC2-DMA), the helper lipid (SOPC), and the PEG lipid (DMG-PEG2000), at either a high or low PEG lipid mass fraction percentage.

FIGS. 5A and 5B show the effects of varying the mass fraction percentage of the ionizable lipid (DLin-KC2-DMA), the helper lipid (SOPC), the polyethylene glycol (PEG) lipid (DMG-PEG2000), or cholesterol, on DNA loading into LNPs.

FIGS. 6A and 6B show the effects of varying the mass fraction percentage of the ionizable lipid (Dlin-KC2-DMA), the helper lipid (SOPC), the polyethylene glycol (PEG) lipid (DMG-PEG2000), or cholesterol, on transfection efficiency.

FIGS. 7A-7D show the effects of varying the mass fraction percentage of the ionizable lipid (Dlin-KC2-DMA), the helper lipid (SOPC), cholesterol, or DNA, respectively, on lipid nanoparticle size.

FIGS. 8A-8D show the effects of varying the mass fraction percentage of the ionizable lipid (Dlin-KC2-DMA), the helper lipid (SOPC), cholesterol, or DNA, respectively, on DNA loading into LNPs.

FIGS. 9A-9D show the effects of varying the mass fraction percentage of the ionizable lipid (Dlin-KC2-DMA), the helper lipid (SOPC), cholesterol, or DNA, respectively, on transfection efficiency.

FIGS. 10A and 10B depict the transfection efficiency when the concentration of the DNA plasmid was 1, 2, 3, or 6 g/mL and the mass fraction percentage of the DNA plasmid was 3.5 (FIG. 10B), 6.0, or 9.0.

FIG. 11 shows the relationship between transfection efficiency and LNP size.

FIG. 12 depicts the relationship between LNP size and mass fraction percentage of the helper lipid.

FIGS. 13A-C show LNP size (FIG. 13A) and DNA loading (FIG. 13B), when LNPs were made with different mole fractions of the PEG lipid. The LNP size and DNA loading graphs are overlaid in FIG. 13C.

FIG. 14 shows the transfection efficiency of different batches of LNPs made with different mole fractions of PEG lipid.

FIGS. 15A-15E show the relationship between LNP size and the mass fraction percentage of the ionizable lipid, the helper lipid, cholesterol, the PEG, or DNA, respectively.

FIGS. 16A-E show the relationship between DNA loading and the mass fraction percentage of the ionizable lipid, the helper lipid, cholesterol, the PEG lipid, or DNA, respectively.

FIG. 17A-E show the relationship between transfection efficiency and the mass fraction percentage of the ionizable lipid, the helper lipid, cholesterol, the PEG lipid mass fraction, or DNA, respectively.

FIG. 18A depicts the DLin-KC2-DMA ionizable lipid and three analogs having alkyl chain linker lengths of 1 (Lipid 3), 3 (Lipid 19), or 4 (Lipid 34).

FIG. 18B depicts the relationship between LNP size and ionizable lipid mass fraction percentage using DLin-KC2-DMA, Lipid 3, Lipid 19, or Lipid 34.

FIG. 18C depicts the relationship between DNA loading and helper lipid mass fraction percentage using DLin-KC2-DMA, Lipid 3, Lipid 19, or Lipid 34.

FIG. 18D depicts the relationship between transfection efficiency and ionizable lipid mass fraction percentage using DLin-KC2-DMA, Lipid 3, Lipid 19, or Lipid 34.

FIG. 19A depicts the DLin-KC2-DMA ionizable lipid and three analogs having a modified heterocyclic headgroup motif (Lipids 11, 12, and 22).

FIG. 19B depicts the relationship between LNP size and the ionizable lipid mass fraction percentage using DLin-KC2-DMA, Lipid 11, Lipid 12, or Lipid 22.

FIG. 19C depicts the relationship between DNA loading and the helper lipid mass fraction percentage using DLin-KC2-DMA, Lipid 11, Lipid 12, or Lipid 22.

FIG. 19D depicts the relationship between transfection efficiency and the ionizable lipid mass fraction percentage, using DLin-KC2-DMA, Lipid 11, Lipid 12, or Lipid 22.

FIG. 20 depicts the relationship between LNP size and the ratio of the aqueous flow rate to the organic flow rate (Aq:Org).

FIG. 21 depicts the relationship between DNA loading and the ratio of the aqueous flow rate to the organic flow rate (Aq:Org).

FIGS. 22A-22D depict the relationship between transfection efficiency and the ratio of the aqueous flow rate to the organic flow rate (Aq:Org), when the concentration of DNA was 0.75 μg/mL, 1.5 μg/mL, 3.0 μg/mL or 6.0 μg/mL, respectively.

FIG. 23 shows predicted transfection efficiency at various aqueous and organic flow rates.

FIG. 24 depicts the number cellular uptake of LNPs (horizontal lines) and the percent of live cells expressing GFP (Ctrl) or an exemplary CAR (vertical bars), following transfection of primary human T cells from six different donors (A-F) with LNPs generated from three formulations (1-3) containing plasmid DNA provided at increasing concentrations (shown left to right on X-axis for each donor).

FIG. 25 shows LNP size prior to sterile filtration and storage (“Pre-Filtration”), after sterile filtration but prior to storage (“Filtered”), and after sterile filtration and storage for one week at 4° Celsius (“1 week storage”).

FIG. 26 DNA loading into LNP before and after storage for one week at 4° Celsius (“1 week storage”).

FIG. 27 shows the percentage of cells expressing GFP that were transfected with either large plasmid (left bars) or nanoplasmid (right bars) 24 hours after transfection.

FIG. 28 shows the percentage of cells expressing GFP (vertical bars), live cells (dotted horizontal lines), and cells incorporating the DiD uptake marker (undotted horizontal lines).

FIG. 29 shows the percentage of cells expressing fluorescent tdTomato (tdT; bars) and live cells (dots).

FIG. 30 shows the percentage of tdT+, GFP+, and tdT+GFP+ cells (bars, left to right) and live cells (dots).

FIG. 31 shows the percentage of cells expressing tdT and GFP.

FIG. 32 shows the percentage of cells exhibiting transient (top) and integrated (bottom) expression of GFP (bars, left) and an anti-CD19 CAR (bars, right).

FIG. 33A shows the percentage of cells exhibiting transient expression of GFP.

FIG. 33B shows the percentage of cells exhibiting integrated expression of GFP, CAR, and GFP/CAR (bars, left to right) and live CD3+ cells.

FIG. 34A shows the percentage of live cells (dots) and cells exhibiting transient expression of GFP (bars, right) and CAR (bars, left).

FIG. 34B shows the percentage of live cells (dots) and cells exhibiting integrated expression of CAR, GFP, and CAR and GFP (bars, left to right).

FIG. 35A-B show the percentage of live cells (dots) and cells exhibiting transient (FIG. 35A) or integrated (FIG. 35B) expression of GFP (bars, right) and CAR (bars, left).

FIGS. 35C-D show the percentage of live cells (dots) and cells exhibiting transient (FIG. 35C) or integrated (FIG. 35D) double positive expression of CAR and GFP (bars) among donors. Each shade (of bars and dots) represents a different donor.

FIG. 36A shows the percentage of live cells (dots) and cells exhibiting transient expression of GFP (bars, right) and CAR (bars, left).

FIG. 36B shows the percentage of live cells (dots) and cells exhibiting integrated expression of GFP (bars, right) and CAR (bars, left).

FIG. 37 shows the percentage of CD3− (bars) and viable (dots) cells.

FIG. 38A depicts the generation of a co-formulated LNP containing RNA and DNA through fusion of a RNA-containing LNP and a DNA-containing LNP.

FIG. 38B depicts the generation of a co-formulated LNP containing RNA and DNA through double sequential fusion.

FIG. 38C depicts the generation of a co-formulated LNP containing RNA and DNA through fusion of two RNA-containing LNPs and one DNA-containing LNP.

FIG. 39A depicts the percentage of CD3− viable cells (dots) and cells exhibiting transient expression of a BCMA CAR (bars).

FIG. 39B depicts the percentage of CD3− viable cells (dots) and cells exhibiting integrated expression of a BCMA CAR (bars).

FIGS. 40A-B show the relationship between the percentage of CD3− cells and the percentage of cells exhibiting integrated BCMA CAR expression following CAR knock-in using a closed end DNA (ceDNA) construct or a DNA nanoplasmid (NP), respectively.

FIG. 41A and FIG. 41 B show the percentage of fully edited cells 7 days and 14 days post-transfection, respectively, following transfection using electroporation, a hybrid approach, co-LNPs or tri-LNPs. Fully edited cells display TCR knockout (TCR−) and expression of CAR T.

FIG. 42A shows fully edited (TCR− CAR+) T cell populations enrich over time following transfection with tri-LNPs.

FIG. 42B shows fully edited (TCR− CAR+) T cell populations enrich over time following electroporation, but to a lesser extent than tri-LNP administration.

FIG. 43A shows CD4/CD8 ratios at various timepoints following transfection of T cells with LNPs. Initially, the CD4 population is enhanced relative to the CD8 population.

FIG. 43B shows CD4/CD8 ratios at various timepoints following transfection of T cells using electroporation.

FIG. 44A and FIG. 44B show the percentage of fully edited cells 7 days and 14 days post-transfection using either electroporation, DNA-LNPs only, or tri-LNP delivery, respectively, for the three different culture methods.

FIG. 45 shows average percentages of fully edited cells from the three donors on various days post-transfection using either electroporation or tri-LNP delivery.

FIG. 46 shows an experimental overview for FRET experiments used to characterize co-LNPs.

FIG. 47 shows a compilation of four independent experiments that demonstrate that significant lipid fusion occurs immediately when LNPs are mixed (0 hr. timepoint) at pH4 (Sample: co-LNP, pH4), and that maximal fusion occurs when LNPs are mixed at pH4 and then neutralized to pH 7 (Sample: co-LNP, pH7).

FIG. 48 shows normalized FRET signals calculated across the four independent signals, for non-fused LNPs (dual LNPs), acidic co-LNPs, and co-LNPs formed following neutralization of the acidic co-LNPs.

### DETAILED DESCRIPTION

Provided herein are lipid nanoparticle (LNP) compositions for delivering nucleic acid molecules, including deoxyribonucleic acid (DNA), ribonucleic acid (RNA), or both, into a lymphocyte, e.g., a T cell, and methods of producing and using the same, such as in connection with cell therapy. In any of the provided embodiments, the lipid nanoparticles contain an ionizable lipid, e.g., an ionizable amino lipid, a helper lipid, a polyethylene glycol (PEG) lipid, cholesterol, and a nucleic acid molecule (e.g., RNA or DNA). In some aspects, the nucleic acid molecule is DNA, such as a DNA molecule comprising a transgene encoding a recombinant receptor (e.g., a T cell receptor (TCR) or a chimeric antigen receptor (CAR)). In particular embodiments, T cells transduced using the LNPs are for use in cell therapy, such as adoptive cell therapy. In some aspects, the nucleic acid molecule is RNA, such as an RNA molecule encoding a recombinant nuclease capable of inducing a DNA break, a guide RNA (gRNA), or a combination, or an RNA molecule encoding a transposase. In some aspects, cells incubated with the LNPs provided herein are genetically edited, such as knocked out and/or knocked-in, at a genomic locus.

Results herein demonstrate that LNPs and compositions thereof are suitable for delivery of nucleic acids, including DNA, into T cells, such as for use in adoptive cell therapies and/or gene editing. In some aspects, the provided LNPs and compositions thereof deliver a DNA molecule comprising a transgene that encodes a recombinant receptor (e.g., a CAR) to genetically engineer T cells to express the receptor. In some aspects, the provided LNPs and compositions thereof deliver an RNA molecule, such as an mRNA encoding a transposase or nuclease, or a guide RNA (gRNA). In some embodiments, the LNP compositions methods are advantageous by virtue of being nonviral, in that the time, labor, and safety risks associated with viral vector-based cell engineering techniques are mitigated or avoided. Further, the LNP compositions provided herein exhibit high transfection and transduction efficiency, with no or limited cell toxicity observed.

In further aspects, LNPs can be co-formulated by the fusion of separate LNPs to contain one or more nucleic acid molecules. For example, a co-formulated LNP can be generated from the fusion of a first LNP containing a first nucleic acid molecule and a second LNP containing a second nucleic acid molecule. For instance, a co-formulated LNP can be generated to contain a DNA molecule and one or more types of RNA molecule (e.g., a transposon and transposase mRNA or repair template and gRNA) by fusing a DNA-containing LNP with a RNA-containing LNP. A co-formulated LNP can also be generated to contain multiple types of RNA molecules by fusing a first LNP containing a first RNA molecule with a second LNP containing a second RNA molecule (e.g., mRNA encoding a nuclease (e.g., Cas) and a gRNA). A co-formulated RNA-containing LNP can be further fused with a DNA-containing LNP to create a co-formulated LNP containing e.g., two types of RNA molecules and a DNA molecule (e.g., mRNA encoding a nuclease, a gRNA, and repair template DNA). In some cases, a co-formulated LNP generated from separate LNPs may result in higher gene editing efficiency, as compared to the separate LNPs. In some cases, it is contemplated that the improved efficiency may be due to reduced competitive uptake, in that the cell is only required to uptake a single type of co-formulated LNP.

While lipid nanoparticles are already used or being investigated for delivery of ribonucleic acid (RNA), drugs, antioxidants, and contrast agents, they can be plagued by issues with dose-limiting toxicities and reproducibility. In addition, many lipid nanoparticle applications require the lipid nanoparticles to incorporate targeting moieties to promote payload uptake into cells (e.g. cationic polymer-based delivery). (Smith et al. (2017) Nature Nanotech., 12:813-20). By contrast, the compositions provided herein, and uses thereof, demonstrate that lipid nanoparticles can be formulated reproducibly, stably, and without targeting moieties to genetically engineer cells in a nontoxic manner. Further, observations herein demonstrate that a non-viral hybrid approach to genetic engineering, such as by delivering RNA into a cell by electroporation, coupled with delivery of DNA into the cell by incubation with DNA-containing LNPs, achieves high rates of transduction efficiency, while resulting in low or negligible cellular toxicity.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Lipid Nanoparticles

Provided herein are lipid nanoparticles (LNPs) and compositions containing the same, such as for delivering a nucleic acid molecule into a cell (e.g. a T cell). In some embodiments, the nucleic acid molecule is an RNA molecule, a DNA molecule, or both. In some embodiments, the LNP comprises an ionizable lipid, such as an ionizable lipid or lipidoid containing a diketopiperazine ring core. In some embodiments, the LNP comprises an ionizable lipid, such as DLin-KC2-DMA or an analog thereof. In some embodiments, the LNP comprises an ionizable lipid, such as DLin-MC3-DMA or an analog thereof. In some embodiments, the LNP further comprise a helper lipid, a polyethylene glycol (PEG)-conjugated lipid, and cholesterol. Also provided herein are methods for producing the LNPs and compositions and uses thereof, such as in connection with cell therapy.

### A. DNA LNPs

### 1. Components

Provided herein are LNPs comprising a deoxyribonucleic acid (DNA) molecule (also referred to as DNA LNPs or DNA-containing LNPs). In some embodiments, the DNA LNP comprises an ionizable lipid; a helper lipid; a polyethylene glycol (PEG)-conjugated lipid; cholesterol; and a DNA molecule. In other embodiments, the DNA LNP comprises a non-ionizable lipid cationic lipid (e.g., Lipid T1); a helper lipid; a polyethylene glycol (PEG)-conjugated lipid; cholesterol; and a DNA molecule.

In some embodiments, the lipid nanoparticle composition contains an ionizable lipid. In some embodiments, the ionizable lipid is positively charged (e.g. a cationic lipid). In some embodiments, the ionizable lipid is a cationic lipid, including but not limited to those described in U.S. Pat. Nos. 9,593,077; 9,365,610; 9,670,152; and 9,458,090. In some embodiments, the ionizable lipid is a cationic lipid, including but not limited to those described in Published PCT application WO2013149140. In some embodiments, the ionizable lipid is a cationic lipid, including but not limited to those described in published US Patent application US2019084965; US2019106379. In some embodiments, the ionizable lipid is a cationic lipid, including but not limited to those described in Published EP application EP2830595.

In some embodiments, the ionizable lipid is positively charged (e.g. a cationic lipid). In some embodiments, the ionizable lipid is selected from the group consisting of D-Lin, 2,2-dilinoleyl-4-dimethylaminoethyl-[1,3]-dioxolane (DLin-KC2-DMA), or an analog thereof, 4-(dimethylamino)-butanoic acid, (10Z,13Z)-1-(9Z,12Z)-9,12-octadecadien-1-yl-10,13-nonadecadien-1-yl ester (DLin-MC3-DMA), or an analog thereof, and OF-C4-Deg-Lin, or an analog thereof, and cKK-E12, or an analog thereof.

In some embodiments, the ionizable lipid is DLin-KC2-DMA or an analog thereof.

In some embodiments, the ionizable lipid has a structure selected from among the following:

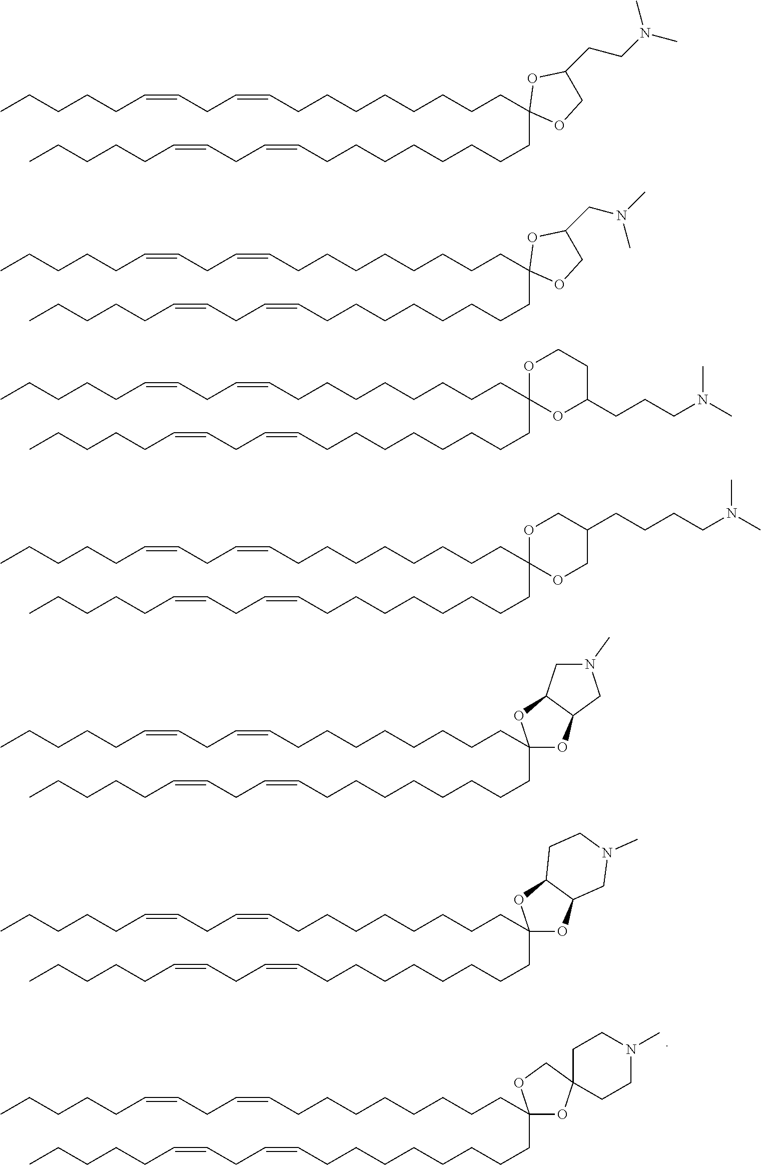

In some cases, the ionizable lipid is DLin-KC2-DMA. In some cases, the ionizable lipid has the structure of

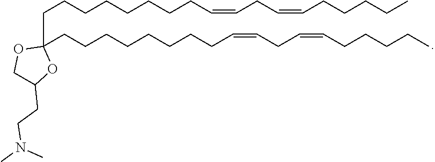 [SMILES: CCCCC/C=C\C/C=C\CCCCCCCCC1(CCCCCCCC/C=C\C/C=C\CCCCC)OCC(CCN(C)C)O1]

In some embodiments, the ionizable lipid is DLin-MC3-DMA or an analog thereof. In some embodiments, the ionizable lipid has the structure of

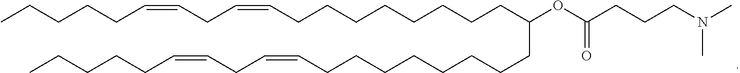 [SMILES: CCCCC/C=C\C/C=C\CCCCCCCCC(CCCCCCCC/C=C\C/C=C\CCCCC)OC(=O)CCCN(C)C]

or an analog thereof.

In some embodiments, the ionizable lipid is DLin-MC3-DMA. In some embodiments, the ionizable lipid has the structure of

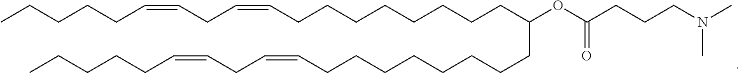 [SMILES: CCCCC/C=C\C/C=C\CCCCCCCCC(CCCCCCCC/C=C\C/C=C\CCCCC)OC(=O)CCCN(C)C]

In some embodiments, the ionizable lipid is positively charged (e.g. a cationic lipid). In some embodiments, the ionizable lipid is a lipid or lipidoid comprising a diketopiperazine ring core. In some embodiments, the ionizable lipid is any as described in Fenton et al., Angew. Chem. Int. Ed. (2018) 57:13582-86, incorporated by reference in its entirety. In some embodiments, the ionizable lipid is selected from OF-C4-Deg-Lin, an analog thereof, cKK-E12, and an analog thereof. In some embodiments, the ionizable lipid has an unsaturated tail. In some embodiments, the ionizable lipid has an unsaturated linoleic tail. In some embodiments, the ionizable lipid is selected from OF-C4-Deg-Lin or analog thereof. In some embodiments, the ionizable lipid has the structure of

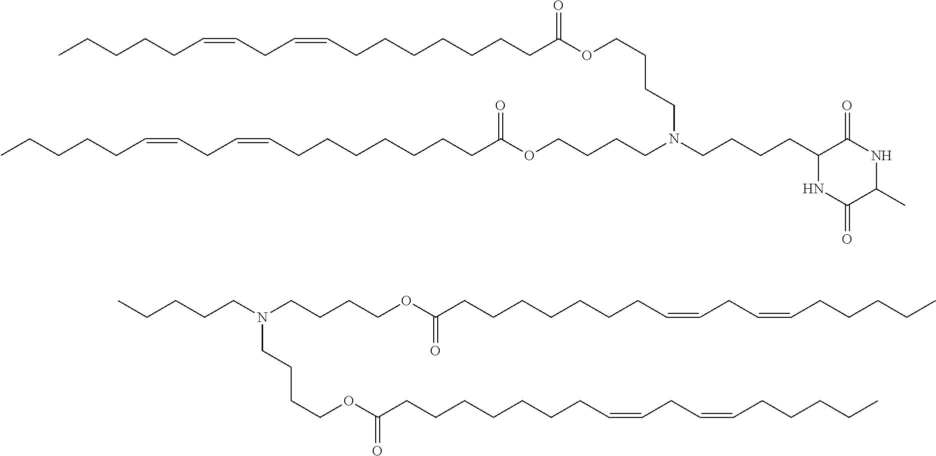

or an analog thereof.

In some embodiments, the ionizable lipid is OF-C4-Deg-Lin. In some embodiments, the ionizable lipid has the structure of

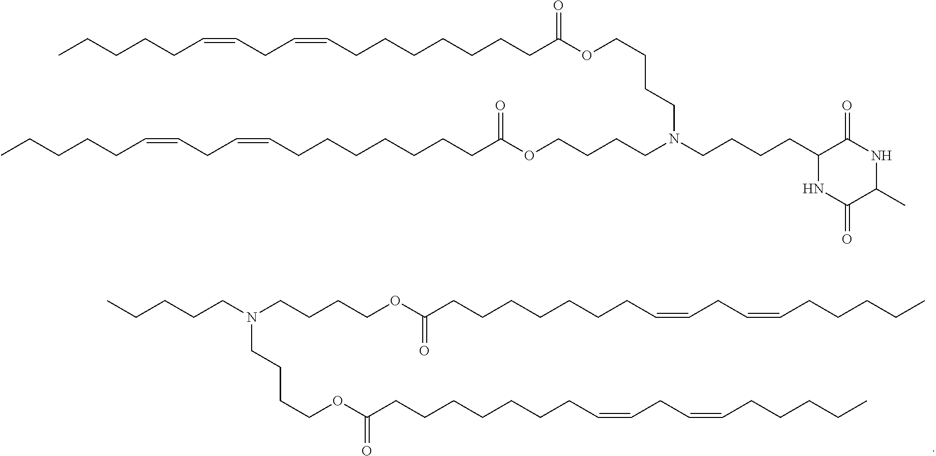

In some embodiments, the ionizable lipid is cKK-E12 or an analog thereof. In some embodiments, the ionizable lipid has the structure of

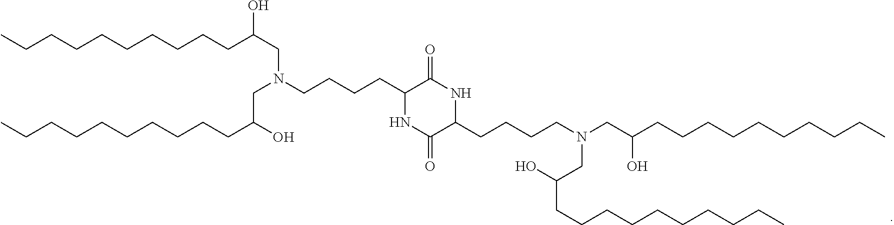 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

or an analog thereof.

In some embodiments, the ionizable lipid is cKK-E12. In some embodiments, the ionizable lipid has the structure of

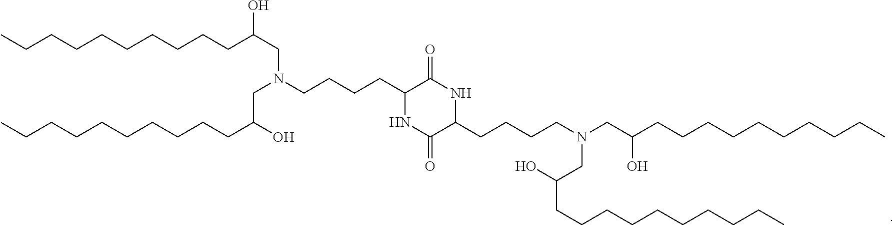 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In some embodiments, the mass fraction of the ionizable lipid in the LNP is between about 30% and about 65%. In some embodiments, the mass fraction of the ionizable lipid in the LNP is between about 32% and about 65%. In some embodiments, the mass fraction of the ionizable lipid in the LNP is between about 40% and about 60%. In some embodiments, the mass fraction of the ionizable lipid in the LNP is between about 35% and about 45%. In some embodiments, the mass fraction of the ionizable lipid in the LNP is between about 40% and about 45%. In some embodiments, the mass fraction of the ionizable lipid in the LNP is between about 50% and about 60%. In some embodiments, the mass fraction of the ionizable lipid is about 30%, about 32%, about 32.5%, about 33%, about 33.5%, about 34%, about 34.5%, about 35%, about 35.5%, about 36%, about 50%, about 51%, about 52%, about 53%, about 54%, about 55%, about 56%, about 57%, about 58%, about 59%, about 60%, about 61%, about 62%, about 63%, about 64%, or about 65%. In some embodiments, the mass fraction of the ionizable lipid is about 32%. In some embodiments, the mass fraction of the ionizable lipid is about 32.5%. In some embodiments, the mass fraction of the ionizable lipid is about 33%. In some embodiments, the mass fraction of the ionizable lipid is about 33.5%. In some embodiments, the mass fraction of the ionizable lipid is about 34%. In some embodiments, the mass fraction of the ionizable lipid is about 34.5%. In some embodiments, the mass fraction of the ionizable lipid is about 35%. In some embodiments, the mass fraction of the ionizable lipid is about 35.5%. In some embodiments, the mass fraction of the ionizable lipid is about 36%. In some embodiments, the mass fraction of the ionizable lipid is about 54%. In some embodiments, the mass fraction of the ionizable lipid is about 55%. In some embodiments, the mass fraction of the ionizable lipid is about 56%. In some embodiments, the mass fraction of the ionizable lipid is about 57%. In some embodiments, the mass fraction of the ionizable lipid is about 58%. In some embodiments, the mass fraction of the ionizable lipid is about 59%. In some embodiments, the mass fraction of the ionizable lipid is about 60%. In some embodiments, the mass fraction of the ionizable lipid is about 61%.

In some embodiments, the ionizable lipid is a lipid or lipidoid comprising a diketopiperazine ring core and the mass fraction of the ionizable lipid in the LNP is between about 30% and about 65%. In other embodiments, the ionizable lipid is a lipid or lipidoid comprising a diketopiperazine ring core and the mass fraction of the ionizable lipid in the LNP is between about 40% and about 60%. In other embodiments, the ionizable lipid is a lipid or lipidoid comprising a diketopiperazine ring core and the mass fraction of the ionizable lipid in the LNP is between about 30% and about 50%. In other embodiments, the ionizable lipid is a lipid or lipidoid comprising a diketopiperazine ring core and the mass fraction of the ionizable lipid in the LNP is between about 40% and about 45%. In some embodiments, the ionizable lipid is a lipid or lipidoid comprising a diketopiperazine ring core and the mass fraction of the ionizable lipid in the LNP is between about 35% and about 45%. In some embodiments, the ionizable lipid is a lipid or lipidoid comprising a diketopiperazine ring core and the mass fraction of the ionizable lipid in the LNP is about 40%, about 45%, about 50%, about 55% or about 60%.

In some embodiments, the ionizable lipid is OF-C4-Deg-Lin and the mass fraction of the ionizable lipid in the LNP is between about 30% and about 65%. In other embodiments, the ionizable lipid is OF-C4-Deg-Lin and the mass fraction of the ionizable lipid in the LNP is between about 40% and about 60%. In other embodiments, the ionizable lipid is OF-C4-Deg-Lin and the mass fraction of the ionizable lipid in the LNP is between about 30% and about 50%. In other embodiments, the ionizable lipid is OF-C4-Deg-Lin and the mass fraction of the ionizable lipid in the LNP is between about 40% and about 45%. In some embodiments, the ionizable lipid is OF-C4-Deg-Lin and the mass fraction of the ionizable lipid in the LNP is between about 35% and about 45%. In some embodiments, the ionizable lipid is OF-C4-Deg-Lin and the mass fraction of the ionizable lipid in the LNP is about 40%, about 45%, about 50%, about 55% or about 60%.

In some embodiments, the ionizable lipid is cKK-E12 and the mass fraction of the ionizable lipid in the LNP is between about 30% and about 65%. In other embodiments, the ionizable lipid is cKK-E12 and the mass fraction of the ionizable lipid in the LNP is between about 40% and about 60%. In other embodiments, the ionizable lipid is cKK-E12 and the mass fraction of the ionizable lipid in the LNP is between about 30% and about 50%. In other embodiments, the ionizable lipid is cKK-E12 and the mass fraction of the ionizable lipid in the LNP is between about 40% and about 45%. In some embodiments, the ionizable lipid is cKK-E12 and the mass fraction of the ionizable lipid in the LNP is between about 35% and about 45%. In some embodiments, the ionizable lipid is cKK-E12 and the mass fraction of the ionizable lipid in the LNP is about 40%, about 45%, about 50%, about 55% or about 60%.

In some embodiments, the ionizable lipid is DLin-KC2-DMA and the mass fraction of the ionizable lipid in the LNP is between about 30% and about 65%. In other embodiments, the ionizable lipid is DLin-KC2-DMA and the mass fraction of the ionizable lipid in the LNP is between about 40% and about 60%. In other embodiments, the ionizable lipid is DLin-KC2-DMA and the mass fraction of the ionizable lipid in the LNP is between about 30% and about 50%. In other embodiments, the ionizable lipid is DLin-KC2-DMA and the mass fraction of the ionizable lipid in the LNP is between about 40% and about 45%. In some embodiments, the ionizable lipid is DLin-KC2-DMA and the mass fraction of the ionizable lipid in the LNP is between about 35% and about 45%. In some embodiments, the ionizable lipid is DLin-KC2-DMA and the mass fraction of the ionizable lipid in the LNP is about 40%, about 45%, about 50%, about 55% or about 60%.

In some embodiments, the LNP comprises more than one cationic lipid. In some embodiments, the LNP comprises two cationic lipids. In some embodiments, the LNP comprises an ionizable cationic lipid and a non-ionizable cationic lipid. The non-ionizable lipid has a higher pKa than the ionizable lipid and would predominantly be charged in both the bloodstream. In some embodiments where the LNP comprises at least one ionizable lipid, the mass fraction of the non-ionizable cationic lipid in the LNP is between about 0.1% and about 40%. In some embodiments where the LNP comprises at least one ionizable lipid, the mass fraction of the non-ionizable cationic lipid in the LNP is between about 0.2% and about 20%. In some embodiments where the LNP comprises at least one ionizable lipid, the mass fraction of the non-ionizable cationic lipid in the LNP is between about 0.2% and about 20%. In some embodiments where the LNP comprises at least one ionizable lipid, the mass fraction of the non-ionizable cationic lipid in the LNP is between about 0.5% and about 10%. In some embodiments where the LNP comprises at least one ionizable lipid, the mass fraction of the non-ionizable cationic lipid in the LNP is between about 1% and about 7%. In some embodiments where the LNP comprises at least one ionizable lipid, the mass fraction of the non-ionizable cationic lipid in the LNP is between about 1% and about 6%. In some embodiments where the LNP comprises at least one ionizable lipid, the mass fraction of the non-ionizable cationic lipid in the LNP is between about 2% and about 5%. In some embodiments where the LNP comprises at least one ionizable lipid, the mass fraction of the non-ionizable cationic lipid in the LNP is between about 0.2% and about 1%.

In some embodiments, the non-ionizable cationic lipid has the following structure:

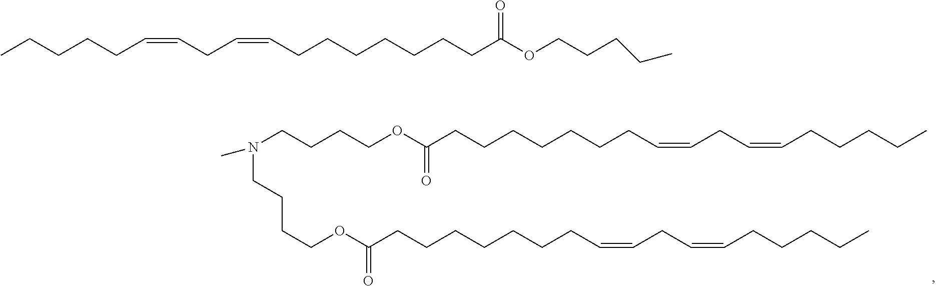

which is referred to herein as Lipid T1. In some embodiments where the LNP comprises at least one ionizable lipid, the mass fraction of Lipid T1 in the LNP is between about 0.1% and about 40%. In some embodiments where the LNP comprises at least one ionizable lipid, the mass fraction of Lipid T1 in the LNP is between about 0.2% and about 20%. In some embodiments where the LNP comprises at least one ionizable lipid, the mass fraction of Lipid T1 in the LNP is between about 0.2% and about 20%. In some embodiments where the LNP comprises at least one ionizable lipid, the mass fraction of Lipid T1 in the LNP is between about 0.5% and about 10%. In some embodiments where the LNP comprises at least one ionizable lipid, the mass fraction of Lipid T1 in the LNP is between about 1% and about 7%. In some embodiments where the LNP comprises at least one ionizable lipid, the mass fraction of Lipid T1 in the LNP is between about 1% and about 6%. In some embodiments where the LNP comprises at least one ionizable lipid, the mass fraction of Lipid T1 in the LNP is between about 2% and about 5%. In some embodiments where the LNP comprises at least one ionizable lipid, the mass fraction of Lipid T1 in the LNP is between about 0.2% and about 1%.

In some cases, the non-ionizable cationic lipid is DOTMA. In some cases, the ionizable lipid has the structure of

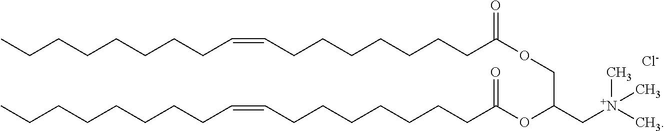 [SMILES: CCCCCCCC/C=C\CCCCCCCC(=O)OCC(C[N+](C)(C)C)OC(=O)CCCCCCC/C=C\CCCCCCCC ; [Cl-]]

In some cases, the non-ionizable cationic lipid is DOTMA. In some cases, the ionizable lipid has the structure of

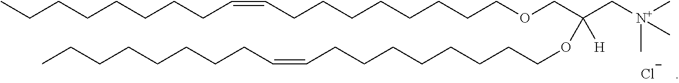 [SMILES: CCCCCCCC/C=C\CCCCCCCCOCC(C[N+](C)(C)C)OCCCCCCCC/C=C\CCCCCCCC ; [Cl-]]

In some cases, the non-ionizable lipid is DODAP. In some cases, the ionizable lipid has the structure of

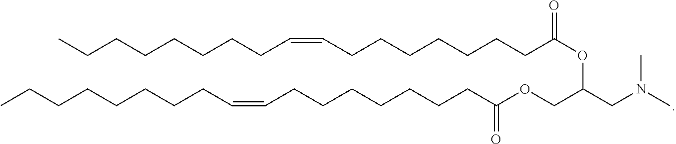 [SMILES: CCCCCCCC/C=C\CCCCCCCC(=O)OCC(CN(C)C)OC(=O)CCCCCCC/C=C\CCCCCCCC]

In some cases, the non-ionizable lipid is DOSPA. In some cases the ionizable lipid has the structure of

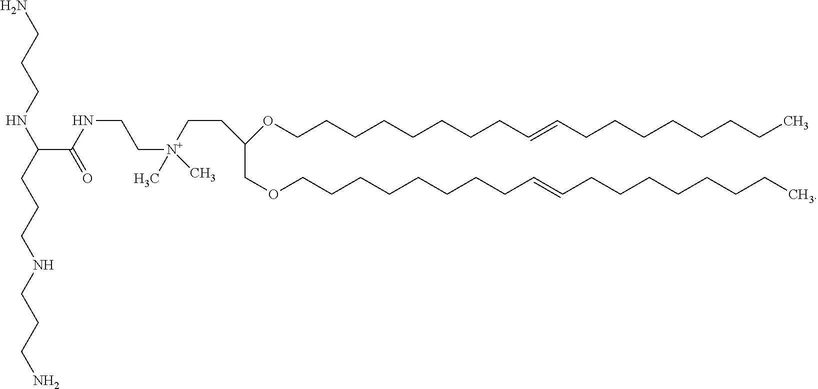 [SMILES: CCCCCCCC/C=C/CCCCCCCCOCC(CC[N+](C)(C)CCNC(=O)C(CCCNCCCN)NCCCN)OCCCCCCCC/C=C/CCCCCCCC]

In some embodiments, the helper lipid is selected from the group consisting of dioleoylphosphatidylethanolamine (DOPE), 1,2-distearoyl-sn-glycero-3-phosphocholine (DSPC), 1-Stearoyl-2-oleoyl-sn-glycero-3-phosphocholine (SOPC), and 1,2-dioleoyl-sn-glycero-sn-3-phosphatidylcholine (DOPC). In some embodiments, the helper lipid is DOPE. In some embodiments, the helper lipid has the structure of

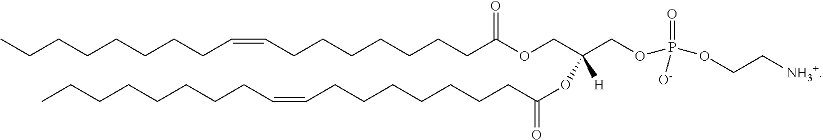

In some embodiments, the helper lipid is DSPC. In some embodiments, the helper lipid has the structure of

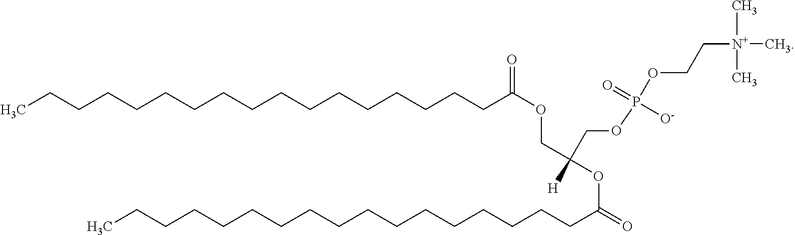 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the helper lipid is SOPC. In some embodiments, the helper lipid has the structure of

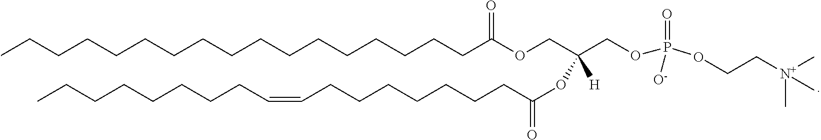 [SMILES: CCCCCCCC/C=C\CCCCCCCC(=O)O[C@H](COC(=O)CCCCCCCCCCCCCCCCC)COP(=O)([O-])OCC[N+](C)(C)C]

In some embodiments, the helper lipid is DOPC. In some embodiments, the helper lipid has the structure of

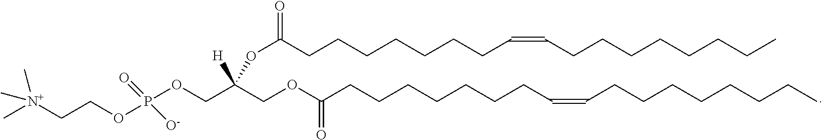 [SMILES: CCCCCCCC/C=C\CCCCCCCC(=O)OC[C@H](COP(=O)([O-])OCC[N+](C)(C)C)OC(=O)CCCCCCC/C=C\CCCCCCCC]

In some embodiments, the mass fraction of the helper lipid in the LNP is between about 15% and about 25%, or between about 18% and about 22%. In some embodiments, the mass fraction of the helper lipid is between about 12% and about 20%, or between about 16% and about 20%. In some embodiments, the mass fraction of the helper lipid is about 15%, about 15.5%, about 16%, about 16.5%, about 17%, about 17.5%, about 18%, about 18.5%, about 19%, about 19.5%, about 20%, about 20.5%, 21%, about 21.5%, about 22%, about 22.5%, about 23%, about 23.5%, about 24%, about 24.5%, or about 25%. In some embodiments, the mass fraction of the helper lipid is about 15%. In some embodiments, the mass fraction of the helper lipid is about 15.5%. In some embodiments, the mass fraction of the helper lipid is about 16%. In some embodiments, the mass fraction of the helper lipid is about 16.5%. In some embodiments, the mass fraction of the helper lipid is about 17%. In some embodiments, the mass fraction of the helper lipid is about 17.5%. In some embodiments, the mass fraction of the helper lipid is about 18%. In some embodiments, the mass fraction of the helper lipid is about 18.5%. In some embodiments, the mass fraction of the helper lipid is about 19%. In some embodiments, the mass fraction of the helper lipid is about 19.5%. In some embodiments, the mass fraction of the helper lipid is about 20%. In some embodiments, the mass fraction of the helper lipid is about 20.5%. In some embodiments, the mass fraction of the helper lipid is about 21%. In some embodiments, the mass fraction of the helper lipid is about 21.5%. In some embodiments, the mass fraction of the helper lipid is about 22%. In some embodiments, the mass fraction of the helper lipid is about 22.5%. In some embodiments, the mass fraction of the helper lipid is about 23%. In some embodiments, the mass fraction of the helper lipid is about 23.5%. In some embodiments, the mass fraction of the helper lipid is about 24%. In some embodiments, the mass fraction of the helper lipid is about 24.5%. In some embodiments, the mass fraction of the helper lipid is about 25%.

In some embodiments, the polyethylene glycol (PEG)-conjugated lipid is DMG-PEG2000. In some embodiments, the PEG-conjugated lipid has the structure of

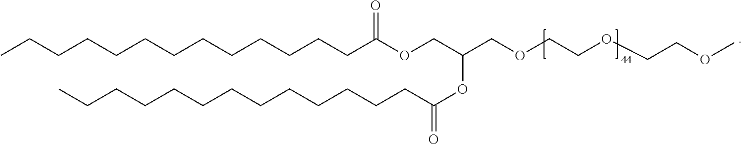

In some embodiments, the mass fraction of the PEG-conjugated lipid in the LNP is between about 2% and about 10%, or between about 3% and about 7%. In some embodiments, the mass fraction of the PEG-conjugated lipid is about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%. In some embodiments, the mass fraction of the PEG-conjugated lipid is about 2%. In some embodiments, the mass fraction of the PEG-conjugated lipid is about 2.5%. In some embodiments, the mass fraction of the PEG-conjugated lipid is about 3%. In some embodiments, the mass fraction of the PEG-conjugated lipid is about 3.5%. In some embodiments, the mass fraction of the PEG-conjugated lipid is about 4%. In some embodiments, the mass fraction of the PEG-conjugated lipid is about 4.5%. In some embodiments, the mass fraction of the PEG-conjugated lipid is about 5%. In some embodiments, the mass fraction of the PEG-conjugated lipid is about 5.5%. In some embodiments, the mass fraction of the PEG-conjugated lipid is about 6%. In some embodiments, the mass fraction of the PEG-conjugated lipid is about 6.5%. In some embodiments, the mass fraction of the PEG-conjugated lipid is about 7%. In some embodiments, the mass fraction of the PEG-conjugated lipid is about 7.5%. In some embodiments, the mass fraction of the PEG-conjugated lipid is about 8%. In some embodiments, the mass fraction of the PEG-conjugated lipid is about 8.5%.

In some embodiments, cholesterol has the structure of

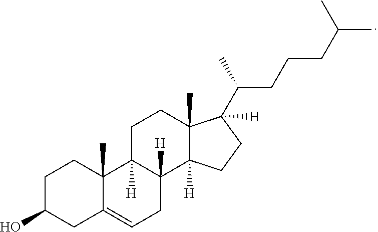

In some embodiments, the mass fraction of cholesterol in the LNP is between about 30% and about 45% or between about 33% and 37%. In some embodiments, the mass fraction of cholesterol is about 30%, about 31%, about 32%, about 33%, about 34%, about 35%, about 36%, about 37%, about 38%, about 39%, about 40%, about 41%, about 42%, about 43%, about 44%, or about 45%. In some embodiments, the mass fraction of cholesterol is about 30%. In some embodiments, the mass fraction of cholesterol is about 31%. In some embodiments, the mass fraction of cholesterol is about 32%. In some embodiments, the mass fraction of cholesterol is about 33%. In some embodiments, the mass fraction of cholesterol is about 34%. In some embodiments, the mass fraction of cholesterol is about 35%. In some embodiments, the mass fraction of cholesterol is about 36%. In some embodiments, the mass fraction of cholesterol is about 37%. In some embodiments, the mass fraction of cholesterol is about 38%. In some embodiments, the mass fraction of cholesterol is about 39%. In some embodiments, the mass fraction of cholesterol is about 40%. In some embodiments, the mass fraction of cholesterol is about 41%. In some embodiments, the mass fraction of cholesterol is about 42%. In some embodiments, the mass fraction of cholesterol is about 43%. In some embodiments, the mass fraction of cholesterol is about 44%. In some embodiments, the mass fraction of cholesterol is about 45%.

In some embodiments, the lipid nanoparticle (LNP) comprises: (1) Lipid T1 and (2) a deoxyribonucleic acid (DNA) molecule. In some embodiments, the mass fraction of Lipid T1 in the LNP is between about 0.1% and about 60%. In some embodiments, the mass fraction of Lipid T1 in the LNP is between about 1% and about 40%. In some embodiments, the mass fraction of Lipid T1 in the LNP is between about 0.2% and about 20%. In some embodiments, the mass fraction of Lipid T1 in the LNP is between about 0.2% and about 20%. In some embodiments, the mass fraction of Lipid T1 in the LNP is between about 0.5% and about 10%. In some embodiments, the mass fraction of Lipid T1 in the LNP is between about 1% and about 7%. In some embodiments, the mass fraction of Lipid T1 in the LNP is between about 1% and about 6%. In some embodiments, the mass fraction of Lipid T1 in the LNP is between about 2% and about 5%. In some embodiments, the mass fraction of Lipid T1 in the LNP is between about 0.2% and about 1%. In some embodiments, the mass fraction of Lipid T1 in the LNP is between about 10% and about 55%. In some embodiments, the mass fraction of Lipid T1 in the LNP is between about 20% and about 50%. In some embodiments, the mass fraction of Lipid T1 in the LNP is between about 35% and about 45%. In some embodiments, the LNP does not comprise another cationic or ionizable lipid.

In some embodiments, the DNA molecule comprises a transgene. In some embodiments, the transgene encodes a recombinant receptor. In some embodiments, the recombinant receptor is a chimeric antigen receptor (CAR) or a T cell receptor (TCR). In some embodiments, the recombinant receptor is a CAR. In some embodiments, the CAR comprises an extracellular antigen-binding domain, a transmembrane domain, and an intracellular region. In some embodiments, the extracellular antigen-binding domain is an antibody or an antigen-binding fragment thereof that binds to an antigen that is associated with, or expressed on a cell or tissue of a disease or condition. In some embodiments, the intracellular region comprises an intracellular signaling domain that is or comprises an intracellular signaling domain of a CD3 chain, or a signaling portion thereof. In some embodiments, the intracellular region comprises one or more costimulatory signaling domain(s) comprising an intracellular signaling domain selected from the group consisting of: a CD28, a 4-1BB, an ICOS, or a signaling portion thereof. In some embodiments, the CAR is a single antigen directed CAR. In some embodiments, the CAR is a bispecific CAR. In some embodiments, the DNA (e.g., ceDNA) molecule encoding the bispecific CAR is at least 6 kilobases, at least 7 kilobases, or at least 8 kilobases. In some embodiments, the bispecific CAR is between about 6 kilobases and about 8 kilobases. In some embodiments, the bispecific CAR is about 8 kilobases.

In some embodiments, the antigen is a tumor antigen. In some embodiments, the antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR VIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRCSA), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1). In some embodiments, the antigen is BCMA. In some embodiments, the antigen is CD19.

In some embodiments, the DNA molecule comprises a transgene. In some embodiments, the transgene is operably linked to a promoter and positioned between inverted terminal repeats (ITRs). In some embodiments, the DNA molecule is a closed end DNA vector. In some embodiments, the transgene is positioned between protelomerase binding sequences. In some embodiments, the DNA molecule is a doggybone DNA vector.

In some embodiments, the DNA molecule comprises a single-stranded DNA oligonucleotide (ssODN) or a double-stranded DNA oligonucleotide (dsODN). In some embodiments, the DNA molecule comprises a single-stranded DNA oligonucleotide (ssODN). In some embodiments, the ssODN comprises a nucleotide sequence that is homologous to a target genomic locus. In some embodiments, the DNA molecule comprises a double-stranded DNA oligonucleotide (dsODN). In some embodiments, the dsODN comprises a nucleotide sequence that is homologous to a target genomic locus.

In some embodiments, the mass fraction of the DNA in the LNP is between about 1% and about 10%, between about 2% and about 6%, or between about 3% and about 5%. In some embodiments, the mass fraction of the DNA is about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, or about 13%. In some embodiments, the mass fraction of the DNA is about 3%. In some embodiments, the mass fraction of the DNA is about 3.5%. In some embodiments, the mass fraction of the DNA is about 4%. In some embodiments, the mass fraction of the DNA is about 5%.

In some embodiments, the total DNA concentration in the LNP is about between about 0.5 ug/mL and about 10 ug/mL, between about 0.75 ug/mL and about 8 ug/mL, or between about 1.5 ug/mL and about 6 ug/mL. In some embodiments, the total DNA concentration is about 0.5 ug/mL. In some embodiments, the total DNA concentration is about 0.75 ug/mL. In some embodiments, the total DNA concentration is about 1 μg/mL. In some embodiments, the total DNA concentration is about 1.25 ug/mL. In some embodiments, the total DNA concentration is about 1.5 ug/mL. In some embodiments, the total DNA concentration is about 1.75 ug/mL. In some embodiments, the total DNA concentration is about 2 ug/mL. In some embodiments, the total DNA concentration is about 2.25 ug/mL. In some embodiments, the total DNA concentration is about 2.5 ug/mL. In some embodiments, the total DNA concentration is about 2.75 ug/mL. In some embodiments, the total DNA concentration is about 3 ug/mL. In some embodiments, the total DNA concentration is about 3.25 ug/mL. In some embodiments, the total DNA concentration is about 3.5 ug/mL. In some embodiments, the total DNA concentration is about 3.75 ug/mL. In some embodiments, the total DNA concentration is about 4 ug/mL. In some embodiments, the total DNA concentration is about 4.25 ug/mL. In some embodiments, the total DNA concentration is about 4.5 ug/mL. In some embodiments, the total DNA concentration is about 4.75 ug/mL. In some embodiments, the total DNA concentration is about 5 ug/mL. In some embodiments, the total DNA concentration is about 5.25 ug/mL. In some embodiments, the total DNA concentration is about 5.5 ug/mL. In some embodiments, the total DNA concentration is about 5.75 ug/mL. In some embodiments, the total DNA concentration is about 6 ug/mL. In some embodiments, the total DNA concentration is about 6.25 ug/mL. In some embodiments, the total DNA concentration is about 6.5 ug/mL. In some embodiments, the total DNA concentration is about 6.75 ug/mL. In some embodiments, the total DNA concentration is about 7 ug/mL. In some embodiments, the total DNA concentration is about 7.25 ug/mL. In some embodiments, the total DNA concentration is about 7.5 ug/mL. In some embodiments, the total DNA concentration is about 7.75 ug/mL. In some embodiments, the total DNA concentration is about 8 ug/mL.

### 2. Formulations

Provided herein are LNPs containing: (a) an ionizable lipid selected from the group consisting of DLin-KC2-DMA, or an analog thereof, OF-C4-Deg-Lin or analog thereof, and cKK-E12 or an analog thereof; (b) a helper lipid that is 1-stearoyl-2-oleoyl-sn-glycero-3-phosphocholine (SOPC); (c) a polyethylene glycol (PEG)-conjugated lipid that is DMG-PEG2000; (d) cholesterol; and (e) a deoxyribonucleic acid (DNA) molecule.

In some embodiments, the ionizable lipid is DLin-KC2-DMA or an analog thereof. In some embodiments, the ionizable lipid is DLin-KC2-DMA. In some embodiments, the mass fraction of DLin-KC2-DMA is between about 35% and about 45%. In some embodiments, the mass fraction of DLin-KC2-DMA is between about 30% and about 40%. In some embodiments, the mass fraction of DLin-KC2-DMA is between about 35% and about 40%. In some embodiments, the mass fraction of DLin-KC2-DMA is between about 30% and about 35%. In some embodiments, the mass fraction of SOPC is between about 15% and about 25%. In some embodiments, the mass fraction of DMG-PEG2000 is between about 2% and about 3%. In some embodiments, the mass fraction of cholesterol is between about 30% and about 40%. In some embodiments, the mass fraction of the DNA molecule is between about 3% and about 4%.

In some embodiments, the LNP further comprises a non-ionizable cationic lipid. In some embodiments, the mass fraction of the non-ionizable cationic lipid is between about 1% and 6% or between about 2% and about 5%. In some embodiments, the non-ionizable cationic lipid is Lipid T1.

In some embodiments, the LNP contains: (a) DLin-KC2-DMA with a mass fraction of between about 35% and about 45%; (b) SOPC with a mass fraction of between about 15% and about 25%; (c) DMG-PEG2000 with a mass fraction of between about 2% and about 3%; (d) cholesterol with a mass fraction of between about 30% and about 40%; and (e) a deoxyribonucleic acid (DNA) molecule. In some embodiments, the LNP contains: (a) DLin-KC2-DMA with a mass fraction of between about 30% and about 40%; (b) SOPC with a mass fraction of between about 15% and about 25%; (c) DMG-PEG2000 with a mass fraction of between about 2% and about 3%; (d) cholesterol with a mass fraction of between about 30% and about 40%; and (e) a deoxyribonucleic acid (DNA) molecule. In some embodiments, the LNP contains: (a) DLin-KC2-DMA with a mass fraction of about 40%; (b) SOPC with a mass fraction of about 19%; (c) DMG-PEG2000 with a mass fraction of about 2.5%; (d) cholesterol with a mass fraction of about 35%; and (e) a deoxyribonucleic acid (DNA) molecule with a mass fraction of about 3.5%. In some embodiments, the LNP further comprise a non-ionizable cationic lipid. In some embodiments, the mass fraction of the non-ionizable cationic lipid is between about 1% and 6% or between about 2% and about 5%. In some embodiments, the non-ionizable cationic lipid is Lipid T1,

In some embodiments, the ionizable lipid is a lipidoid comprising a diketopiperazine amino core. In some embodiments, the ionizable lipid is selected from OF-C4-Deg-Lin or analog thereof and cKK-E12 or an analog thereof. In some embodiments, the ionizable lipid comprises an unsaturated tail, optionally an unsaturated linoleic tail. In some embodiments, the ionizable lipid is selected from OF-C4-Deg-Lin or analog thereof.

In some embodiments, the ionizable lipid is OF-C4-Deg-Lin or an analog thereof. In some embodiments, the ionizable lipid is OF-C4-Deg-Lin. In some embodiments, the mass fraction of OF-C4-Deg-Lin is between about 35% and about 45%. In some embodiments, the mass fraction of OF-C4-Deg-Lin is between about 30% and about 40%. In some embodiments, the mass fraction of OF-C4-Deg-Lin is between about 35% and about 40%. In some embodiments, the mass fraction of OF-C4-Deg-Lin is between about 30% and about 35%. In some embodiments, the mass fraction of SOPC is between about 15% and about 25%. In some embodiments, the mass fraction of DMG-PEG2000 is between about 2% and about 3%. In some embodiments, the mass fraction of cholesterol is between about 30% and about 40%. In some embodiments, the mass fraction of the DNA molecule is between about 3% and about 4%. In some embodiments, the LNP further comprise a non-ionizable cationic lipid. In some embodiments, the mass fraction of the non-ionizable cationic lipid is between about 1% and 6% or between about 2% and about 5%. In some embodiments, the non-ionizable cationic lipids is Lipid T1,

In some embodiments, the LNP contains: (a) OF-C4-Deg-Lin with a mass fraction of between about 35% and about 45%; (b) SOPC with a mass fraction of between about 15% and about 25%; (c) DMG-PEG2000 with a mass fraction of between about 2% and about 3%; (d) cholesterol with a mass fraction of between about 30% and about 40%; and (e) a deoxyribonucleic acid (DNA) molecule. In some embodiments, the LNP contains: (a) OF-C4-Deg-Lin with a mass fraction of between about 30% and about 40%; (b) SOPC with a mass fraction of between about 15% and about 25%; (c) DMG-PEG2000 with a mass fraction of between about 2% and about 3%; (d) cholesterol with a mass fraction of between about 30% and about 40%; and (e) a deoxyribonucleic acid (DNA) molecule. In some embodiments, the LNP contains: (a) OF-C4-Deg-Lin with a mass fraction of about 40%; (b) SOPC with a mass fraction of about 19%; (c) DMG-PEG2000 with a mass fraction of about 2.5%; (d) cholesterol with a mass fraction of about 35%; and (e) a deoxyribonucleic acid (DNA) molecule with a mass fraction of about 3.5%. In some embodiments, the LNP further comprise a non-ionizable cationic lipid. In some embodiments, the mass fraction of the non-ionizable cationic lipid is between about 1% and 6% or between about 2% and about 5%. In some embodiments, the non-ionizable cationic lipid is Lipid T1,

In some embodiments, the ionizable lipid is cKK-E12 or an analog thereof. In some embodiments, the ionizable lipid is cKK-E12. In some embodiments, the mass fraction of cKK-E12 is between about 35% and about 45%. In some embodiments, the mass fraction of cKK-E12 is between about 30% and about 40%. In some embodiments, the mass fraction of cKK-E12 is between about 35% and about 40%. In some embodiments, the mass fraction of cKK-E12 is between about 30% and about 35%. In some embodiments, the mass fraction of SOPC is between about 15% and about 25%. In some embodiments, the mass fraction of DMG-PEG2000 is between about 2% and about 3%. In some embodiments, the mass fraction of cholesterol is between about 30% and about 40%. In some embodiments, the mass fraction of the DNA molecule is between about 3% and about 4%. In some embodiments, the LNP further comprise a non-ionizable cationic lipid. In some embodiments, the mass fraction of the non-ionizable cationic lipid is between about 1% and 6% or between about 2% and about 5%. In some embodiments, the non-ionizable cationic lipid is Lipid T1,

In some embodiments, the LNP contains: (a) cKK-E12 with a mass fraction of between about 35% and about 45%; (b) SOPC with a mass fraction of between about 15% and about 25%; (c) DMG-PEG2000 with a mass fraction of between about 2% and about 3%; (d) cholesterol with a mass fraction of between about 30% and about 40%; and (e) a deoxyribonucleic acid (DNA) molecule. In some embodiments, the LNP contains: (a) cKK-E12 with a mass fraction of between about 30% and about 40%; (b) SOPC with a mass fraction of between about 15% and about 25%; (c) DMG-PEG2000 with a mass fraction of between about 2% and about 3%; (d) cholesterol with a mass fraction of between about 30% and about 40%; and (e) a deoxyribonucleic acid (DNA) molecule. In some embodiments, the LNP contains: (a) cKK-E12 with a mass fraction of about 40%; (b) SOPC with a mass fraction of about 19%; (c) DMG-PEG2000 with a mass fraction of about 2.5%; (d) cholesterol with a mass fraction of about 35%; and (e) a deoxyribonucleic acid (DNA) molecule with a mass fraction of about 3.5%. In some embodiments, the LNP further comprise a non-ionizable cationic lipid. In some embodiments, the mass fraction of the non-ionizable cationic lipid is between about 1% and 6% or between about 2% and about 5%. In some embodiments, the non-ionizable cationic lipid is Lipid T1.

### B. RNA LNPs

### 1. Components

Provided herein are LNPs comprising a ribonucleic acid (RNA) molecule (also referred to as RNA LNPs or RNA-containing LNPs). In some embodiments, the RNA LNP comprises an ionizable lipid; a helper lipid; a polyethylene glycol (PEG)-conjugated lipid; cholesterol; and an RNA molecule.

In some embodiments, the lipid nanoparticle composition contains an ionizable lipid. In some embodiments, the ionizable lipid is positively charged (e.g. a cationic lipid). In some embodiments, the ionizable lipid is a cationic lipid, including but not limited to those described in U.S. Pat. Nos. 9,593,077; 9,365,610; 9,670,152; and 9,458,090. In some embodiments, the ionizable lipid is a cationic lipid, including but not limited to those described in Published PCT application WO2013149140. In some embodiments, the ionizable lipid is a cationic lipid, including but not limited to those described in published US Patent application US2019084965; US2019106379. In some embodiments, the ionizable lipid is a cationic lipid, including but not limited to those described in Published EP application EP2830595.

In some embodiments, the ionizable lipid is positively charged (e.g. a cationic lipid). In some embodiments, the ionizable lipid is selected from the group consisting of D-Lin, 2,2-dilinoleyl-4-dimethylaminoethyl-[1,3]-dioxolane (DLin-KC2-DMA), or an analog thereof, OF-C4-Deg-Lin, or an analog thereof, and cKK-E12, or an analog thereof.

In some embodiments, the ionizable lipid is DLin-KC2-DMA or an analog thereof. In some embodiments, the ionizable lipid has a structure selected from among the following:

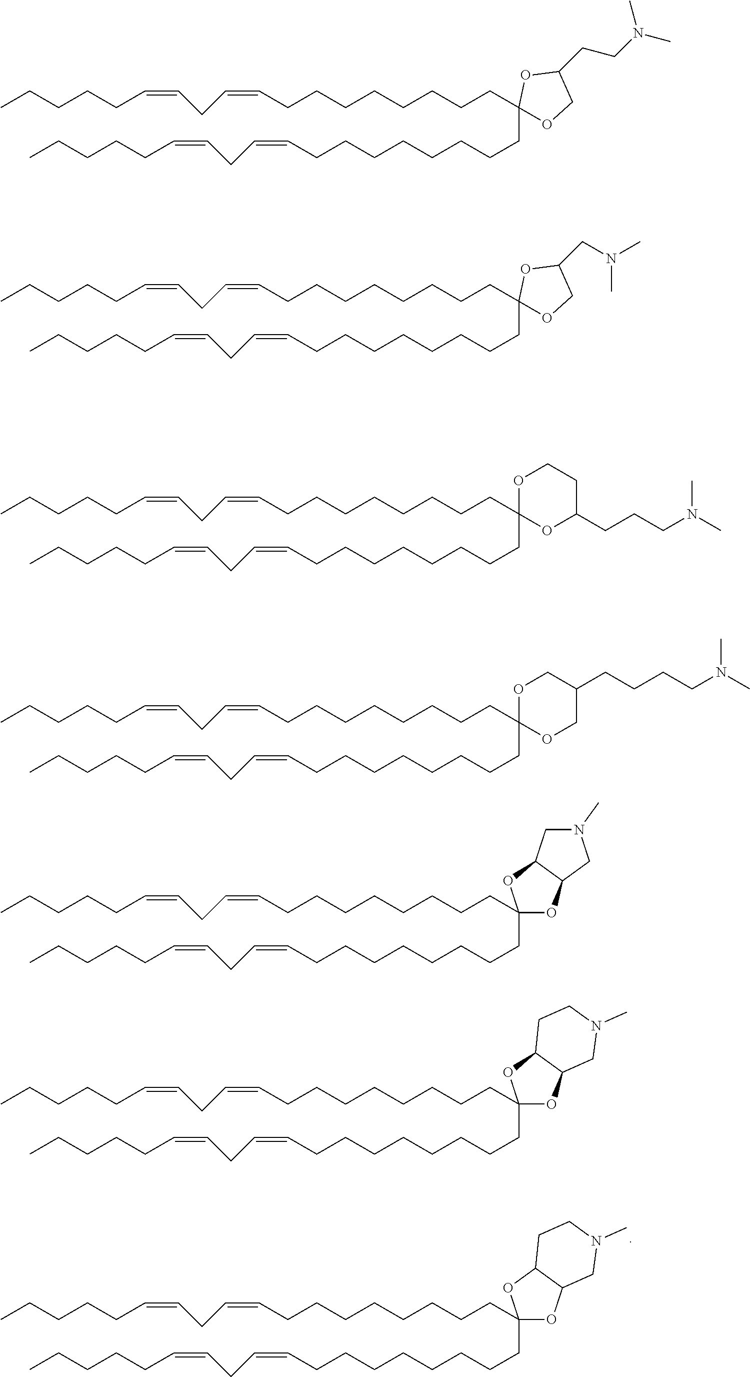

In some cases, the ionizable lipid is DLin-KC2-DMA. In some cases, the ionizable lipid has the structure of

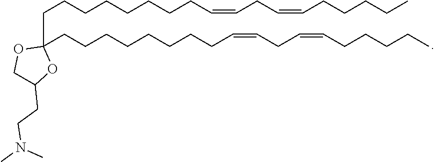 [SMILES: CCCCC/C=C\C/C=C\CCCCCCCCC1(CCCCCCCC/C=C\C/C=C\CCCCC)OCC(CCN(C)C)O1]

In some embodiments, the ionizable lipid is DLin-MC3-DMA or an analog thereof. In some embodiments, the ionizable lipid has the structure of

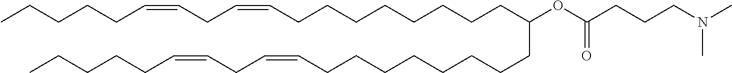 [SMILES: CCCCC/C=C\C/C=C\CCCCCCCCC(CCCCCCCC/C=C\C/C=C\CCCCC)OC(=O)CCCN(C)C]

or an analog thereof.

In some embodiments, the ionizable lipid is DLin-MC3-DMA. In some embodiments, the ionizable lipid has the structure of

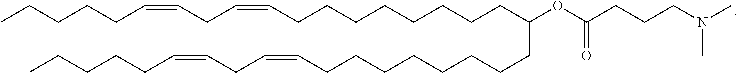 [SMILES: CCCCC/C=C\C/C=C\CCCCCCCCC(CCCCCCCC/C=C\C/C=C\CCCCC)OC(=O)CCCN(C)C]

In some embodiments, the ionizable lipid is positively charged (e.g. a cationic lipid). In some embodiments, the ionizable lipid is a lipid or lipidoid comprising a diketopiperazine ring core. In some embodiments, the ionizable lipid is any as described in Fenton et al., Angew. Chem. Int. Ed. (2018) 57:13582-86, incorporated by reference in its entirety. In some embodiments, the ionizable lipid is selected from OF-C4-Deg-Lin, an analog thereof. In some embodiments, the ionizable lipid has an unsaturated tail. In some embodiments, the ionizable lipid has an unsaturated linoleic tail. In some embodiments, the ionizable lipid is selected from OF-C4-Deg-Lin or analog thereof. In some embodiments, the ionizable lipid has the structure of

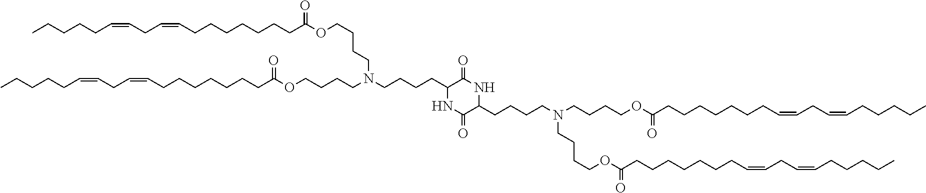 [SMILES: CCCCC/C=C\C/C=C\CCCCCCCC(=O)OCCCCN(CCCCOC(=O)CCCCCCC/C=C\C/C=C\CCCCC)CCCCC1NC(=O)C(CCCCN(CCCCOC(=O)CCCCCCC/C=C\C/C=C\CCCCC)CCCCOC(=O)CCCCCCC/C=C\C/C=C\CCCCC)NC1=O]

or an analog thereof. In some embodiments, the ionizable lipid is OF-C4-Deg-Lin. In some embodiments, the ionizable lipid has the structure of

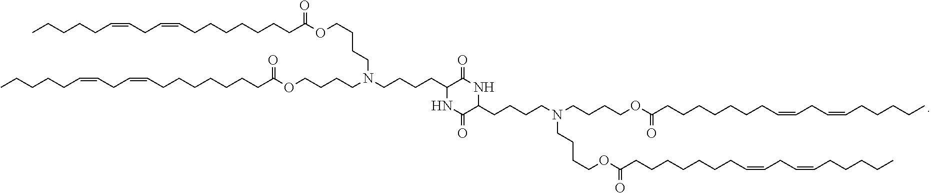 [SMILES: CCCCC/C=C\C/C=C\CCCCCCCC(=O)OCCCCN(CCCCOC(=O)CCCCCCC/C=C\C/C=C\CCCCC)CCCCC1NC(=O)C(CCCCN(CCCCOC(=O)CCCCCCC/C=C\C/C=C\CCCCC)CCCCOC(=O)CCCCCCC/C=C\C/C=C\CCCCC)NC1=O]

In some embodiments, the ionizable lipid is cKK-E12 or an analog thereof. In some embodiments, the ionizable lipid has the structure of

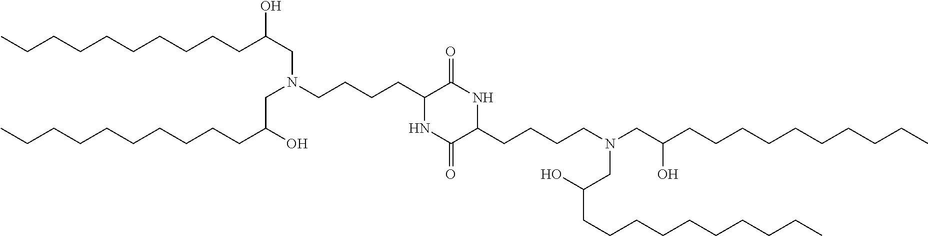 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

or an analog thereof. In some embodiments, the ionizable lipid is cKK-E12.

In some embodiments, the ionizable lipid has the structure of

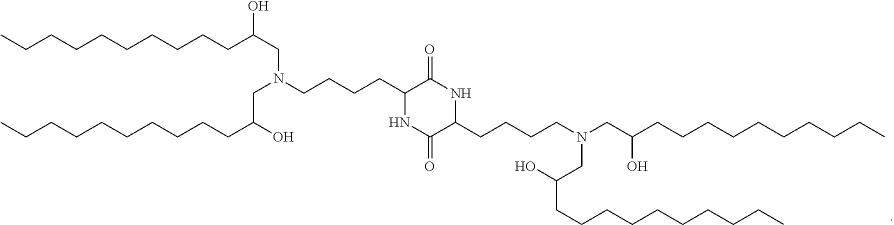 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In some embodiments, the mass fraction of the ionizable lipid in the LNP is between about 32% and about 65%. In some embodiments, the mass fraction of the ionizable lipid is about 32%, about 32.5%, about 33%, about 33.5%, about 34%, about 34.5%, about 35%, about 35.5%, about 36%, about 50%, about 51%, about 52%, about 53%, about 54%, about 55%, about 56%, about 57%, about 58%, about 59%, about 60%, about 61%, about 62%, about 63%, about 64%, or about 65%. In some embodiments, the mass fraction of the ionizable lipid is about 32%. In some embodiments, the mass fraction of the ionizable lipid is about 32.5%. In some embodiments, the mass fraction of the ionizable lipid is about 33%. In some embodiments, the mass fraction of the ionizable lipid is about 33.5%. In some embodiments, the mass fraction of the ionizable lipid is about 34%. In some embodiments, the mass fraction of the ionizable lipid is about 34.5%. In some embodiments, the mass fraction of the ionizable lipid is about 35%. In some embodiments, the mass fraction of the ionizable lipid is about 35.5%. In some embodiments, the mass fraction of the ionizable lipid is about 36%. In some embodiments, the mass fraction of the ionizable lipid is about 54%. In some embodiments, the mass fraction of the ionizable lipid is about 55%. In some embodiments, the mass fraction of the ionizable lipid is about 56%. In some embodiments, the mass fraction of the ionizable lipid is about 57%. In some embodiments, the mass fraction of the ionizable lipid is about 58%. In some embodiments, the mass fraction of the ionizable lipid is about 59%. In some embodiments, the mass fraction of the ionizable lipid is about 60%. In some embodiments, the mass fraction of the ionizable lipid is about 61%.

In some embodiments, the LNP comprises more than one cationic lipid. In some embodiments, the LNP comprises two cationic lipids. In some embodiments, the LNP comprises an ionizable cationic lipid a non-ionizable cationic lipid. In some embodiments, the mass fraction of the non-ionizable cationic lipid in the LNP is between about 0.5% and about 7%. In some embodiments, the mass fraction of the non-ionizable cationic lipid in the LNP is between about 0.5% and about 5%. In some embodiments, the mass fraction of the non-ionizable cationic lipid in the LNP is between about 1% and about 6%. In other embodiments, the mass fraction of the non-ionizable cationic lipid in the LNP is between about 2% and about 5%.

In some embodiments, the non-ionizable cationic lipid has the following structure:

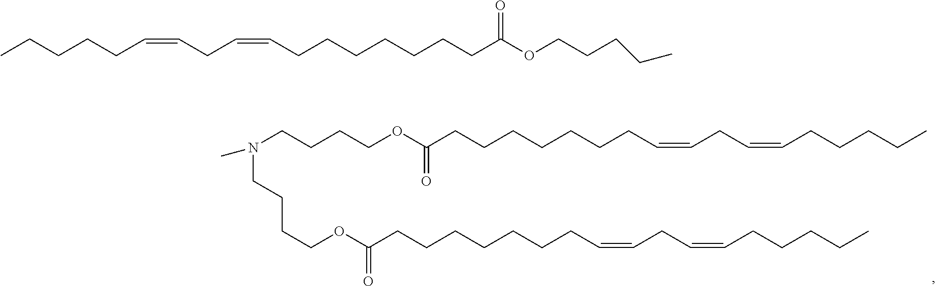

which is referred to herein as Lipid T1. In some embodiments where the LNP comprises at least one ionizable lipid, the mass fraction of Lipid T1 in the LNP is between about 0.1% and about 40%. In some embodiments where the LNP comprises at least one ionizable lipid, the mass fraction of Lipid T1 in the LNP is between about 0.2% and about 20%. In some embodiments where the LNP comprises at least one ionizable lipid, the mass fraction of Lipid T1 in the LNP is between about 0.2% and about 20%. In some embodiments where the LNP comprises at least one ionizable lipid, the mass fraction of Lipid T1 in the LNP is between about 0.5% and about 10%. In some embodiments where the LNP comprises at least one ionizable lipid, the mass fraction of Lipid T1 in the LNP is between about 1% and about 7%. In some embodiments where the LNP comprises at least one ionizable lipid, the mass fraction of Lipid T1 in the LNP is between about 1% and about 6%. In some embodiments where the LNP comprises at least one ionizable lipid, the mass fraction of Lipid T1 in the LNP is between about 2% and about 5%. In some embodiments where the LNP comprises at least one ionizable lipid, the mass fraction of Lipid T1 in the LNP is between about 0.2% and about 1%.

In some cases, the non-ionizable cationic lipid is DOTAP. In some cases, the ionizable lipid has the structure of

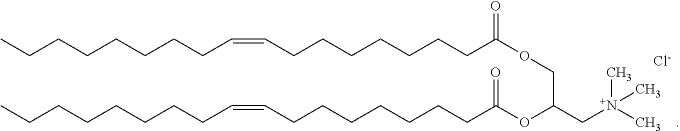 [SMILES: CCCCCCCC/C=C\CCCCCCCC(=O)OCC(C[N+](C)(C)C)OC(=O)CCCCCCC/C=C\CCCCCCCC ; [Cl-]]

In some cases, the non-ionizable cationic lipid is DOTMA. In some cases, the ionizable lipid has the structure of

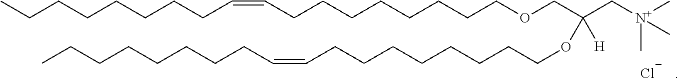 [SMILES: CCCCCCCC/C=C\CCCCCCCCOCC(C[N+](C)(C)C)OCCCCCCCC/C=C\CCCCCCCC ; [Cl-]]

In some cases, the non-ionizable lipid is DODAP In some cases the ionizable lipid has the structure of

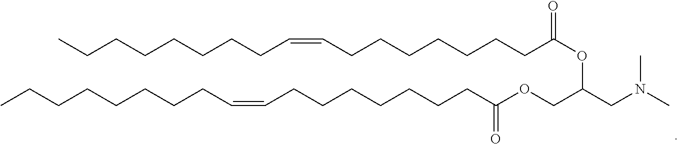 [SMILES: CCCCCCCC/C=C\CCCCCCCC(=O)OCC(CN(C)C)OC(=O)CCCCCCC/C=C\CCCCCCCC]

In some cases, the non-ionizable lipid is DOSPA In some cases the ionizable lipid has the structure of

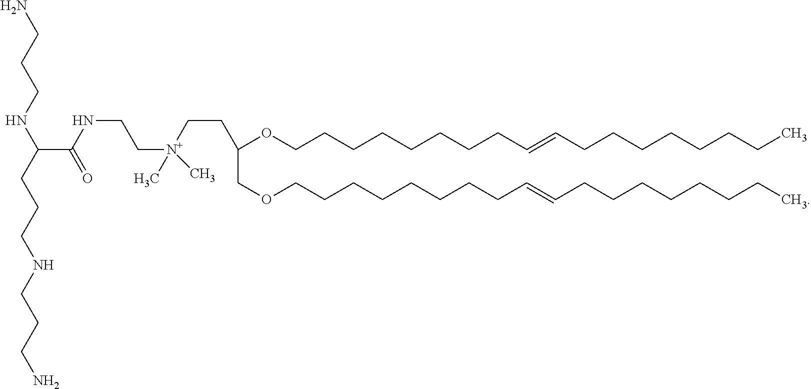 [SMILES: CCCCCCCC/C=C/CCCCCCCCOCC(CC[N+](C)(C)CCNC(=O)C(CCCNCCCN)NCCCN)OCCCCCCCC/C=C/CCCCCCCC]

In some embodiments, the helper lipid is selected from the group consisting of dioleoylphosphatidylethanolamine (DOPE), 1,2-distearoyl-sn-glycero-3-phosphocholine (DSPC), 1-Stearoyl-2-oleoyl-sn-glycero-3-phosphocholine (SOPC), and 1,2-dioleoyl-sn-glycero-sn-3-phosphatidylcholine (DOPC). In some embodiments, the helper lipid is DOPE. In some embodiments, the helper lipid has the structure of

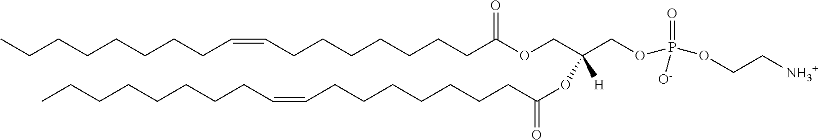 [SMILES: CCCCCCCC/C=C\CCCCCCCC(=O)OC[C@H](COP(=O)([O-])OCC[NH3+])OC(=O)CCCCCCC/C=C\CCCCCCCC]

In some embodiments, the helper lipid is DSPC. In some embodiments, the helper lipid has the structure of

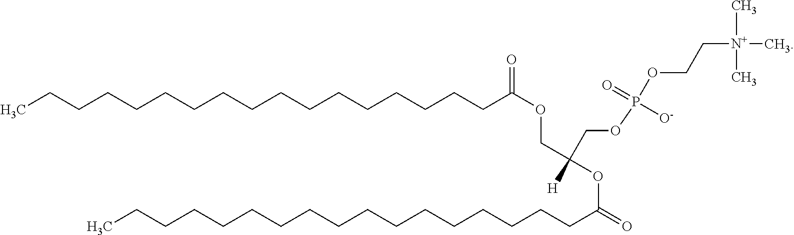 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the helper lipid is SOPC. In some embodiments the helper lipid has the structure of

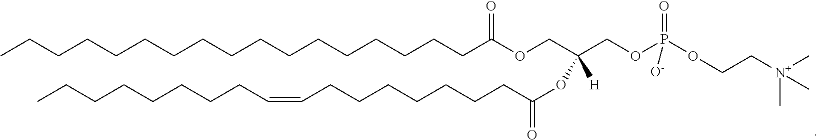 [SMILES: CCCCCCCC/C=C\CCCCCCCC(=O)O[C@H](COC(=O)CCCCCCCCCCCCCCCCC)COP(=O)([O-])OCC[N+](C)(C)C]

In some embodiments, the helper lipid is DOPC. In some embodiments, the helper lipid has the structure of

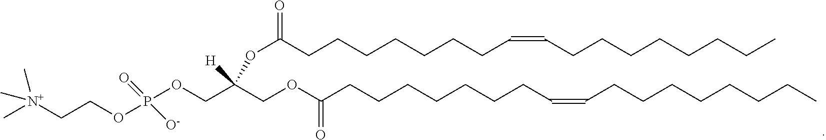 [SMILES: CCCCCCCC/C=C\CCCCCCCC(=O)OC[C@H](COP(=O)([O-])OCC[N+](C)(C)C)OC(=O)CCCCCCC/C=C\CCCCCCCC]

In some embodiments, the mass fraction of the helper lipid in the LNP is between about 4% and about 20%. In some embodiments, the mass fraction of the helper lipid is about 4%, about 4.5%, about 5%, about 5.5%, about 6%, about 6.5%, about 7%, about 7.5%, about 8%, about 8.5%, about 9%, about 9.5%, 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20%. In some embodiments, the mass fraction of the helper lipid is about 4%. In some embodiments, the mass fraction of the helper lipid is about 4.5%. In some embodiments, the mass fraction of the helper lipid is about 5%. In some embodiments, the mass fraction of the helper lipid is about 5.5%. In some embodiments, the mass fraction of the helper lipid is about 6%. In some embodiments, the mass fraction of the helper lipid is about 6.5%. In some embodiments, the mass fraction of the helper lipid is about 7%. In some embodiments, the mass fraction of the helper lipid is about 7.5%. In some embodiments, the mass fraction of the helper lipid is about 8%. In some embodiments, the mass fraction of the helper lipid is about 12%. In some embodiments, the mass fraction of the helper lipid is about 12.5%. In some embodiments, the mass fraction of the helper lipid is about 13%. In some embodiments, the mass fraction of the helper lipid is about 13.5%. In some embodiments, the mass fraction of the helper lipid is about 14%. In some embodiments, the mass fraction of the helper lipid is about 14.5%. In some embodiments, the mass fraction of the helper lipid is about 15%. In some embodiments, the mass fraction of the helper lipid is about 15.5%. In some embodiments, the mass fraction of the helper lipid is about 16%. In some embodiments, the mass fraction of the helper lipid is about 16.5%. In some embodiments, the mass fraction of the helper lipid is about 17%. In some embodiments, the mass fraction of the helper lipid is about 17.5. In some embodiments, the mass fraction of the helper lipid is about 18%. In some embodiments, the mass fraction of the helper lipid is about 18.5%. In some embodiments, the mass fraction of the helper lipid is about 19%. In some embodiments, the mass fraction of the helper lipid is about 19.5%. In some embodiments, the mass fraction of the helper lipid is about 20%.

In some embodiments, the polyethylene glycol (PEG)-conjugated lipid is DMG-PEG2000. In some embodiments, the PEG-conjugated lipid has the structure of

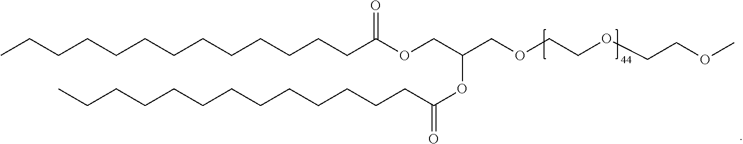

In some embodiments, the mass fraction of the PEG-conjugated lipid in the LNP is between about 2% and about 10%, or between about 3% and about 7%. In some embodiments, the mass fraction of the PEG-conjugated lipid is about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%. In some embodiments, the mass fraction of the PEG-conjugated lipid is about 2%. In some embodiments, the mass fraction of the PEG-conjugated lipid is about 2.5%. In some embodiments, the mass fraction of the PEG-conjugated lipid is about 3%. In some embodiments, the mass fraction of the PEG-conjugated lipid is about 3.5%. In some embodiments, the mass fraction of the PEG-conjugated lipid is about 4%. In some embodiments, the mass fraction of the PEG-conjugated lipid is about 4.5%. In some embodiments, the mass fraction of the PEG-conjugated lipid is about 5%. In some embodiments, the mass fraction of the PEG-conjugated lipid is about 5.5%. In some embodiments, the mass fraction of the PEG-conjugated lipid is about 6%. In some embodiments, the mass fraction of the PEG-conjugated lipid is about 6.5%. In some embodiments, the mass fraction of the PEG-conjugated lipid is about 7%. In some embodiments, the mass fraction of the PEG-conjugated lipid is about 7.5%. In some embodiments, the mass fraction of the PEG-conjugated lipid is about 8%. In some embodiments, the mass fraction of the PEG-conjugated lipid is about 8.5%.

In some embodiments, cholesterol has the structure of

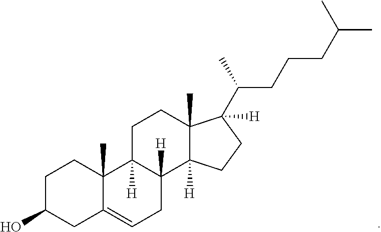

In some embodiments, the mass fraction of cholesterol in the LNP is between about 15% and about 45%. In some embodiments, the mass fraction of cholesterol is about 15%, about 16%, about 17%, about 18%, about 19%, about 20%, about 25%, about 30%, 35%, about 36%, about 37%, about 38%, about 39%, about 40%, about 41%, about 42%, about 43%, about 44%, or about 45%. In some embodiments, the mass fraction of cholesterol is about 15%. In some embodiments, the mass fraction of cholesterol is about 16%. In some embodiments, the mass fraction of cholesterol is about 17%. In some embodiments, the mass fraction of cholesterol is about 18%. In some embodiments, the mass fraction of cholesterol is about 19%. In some embodiments, the mass fraction of cholesterol is about 20%. In some embodiments, the mass fraction of cholesterol is about 35%. In some embodiments, the mass fraction of cholesterol is about 36%. In some embodiments, the mass fraction of cholesterol is about 37%. In some embodiments, the mass fraction of cholesterol is about 38%. In some embodiments, the mass fraction of cholesterol is about 39%. In some embodiments, the mass fraction of cholesterol is about 40%. In some embodiments, the mass fraction of cholesterol is about 41%. In some embodiments, the mass fraction of cholesterol is about 42%. In some embodiments, the mass fraction of cholesterol is about 43%. In some embodiments, the mass fraction of cholesterol is about 44%. In some embodiments, the mass fraction of cholesterol is about 45%.

In some embodiments, the lipid nanoparticle (LNP) comprises: (1) Lipid T1 and (2) a deoxyribonucleic acid (DNA) molecule. In some embodiments, the mass fraction of Lipid T1 in the LNP is between about 0.1% and about 60%. In some embodiments, the mass fraction of Lipid T1 in the LNP is between about 1% and about 40%. In some embodiments, the mass fraction of Lipid T1 in the LNP is between about 0.2% and about 20%. In some embodiments, the mass fraction of Lipid T1 in the LNP is between about 0.2% and about 20%. In some embodiments, the mass fraction of Lipid T1 in the LNP is between about 0.5% and about 10%. In some embodiments, the mass fraction of Lipid T1 in the LNP is between about 1% and about 7%. In some embodiments, the mass fraction of Lipid T1 in the LNP is between about 1% and about 6%. In some embodiments, the mass fraction of Lipid T1 in the LNP is between about 2% and about 5%. In some embodiments, the mass fraction of Lipid T1 in the LNP is between about 0.2% and about 1%. In some embodiments, the mass fraction of Lipid T1 in the LNP is between about 10% and about 55%. In some embodiments, the mass fraction of Lipid T1 in the LNP is between about 20% and about 50%. In some embodiments, the mass fraction of Lipid T1 in the LNP is between about 35% and about 45%. In some embodiments, the LNP does not comprise another cationic or ionizable lipid.

In some embodiments, the RNA molecule is selected from the group consisting of a messenger RNA (mRNA) encoding for a recombinant nuclease, mRNA encoding for a transposase, and guide RNA (gRNA). In some embodiments, the gRNA is a single guide RNA (sgRNA) comprising a crispr RNA (crRNA) and a tracrRNA.

In some embodiments, the RNA molecule is mRNA encoding for a recombinant nuclease. In some embodiments, the recombinant nuclease is capable of inducing a break in DNA. In some embodiments, the recombinant nuclease is a zinc finger nuclease (ZFN), a transcription activator-like effector nuclease (TALEN), or a CRISPR-associated nuclease (Cas). In some embodiments, the recombinant nuclease is a ZFN. In some embodiments, the recombinant nuclease is a TALEN. In some embodiments, the recombinant nuclease is a Cas. In some embodiments, Cas is Cas9 or Cas12.

In some embodiments, the RNA molecule is mRNA encoding for a transposase. In some embodiments, the transposase is a Class II transposase. In some embodiments, the transposase is selected from the group consisting of: Sleeping Beauty, piggyBac, TcBuster, Frog Prince, Tol2, Tc1/mariner, or a derivative thereof having transposase activity. In some embodiments, the transposase is Sleeping Beauty, PiggyBac, or TcBuster. In some embodiments, the transposase is Sleeping Beauty. In some embodiments, the transposase is PiggyBac. In some embodiments, the transposase is TcBuster.

In some embodiments, the RNA molecule is gRNA. In some embodiments, the gRNA is a single guide RNA (sgRNA) comprising a crispr RNA (crRNA) and a tracrRNA.

In some embodiments, the mass fraction of the RNA in the LNP is between about 2% and about 12%, between about 3% and about 11%, or between about 3.5% and about 10%. In some embodiments, the mass fraction of the RNA is about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, or about 13%. In some embodiments, the mass fraction of the RNA is about 3.5%. In some embodiments, the mass fraction of the RNA is about 10%.

In some embodiments, the total RNA concentration in the LNP is between about 4 ug/mL and about 200 ug/mL, between about 5 ug/mL and about 50 ug/mL, or between about 50 ug/mL and about 150 ug/mL. In some embodiments, the total RNA concentration is about 4 ug/mL. In some embodiments, the total RNA concentration is about 4.5 ug/mL. In some embodiments, the total RNA concentration is about 5 ug/mL. In some embodiments, the total RNA concentration is about 6 ug/mL. In some embodiments, the total RNA concentration is about 6.5 ug/mL. In some embodiments, the total RNA concentration is about 7 ug/mL. In some embodiments, the total RNA concentration is about 7.5 ug/mL. In some embodiments, the total RNA concentration is about 8 ug/mL. In some embodiments, the total RNA concentration is about 8.5 ug/mL. In some embodiments, the total RNA concentration is about 9 ug/mL. In some embodiments, the total RNA concentration is about 10 ug/mL. In some embodiments, the total RNA concentration is about 20 ug/mL. In some embodiments, the total RNA concentration is about 30 ug/mL. In some embodiments, the total RNA concentration is about 40 ug/mL. In some embodiments, the total RNA concentration is about 50 ug/mL. In some embodiments, the total RNA concentration is about 55 ug/mL. In some embodiments, the total RNA concentration is about 60 ug/mL. In some embodiments, the total RNA concentration is about 65 ug/mL. In some embodiments, the total RNA concentration is about 70 ug/mL. In some embodiments, the total RNA concentration is about 75 ug/mL. In some embodiments, the total RNA concentration is about 80 ug/mL. In some embodiments, the total RNA concentration is about 85 ug/mL. In some embodiments, the total RNA concentration is about 90 ug/mL. In some embodiments, the total RNA concentration is about 95 ug/mL. In some embodiments, the total RNA concentration is about 100 ug/mL. In some embodiments, the total RNA concentration is about 125 ug/mL. In some embodiments, the total RNA concentration is about 150 ug/mL.

### 2. Formulations

Provided herein are LNPs containing: (a) an ionizable lipid selected from the group consisting of DLin-MC3-DMA, or an analog thereof, OF-C4-Deg-Lin or analog thereof, and cKK-E12 or an analog thereof; (b) a helper lipid that is 1,2-distearoyl-sn-glycero-3-phosphocholine (DSPC); (c) a polyethylene glycol (PEG)-conjugated lipid that is DMG-PEG2000; (d) cholesterol; and (e) a ribonucleic acid (RNA) molecule.

In some embodiments, the ionizable lipid is DLin-MC3-DMA or an analog thereof. In some embodiments, the ionizable lipid is DLin-MC3-DMA. In some embodiments, the mass fraction of DLin-MC3-DMA is between about 35% and about 40%. In some embodiments, the mass fraction of DSPC is between about 10% and about 20%. In some embodiments, the mass fraction of DMG-PEG2000 is between about 3% and about 5%. In some embodiments, the mass fraction of cholesterol is between about 35% and about 45%. In some embodiments, the mass fraction of the RNA molecule is between about 2.5% and about 3%. In some embodiments, the mass fraction of the RNA molecule is between about 3% and about 4%.

In some embodiments, the LNP contains: (a) DLin-MC3-DMA with a mass fraction of between about 35% and about 40%; (b) DSPC with a mass fraction of between about 10% and about 20%; (c) DMG-PEG2000 with a mass fraction of between about 3% and about 5%; (d) cholesterol with a mass fraction of between about 35% and about 45%; and (e) a ribonucleic acid (RNA) molecule. In some embodiments, the LNP contains: (a) DLin-MC3-DMA with a mass fraction of about 37.8%; (b) DSPC with a mass fraction of about 15.2%; (c) DMG-PEG2000 with a mass fraction of about 3.5%; (d) cholesterol with a mass fraction of about 40%; and (e) a ribonucleic acid (RNA) molecule with a mass fraction of about 3.5%.

In some embodiments, the ionizable lipid is DLin-MC3-DMA or an analog thereof. In some embodiments, the ionizable lipid is DLin-MC3-DMA. In some embodiments, the mass fraction of DLin-MC3-DMA is between about 32% and about 36%. In some embodiments, the mass fraction of DSPC is between about 15% and about 20%. In some embodiments, the mass fraction of DMG-PEG2000 is between about 3.5% and about 5.5%. In some embodiments, the mass fraction of cholesterol is between about 35% and about 45%. In some embodiments, the mass fraction of the RNA molecule is between about 2.5% and about 3%. In some embodiments, the mass fraction of the RNA molecule is between about 3% and about 4%.

In some embodiments, the LNP contains: (a) DLin-MC3-DMA with a mass fraction of between about 32% and about 36%; (b) DSPC with a mass fraction of between about 15% and about 20%; (c) DMG-PEG2000 with a mass fraction of between about 3.5% and about 5.5%; (d) cholesterol with a mass fraction of between about 35% and about 45%; and (e) a ribonucleic acid (RNA) molecule. In some embodiments, the LNP contains: (a) DLin-MC3-DMA with a mass fraction of about 34.4%; (b) DSPC with a mass fraction of about 17.6%; (c) DMG-PEG2000 with a mass fraction of about 4.5%; (d) cholesterol with a mass fraction of about 40%; and (e) a ribonucleic acid (RNA) molecule with a mass fraction of about 3.5%.

In some embodiments, the ionizable lipid is a lipidoid comprising a diketopiperazine amino core. In some embodiments, the ionizable lipid is selected from OF-C4-Deg-Lin or analog thereof and cKK-E12 or an analog thereof. In some embodiments, the ionizable lipid comprises an unsaturated tail, optionally an unsaturated linoleic tail. In some embodiments, the ionizable lipid is selected from OF-C4-Deg-Lin or analog thereof.

In some embodiments, the LNP contains: (a) OF-C4-Deg-Lin with a mass fraction of between about 32% and about 36%; (b) DSPC with a mass fraction of between about 15% and about 20%; (c) DMG-PEG2000 with a mass fraction of between about 3.5% and about 5.5%; (d) cholesterol with a mass fraction of between about 35% and about 45%; and (e) a ribonucleic acid (RNA) molecule. In some embodiments, the LNP contains: (a) OF-C4-Deg-Lin with a mass fraction of about 34.4%; (b) DSPC with a mass fraction of about 17.6%; (c) DMG-PEG2000 with a mass fraction of about 4.5%; (d) cholesterol with a mass fraction of about 40%; and (e) a ribonucleic acid (RNA) molecule with a mass fraction of about 3.5%.

In some embodiments, the LNP contains: (a) cKK-E12 with a mass fraction of between about 32% and about 36%; (b) DSPC with a mass fraction of between about 15% and about 20%; (c) DMG-PEG2000 with a mass fraction of between about 3.5% and about 5.5%; (d) cholesterol with a mass fraction of between about 35% and about 45%; and (e) a ribonucleic acid (RNA) molecule. In some embodiments, the LNP contains: (a) cKK-E12 with a mass fraction of about 34.4%; (b) DSPC with a mass fraction of about 17.6%; (c) DMG-PEG2000 with a mass fraction of about 4.5%; (d) cholesterol with a mass fraction of about 40%; and (e) a ribonucleic acid (RNA) molecule with a mass fraction of about 3.5%.

In some embodiments, the LNP contains: (a) OF-C4-Deg-Lin with a mass fraction of between about 50% and about 65%; (b) DSPC with a mass fraction of between about 12% and about 24%; (c) DMG-PEG2000 with a mass fraction of between about 6% and about 7%; (d) cholesterol with a mass fraction of between about 17% and about 20%; and (e) a ribonucleic acid (RNA) molecule. In some embodiments, the LNP contains: (a) OF-C4-Deg-Lin with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 3.5%. In some embodiments, the LNP contains: (a) OF-C4-Deg-Lin with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 5%. In some embodiments, the LNP contains: (a) OF-C4-Deg-Lin with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 10%. In some embodiments, the LNP contains: (a) OF-C4-Deg-Lin with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 3.5%. In some embodiments, the LNP contains: (a) OF-C4-Deg-Lin with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 10%.

In some embodiments, the LNP contains: (a) cKK-E12 with a mass fraction of between about 50% and about 65%; (b) DSPC with a mass fraction of between about 12% and about 24%; (c) DMG-PEG2000 with a mass fraction of between about 6% and about 7%; (d) cholesterol with a mass fraction of between about 17% and about 20%; and (e) a ribonucleic acid (RNA) molecule. In some embodiments, the LNP contains: (a) cKK-E12 with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 3.5%. In some embodiments, the LNP contains: (a) cKK-E12 with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 5%. In some embodiments, the LNP contains: (a) cKK-E12 with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 10%. In some embodiments, the LNP contains: (a) cKK-E12 with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 3.5%. In some embodiments, the LNP contains: (a) cKK-E12 with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 10%.

### C. Method for Producing LNPs

Provided herein are methods for producing LNPs for delivering a nucleic acid molecule into a T cell and uses thereof, the lipid nanoparticles containing an ionizable lipid, a helper lipid, a polyethylene glycol (PEG) lipid, cholesterol, and a DNA molecule or an RNA molecule. In some embodiments, the RNA and DNA LNPs described herein may be produced by methods described herein.

In some embodiments, the method includes (1) adding to an organic solvent comprising ethanol (a) an ionizable lipid, wherein the lipid is selected from the group consisting of OF-C4-Deg-Lin or an analog thereof, cKK-E12 or an analog thereof, and DLin-KC2-DMA or an analog thereof; (b) a helper lipid; (c) a polyethylene glycol (PEG)-conjugated lipid; and (d) cholesterol, thereby generating an organic phase; (2) adding to an aqueous solvent having an acidic pH, a deoxyribonucleic acid (DNA) molecule, thereby generating an aqueous phase; and (3) combining the organic phase and the aqueous phase by laminar flow mixing in a device, thereby generating a LNP containing DNA.

In some embodiments, the ionizable lipid is DLin-KC2-DMA or an analog thereof. In some embodiments, the ionizable lipid is DLin-KC2-DMA. In some embodiments, the ionizable lipid is OF-C4-Deg-Lin or an analog thereof. In some embodiments, the ionizable lipid is OF-C4-Deg-Lin. In some embodiments, the ionizable lipid is cKK-E12 or an analog thereof. In some embodiments, the ionizable lipid is cKK-E12.

In some embodiments, the method includes (1) adding to an organic solvent comprising ethanol (a) an ionizable lipid, wherein the lipid is selected from the group consisting of OF-C4-Deg-Lin or an analog thereof, cKK-E12 or an analog thereof, DLin-MC3-DMA or an analog thereof, and DLin-KC2-DMA or an analog thereof; (b) a helper lipid; (c) a polyethylene glycol (PEG)-conjugated lipid; and (d) cholesterol, thereby generating an organic phase; (2) adding to an aqueous solvent having an acidic pH, a ribonucleic acid (RNA) molecule, thereby generating an aqueous phase; and (3) combining the organic phase and the aqueous phase by laminar flow mixing in a device, thereby generating a LNP containing RNA.

In some embodiments, the ionizable lipid is DLin-MC3-DMA or an analog thereof. In some embodiments, the ionizable lipid is DLin-MC3-DMA. In some embodiments, the ionizable lipid is DLin-KC2-DMA or an analog thereof. In some embodiments, the ionizable lipid is DLin-KC2-DMA. In some embodiments, the ionizable lipid is OF-C4-Deg-Lin or an analog thereof. In some embodiments, the ionizable lipid is OF-C4-Deg-Lin. In some embodiments, the ionizable lipid is cKK-E12 or an analog thereof. In some embodiments, the ionizable lipid is cKK-E12.

In some embodiments, the flow rate of the aqueous phase in the device is between about 8 mL/min and about 10 mL/min. In some embodiments, the flow rate of the aqueous phase in the device is about 8 mL/min. In some embodiments, the flow rate of the aqueous phase in the device is about 9 mL/min. In some embodiments, the flow rate of the aqueous phase in the device is about 10 mL/min. In some embodiments, the flow rate of the organic phase in the device is between about 2 mL/min and about 4 mL/min. In some embodiments, the flow rate of the organic phase in the device is about 2 mL/min. In some embodiments, the flow rate of the organic phase in the device is about 3 mL/min. In some embodiments, the flow rate of the organic phase in the device is about 4 mL/min. In some embodiments, the ratio of the aqueous phase flow rate to the organic phase flow rate is about 3:1. In some embodiments, the flow rate of the aqueous phase is about 9 mL/min, and the flow rate of the organic phase is about 3 mL/min.

In some embodiments, the aqueous solvent is an acetate buffer. In some embodiments, the pH of the acetate buffer is between about 3.0 and about 4.5. In some embodiments, the pH of the acetate buffer is about 3.0. In some embodiments, the pH of the acetate buffer is about 3.5. In some embodiments, the pH of the acetate buffer is about 4.0. In some embodiments, the pH of the acetate buffer is about 4.5.

In some embodiments, the molarity of the acetate buffer is between about 10 mM and about 300 mM, between about 15 mM and about 275 mM, between about 20 mM and about 250 mM, between about 25 mM and about 200 mM, or between about 30 mM mL/min and about 150 mM. In some embodiments, the molarity of the acetate buffer is between about 10 mM and about 300 mM. In some embodiments, the molarity of the acetate buffer is between about 15 mM and about 275 mM. In some embodiments, the molarity of the acetate buffer is between about 20 mM and about 250 mM. In some embodiments, the molarity of the acetate buffer is between about 25 mM and about 200 mM. In some embodiments, the molarity of the acetate buffer is between about 30 mM and about 150 mM.

In some embodiments, the molarity of the acetate buffer is about 25 mM, about 112.5 mM, or about 200 mM. In some embodiments, the molarity of the acetate buffer is about 25 mM. In some embodiments, the molarity of the acetate buffer is about 112.5 mM. In some embodiments, the molarity of the acetate buffer is about 200 mM.

In some embodiments, the method comprises collecting the generated LNP from the device in the acetate buffer. In some embodiments, the method comprises washing the collected LNP with an isotonic buffer. In some embodiments, isotonic buffer is phosphate buffered saline (PBS). In some embodiments, the pH of the isotonic buffer is about 7.4.

In some embodiments, the method is carried out at about room temperature. In some embodiments, the method is carried out between about 20 degrees Celsius and about 25 degrees Celsius. In some embodiments, the method is carried out at about 20 degrees Celsius.

In some embodiments, the method includes storing the generated LNP at about 4 degrees Celsius for at least about an hour, at least about 2 hours, at least about 4 hours, at least about 6 hours, at least about 8 hours, or at least about 12 hours. In some embodiments, the method includes storing the generated LNP at about 4 degrees Celsius for at least about an hour. In some embodiments, the method includes storing the generated LNP at about 4 degrees Celsius for at least about 2 hours. In some embodiments, the method includes storing the generated LNP at about 4 degrees Celsius for at least about 4 hours. In some embodiments, the method includes storing the generated LNP at about 4 degrees Celsius for at least about 6 hours. In some embodiments, the method includes storing the generated LNP at about 4 degrees Celsius for at least about 8 hours. In some embodiments, the method includes storing the generated LNP at about 4 degrees Celsius for about 12 hours. In some embodiments, the method includes storing the generated LNP at about 4 degrees Celsius for about 18 hours. In some embodiments, the method includes storing the generated LNP at about 4 degrees Celsius for about 24 hours. In some embodiments, the method includes storing the generated LNP at about 12 degrees Celsius for between about 2 hours and about 12 hours. In some embodiments, the method includes storing the generated LNP at about 12 degrees Celsius for at least about 2 hours.

In some embodiments, the method includes sterile filtering the LNPs, or a composition thereof. In some embodiments, the method includes sterile filtering the LNPs or a composition thereof following storage of the lipid nanoparticle composition at 4 degrees Celsius.

### D. Exemplary Features of LNPs

In some embodiments, the size of an LNP provided herein is measured by dynamic light scattering (DLS).

In some embodiments, the LNP comprises a DNA molecule. In some embodiments, the size of the LNP as measured by DLS (Z-ave) is between about 50 nm and about 200 nm, between about 75 nm and about 175 nm, or between about 100 nm and about 150 nm. In some embodiments, the size of the LNP (Z-ave) is between about 50 nm and about 200 nm. In some embodiments, the size of the LNP (Z-ave) is between about 75 nm and about 175 nm. In some embodiments, the size of the LNP (Z-ave) is between about 100 nm and about 150 nm. In some embodiments, the size of the LNP (Z-ave) is about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the size of the LNP (Z-ave) is about 50 nm. In some embodiments, the size of the LNP (Z-ave) is about 60 nm. In some embodiments, the size of the LNP (Z-ave) is about 70 nm. In some embodiments, the size of the LNP (Z-ave) is about 80 nm. In some embodiments, the size of the LNP (Z-ave) is about 90 nm. In some embodiments, the size of the LNP (Z-ave) is about 100 nm. In some embodiments, the size of the LNP (Z-ave) is about 110 nm. In some embodiments, the size of the LNP (Z-ave) is about 120 nm. In some embodiments, the size of the LNP (Z-ave) is about 130 nm. In some embodiments, the size of the LNP (Z-ave) is about 140 nm. In some embodiments, the size of the LNP (Z-ave) is about 150 nm. In some embodiments, the size of the LNP (Z-ave) is about 160 nm. In some embodiments, the size of the LNP (Z-ave) is about 170 nm. In some embodiments, the size of the LNP (Z-ave) is about 180 nm. In some embodiments, the size of the LNP (Z-ave) is about 190 nm. In some embodiments, the size of the LNP (Z-ave) is about 200 nm.

In some embodiments, the LNP comprises an RNA molecule. In some embodiments, the size of the LNP as measured by DLS (Z-ave) is between about 25 nm and about 175 nm, between about 50 nm and about 125 nm, or between about 75 nm and about 100 nm. In some embodiments, the size of the LNP (Z-ave) is between about 25 nm and about 175 nm. In some embodiments, the size of the LNP (Z-ave) is between about 50 nm and about 125 nm. In some embodiments, the size of the LNP (Z-ave) is between about 75 nm and about 100 nm. In some embodiments, the size of the LNP (Z-ave) is about 25 nm, about 30 nm, about 35 nm, about 40 nm, about 45 nm, about 50 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm. In some embodiments, the size of the LNP (Z-ave) is about 25 nm. In some embodiments, the size of the LNP (Z-ave) is about 30 nm. In some embodiments, the size of the LNP (Z-ave) is about 35 nm. In some embodiments, the size of the LNP (Z-ave) is about 40 nm. In some embodiments, the size of the LNP (Z-ave) is about 45 nm. In some embodiments, the size of the LNP (Z-ave) is about 50 nm. In some embodiments, the size of the LNP (Z-ave) is about 55 nm. In some embodiments, the size of the LNP (Z-ave) is about 60 nm. In some embodiments, the size of the LNP (Z-ave) is about 65 nm. In some embodiments, the size of the LNP (Z-ave) is about 70 nm. In some embodiments, the size of the LNP (Z-ave) is about 75 nm. In some embodiments, the size of the LNP (Z-ave) is about 80 nm. In some embodiments, the size of the LNP (Z-ave) is about 85 nm. In some embodiments, the size of the LNP (Z-ave) is about 90 nm. In some embodiments, the size of the LNP (Z-ave) is about 95 nm. In some embodiments, the size of the LNP (Z-ave) is about 100 nm. In some embodiments, the size of the LNP (Z-ave) is about 105 nm. In some embodiments, the size of the LNP (Z-ave) is about 110 nm. In some embodiments, the size of the LNP (Z-ave) is about 115 nm. In some embodiments, the size of the LNP (Z-ave) is about 120 nm. In some embodiments, the size of the LNP (Z-ave) is about 125 nm.

In some embodiments, the LNPs and compositions thereof provided herein are stable at temperatures lower than room temperature. In some embodiments, the size of the LNPs provided herein are stable at temperatures lower than room temperature. In some embodiments, the size of the LNP is assessed by any technique known in the art, such as dynamic light scattering (DLS). In some embodiments, the amount of nucleic acid contained in the LNPs provided herein are stable at temperatures lower than room temperature.

In some embodiments, the size of the LNP does not change more than about 10% when the LNP is stored at about 4 degrees Celsius for at least about one day, at least about 3 days, at least about 5 days, or at least about 7 days. In some embodiments, the size of the LNP does not change more than about 10% when the LNP is stored at about 4 degrees Celsius for at least about 3 days. In some embodiments, the size of the LNP does not change more than about 10% when the LNP is stored at about 4 degrees Celsius for at least about 5 days. In some embodiments, the size of the LNP does not change more than about 10% when the LNP is stored at about 4 degrees Celsius for at least about 7 days. In some embodiments, the size of the LNP does not change more than about 10% when the LNP is stored at about 4 degrees Celsius for about 3 days. In some embodiments, the size of the LNP does not change more than about 10% when the LNP is stored at about 4 degrees Celsius for about 5 days. In some embodiments, the size of the LNP does not change more than about 10% when the LNP is stored at about 4 degrees Celsius for about 7 days. In some embodiments, the size of the LNP does not change more than about 10% when the LNP is stored at about 4 degrees Celsius for about 10 days. In some embodiments, the size of the LNP does not change more than about 10% when the LNP is stored at about 4 degrees Celsius for about 14 days.

In some embodiments, the size of the LNP does not change more than about 5% when the LNP is stored at about 4 degrees Celsius for at least about one day, at least about 3 days, at least about 5 days, or at least about 7 days. In some embodiments, the size of the LNP does not change more than about 5% when the LNP is stored at about 4 degrees Celsius for at least about 3 days. In some embodiments, the size of the LNP does not change more than about 5% when the LNP is stored at about 4 degrees Celsius for at least about 5 days. In some embodiments, the size of the LNP does not change more than about 5% when the LNP is stored at about 4 degrees Celsius for at least about 7 days. In some embodiments, the size of the LNP does not change more than about 5% when the LNP is stored at about 4 degrees Celsius for about 3 days. In some embodiments, the size of the LNP does not change more than about 5% when the LNP is stored at about 4 degrees Celsius for about 5 days. In some embodiments, the size of the LNP does not change more than about 5% when the LNP is stored at about 4 degrees Celsius for about 7 days. In some embodiments, the size of the LNP does not change more than about 5% when the LNP is stored at about 4 degrees Celsius for about 10 days. In some embodiments, the size of the LNP does not change more than about 5% when the LNP is stored at about 4 degrees Celsius for about 14 days.

In some embodiments, the size of the LNP does not change more than about 1% when the LNP is stored at about 4 degrees Celsius for at least about one day, at least about 3 days, at least about 5 days, or at least about 7 days. In some embodiments, the size of the LNP does not change more than about 1% when the LNP is stored at about 4 degrees Celsius for at least about 3 days. In some embodiments, the size of the LNP does not change more than about 1% when the LNP is stored at about 4 degrees Celsius for at least about 5 days. In some embodiments, the size of the LNP does not change more than about 1% when the LNP is stored at about 4 degrees Celsius for at least about 7 days. In some embodiments, the size of the LNP does not change more than about 1% when the LNP is stored at about 4 degrees Celsius for about 3 days. In some embodiments, the size of the LNP does not change more than about 1% when the LNP is stored at about 4 degrees Celsius for about 5 days. In some embodiments, the size of the LNP does not change more than about 1% when the LNP is stored at about 4 degrees Celsius for about 7 days. In some embodiments, the size of the LNP does not change more than about 1% when the LNP is stored at about 4 degrees Celsius for about 10 days. In some embodiments, the size of the LNP does not change more than about 1% when the LNP is stored at about 4 degrees Celsius for about 14 days.

In some embodiments, the amount of nucleic acid contained in the LNP does not change more than about 10% when the LNP is stored at about 4 degrees Celsius for at least about one day, at least about 3 days, at least about 5 days, or at least about 7 days. In some embodiments, the amount of nucleic acid contained in the LNP (e.g. DNA or RNA load) does not change more than about 10% when the LNP is stored at about 4 degrees Celsius for at least about one day. In some embodiments, the amount of nucleic acid contained in the LNP (e.g. DNA or RNA load) does not change more than about 10% when the LNP is stored at about 4 degrees Celsius for at least about 3 days. In some embodiments, the amount of nucleic acid contained in the LNP (e.g. DNA or RNA load) does not change more than about 10% when the LNP is stored at about 4 degrees Celsius for at least about 5 days. In some embodiments, the amount of nucleic acid contained in the LNP (e.g. DNA or RNA load) does not change more than about 10% when the LNP is stored at about 4 degrees Celsius for at least about 7 days. In some embodiments, the amount of nucleic acid contained in the LNP (e.g. DNA or RNA load) does not change more than about 10% when the LNP is stored at about 4 degrees Celsius for about one day. In some embodiments, the amount of nucleic acid contained in the LNP (e.g. DNA or RNA load) does not change more than about 10% when the LNP is stored at about 4 degrees Celsius for about 3 days. In some embodiments, the amount of nucleic acid contained in the LNP (e.g. DNA or RNA load) does not change more than about 10% when the LNP is stored at about 4 degrees Celsius for about 5 days. In some embodiments, the amount of nucleic acid contained in the LNP (e.g. DNA or RNA load) does not change more than about 10% when the LNP is stored at about 4 degrees Celsius for about 7 days. In some embodiments, the amount of nucleic acid contained in the LNP (e.g. DNA or RNA load) does not change more than about 10% when the LNP is stored at about 4 degrees Celsius for about 10 days. In some embodiments, the amount of nucleic acid contained in the LNP (e.g. DNA or RNA load) does not change more than about 10% when the LNP is stored at about 4 degrees Celsius for about 14 days.

In some embodiments, the amount of nucleic acid contained in the LNP does not change more than about 5% when the LNP is stored at about 4 degrees Celsius for at least about one day, at least about 3 days, at least about 5 days, or at least about 7 days. In some embodiments, the amount of nucleic acid contained in the LNP (e.g. DNA or RNA load) does not change more than about 5% when the LNP is stored at about 4 degrees Celsius for at least about one day. In some embodiments, the amount of nucleic acid contained in the LNP (e.g. DNA or RNA load) does not change more than about 5% when the LNP is stored at about 4 degrees Celsius for at least about 3 days. In some embodiments, the amount of nucleic acid contained in the LNP (e.g. DNA or RNA load) does not change more than about 5% when the LNP is stored at about 4 degrees Celsius for at least about 5 days. In some embodiments, the amount of nucleic acid contained in the LNP (e.g. DNA or RNA load) does not change more than about 5% when the LNP is stored at about 4 degrees Celsius for at least about 7 days. In some embodiments, the amount of nucleic acid contained in the LNP (e.g. DNA or RNA load) does not change more than about 5% when the LNP is stored at about 4 degrees Celsius for about one day. In some embodiments, the amount of nucleic acid contained in the LNP (e.g. DNA or RNA load) does not change more than about 5% when the LNP is stored at about 4 degrees Celsius for about 3 days. In some embodiments, the amount of nucleic acid contained in the LNP (e.g. DNA or RNA load) does not change more than about 5% when the LNP is stored at about 4 degrees Celsius for about 5 days. In some embodiments, the amount of nucleic acid contained in the LNP (e.g. DNA or RNA load) does not change more than about 5% when the LNP is stored at about 4 degrees Celsius for about 7 days. In some embodiments, the amount of nucleic acid contained in the LNP (e.g. DNA or RNA load) does not change more than about 5% when the LNP is stored at about 4 degrees Celsius for about 10 days. In some embodiments, the amount of nucleic acid contained in the LNP (e.g. DNA or RNA load) does not change more than about 5% when the LNP is stored at about 4 degrees Celsius for about 14 days.

In some embodiments, the amount of nucleic acid contained in the LNP does not change more than about 1% when the LNP is stored at about 4 degrees Celsius for at least about one day, at least about 3 days, at least about 5 days, or at least about 7 days. In some embodiments, the amount of nucleic acid contained in the LNP (e.g. DNA or RNA load) does not change more than about 1% when the LNP is stored at about 4 degrees Celsius for at least about one day. In some embodiments, the amount of nucleic acid contained in the LNP (e.g. DNA or RNA load) does not change more than about 1% when the LNP is stored at about 4 degrees Celsius for at least about 3 days. In some embodiments, the amount of nucleic acid contained in the LNP (e.g. DNA or RNA load) does not change more than about 1% when the LNP is stored at about 4 degrees Celsius for at least about 5 days. In some embodiments, the amount of nucleic acid contained in the LNP (e.g. DNA or RNA load) does not change more than about 1% when the LNP is stored at about 4 degrees Celsius for at least about 7 days. In some embodiments, the amount of nucleic acid contained in the LNP (e.g. DNA or RNA load) does not change more than about 1% when the LNP is stored at about 4 degrees Celsius for about one day. In some embodiments, the amount of nucleic acid contained in the LNP (e.g. DNA or RNA load) does not change more than about 1% when the LNP is stored at about 4 degrees Celsius for about 3 days. In some embodiments, the amount of nucleic acid contained in the LNP (e.g. DNA or RNA load) does not change more than about 1% when the LNP is stored at about 4 degrees Celsius for about 5 days. In some embodiments, the amount of nucleic acid contained in the LNP (e.g. DNA or RNA load) does not change more than about 1% when the LNP is stored at about 4 degrees Celsius for about 7 days. In some embodiments, the amount of nucleic acid contained in the LNP (e.g. DNA or RNA load) does not change more than about 1% when the LNP is stored at about 4 degrees Celsius for about 10 days. In some embodiments, the amount of nucleic acid contained in the LNP (e.g. DNA or RNA load) does not change more than about 1% when the LNP is stored at about 4 degrees Celsius for about 14 days.

### II. Co-Formulated Lipid Nanoparticles (Co-LNPS)

Provided herein are co-formulated lipid nanoparticles (co-LNPs) and compositions containing the same, such as for delivering a nucleic acid molecule into a cell (e.g. a T cell). In some embodiments, a co-formulated LNP is generated by fusion (e.g., pH neutralization-mediated fusion) of more than one LNP into a single, co-formulated LNP.

In some embodiments, the nucleic acid molecule is an RNA molecule, a DNA molecule, or both. In some embodiments, the co-formulated LNP comprises an ionizable lipid, such as an ionizable lipid or lipidoid containing a diketopiperazine ring core. In some embodiments, the LNP comprises an ionizable lipid, such as DLin-KC2-DMA or an analog thereof. In some embodiments, the LNP comprises an ionizable lipid, such as DLin-MC3-DMA or an analog thereof. In some embodiments, the LNP further comprise a helper lipid, a polyethylene glycol (PEG)-conjugated lipid, and cholesterol. Also provided herein are methods for producing the co-formulated LNPs and compositions and uses thereof, such as in connection with cell therapy.

### A. DNA and RNA Co-LNPs

### 1. Components

Provided herein are co-formulated LNPs (co-LNPs) comprising a ribonucleic acid (RNA) molecule and a deoxyribonucleic acid (DNA) molecule. In some cases, the co-LNP can be generated to contain one or more types of RNA molecules and/or one or more types of DNA molecules. In some cases, a co-formulated LNP is generated from one or more RNA LNPs, including any of those as described in Section I.B., and one or more DNA LNPs, including any of those as described in Section I.A. It is contemplated herein that the provision of a co-LNP containing RNA and DNA may result in higher gene editing efficiency, as compared to the provision of separate RNA LNPs and DNA LNPs. In some cases, it is contemplated that the improved efficiency may be due to reduced competitive uptake, in that the cell is only required to uptake a co-LNP, rather than multiple types of LNPs (e.g., RNA LNP and DNA LNP).

In some embodiments, the co-LNP comprises a DNA molecule, an RNA molecule, a first ionizable lipid, and a second ionizable lipid. In some embodiments, the co-LNP further comprises a third ionizable lipid (i.e., a tri-LNP). In some embodiments, the DNA molecule is associated with the first ionizable lipid, and the RNA molecule is associate with the second and/or third ionizable lipid.

Also provided herein are co-LNPs comprising a fusion of a first LNP and a second LNP, wherein: (1) the first LNP comprises a DNA molecule and a first ionizable lipid; and (2) the second LNP comprises an RNA molecule and a second ionizable lipid. In some embodiments, the fusion of the first LNP and the second LNP is performed by methods disclosed herein. In some embodiments, the volumetric ratio of the first LNP to the second LNP is between about 1:3 and about 3:1. In some embodiments, the volumetric ratio of the first LNP to the second LNP is about 1:3. In some embodiments, the volumetric ratio of the first LNP to the second LNP is about 1:2. In some embodiments, the volumetric ratio of the first LNP to the second LNP is about 1:1. In some embodiments, the volumetric ratio of the first LNP to the second LNP is about 2:1. In some embodiments, the volumetric ratio of the first LNP to the second LNP is about 3:1.

In some embodiments, the first LNP and second LNP, prior to fusion, are precursor LNPs (also referred to herein as acidic LNPs) that are not fully formed. As set forth herein, precursor LNPs are generated in an acidic environment (e.g., at a pH between about 4 and about 5). It will be understood that in an acidic environment, the individual lipids that ultimately comprise the shell of LNP are loosely associated (aggregated). Following pH neutralization, the extent of lipid association increases, hence generating the fully formed fused LNP. Nonetheless, in the precursor LNPs, the extent of association between the ionizable lipid and the nucleic acid molecule would be high owing to the acidic nature of the medium. Without being bound by theory, it is expected that after pH neutralization and fusion of the first and second LNP, the nucleic acid molecule from each of the precursor LNPs remains substantially associated with the ionizable lipid to which it was bound in the individual precursor LNPs. For instance, the DNA molecule would remain substantially associated with the first ionizable lipid and the RNA molecule would remain substantially associated with the second ionizable lipid. Accordingly, the fused co-LNPs would not demonstrate substantial exchange of the nucleic acids from one ionizable lipid to another upon mixing and neutralization.

In some embodiments, the first ionizable lipid of the first LNP forms an ionic bond with the DNA molecule and the second ionizable lipid of the second LNP forms an ionic bond with the RNA molecule. In some embodiments, following fusion of the first and second precursor LNPs to form the fused co-LNP, the first ionizable lipid remains substantially associated (complexed) with the DNA molecule and the second ionizable lipid remains substantially associated (complexed) with the RNA molecule.

In some embodiments, following fusion of the first and second precursor LNPs to form the fused co-LNP, the first ionizable lipid remains substantially associated (complexed) with the DNA molecule and the second ionizable lipid remains substantially associated (complexed) with the RNA molecule. For instance, in some embodiments, more than 75% of the first ionizable lipid remains associated with the DNA molecule and more than 75% of the second ionizable lipid remains associated with the RNA molecule in the fused co-LNP. In some embodiments, more than 80% of the first ionizable lipid remains associated with the DNA molecule and more than 80% of the second ionizable lipid remains associated with the RNA molecule in the fused co-LNP. In some embodiments, more than 85% of the first ionizable lipid remains associated with the DNA molecule and more than 85% of the second ionizable lipid remains associated with the RNA molecule in the fused co-LNP. In some embodiments, more than 90% of the first ionizable lipid remains associated with the DNA molecule and more than 90% of the second ionizable lipid remains associated with the RNA molecule in the fused co-LNP. In some embodiments, more than 95% of the first ionizable lipid remains associated with the DNA molecule and more than 95% of the second ionizable lipid remains associated with the RNA molecule in the fused co-LNP. In some embodiments, more than 99% of the first ionizable lipid remains associated with the DNA molecule and more than 99% of the second ionizable lipid remains associated with the RNA molecule in the fused co-LNP. In some embodiments, all of the first ionizable lipid remains associated with the DNA molecule and all of the second ionizable lipid remains associated with the RNA molecule in the fused co-LNP. In some embodiments, more than 75% of the first ionizable lipid remains associated with the DNA molecule and more than 75% of the second ionizable lipid remains associated with the RNA molecule in the fused co-LNP. In some embodiments, between about 75% and about 90% of the first ionizable lipid remains associated with the DNA molecule and between about 75% and about 90% of the second ionizable lipid remains associated with the RNA molecule in the fused co-LNP. In some embodiments, between about 75% and about 99% of the first ionizable lipid remains associated with the DNA molecule and between about 75% and about 99% of the second ionizable lipid remains associated with the RNA molecule in the fused co-LNP.

In some embodiments, the shell of the fused co-LNP comprises a mixture of lipids from each of the precursor LNPs. In some embodiments, the shell of the fused co-LNP is a hybrid of the lipids that comprise the two precursor LNPs. For instance, in some embodiments where the first LNP (prior to fusion) comprises a mass fraction of about 20% of a first helper lipid and the second LNP (prior to fusion) comprises a mass fraction of about 8% of a second helper lipid, the fused co-LNP comprises a mass fraction of greater than 8% and less than 20% total helper lipid, wherein the shell of the co-LNP comprises the first and the second helper lipid. In some such embodiments, the shell of the co-LNP comprises a mass fraction from about 14% to 16% helper lipid.

In some embodiments, the fusion further comprises (3) a third LNP comprising an RNA molecule and a third ionizable lipid. Such co-LNPs are also referred to herein as tri-LNPs. In some embodiments, the volumetric ratio of the first LNP to the second and third LNPs is between about 1:3 and about 3:1. In some embodiments, the volumetric ratio of the first LNP to the second and third LNPs is about 1:3. In some embodiments, the volumetric ratio of the first LNP to the second and third LNPs is about 1:2. In some embodiments, the volumetric ratio of the first LNP to the second and third LNPs is about 1:1. In some embodiments, the volumetric ratio of the first LNP to the second and third LNPs is about 2:1. In some embodiments, the volumetric ratio of the first LNP to the second and third LNPs is about 3:1.

In some embodiments, the co-LNP comprises (i) a guide RNA (gRNA) and/or mRNA encoding a recombinant nuclease capable of inducing a DNA break; and (ii) HDR template (HDRt) DNA. In some embodiments, the co-LNP comprises (i) a guide RNA (gRNA) and mRNA encoding a recombinant nuclease capable of inducing a DNA break; and (ii) HDR template (HDRt) DNA. In some embodiments, the co-LNP comprises (i) a guide RNA (gRNA) complexed with a recombinant nuclease capable of inducing a DNA break; and (ii) HDR template (HDRt) DNA. In some embodiments, the gRNA is a single guide RNA (sgRNA) comprising a crispr RNA (crRNA) and a tracrRNA.

In some embodiments, the HDRt DNA encodes a recombinant receptor (e.g., a CAR). In some embodiments, the recombinant nuclease is a zinc finger nuclease (ZFN), a transcription activator-like effector nuclease (TALEN), or a CRISPR-associated nuclease (Cas). In some embodiments, the recombinant nuclease is a zinc finger nuclease (ZFN). In some embodiments, the recombinant nuclease is a transcription activator-like effector nuclease (TALEN). In some embodiments, the recombinant nuclease is a CRISPR-associated nuclease (Cas). In some of any such embodiments, the Cas nuclease is selected from the group consisting of Cas3, Cas9, Cas10, Cas12, Cas12a, and Cas13. In some of any such embodiments, the Cas nuclease is Cas9. In some of any such embodiments, the Cas nuclease is Cas9 or a variant thereof. In some embodiments, the Cas nuclease is an enhanced specificity Cas9 (eSpCas9). In some embodiments, the Cas nuclease is a high fidelity Cas9 (HiFi Cas9). In some of any such embodiments, the Cas9 is from a bacteria selected from the group consisting of Streptococcus pyogenes, Staphylococcus aureus, Neisseria meningitides, Campylobacter jejuni, and Streptococcus thermophilis. In some of any such embodiments, the Cas9 is from Streptococcus pyogenes. In some of any such embodiments, the Cas9 or a variant thereof is from Streptococcus pyogenes. In some embodiments, the Cas is Cas12a.

In some embodiments, the co-LNP comprises (i) mRNA encoding a transposase; and (ii) a transposon. In some embodiments, the transposase is selected from the group consisting of: Sleeping Beauty, piggyBac, TcBuster, Frog Prince, Tol2, Tc1/mariner, or a derivative thereof having transposase activity. In some embodiments, the transposase is Sleeping Beauty, PiggyBac, or TcBuster. In some embodiments, the transposase is Sleeping Beauty. In some embodiments, the transposase is PiggyBac. In some embodiments, the transposase is TcBuster. In some embodiments, the transposon comprises a transgene encoding for a recombinant receptor (e.g., a CAR).

In some embodiments, the co-LNP comprises a first and second ionizable lipid. In some embodiments, the co-LNP further comprises a third ionizable lipid. In some embodiments, the co-LNP is generated from two LNPs, such that the co-LNP comprises a first and a second ionizable lipid. In some embodiments, the co-LNP is generated from three LNPs, such that the co-LNP comprises a first, second, and third ionizable lipid.

In some embodiments, the first, second, and/or third ionizable lipid is positively charged (e.g. a cationic lipid). In some embodiments, the first, second, and/or third ionizable lipid is a cationic lipid, including but not limited to those described in U.S. Pat. Nos. 9,593,077; 9,365,610; 9,670,152; and 9,458,090. In some embodiments, the first, second and/or third ionizable lipid is a cationic lipid, including but not limited to those described in Published PCT application WO2013149140. In some embodiments, the first, second, and/or third ionizable lipid is a cationic lipid, including but not limited to those described in published US Patent application US2019084965; US2019106379. In some embodiments, the first, second, and/or third ionizable lipid is a cationic lipid, including but not limited to those described in Published EP application EP2830595.

In some embodiments, the first, second, and/or third ionizable lipid is selected from the group consisting of D-Lin, 2,2-dilinoleyl-4-dimethylaminoethyl-[1,3]-dioxolane (DLin-KC2-DMA), or an analog thereof, 4-(dimethylamino)-butanoic acid, (10Z,13Z)-1-(9Z,12Z)-9,12-octadecadien-1-yl-10,13-nonadecadien-1-yl ester (DLin-MC3-DMA), or an analog thereof, 1 OF-C4-Deg-Lin, or an analog thereof, and cKK-E12, or an analog thereof.

In some embodiments, the first ionizable lipid is an amino lipidoid comprising a diketopiperazine ring core. In some embodiments, the first ionizable lipid comprises an unsaturated tail. In some embodiments, the first ionizable lipid comprises an unsaturated linoleic tail. In some embodiments, the first ionizable lipid is: (a) OF-C4-Deg-Lin, or an analog thereof; or (b) cKK-E12, or an analog thereof. In some embodiments, the first ionizable lipid is OF-C4-Deg-Lin, or an analog thereof. In some embodiments, the first ionizable lipid is OF-C4-Deg-Lin. In some embodiments, wherein the first ionizable lipid is cKK-E12, or an analog thereof. In some embodiments, wherein the first ionizable lipid is cKK-E12. In some embodiments, the first ionizable lipid is DLin-MC3-DMA, or an analog thereof. In some embodiments, the first ionizable lipid is DLin-MC3-DMA. In some embodiments, the first ionizable lipid is DLin-KC2-DMA, or an analog thereof. In some embodiments, the first ionizable lipid is DLin-KC2-DMA.

In some embodiments, the second ionizable lipid is an amino lipidoid comprising a diketopiperazine ring core. In some embodiments, the second ionizable lipid comprises an unsaturated tail. In some embodiments, the second ionizable lipid comprises an unsaturated linoleic tail. In some embodiments, the second ionizable lipid is: (a) OF-C4-Deg-Lin, or an analog thereof; or (b) cKK-E12, or an analog thereof. In some embodiments, the second ionizable lipid is OF-C4-Deg-Lin, or an analog thereof. In some embodiments, the second ionizable lipid is OF-C4-Deg-Lin. In some embodiments, wherein the second ionizable lipid is cKK-E12, or an analog thereof. In some embodiments, wherein the second ionizable lipid is cKK-E12. In some embodiments, the second ionizable lipid is DLin-MC3-DMA, or an analog thereof. In some embodiments, the second ionizable lipid is DLin-MC3-DMA. In some embodiments, the second ionizable lipid is DLin-KC2-DMA, or an analog thereof. In some embodiments, the second ionizable lipid is DLin-KC2-DMA.

In some embodiments, the third ionizable lipid is an amino lipidoid comprising a diketopiperazine ring core. In some embodiments, the third ionizable lipid comprises an unsaturated tail. In some embodiments, the third ionizable lipid comprises an unsaturated linoleic tail. In some embodiments, the third ionizable lipid is: (a) OF-C4-Deg-Lin, or an analog thereof; or (b) cKK-E12, or an analog thereof. In some embodiments, the third ionizable lipid is OF-C4-Deg-Lin, or an analog thereof. In some embodiments, the third ionizable lipid is OF-C4-Deg-Lin. In some embodiments, wherein the third ionizable lipid is cKK-E12, or an analog thereof. In some embodiments, wherein the third ionizable lipid is cKK-E12. In some embodiments, the third ionizable lipid is DLin-MC3-DMA, or an analog thereof. In some embodiments, the third ionizable lipid is DLin-MC3-DMA. In some embodiments, the third ionizable lipid is DLin-KC2-DMA, or an analog thereof. In some embodiments, the third ionizable lipid is DLin-KC2-DMA.

In some embodiments, the first ionizable lipid and the second ionizable lipid are the same. In some embodiments, the first ionizable lipid and the second ionizable lipid are different. In some embodiments, the second ionizable lipid and the third ionizable lipid are the same. In some embodiments, the second ionizable lipid and the third ionizable lipid are different. In some embodiments, the first, second, and third ionizable lipids are the same. In some embodiments, each of the first, second, and third ionizable lipids are different.

In some embodiments, the co-LNP is generated from the fusion of a RNA LNP and a DNA LNP, such that the co-LNP comprises a first and second ionizable lipid. In some embodiments, prior to fusion with the DNA LNP, the RNA LNP comprised DLin-MC3-DMA. In some embodiments, prior to fusion with the DNA LNP, the RNA LNP comprised cKK-E12. In some embodiments, prior to fusion with the DNA LNP, the RNA LNP comprised OF-C4-Deg-Lin. In some embodiments, prior to fusion with the RNA LNP, the DNA LNP comprised DLin-KC2-DMA. In some embodiments, prior to fusion with the RNA LNP, the DNA LNP comprised cKK-E12. In some embodiments, prior to fusion with the RNA LNP, the DNA LNP comprised OF-C4-Deg-Lin.

In some embodiments, the co-LNP is generated from the fusion of two separate RNA LNPs and a DNA LNP, such that the co-LNP comprises a first, second, and third ionizable lipid. In some embodiments, prior to fusion with the DNA LNP, one of the RNA LNPs comprised DLin-MC3-DMA. In some embodiments, prior to fusion with the DNA LNP, one of the RNA LNPs comprised cKK-E12. In some embodiments, prior to fusion with the DNA LNP, one of the RNA LNPs comprised OF-C4-Deg-Lin. In some embodiments, prior to fusion with the DNA LNP, the other of the RNA LNPs comprised DLin-MC3-DMA. In some embodiments, prior to fusion with the DNA LNP the other of the RNA LNPs comprised cKK-E12. In some embodiments, prior to fusion with the DNA LNP, the other of the RNA LNPs comprised OF-C4-Deg-Lin. In some embodiments, prior to fusion with the RNA LNPs, the DNA LNP comprised DLin-KC2-DMA. In some embodiments, prior to fusion with the RNA LNPs, the DNA LNP comprised cKK-E12. In some embodiments, prior to fusion with the RNA LNPs, the DNA LNP comprised OF-C4-Deg-Lin.

In some embodiments, the co-LNP is generated from the fusion of a DNA LNP and a RNA LNP, such that the co-LNP comprises a first helper lipid and a second helper lipid.

In some embodiments, each of the first and second helper lipids is independently selected from the group consisting of dioleoylphosphatidylethanolamine (DOPE), 1,2-distearoyl-sn-glycero-3-phosphocholine (DSPC), 1-stearoyl-2-oleoyl-sn-glycero-3-phosphocholine (SOPC), and 1,2-dioleoyl-sn-glycero-sn-3-phosphatidylcholine (DOPC). In some embodiments, the first and/or second helper lipid is DOPE. In some embodiments, the first and/or second helper lipid has the structure of

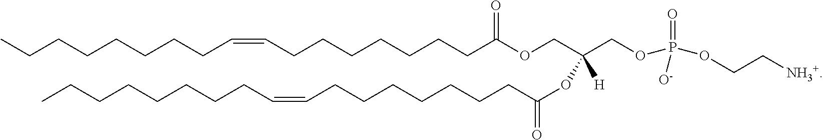

In some embodiments, the first and/or second helper lipid is DSPC. In some embodiments, the first and/or second helper lipid has the structure of

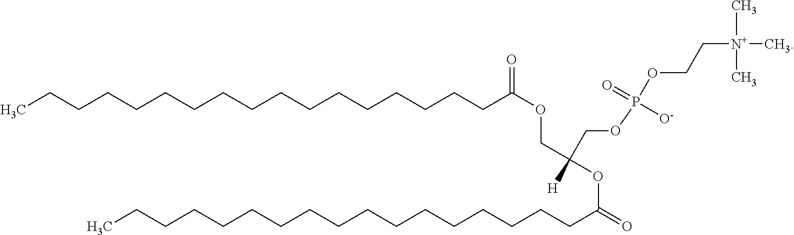 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the first and/or second helper lipid is SOPC. In some embodiments, the first and/or second helper lipid has the structure of

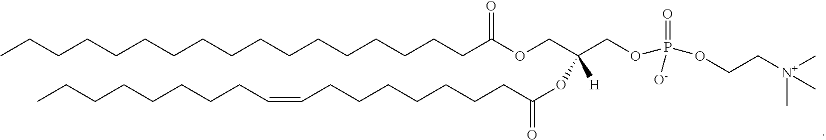 [SMILES: CCCCCCCC/C=C\CCCCCCCC(=O)O[C@H](COC(=O)CCCCCCCCCCCCCCCCC)COP(=O)([O-])OCC[N+](C)(C)C]

In some embodiments, the first and/or second helper lipid is DOPC. In some embodiments, the first and/or second helper lipid has the structure of

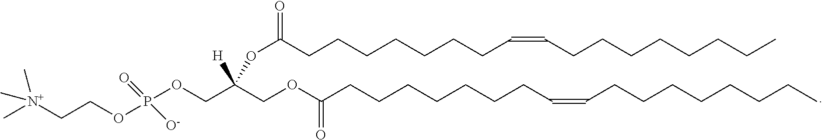 [SMILES: CCCCCCCC/C=C\CCCCCCCC(=O)OC[C@H](COP(=O)([O-])OCC[N+](C)(C)C)OC(=O)CCCCCCC/C=C\CCCCCCCC]

In some embodiments, the co-LNP is generated from the fusion of two separate RNA LNPs and a DNA LNP, such that the co-LNP comprises a first, second, and third helper lipid.

In some embodiments, each of the first, second, and third helper lipids is independently selected from the group consisting of dioleoylphosphatidylethanolamine (DOPE), 1,2-distearoyl-sn-glycero-3-phosphocholine (DSPC), 1-stearoyl-2-oleoyl-sn-glycero-3-phosphocholine (SOPC), and 1,2-dioleoyl-sn-glycero-sn-3-phosphatidylcholine (DOPC). In some embodiments, the first and/or second helper lipid is DOPE. In some embodiments, the first and/or second helper lipid has the structure of

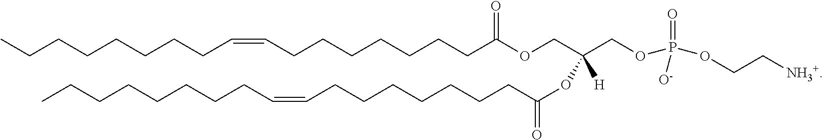

In some embodiments, the first, second, and/or third helper lipid is DSPC. In some embodiments, the first, second, and/or third helper lipid has the structure of

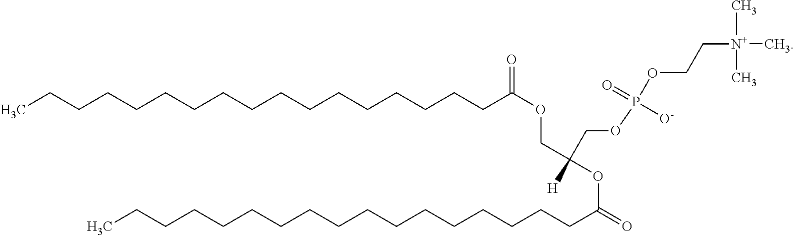 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the first, second, and/or third helper lipid is SOPC. In some embodiments, the first, second, and/or third helper lipid has the structure of

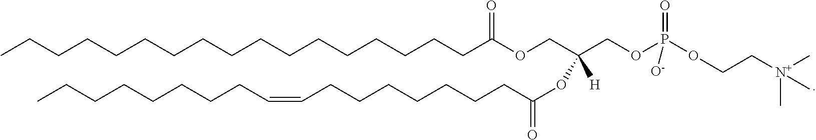 [SMILES: CCCCCCCC/C=C\CCCCCCCC(=O)O[C@H](COC(=O)CCCCCCCCCCCCCCCCC)COP(=O)([O-])OCC[N+](C)(C)C]

In some embodiments, the first, second, and/or third helper lipid is DOPC. In some embodiments, the first, second, and/or third helper lipid has the structure of

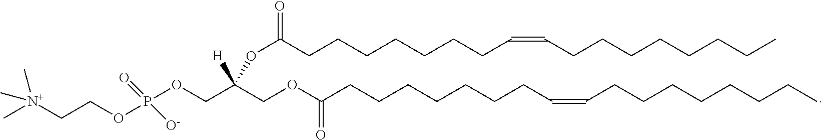 [SMILES: CCCCCCCC/C=C\CCCCCCCC(=O)OC[C@H](COP(=O)([O-])OCC[N+](C)(C)C)OC(=O)CCCCCCC/C=C\CCCCCCCC]

In some embodiments, the co-LNP is generated from the fusion of a RNA LNP and a DNA LNP, such that the co-LNP comprises a first and second helper lipid. In some embodiments, prior to fusion with the DNA LNP, the RNA LNP comprised DSPC. In some embodiments, prior to fusion with the RNA LNP, the DNA LNP comprised SOPC.

In some embodiments, the co-LNP is generated from the fusion of two separate RNA LNPs and a DNA LNP, such that the co-LNP comprises a first, second, and third ionizable lipid. In some embodiments, prior to fusion with the DNA LNP, one of the RNA LNPs comprised DSPC. In some embodiments, prior to fusion with the DNA LNP, the other of the RNA LNPs comprised DSPC. In some embodiments, prior to fusion with the RNA LNPs, the DNA LNP comprised SOPC.

In some embodiments, the co-LNP comprises a non-ionizable cationic lipid. In some embodiments, the mass fraction of the non-ionizable cationic lipid in the co-LNP (or tri-LNP) is between about 0.5% and about 7%. In some embodiments, the mass fraction of the non-ionizable cationic lipid in the co-LNP (or tri-LNP) is between about 0.5% and about 5%. In other embodiments, the mass fraction of the non-ionizable cationic lipid in the co-LNP (or tri-LNP) is between about 1% and about 6%. In other embodiments, the mass fraction of the non-ionizable cationic lipid in the co-LNP (or tri-LNP) is between about 2% and about 5%.

In some embodiments, the non-ionizable cationic lipid is Lipid T1. In some embodiments, the mass fraction of Lipid T1 in the co-LNP is between about 0.1% and about 60%. In some embodiments, the mass fraction of Lipid T1 in the co-LNP is between about 0.1% and about 40%. In some embodiments, the mass fraction of Lipid T1 in the co-LNP is between about 0.2% and about 20%. In some embodiments, the mass fraction of Lipid T1 in the co-LNP is between about 0.2% and about 20%. In some embodiments, the mass fraction of Lipid T1 in the co-LNP is between about 0.5% and about 10%. In some embodiments, the mass fraction of Lipid T1 in the co-LNP is between about 1% and about 7%. In some embodiments, the mass fraction of Lipid T1 in the co-LNP is between about 1% and about 6%. In some embodiments, the mass fraction of Lipid T1 in the co-LNP is between about 2% and about 5%. In some embodiments, the mass fraction of Lipid T1 in the co-LNP is between about 0.2% and about 1%.

In some embodiments, the co-LNP comprises a PEG-conjugated lipid. In some embodiments, the polyethylene glycol (PEG)-conjugated lipid is DMG-PEG2000. In some embodiments, the PEG-conjugated lipid has the structure of

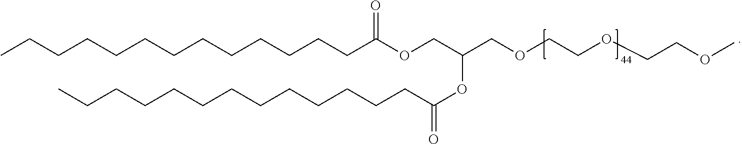

In some embodiments, the co-LNP comprises cholesterol. In some embodiments, cholesterol has the structure of

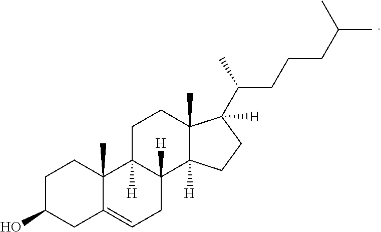

In some embodiments, the size of the co-LNP as measured by DLS (Z-ave) is between about 50 nm and about 150 nm, or between about 75 nm and about 125 nm. In some embodiments, the size of the co-LNP (Z-ave) is between about 50 nm and about 150 nm. In some embodiments, the size of the co-LNP (Z-ave) is between about 75 nm and about 125 nm. In some embodiments, the size of the co-LNP (Z-ave) is about 50 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm. In some embodiments, the size of the co-LNP (Z-ave) is about 50 nm. In some embodiments, the size of the co-LNP (Z-ave) is about 55 nm. In some embodiments, the size of the co-LNP (Z-ave) is about 60 nm. In some embodiments, the size of the co-LNP (Z-ave) is about 65 nm. In some embodiments, the size of the co-LNP (Z-ave) is about 70 nm. In some embodiments, the size of the co-LNP (Z-ave) is about 75 nm. In some embodiments, the size of the co-LNP (Z-ave) is about 80 nm. In some embodiments, the size of the co-LNP (Z-ave) is about 85 nm. In some embodiments, the size of the co-LNP (Z-ave) is about 90 nm. In some embodiments, the size of the co-LNP (Z-ave) is about 95 nm. In some embodiments, the size of the co-LNP (Z-ave) is about 100 nm. In some embodiments, the size of the co-LNP (Z-ave) is about 105 nm. In some embodiments, the size of the co-LNP (Z-ave) is about 110 nm. In some embodiments, the size of the co-LNP (Z-ave) is about 115 nm. In some embodiments, the size of the co-LNP (Z-ave) is about 120 nm. In some embodiments, the size of the co-LNP (Z-ave) is about 125 nm. In some embodiments, the size of the co-LNP (Z-ave) is about 130 nm. In some embodiments, the size of the co-LNP (Z-ave) is about 135 nm. In some embodiments, the size of the co-LNP (Z-ave) is about 140 nm. In some embodiments, the size of the co-LNP (Z-ave) is about 145 nm. In some embodiments, the size of the co-LNP (Z-ave) is about 150 nm.

In some embodiments, the co-LNPs formed by fusion methods described herein show a fluorescence energy transfer (FRET). In particular embodiments, FRET is demonstrated by attaching individual fluorescent dyes, a donor and acceptor, to each of the precursor LNPs prior to mixing (under acidic conditions) and neutralization. Fluorescence emission from the acceptor dye indicates the level of fusion. In some embodiments, the FRET emission signal of the fused co-LNP is greater that the fluorescence emission signal of a mixture of two individual LNPs that are not fused together.

In some embodiments, the normalized FRET signal immediately following neutralization is greater than 0.3. In some embodiments, the normalized FRET signal immediately following neutralization is greater than 0.35. In some embodiments, the normalized FRET signal immediately following neutralization is greater than 0.38. In some embodiments, the normalized FRET signal immediately following neutralization is greater than 0.4. In some embodiments, the normalized FRET signal immediately following neutralization is between about 0.35 and 0.42. In other embodiments, the normalized FRET signal immediately following neutralization is between about 0.38 and 0.42. In any of the foregoing embodiments, the normalized FRET signal may be calculated by the method described in Example 16.

### 2. Methods for Producing DNA and RNA Co-LNPs

Provided herein methods for producing a co-formulated LNP (co-LNP). In some embodiments, the co-LNP comprises an RNA molecule and a DNA molecule.

In some embodiments, the co-LNP is generated from the fusion of a first LNP comprising an RNA molecule and a second LNP comprising a DNA molecule. In some embodiments, the first RNA molecule comprises a guide RNA (gRNA). In some embodiments, the gRNA is a single guide RNA (sgRNA) comprising a crispr RNA (crRNA) and a tracrRNA. In some embodiments, the gRNA is complexed with a recombinant nuclease capable of inducing a DNA break. In some embodiments, the co-LNP is generated from the fusion of a first LNP comprising an RNA molecule and a second LNP comprising a DNA molecule. In some embodiments, the RNA molecule comprises a gRNA and mRNA encoding a recombinant nuclease capable of inducing a DNA break. In some embodiments, the DNA molecule comprises homology-directed repair template (HDRt) DNA.

In some embodiments, the co-LNP is generated from the fusion of a first LNP comprising an RNA molecule and a second LNP comprising a DNA molecule. In some embodiments, the RNA molecule comprises mRNA encoding a transposase. In some embodiments, the DNA molecule comprises a transposon encoding a recombinant receptor (e.g., a CAR). In some embodiments, the CAR is a single antigen directed CAR. In some embodiments, the CAR is a bispecific CAR. In some embodiments, the DNA (e.g., ceDNA) molecule encoding the bispecific CAR is at least 6 kilobases, at least 7 kilobases, or at least 8 kilobases. In some embodiments, the bispecific CAR is between about 6 kilobases and about 8 kilobases. In some embodiments, the bispecific CAR is about 8 kilobases.

In some embodiments, the co-LNP is generated from the fusion of a first LNP comprising an RNA molecule, a second LNP comprising an RNA molecule, and a third LNP comprising a DNA molecule. In some embodiments, the one of the first and second RNA LNPs comprises mRNA encoding a recombinant nuclease capable of inducing a DNA break. In some embodiments, the other of the first and second RNA LNPs comprises gRNA. In some embodiments, the gRNA is a single guide RNA (sgRNA) comprising a crispr RNA (crRNA) and a tracrRNA. In some embodiments, the DNA LNP comprises HDRt DNA.

In some embodiments, a co-LNP is generated from the fusion of any of the RNA LNPs described herein with any of the DNA LNPs described herein.

Also provided herein is a co-LNP produced by any of the methods provided herein.

a. Co-Formulation

Provided herein are methods of producing co-LNPs comprising fusing a RNA LNP comprising an RNA molecule with a DNA LNP comprising a DNA molecule to produce a co-LNP comprising RNA and DNA. In some embodiments, the method comprises mixing a composition comprising a RNA LNP with a composition comprising a DNA LNP, wherein the RNA LNP composition and the DNA LNP composition are both acidic. In some embodiments, the pH of the RNA LNP composition is about 4.0. In some embodiments, the pH of the DNA LNP composition is about 4.0. In some embodiments, the RNA LNPs are mixed with the DNA LNPs at a volume ratio of about 1:3 (RNA LNPs:DNA LNPs). In some embodiments, following mixing of the RNA and DNA LNPs, at least about six parts by volume of an isotonic buffer are added to the mixture of RNA and DNA LNPs. In some embodiments, about six parts by volume of an isotonic buffer are added to the mixture of RNA and DNA LNPs. In some embodiments, about seven parts by volume of an isotonic buffer are added to the mixture of RNA and DNA LNPs. In some embodiments, the isotonic buffer is about pH 7.4. In some embodiments, the isotonic buffer is phosphate buffered saline. Thus, in some embodiments, the method comprises: (1) mixing the RNA LNP composition and DNA LNP composition at a volume ratio of about 1:3 (RNA LNPs:DNA LNPs); and (2) adding at least about six parts by volume of an isotonic buffer (e.g. PBS) to the mixture of RNA and DNA LNPs, thereby generating a co-LNP comprising the RNA and DNA molecules. It is contemplated herein that neutralization of the mixed composition by the isotonic buffer mediates fusion of the RNA LNP and the DNA LNP.

In some embodiments, the RNA LNP comprises a gRNA complexed with a recombinant nuclease capable of inducing a DNA break. In some embodiments, the RNA LNP comprises mRNA encoding a recombinant nuclease capable of inducing a DNA break and a gRNA. In some embodiments, the DNA LNP comprises HDRt DNA. In some embodiments, the RNA LNP comprises a gRNA complexed with a recombinant nuclease capable of inducing a DNA break, and the DNA LNP comprises HDRt DNA. In some embodiments, the gRNA is a single guide RNA (sgRNA) comprising a crispr RNA (crRNA) and a tracrRNA. Thus, in some embodiments, the co-LNP comprises a gRNA complexed with a recombinant nuclease capable of inducing a DNA break and HDRt DNA. In some embodiments, the RNA LNP comprises mRNA encoding a recombinant nuclease capable of inducing a DNA break and a gRNA, and the DNA LNP comprises HDRt DNA. Thus, in some embodiments, the co-LNP comprises a gRNA, mRNA encoding for a recombinant nuclease capable of inducing a DNA break, and HDRt DNA.

In some embodiments, the RNA LNP comprises mRNA encoding a transposase. In some embodiments, the DNA LNP comprises a transposon encoding a recombinant receptor (e.g., a CAR). In some embodiments, the RNA LNP comprises mRNA encoding a transposase, and the DNA LNP comprises a transposon encoding a recombinant receptor (e.g., a CAR). Thus, in some embodiments, the co-LNP comprises mRNA encoding a transposase and a transposon, e.g. encoding a recombinant receptor.

b. Double Sequential Fusion

Provided herein are methods of producing co-LNPs by a double sequential fusion. In some embodiments, the methods comprise fusing a first RNA LNP comprising a first RNA molecule with a second RNA LNP comprising a second RNA molecule to generate a RNA co-LNP, which is then fused with DNA LNP to produce a co-LNP comprising RNA and DNA. In some embodiments, the method comprises mixing a first composition comprising a first RNA LNP with a second composition comprising a second RNA LNP, wherein the first RNA LNP composition and the second RNA LNP composition are both acidic. In some embodiments, the RNA LNPs compositions are acidic by virtue of comprising an acetate buffer. In some embodiments, the pH of the first and second RNA LNP compositions is about 4.0. In some embodiments, the first and second RNA LNP compositions are mixed together at a volume ratio of about 1:1 (first RNA LNPs:second RNA LNPs). In some embodiments, following mixing of the first and second RNA LNPs, about one part by volume of an isotonic buffer is added to the mixture of the first and second RNA LNPs. In some embodiments, the isotonic buffer is about pH 7.4. In some embodiments, the isotonic buffer is phosphate buffered saline (PBS). In some embodiments, this process generates a RNA co-LNP comprising the first and second RNA molecules in a single RNA co-LNP. In some embodiments, the RNA co-LNP is subsequently subjected to buffer exchange, such that the composition comprising the RNA co-LNP is about pH 4.0. In some embodiments, an acetate buffer is exchanged for the isotonic buffer. In some embodiments, the RNA LNPs are mixed with a composition of DNA LNPs having a pH of about 4.0 at a volume ratio of about 1:3 (RNA LNPs:DNA LNPs). In some embodiments, the DNA LNP composition is acidic by virtue of comprising an acetate buffer. In some embodiments, following mixing of the RNA and DNA LNPs, at least about six parts by volume of an isotonic buffer (e.g. PBS) are added to the mixture of RNA and DNA LNPs. In some embodiments, about six parts by volume of an isotonic buffer are added to the mixture of RNA and DNA LNPs. In some embodiments, about seven parts by volume of an isotonic buffer are added to the mixture of RNA and DNA LNPs. In some embodiments, the isotonic buffer is about pH 7.4. Thus, in some embodiments, the method comprises: (1) mixing the first RNA LNP composition and the second RNA LNP composition at a volume ratio of about 1:1; (2) adding about one part by volume of an isotonic buffer (e.g. PBS), thereby generating a RNA co-LNP; (3) exchanging the buffer of the RNA co-LNP composition for a buffer of about pH 4.0; (4) mixing the RNA co-LNP composition with the DNA LNP composition at a volume ratio of about 1:3 (RNA LNPs:DNA LNPs); and (5) adding at least about six parts by volume of an isotonic buffer (e.g. PBS) to the mixture of RNA and DNA LNPs, thereby generating a co-LNP comprising the RNA and DNA molecules. It is contemplated herein that neutralization of the mixed composition by the isotonic buffer mediates fusion of the RNA LNPs, or of the RNA LNP and the DNA LNP.

In some embodiments, one of the RNA LNPs comprises a gRNA. In some embodiments, the gRNA is a single guide RNA (sgRNA) comprising a crispr RNA (crRNA) and a tracrRNA. In some embodiments, the other of the RNA LNPs comprises mRNA encoding a recombinant nuclease capable of inducing a DNA break. In some embodiments, the DNA LNP comprises HDRt DNA. Thus, in some embodiments, the co-LNP generated by the double sequential fusion method comprises a gRNA, mRNA encoding a recombinant nuclease capable of inducing a DNA break, and HDRt DNA.

c. Trifusion

Provided herein are methods of producing co-LNPs comprising a tri-fusion LNP or “tri-LNP”. In some embodiments, the methods comprise fusing a first RNA LNP comprising a first RNA molecule with a second RNA LNP comprising a second RNA molecule and a DNA LNP comprising a DNA molecule to generate a co-LNP containing RNA and DNA molecules. In some embodiments, the method comprises mixing a first composition comprising a first RNA LNP with a second composition comprising a second RNA LNP and a third composition comprising a DNA molecule, wherein each of the compositions is acidic. In some embodiments, each of the composition is acidic by virtue of comprising an acetate buffer. In some embodiments, the pH of each composition is about 4.0. In some embodiments, the first and second RNA LNP compositions and the DNA LNP composition are mixed together at a volume ratio of about 1:2 (RNA LNPs:DNA LNPs). In some embodiments, following mixing of the first and second RNA LNPs and the DNA LNPs, at least about six parts by volume of an isotonic buffer are added to the mixture of the first and second RNA LNPs and DNA LNPs. In some embodiments, the isotonic buffer is about pH 7.4. In some embodiments, the isotonic buffer is phosphate buffered saline (PBS). In some embodiments, this process generates a co-LNP comprising the first and second RNA molecules and the DNA molecule in a single co-LNP. Thus, in some embodiments, the method comprises: (1) mixing the first RNA LNP composition, the second RNA LNP composition, and the DNA LNP composition at a volume ratio of about 1:2 (RNA LNPs:DNA LNPs); (2) adding between about six and seven parts by volume of an isotonic buffer (e.g. PBS), thereby generating a co-LNP comprising the RNA and DNA molecules. It is contemplated herein that neutralization of the mixed composition by the isotonic buffer mediates fusion of the RNA LNPs and the DNA LNP.

In some embodiments, one of the RNA LNPs comprises a gRNA. In some embodiments, the gRNA is a single guide RNA (sgRNA) comprising a crispr RNA (crRNA) and a tracrRNA. In some embodiments, the other of the RNA LNPs comprises mRNA encoding a recombinant nuclease capable of inducing a DNA break. In some embodiments, the DNA LNP comprises HDRt DNA. Thus, in some embodiments, a co-LNP generated by the trifusion method comprises a gRNA, mRNA encoding a recombinant nuclease capable of inducing a DNA break, and HDRt DNA.

### B. RNA Co-LNPs

### 1. Components

Provided herein are co-formulated LNPs (co-LNPs) comprising a ribonucleic acid (RNA) molecule (also referred to as RNA co-LNPs or RNA-containing co-LNPs). In some embodiments, the RNA LNP comprises an ionizable lipid; a helper lipid; a polyethylene glycol (PEG)-conjugated lipid; cholesterol; and an RNA molecule. In some embodiments, the RNA co-LNP comprises a first RNA molecule and a second RNA molecule. In some embodiments, the RNA co-LNP comprises a first ionizable lipid and a second ionizable lipid.

In some embodiments, the RNA co-LNP comprises (1) a first RNA molecule and a second RNA molecule; and (2) a first ionizable lipid and a second ionizable lipid. In some embodiments, one of the first and second RNA molecules encodes a recombinant nuclease capable of inducing a DNA break, and the other of the first and second RNA molecules is a guide RNA (gRNA). In some embodiments, the gRNA is a single guide RNA (sgRNA) comprising a crispr RNA (crRNA) and a tracrRNA.

In some embodiments, the RNA co-LNP comprises a fusion of any two of the LNPs as described in Section I.B. In some embodiments, the RNA co-LNP comprises a fusion of any three of the LNPs as described in Section I.B.

In some embodiments, the RNA co-LNP comprises a fusion of a first LNP and a second LNP, wherein: (1) the first LNP comprises: an RNA molecule and a first ionizable lipid; and (2) the second LNP comprises an RNA molecule and a second ionizable lipid. In some embodiments, one of the first and second RNA molecules encodes a recombinant nuclease capable of inducing a DNA break, and the other of the first and second RNA molecules is a guide RNA (gRNA). In some embodiments, the RNA co-LNP comprises a volumetric ratio of the first LNP to the second LNP that is about 1:1.

In some embodiments, the recombinant nuclease is a zinc finger nuclease (ZFN), a transcription activator-like effector nuclease (TALEN), or a CRISPR-associated nuclease (Cas). In some embodiments, the recombinant nuclease is a zinc finger nuclease (ZFN). In some embodiments, the recombinant nuclease is a transcription activator-like effector nuclease (TALEN). In some embodiments, the recombinant nuclease is a CRISPR-associated nuclease (Cas). In some of any such embodiments, the Cas nuclease is selected from the group consisting of Cas3, Cas9, Cas10, Cas12, Cas12a, and Cas13. In some of any such embodiments, the Cas nuclease is Cas9. In some of any such embodiments, the Cas nuclease is Cas9 or a variant thereof. In some embodiments, the Cas nuclease is an enhanced specificity Cas9 (eSpCas9). In some embodiments, the Cas nuclease is a high fidelity Cas9 (HiFi Cas9). In some of any such embodiments, the Cas9 is from a bacteria selected from the group consisting of Streptococcus pyogenes, Staphylococcus aureus, Neisseria meningitides, Campylobacter jejuni, and Streptococcus thermophilis. In some of any such embodiments, the Cas9 is from Streptococcus pyogenes. In some of any such embodiments, the Cas9 or a variant thereof is from Streptococcus pyogenes. In some embodiments, the Cas is Cas12a.

In some embodiments, the RNA co-LNP comprises a first and second ionizable lipid. In some embodiments, the first and/or second ionizable lipid is positively charged (e.g. a cationic lipid). In some embodiments, the first and/or second ionizable lipid is a cationic lipid, including but not limited to those described in U.S. Pat. Nos. 9,593,077; 9,365,610; 9,670,152; and 9,458,090. In some embodiments, the first and/or second ionizable lipid is is a cationic lipid, including but not limited to those described in Published PCT application WO2013149140. In some embodiments, the first and/or second ionizable lipid is a cationic lipid, including but not limited to those described in published US Patent application US2019084965; US2019106379. In some embodiments, the first and/or second ionizable lipid is a cationic lipid, including but not limited to those described in Published EP application EP2830595.

In some embodiments, the first and/or second ionizable lipid is positively charged (e.g. a cationic lipid). In some embodiments, the first and/or second ionizable lipid is selected from the group consisting of D-Lin, 2,2-dilinoleyl-4-dimethylaminoethyl-[1,3]-dioxolane (DLin-KC2-DMA), or an analog thereof, 4-(dimethylamino)-butanoic acid, (10Z,13Z)-1-(9Z,12Z)-9,12-octadecadien-1-yl-10,13-nonadecadien-1-yl ester (DLin-MC3-DMA), or an analog thereof, 1 OF-C4-Deg-Lin, or an analog thereof, and cKK-E12, or an analog thereof.

In some embodiments, the first ionizable lipid is an amino lipidoid comprising a diketopiperazine ring core. In some embodiments, the first ionizable lipid comprises an unsaturated tail. In some embodiments, the first ionizable lipid comprises an unsaturated linoleic tail. In some embodiments, the first ionizable lipid is: (a) OF-C4-Deg-Lin, or an analog thereof; or (b) cKK-E12, or an analog thereof. In some embodiments, the first ionizable lipid is OF-C4-Deg-Lin, or an analog thereof. In some embodiments, wherein the first ionizable lipid is cKK-E12, or an analog thereof. In some embodiments, the first ionizable lipid is DLin-MC3-DMA, or an analog thereof.

In some embodiments, the first ionizable lipid and the second ionizable lipid are the same. In some embodiments, the first ionizable lipid and the second ionizable lipid are different.

In some embodiments, the second ionizable lipid is an amino lipidoid comprising a diketopiperazine ring core. In some embodiments, the second ionizable lipid comprises an unsaturated tail. In some embodiments, the second ionizable lipid comprises an unsaturated linoleic tail. In some embodiments, the second ionizable lipid is: (a) OF-C4-Deg-Lin, or an analog thereof; or (b) cKK-E12, or an analog thereof. In some embodiments, the second ionizable lipid is OF-C4-Deg-Lin, or an analog thereof. In some embodiments, wherein the second ionizable lipid is cKK-E12, or an analog thereof. In some embodiments, the second ionizable lipid is DLin-MC3-DMA, or an analog thereof.

In some embodiments, the mass fraction of the ionizable lipid in the RNA co-LNP is between about 32% and about 65%. In some embodiments, the mass fraction of the ionizable lipid is about 32%, about 32.5%, about 33%, about 33.5%, about 34%, about 34.5%, about 35%, about 35.5%, about 36%, about 50%, about 51%, about 52%, about 53%, about 54%, about 55%, about 56%, about 57%, about 58%, about 59%, about 60%, about 61%, about 62%, about 63%, about 64%, or about 65%. In some embodiments, the mass fraction of the ionizable lipid is about 32%. In some embodiments, the mass fraction of the ionizable lipid is about 32.5%. In some embodiments, the mass fraction of the ionizable lipid is about 33%. In some embodiments, the mass fraction of the ionizable lipid is about 33.5%. In some embodiments, the mass fraction of the ionizable lipid is about 34%. In some embodiments, the mass fraction of the ionizable lipid is about 34.5%. In some embodiments, the mass fraction of the ionizable lipid is about 35%. In some embodiments, the mass fraction of the ionizable lipid is about 35.5%. In some embodiments, the mass fraction of the ionizable lipid is about 36%. In some embodiments, the mass fraction of the ionizable lipid is about 54%. In some embodiments, the mass fraction of the ionizable lipid is about 55%. In some embodiments, the mass fraction of the ionizable lipid is about 56%. In some embodiments, the mass fraction of the ionizable lipid is about 57%. In some embodiments, the mass fraction of the ionizable lipid is about 58%. In some embodiments, the mass fraction of the ionizable lipid is about 59%. In some embodiments, the mass fraction of the ionizable lipid is about 60%. In some embodiments, the mass fraction of the ionizable lipid is about 61%.

In some embodiments, the helper lipid is selected from the group consisting of dioleoylphosphatidylethanolamine (DOPE), 1,2-distearoyl-sn-glycero-3-phosphocholine (DSPC), 1-Stearoyl-2-oleoyl-sn-glycero-3-phosphocholine (SOPC), and 1,2-dioleoyl-sn-glycero-sn-3-phosphatidylcholine (DOPC). In some embodiments, the helper lipid is DOPE. In some embodiments, the helper lipid has the structure of

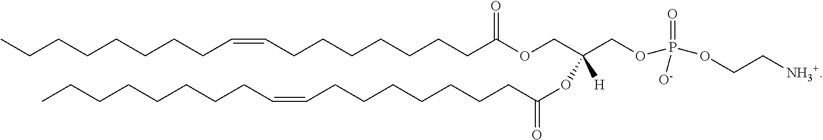

In some embodiments, the helper lipid is DSPC. In some embodiments, the helper lipid has the structure of

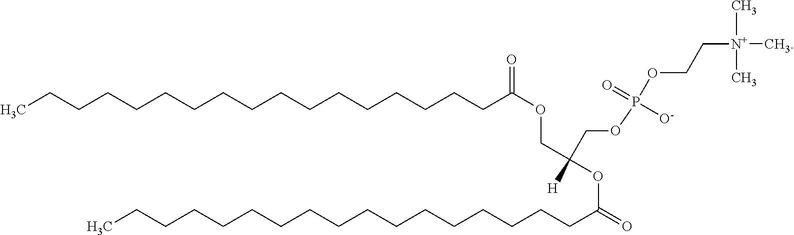 [SMILES: CCCCCCCCCCCCCCCCCC(=O)OC[C@H](COP(=O)([O-])OCC[N+](C)(C)C)OC(=O)CCCCCCCCCCCCCCCCC]

In some embodiments, the helper lipid is SOPC. In some embodiments, the helper lipid has the structure of

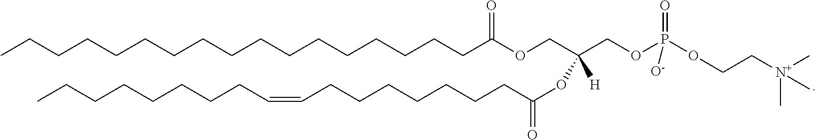 [SMILES: CCCCCCCC/C=C\CCCCCCCC(=O)O[C@H](COC(=O)CCCCCCCCCCCCCCCCC)COP(=O)([O-])OCC[N+](C)(C)C]

In some embodiments, the helper lipid is DOPC. In some embodiments, the helper lipid has the structure of

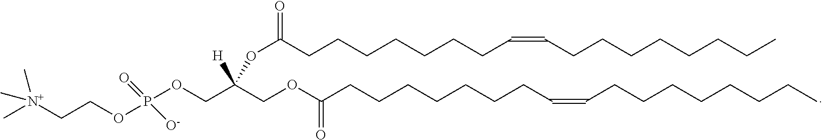 [SMILES: CCCCCCCC/C=C\CCCCCCCC(=O)OC[C@H](COP(=O)([O-])OCC[N+](C)(C)C)OC(=O)CCCCCCC/C=C\CCCCCCCC]

In some embodiments, the mass fraction of the helper lipid in the RNA co-LNP is between about 4% and about 20%. In some embodiments, the mass fraction of the helper lipid is about 4%, about 4.5%, about 5%, about 5.5%, about 6%, about 6.5%, about 7%, about 7.5%, about 8%, about 8.5%, about 9%, about 9.5%, 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20%. In some embodiments, the mass fraction of the helper lipid is about 4%. In some embodiments, the mass fraction of the helper lipid is about 4.5%. In some embodiments, the mass fraction of the helper lipid is about 5%. In some embodiments, the mass fraction of the helper lipid is about 5.5%. In some embodiments, the mass fraction of the helper lipid is about 6%. In some embodiments, the mass fraction of the helper lipid is about 6.5%. In some embodiments, the mass fraction of the helper lipid is about 7%. In some embodiments, the mass fraction of the helper lipid is about 7.5%. In some embodiments, the mass fraction of the helper lipid is about 8%. In some embodiments, the mass fraction of the helper lipid is about 12%. In some embodiments, the mass fraction of the helper lipid is about 12.5%. In some embodiments, the mass fraction of the helper lipid is about 13%. In some embodiments, the mass fraction of the helper lipid is about 13.5%. In some embodiments, the mass fraction of the helper lipid is about 14%. In some embodiments, the mass fraction of the helper lipid is about 14.5%. In some embodiments, the mass fraction of the helper lipid is about 15%. In some embodiments, the mass fraction of the helper lipid is about 15.5%. In some embodiments, the mass fraction of the helper lipid is about 16%. In some embodiments, the mass fraction of the helper lipid is about 16.5%. In some embodiments, the mass fraction of the helper lipid is about 17%. In some embodiments, the mass fraction of the helper lipid is about 17.5. In some embodiments, the mass fraction of the helper lipid is about 18%. In some embodiments, the mass fraction of the helper lipid is about 18.5%. In some embodiments, the mass fraction of the helper lipid is about 19%. In some embodiments, the mass fraction of the helper lipid is about 19.5%. In some embodiments, the mass fraction of the helper lipid is about 20%.

In some embodiments, the polyethylene glycol (PEG)-conjugated lipid is DMG-PEG2000. In some embodiments, the PEG-conjugated lipid has the structure of

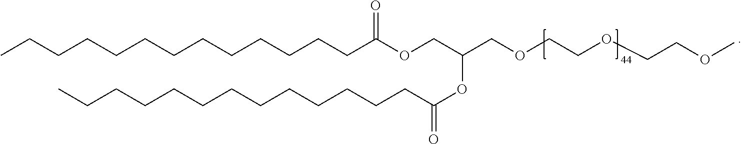

In some embodiments, the mass fraction of the PEG-conjugated lipid in the RNA co-LNP is between about 2% and about 10%, or between about 3% and about 7%. In some embodiments, the mass fraction of the PEG-conjugated lipid is about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5% or about 10%. In some embodiments, the mass fraction of the PEG-conjugated lipid is about 2%. In some embodiments, the mass fraction of the PEG-conjugated lipid is about 2.5%. In some embodiments, the mass fraction of the PEG-conjugated lipid is about 3%. In some embodiments, the mass fraction of the PEG-conjugated lipid is about 3.5%. In some embodiments, the mass fraction of the PEG-conjugated lipid is about 4%. In some embodiments, the mass fraction of the PEG-conjugated lipid is about 4.5%. In some embodiments, the mass fraction of the PEG-conjugated lipid is about 5%. In some embodiments, the mass fraction of the PEG-conjugated lipid is about 5.5%. In some embodiments, the mass fraction of the PEG-conjugated lipid is about 6%. In some embodiments, the mass fraction of the PEG-conjugated lipid is about 6.5%. some embodiments, the mass fraction of the PEG-conjugated lipid is about 7%. In some embodiments, the mass fraction of the PEG-conjugated lipid is about 7.5%. In some embodiments, the mass fraction of the PEG-conjugated lipid is about 8%. In some embodiments, the mass fraction of the PEG-conjugated lipid is about 8.5%.

In some embodiments, cholesterol has the structure of

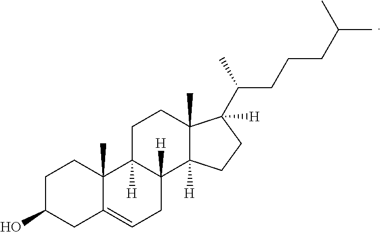

In some embodiments, the mass fraction of cholesterol in the RNA co-LNP is between about 15% and about 45%. In some embodiments, the mass fraction of cholesterol is about 15%, about 16%, about 17%, about 18%, about 19%, about 20%, about 25%, about 30%, 35%, about 36%, about 37%, about 38%, about 39%, about 40%, about 41%, about 42%, about 43%, about 44%, or about 45%. In some embodiments, the mass fraction of cholesterol is about 15%. In some embodiments, the mass fraction of cholesterol is about 16%. In some embodiments, the mass fraction of cholesterol is about 17%. In some embodiments, the mass fraction of cholesterol is about 18%. In some embodiments, the mass fraction of cholesterol is about 19%. In some embodiments, the mass fraction of cholesterol is about 20%. In some embodiments, the mass fraction of cholesterol is about 35%. In some embodiments, the mass fraction of cholesterol is about 36%. In some embodiments, the mass fraction of cholesterol is about 37%. In some embodiments, the mass fraction of cholesterol is about 38%. In some embodiments, the mass fraction of cholesterol is about 39%. In some embodiments, the mass fraction of cholesterol is about 40%. In some embodiments, the mass fraction of cholesterol is about 41%. In some embodiments, the mass fraction of cholesterol is about 42%. In some embodiments, the mass fraction of cholesterol is about 43%. In some embodiments, the mass fraction of cholesterol is about 44%. In some embodiments, the mass fraction of cholesterol is about 45%.

In some embodiments, the mass fraction of the RNA in the RNA co-LNP is between about 2% and about 12%, between about 3% and about 11%, or between about 3.5% and about 10%. In some embodiments, the mass fraction of the RNA is about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, or about 13%. In some embodiments, the mass fraction of the RNA is about 3.5%. In some embodiments, the mass fraction of the RNA is about 10%.

In some embodiments, the total RNA concentration in the RNA co-LNP is between about 4 ug/mL and about 200 ug/mL, between about 5 ug/mL and about 50 ug/mL, or between about 50 ug/mL and about 150 ug/mL. In some embodiments, the total RNA concentration is about 4 ug/mL. In some embodiments, the total RNA concentration is about 4.5 ug/mL. In some embodiments, the total RNA concentration is about 5 ug/mL. In some embodiments, the total RNA concentration is about 6 ug/mL. In some embodiments, the total RNA concentration is about 6.5 ug/mL. In some embodiments, the total RNA concentration is about 7 ug/mL. In some embodiments, the total RNA concentration is about 7.5 ug/mL. In some embodiments, the total RNA concentration is about 8 ug/mL. In some embodiments, the total RNA concentration is about 8.5 ug/mL. In some embodiments, the total RNA concentration is about 9 ug/mL. In some embodiments, the total RNA concentration is about 10 ug/mL. In some embodiments, the total RNA concentration is about 20 ug/mL. In some embodiments, the total RNA concentration is about 30 ug/mL. In some embodiments, the total RNA concentration is about 40 ug/mL. In some embodiments, the total RNA concentration is about 50 ug/mL. In some embodiments, the total RNA concentration is about 55 ug/mL. In some embodiments, the total RNA concentration is about 60 ug/mL. In some embodiments, the total RNA concentration is about 65 ug/mL. In some embodiments, the total RNA concentration is about 70 ug/mL. In some embodiments, the total RNA concentration is about 75 ug/mL. In some embodiments, the total RNA concentration is about 80 ug/mL. In some embodiments, the total RNA concentration is about 85 ug/mL. In some embodiments, the total RNA concentration is about 90 ug/mL. In some embodiments, the total RNA concentration is about 95 ug/mL. In some embodiments, the total RNA concentration is about 100 ug/mL. In some embodiments, the total RNA concentration is about 125 ug/mL. In some embodiments, the total RNA concentration is about 150 ug/mL.

### 2. Formulations

Provided herein are LNPs containing: (a) a first and second ionizable lipid, each independently selected from the group consisting of DLin-MC3-DMA, or an analog thereof, OF-C4-Deg-Lin or analog thereof, and cKK-E12 or an analog thereof; (b) a helper lipid that is 1,2-distearoyl-sn-glycero-3-phosphocholine (DSPC); (c) a polyethylene glycol (PEG)-conjugated lipid that is DMG-PEG2000; (d) cholesterol; and (e) a first and second RNA molecule.

In some embodiments, the first and/or second ionizable lipid is DLin-MC3-DMA or an analog thereof. In some embodiments, the first and/or second ionizable lipid is DLin-MC3-DMA. In some embodiments, the first and second ionizable lipid is DLin-MC3-DMA. In some embodiments, the mass fraction of DLin-MC3-DMA in the RNA co-LNP is between about 35% and about 40%. In some embodiments, the mass fraction of DSPC in the RNA co-LNP is between about 10% and about 20%. In some embodiments, the mass fraction of DMG-PEG2000 is between about 3% and about 5%. In some embodiments, the mass fraction of cholesterol in the RNA co-LNP is between about 35% and about 45%. In some embodiments, the mass fraction of the RNA molecule in the RNA co-LNP is between about 3% and about 4%.

In some embodiments, the RNA co-LNP contains: (a) DLin-MC3-DMA with a mass fraction of between about 35% and about 40%; (b) DSPC with a mass fraction of between about 10% and about 20%; (c) DMG-PEG2000 with a mass fraction of between about 3% and about 5%; (d) cholesterol with a mass fraction of between about 35% and about 45%; and (e) a ribonucleic acid (RNA) molecule. In some embodiments, the RNA co-LNP contains: (a) DLin-MC3-DMA with a mass fraction of about 37.8%; (b) DSPC with a mass fraction of about 15.2%; (c) DMG-PEG2000 with a mass fraction of about 3.5%; (d) cholesterol with a mass fraction of about 40%; and (e) a ribonucleic acid (RNA) molecule with a mass fraction of about 3.5%.

In some embodiments, the first and/or second ionizable lipid is DLin-MC3-DMA or an analog thereof. In some embodiments, the first and/or second ionizable lipid is DLin-MC3-DMA. In some embodiments, the first and second ionizable lipid is DLin-MC3-DMA. In some embodiments, the mass fraction of DLin-MC3-DMA in the RNA co-LNP is between about 32% and about 36%. In some embodiments, the mass fraction of DSPC in the RNA co-LNP is between about 15% and about 20%. In some embodiments, the mass fraction of DMG-PEG2000 in the RNA co-LNP is between about 3.5% and about 5.5%. In some embodiments, the mass fraction of cholesterol in the RNA co-LNP is between about 35% and about 45%. In some embodiments, the mass fraction of the RNA molecule in the RNA co-LNP is between about 3% and about 4%.

In some embodiments, the RNA co-LNP contains: (a) DLin-MC3-DMA with a mass fraction of between about 32% and about 36%; (b) DSPC with a mass fraction of between about 15% and about 20%; (c) DMG-PEG2000 with a mass fraction of between about 3.5% and about 5.5%; (d) cholesterol with a mass fraction of between about 35% and about 45%; and (e) a ribonucleic acid (RNA) molecule. In some embodiments, the RNA co-LNP contains: (a) DLin-MC3-DMA with a mass fraction of about 34.4%; (b) DSPC with a mass fraction of about 17.6%; (c) DMG-PEG2000 with a mass fraction of about 4.5%; (d) cholesterol with a mass fraction of about 40%; and (e) a ribonucleic acid (RNA) molecule with a mass fraction of about 3.5%.

In some embodiments, the first and/or second ionizable lipid is a lipidoid comprising a diketopiperazine amino core. In some embodiments, the first and/or second ionizable lipid is selected from OF-C4-Deg-Lin or analog thereof and cKK-E12 or an analog thereof. In some embodiments, the first and/or second ionizable lipid comprises an unsaturated tail, optionally an unsaturated linoleic tail. In some embodiments, the first and/or second ionizable lipid is selected from OF-C4-Deg-Lin or analog thereof. In some embodiments, the first and second ionizable lipid is selected from OF-C4-Deg-Lin or analog thereof. In some embodiments, the first and second ionizable lipid is OF-C4-Deg-Lin.

In some embodiments, the RNA co-LNP contains: (a) OF-C4-Deg-Lin with a mass fraction of between about 32% and about 36%; (b) DSPC with a mass fraction of between about 15% and about 20%; (c) DMG-PEG2000 with a mass fraction of between about 3.5% and about 5.5%; (d) cholesterol with a mass fraction of between about 35% and about 45%; and (e) a ribonucleic acid (RNA) molecule. In some embodiments, the RNA co-LNP contains: (a) OF-C4-Deg-Lin with a mass fraction of about 34.4%; (b) DSPC with a mass fraction of about 17.6%; (c) DMG-PEG2000 with a mass fraction of about 4.5%; (d) cholesterol with a mass fraction of about 40%; and (e) a ribonucleic acid (RNA) molecule with a mass fraction of about 3.5%.

In some embodiments, the RNA co-LNP contains: (a) cKK-E12 with a mass fraction of between about 32% and about 36%; (b) DSPC with a mass fraction of between about 15% and about 20%; (c) DMG-PEG2000 with a mass fraction of between about 3.5% and about 5.5%; (d) cholesterol with a mass fraction of between about 35% and about 45%; and (e) a ribonucleic acid (RNA) molecule. In some embodiments, the RNA co-LNP contains: (a) cKK-E12 with a mass fraction of about 34.4%; (b) DSPC with a mass fraction of about 17.6%; (c) DMG-PEG2000 with a mass fraction of about 4.5%; (d) cholesterol with a mass fraction of about 40%; and (e) a ribonucleic acid (RNA) molecule with a mass fraction of about 3.5%.

In some embodiments, the RNA co-LNP contains: (a) OF-C4-Deg-Lin with a mass fraction of between about 50% and about 65%; (b) DSPC with a mass fraction of between about 12% and about 24%; (c) DMG-PEG2000 with a mass fraction of between about 6% and about 7%; (d) cholesterol with a mass fraction of between about 17% and about 20%; and (e) a ribonucleic acid (RNA) molecule. In some embodiments, the RNA co-LNP contains: (a) OF-C4-Deg-Lin with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 3.5%. In some embodiments, the RNA co-LNP contains: (a) OF-C4-Deg-Lin with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 5%. In some embodiments, the RNA co-LNP contains: (a) OF-C4-Deg-Lin with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 10%. In some embodiments, the RNA co-LNP contains: (a) OF-C4-Deg-Lin with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 3.5%. In some embodiments, the RNA coLNP contains: (a) OF-C4-Deg-Lin with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 10%.

In some embodiments, the RNA co-LNP contains: (a) cKK-E12 with a mass fraction of between about 50% and about 65%; (b) DSPC with a mass fraction of between about 12% and about 24%; (c) DMG-PEG2000 with a mass fraction of between about 6% and about 7%; (d) cholesterol with a mass fraction of between about 17% and about 20%; and (e) a ribonucleic acid (RNA) molecule. In some embodiments, the RNA co-LNP contains: (a) cKK-E12 with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 3.5%. In some embodiments, the RNA co-LNP contains: (a) cKK-E12 with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 5%. In some embodiments, the RNA co-LNP contains: (a) cKK-E12 with a mass fraction of about 60.21%; (b) DSPC with a mass fraction of about 13.64%; (c) DMG-PEG2000 with a mass fraction of about 6.77%; (d) cholesterol with a mass fraction of about 19.39%; and (e) a ribonucleic acid (RNA) molecule with a mass fraction of about 10%. In some embodiments, the RNA co-LNP contains: (a) cKK-E12 with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 3.5%. In some embodiments, the RNA co-LNP contains: (a) cKK-E12 with a mass fraction of about 54.19%; (b) DSPC with a mass fraction of about 12.28%; (c) DMG-PEG2000 with a mass fraction of about 6.08%; (d) cholesterol with a mass fraction of about 17.45%; and (e) a ribonucleic acid (RNA) molecule with a mass fraction of about 10%.

### 3. Methods for Producing RNA Co-LNPs

Provided herein are methods for producing RNA co-LNPs, including any as described in the preceding section.

In some embodiments, the method comprises mixing, in an acidic buffer: (a) a first RNA LNP comprising a first ionizable lipid and a first RNA molecule; and (b) a second RNA LNP comprising a second ionizable lipid and a second RNA molecule. In some embodiments, the mixing generates a composition comprising the first LNP and the second LNP. In some embodiments, the method comprises neutralizing the composition comprising the first LNP and the second LNP. In some embodiments, the neutralizing generates a co-LNP, which is a fusion of the first LNP and the second LNP. In some embodiments, the method comprises (1) mixing, in an acidic buffer (a) a first RNA LNP comprising a first ionizable lipid and a first RNA molecule and (b) a second RNA LNP comprising a second ionizable lipid and a second RNA molecule; thereby generating a composition comprising the first LNP and the second LNP; and (2) neutralizing the composition comprising the first LNP and the second LNP, thereby generating a co-LNP, which is a fusion of the first LNP and the second LNP.

In some embodiments, the volumetric ratio of the first LNP to the second LNP in the composition is between about 3:1 or about 1:3. In some embodiments, the volumetric ratio of the first LNP to the second LNP in the composition is about 3:1. In some embodiments, the volumetric ratio of the first LNP to the second LNP in the composition is about 2:1. In some embodiments, the volumetric ratio of the first LNP to the second LNP in the composition is about 1:1. In some embodiments, the volumetric ratio of the first LNP to the second LNP in the composition is about 1:2. In some embodiments, the volumetric ratio of the first LNP to the second LNP in the composition is about 1:3.

In some embodiments, the acidic buffer is an acetate buffer. In some embodiments, the acidic buffer has a pH of between about 3.0 and about 4.5, or of 4.0. In some embodiments, the acidic buffer has a pH of about 3.0. In some embodiments, the acidic buffer has a pH of about 3.5. In some embodiments, the acidic buffer has a pH of about 4.0. In some embodiments, the acidic buffer has a pH of about 4.5. In some embodiments, the acidic buffer is neutralized to a pH of between about 6.0 and about 7.5, or between about 6.5 and about 7.0. In some embodiments, the acidic buffer is neutralized to a pH of about 6.0. In some embodiments, the acidic buffer is neutralized to a pH of about 6.5. In some embodiments, the acidic buffer is neutralized to a pH of about 7.0. In some embodiments, the acidic buffer is neutralized to a pH of about 7.5.

In some embodiments, neutralizing the composition comprising the first LNP and the second LNP comprises adding an isotonic buffer. In some embodiments, the isotonic buffer has a pH of about 7.4. In some embodiments, the isotonic buffer is phosphate buffered saline (PBS). In some embodiments, neutralizing the composition comprising the first LNP and the second LNP comprises adding at least about 6 parts of the isotonic buffer to 1 part of the acidic buffer. In some embodiments, neutralizing the composition comprising the first LNP and the second LNP comprises adding between about 6 parts and about 7 parts of the isotonic buffer to 1 part of the acidic buffer. In some embodiments, neutralizing the composition comprising the first LNP and the second LNP comprises adding about 6 parts of the isotonic buffer to 1 part of the acidic buffer. In some embodiments, neutralizing the composition comprising the first LNP and the second LNP comprises adding about 7 parts of the isotonic buffer to 1 part of the acidic buffer.

Also provided herein is a RNA co-LNP produced by any of the methods provided herein.

Provided herein are methods of producing co-LNPs comprising a tri-fusion LNP or “tri-LNP” of three RNA LNPs. In some embodiments, the methods comprise fusing a first RNA LNP comprising a first RNA molecule with a second RNA LNP comprising a second RNA molecule and a third RNA LNP comprising a third RNA molecule to generate a co-LNP containing three RNA molecules. In some embodiments, the method comprises mixing a first composition comprising a first RNA LNP with a second composition comprising a second RNA LNP and a third composition comprising a third RNA LNP, wherein each of the compositions is acidic. In some embodiments, each of the composition is acidic by virtue of comprising an acetate buffer. In some embodiments, the pH of each composition is about 4.0. In some embodiments, following mixing of the three RNA LNPs, at least about six parts by volume of an isotonic buffer are added to the mixture. In some embodiments, the isotonic buffer is about pH 7.4. In some embodiments, the isotonic buffer is phosphate buffered saline (PBS). In some embodiments, this process generates a co-LNP comprising the three RNA molecules in a single co-LNP. It is contemplated herein that neutralization of the mixed composition by the isotonic buffer mediates fusion of the RNA LNPs.

### C. DNA Co-LNPs

Provided herein are co-formulated LNPs (co-LNPs) comprising a deoxyribonucleic acid (DNA) molecule (also referred to as DNA co-LNPs or DNA-containing co-LNPs). In some embodiments, the DNA LNP comprises an ionizable lipid; a helper lipid; a polyethylene glycol (PEG)-conjugated lipid; cholesterol; and a DNA molecule. In some embodiments, the DNA co-LNP comprises a first DNA molecule and a second DNA molecule. In some embodiments, the DNA co-LNP comprises a first ionizable lipid and a second ionizable lipid. In some embodiments, the co-LNP is generated from the fusion of a first DNA LNP comprising a first DNA molecule and a second DNA LNP comprising a second DNA molecule.

In some embodiments, the co-LNP is generated from the fusion of a first DNA LNP comprising a first DNA molecule, a second DNA LNP comprising a second DNA molecule, and a third DNA LNP comprising a third DNA molecule. In certain embodiments, such a DNA tri-LNP can be generated by a double sequential fusion or tri-fusion described herein. In some embodiments, the method comprises mixing a first composition comprising a first DNA LNP with a second composition comprising a second DNA LNP and a third composition comprising a third DNA LNP, wherein each of the compositions is acidic. In some embodiments, each of the composition is acidic by virtue of comprising an acetate buffer. In some embodiments, the pH of each composition is about 4.0. In some embodiments, following mixing of the three DNA LNPs, at least about six parts by volume of an isotonic buffer are added to the mixture. In some embodiments, the isotonic buffer is about pH 7.4. In some embodiments, the isotonic buffer is phosphate buffered saline (PBS). In some embodiments, this process generates a co-LNP comprising the three DNA molecules in a single co-LNP. It is contemplated herein that neutralization of the mixed composition by the isotonic buffer mediates fusion of the DNA LNPs.

Any of the DNA LNPs described herein can be used to generate a DNA co-LNP comprising two or three DNA molecules.

### III. Methods for Introducing DNA into Cells

In some embodiments, the LNPs or compositions containing the same, as produced by the methods described herein, are used to deliver a nucleic acid molecule into a T cell. In some embodiments, delivering a nucleic acid molecule into a T cell using a LNP described herein includes (a) incubating a composition containing T cells (“T cell composition”) under stimulating conditions; and (b) incubating the stimulated T cell composition with the LNPs, wherein the LNPs contain the nucleic acid molecule. In some embodiments, the LNPs are fused co-LNPs or tri-LNPs, as described in Section II.

In some embodiments, incubating the T cell composition under stimulating conditions includes exposing the T cells to anti-CD3/anti-CD28 antibodies or fragments thereof.

In some embodiments, the method further includes exposing the T cell composition to Apolipoprotein E (ApoE) before, during, or after incubation with the LNPs. In some embodiments, the T cell composition is exposed to ApoE before incubation with the LNPs. In some embodiments, the T cell composition is exposed to ApoE during at least a portion of incubation with the LNPs. In some embodiments, the ApoE is ApoE2. In some embodiments, the ApoE is ApoE3. In some embodiments, the ApoE is ApoE4. In some embodiments, the T cell composition is exposed to between about 0.1 □g/mL and about 10 g/mL ApoE, between about 0.5 μg/mL and about 5 μg/mL ApoE, or between about 0.75 μg/mL and about 1.5 μg/mL ApoE. In some embodiments, the T cell composition is exposed to about 1 μg/mL ApoE. In some embodiments, the T cell composition is exposed to about 1 μg/mL ApoE4.

In some embodiments, the LNPs or compositions containing the same, as produced by the methods described herein, are administered directly to a patient (i.e., in vivo administration). In some embodiments, the LNPs are used to deliver a nucleic acid molecule to a patient in need thereof. In some embodiments, the LNPs or compositions containing the same are administered directly to a patient transduce T cells of the patient and deliver a payload to the T cells of the patient. In some embodiments, the payload is a DNA and/or RNA molecule, as described herein. In particular embodiments, co-LNPs or tri-LNPs are administered directly to a patient. The LNP or LNP compositions can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration

In some embodiments, the nucleic acid is DNA or RNA. In some embodiments, the nucleic acid is DNA. In some embodiments, the nucleic acid is RNA.

In some embodiments, the nucleic acid encodes a recombinant receptor. In some embodiments, the recombinant receptor is a T cell receptor (TCR). In some embodiments, the recombinant receptor is a chimeric antigen receptor (CAR). In some embodiments, the CAR includes an extracellular antigen-recognition domain that specifically binds to the antigen and an intracellular signaling domain comprising an ITAM. In some embodiments, the CAR is a single antigen directed CAR. In some embodiments, the CAR is a bispecific CAR. In some embodiments, the DNA (e.g., ceDNA) molecule encoding the bispecific CAR is at least 6 kilobases, at least 7 kilobases, or at least 8 kilobases.

In some embodiments, the antigen is a tumor antigen. In some embodiments, the antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR VIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1). In some embodiments, the antigen is BCMA. In some embodiments, the antigen is CD19.

In some embodiments, the intracellular signaling domain includes an intracellular domain of a CD3-zeta (CD3ζ) chain. In some embodiments, the intracellular signaling region further includes a costimulatory signaling region. In some embodiments, the costimulatory signaling region includes a signaling domain of CD28, such as a human CD28. In some embodiments, the costimulatory signaling region includes a signaling domain of 4-1BB, such as a human 4-1BB. In some embodiments, the co-stimulatory domain is or includes a signaling domain of CD28.

In some embodiments, the cells produced by the methods described herein (e.g. cells having the nucleic acid) are used for the treatment of cancer. In some embodiments, the cells produced by the methods described herein (e.g. cells having the nucleic acid) are used for treating a subject having a cancer, such as for use in adoptive cell therapy. In some embodiments, the cells produced by the methods described herein (e.g. cells having the nucleic acid) are for use as a medicament for treatment of a cancer. In some embodiments, the cells produced by the methods described herein (e.g. cells having the nucleic acid) are used in the manufacture of a medicament for treatment of a cancer.

### A. Cells and Preparation of Cells for Genetic Engineering

In some embodiments, provided are engineered cells, e.g., genetically engineered or modified cells, and methods of engineering cells (e.g., T cells). In some embodiments, one or more polynucleotides, e.g., encoding a recombinant receptor and/or additional polypeptide(s), such as any described herein, are introduced into a cell (e.g. a T cell) for engineering. In some aspects, the polynucleotides and/or portions thereof are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acid sequences are not naturally occurring, such as a nucleic acid sequences not found in nature or is modified from a nucleic acid sequence found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as iPSCs. In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human T cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some embodiments, the enrichment for central memory T (T_{CM}) cells is based on positive or high surface expression of CD45RO, CD62L, CCR7, CD28, CD3, CD27, and/or CD127. In some embodiments, the enrichment for central memory T (T_{CM}) cells is based on positive or high surface expression of CD62L, CCR7, CD28, and/or CD27. In some embodiments, the enrichment for central memory T (T_{CM}) cells is based on positive or high surface expression of CCR7, CD28, and/or CD27. In some embodiments, the enrichment for central memory T (T_{CM}) cells is based on positive or high surface expression of CD28 and CD27. In some aspects, isolation of a CD8⁺ population enriched for T_{CM} cells is carried out by depletion of cells expressing CD4, CD14, CD45RA, and positive selection or enrichment for cells expressing CD62L. In some aspects, isolation of a CD8⁺ population enriched for T_{CM} cells is carried out by depletion of cells expressing CD4, CD14, CD45RA, and positive selection or enrichment for cells expressing CD27 and CD28. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ).

In some aspects, the sample or population of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Pat. App. Pub. No. WO2009/072003 or US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the populations or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating population feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees Celsius, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

### B. Heterologous Polynucleotides Encoding Recombinant Proteins

In some embodiments, the provided methods are or include introducing a heterologous polynucleotide into cells of a population. In some embodiments, the heterologous polynucleotide encodes a recombinant protein. Such recombinant proteins may include recombinant receptors, such as any described herein. Introduction of the polynucleotides, e.g., heterologous or recombinant polynucleotides, encoding the recombinant protein into the cell may be carried out using the LNPs and compositions and uses thereof described herein. Exemplary methods of using the LNP compositions include those for non-viral introduction of heterologous polynucleotides encoding the receptors into the cells of a population, e.g., transfection. In some embodiments, a population of cells (e.g., T cells) is genetically engineered, such as to introduce a heterologous or recombinant polynucleotide encoding a recombinant receptor, thereby generating a population of transformed cells (also referred to herein as a transformed population of cells).

In certain embodiments, a polynucleotide encoding the recombinant protein, e.g. a recombinant receptor, is introduced to the cells. In certain embodiments, the polynucleotide or nucleic acid molecule is heterologous to the cells. In particular embodiments, the heterologous polynucleotide is not native to the cells. In certain embodiments, the heterologous nucleic acid molecule or heterologous polynucleotide encodes a protein, e.g., a recombinant protein that is not natively expressed by the cell. In particular embodiments, the heterologous nucleic acid molecule or polynucleotide is or contains a nucleic acid sequence that is not found in the cell prior to the contact or introduction.

In particular embodiments, the heterologous polynucleotide encodes a recombinant protein. In certain embodiments, the recombinant protein is a recombinant receptor. In some embodiments, the recombinant protein is a recombinant antigen receptor, such as a recombinant TCR or a chimeric antigen receptor (CAR).

### 1. Recombinant Receptors

In some embodiments, provided herein are LNPs and compositions thereof for use in introducing a nucleic acid (e.g., a DNA plasmid) encoding one or more recombinant receptor(s) into cells (e.g. T cells) of a population, thereby generating engineered cells that express or are engineered to express the one or more recombinant receptor(s). Among the receptors are antigen receptors and receptors containing one or more components thereof. The recombinant receptors may include chimeric receptors, such as those containing ligand-binding domains or binding fragments thereof and intracellular signaling domains or regions, functional non-TCR antigen receptors, chimeric antigen receptors (CARs), T cell receptors (TCRs), such as recombinant or transgenic TCRs, chimeric autoantibody receptor (CAAR) and components of any of the foregoing. The recombinant receptor, such as a CAR, generally includes the extracellular antigen (or ligand) binding domain linked to one or more intracellular signaling components, in some aspects via linkers and/or transmembrane domain(s). In some embodiments, the engineered cells express two or more receptors that contain different components, domains or regions. In some aspects, two or more receptors allows spatial or temporal regulation or control of specificity, activity, antigen (or ligand) binding, function and/or expression of the recombinant receptors.

a. Chimeric Antigen Receptors (CARs)

In some embodiments of the provided compositions and uses thereof, the chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is a stimulating or an activating intracellular domain portion, such as a T cell stimulating or activating domain, providing a primary activation signal or a primary signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

The chimeric receptors, such as CARs, generally include an extracellular antigen binding domain, such as a portion of an antibody molecule, generally a variable heavy (V_{H}) chain region and/or variable light (V_{L}) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen targeted by the receptor is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the antigen or antigen binding domain is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some aspects, the spacer contains only a hinge region of an IgG, such as only a hinge of IgG4 or IgG1. In other embodiments, the spacer is or contains an Ig hinge, e.g., an IgG4-derived hinge, optionally linked to a C_{H}2 and/or C_{H}3 domains. In some embodiments, the spacer is an Ig hinge, e.g., an IgG4 hinge, linked to C_{H}2 and C_{H}3 domains. In some embodiments, the spacer is an Ig hinge, e.g., an IgG4 hinge, linked to a C_{H}3 domain only. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the engineered cells express a CAR that specifically binds to an antigen expressed by a cell or tissue of the disease or condition or associated with the disease or condition.

b. T Cell Receptors (TCRs)

In some embodiments, engineered cells, such as T cells, used in connection with the provided compositions, and methods and uses thereof, are cells that express a T cell receptor (TCR) or antigen-binding portion thereof that recognizes a peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the f-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4⁺ or CD8⁺ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wtilfing, C. and Plickthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids.

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and loaded into a lipid nanoparticle of a lipid nanoparticle composition. The lipid nanoparticle or composition thereof can be used to transfect any suitable cells.

In some embodiments, the suitable cells are immune cells, such as T cells. In some cells, the suitable cells are T cells. In some cells, the suitable cells are CD4+ and/or CD8+ T cells. In some cells, the suitable cells are CD4+ T cells. In some cells, the suitable cells are CD8+ T cells. In some cells, the suitable cells are CD4+ and CD8+ T cells. In some embodiments, the suitable cells are formulated at a 1:1 ratio of CD4+ T cells to CD8+ T cells prior to transfection with the provided lipid nanoparticle compositions.

In some embodiments, recombinant plasmids can be prepared using standard recombinant DNA techniques. In some embodiments, plasmids can contain regulatory sequences, such as transcription and translation initiation and termination codons, which are specific to the type of host (e.g., bacterium, fungus, plant, or animal) into which the plasmid is to be introduced, as appropriate and taking into consideration whether the plasmid is DNA- or RNA-based. In some embodiments, the plasmid can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a plasmid encoding a TCR, the α and β chains are PCR amplified from total cDNA isolated from a T cell clone expressing the TCR of interest and cloned into a plasmid. In some embodiments, the α and β chains are cloned into the same vector. In some embodiments, the α and β chains are cloned into different vectors. In some embodiments, the generated α and β chains are incorporated into a retroviral, e.g. lentiviral, vector.

### C. Genetically Engineered Cells and Methods of Producing Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells (e.g. genetically engineered T cells). The genetic engineering generally involves introduction of a nucleic acid encoding the recombinant or engineered component into a composition containing the cells, such as by any of the methods described herein.

### 1. Nucleic Acids and Vectors

In some embodiments, cells are genetically engineered to express a recombinant receptor by incubation with any of the LNPs provided herein or a composition thereof. In some embodiments, the engineering is carried out by introducing one or more nucleic acid molecule(s) that encode the recombinant receptor or portions or components thereof by incubation with LNPs or a composition thereof. In some embodiments, the engineering is carried out by introducing one or more DNA molecule(s) that encode the recombinant receptor or portions or components thereof by incubation with LNPs or a composition thereof, and by electroporation one or more RNA molecule(s). In some embodiments, the engineering is carried out by introducing one or more DNA molecule(s) such as DNA repair template by incubation with LNPs or a composition thereof, and by electroporation one or more RNA molecule(s), such as a guide RNA, mRNA encoding a recombinant nuclease capable of inducing a break in DNA, or both. In some embodiments, the engineering is carried out by introducing one or more DNA molecule(s) such as a transposon by incubation with LNPs or a composition thereof, and by introducing one or more RNA molecule(s), such as mRNA encoding a transposase. In some embodiments, one or more DNA molecule(s) and one or more RNA molecule(s) are introduced into a cell by incubation with one or more LNPs or a composition thereof. In some embodiments, the cells are incubated with LNPs or a composition thereof comprising both a DNA molecule and an RNA molecule. In some embodiments, the cells are incubated with LNPs or a composition thereof comprising a DNA molecule and LNPs or a composition thereof comprising an RNA molecule. Also provided are nucleic acid molecules encoding a recombinant receptor, and plasmids or constructs containing such nucleic acids and/or polynucleotides.

In some embodiments, the polynucleotide encoding the recombinant receptor is comprised with a plasmid. In some embodiments, the plasmid is between about 2 kilobases (kb) and about 10 kb in size, or between about 3 kb and about 8 kb in size. In some embodiments, the plasmid is less than about 8 kb, less than about 7 kb, less than about 6 kb, less than about 5 kb, or less than about 4.5 kb in size. In some embodiments, the plasmid is less than about 8 kb in size. In some embodiments, the plasmid is less than about 7.5 kb in size. In some embodiments, the plasmid is less than about 7 kb in size. In some embodiments, the plasmid is less than about 6.5 kb in size. In some embodiments, the plasmid is less than about 6 kb in size. In some embodiments, the plasmid is less than about 5.5 kb in size. In some embodiments, the plasmid is less than about 5 kb in size. In some embodiments, the plasmid is less than about 4.5 kb in size. In some embodiments, the plasmid is about 4.5 kb in size. In some embodiments, the plasmid is about 5 kb in size. In some embodiments, the plasmid is about 5.5 kb in size. In some embodiments, the plasmid is about 6.5 kb in size. In some embodiments, the plasmid is about 7 kb in size. In some embodiments, the plasmid is about 6 kb in size. In some embodiments, the plasmid is about 7.5 kb in size. In some embodiments, the plasmid is about 8 kb in size.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor. In some embodiments, polynucleotide can contain regulatory sequences, such as transcription and translation initiation and termination codons, which are specific to the type of host (e.g., bacterium, fungus, plant, or animal) into which the polynucleotide is to be introduced, as appropriate and taking into consideration whether the polynucleotide is DNA- or RNA-based. In some embodiments, the polynucleotide can contain regulatory/control elements, such as a promoter, an enhancer, an intron, a polyadenylation signal, a Kozak consensus sequence, internal ribosome entry sites (IRES), a 2A sequence, and splice acceptor or donor. In some embodiments, the polynucleotide can contain a nonnative promoter operably linked to the nucleotide sequence encoding the recombinant receptor and/or one or more additional polypeptide(s). In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter or a modified form thereof or the MND promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the polynucleotide does not include a regulatory element, e.g. promoter.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of a GMCSFR alpha chain. In some cases, the nucleic acid sequence encoding the recombinant receptor, e.g., chimeric antigen receptor (CAR) contains a signal sequence that encodes a signal peptide.

In some embodiments, the polynucleotide contains a nucleic acid sequence encoding one or more additional polypeptides, e.g., one or more marker(s) and/or one or more effector molecules. In some embodiments, the one or more marker(s) includes a transduction marker, a surrogate marker and/or a selection marker. Among additional nucleic acid sequences introduced, e.g., encoding for one or more additional polypeptide(s), include nucleic acid sequences that can improve the efficacy of therapy, such as by promoting viability and/or function of transferred cells; nucleic acid sequences to provide a genetic marker for selection and/or evaluation of the cells, such as to assess in vivo survival or localization; nucleic acid sequences to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also WO 1992008796 and WO 1994028143 describing the use of bifunctional selectable fusion genes derived from fusing a dominant positive selectable marker with a negative selectable marker, and U.S. Pat. No. 6,040,177.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence. Extrinsic marker genes may in some cases be utilized in connection with engineered cell to permit detection or selection of cells and, in some cases, also to promote cell elimination and/or cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR) or a prostate-specific membrane antigen (PSMA) or modified form thereof, such as a truncated PSMA (tPSMA). In some aspects, tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19.

In some embodiments, the marker is or comprises a detectable protein, such as a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, codon-optimized, stabilized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof. In some aspects, expression of the enzyme can be detected by addition of a substrate that can be detected upon the expression and functional activity of the enzyme.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

Any of the recombinant receptors and/or the additional polypeptide(s) described herein can be encoded by one or more polynucleotides containing one or more nucleic acid sequences encoding recombinant receptors, in any combinations, orientation or arrangements. For example, one, two, three or more polynucleotides can encode one, two, three or more different polypeptides, e.g., recombinant receptors or portions or components thereof, and/or one or more additional polypeptide(s), e.g., a marker and/or an effector molecule. In some embodiments, one polynucleotide contains a nucleic acid sequence encoding a recombinant receptor, e.g., CAR, or portion or components thereof, and a nucleic acid sequence encoding one or more additional polypeptide(s). In some embodiments, one vector or construct contains a nucleic acid sequence encoding a recombinant receptor, e.g., CAR, or portion or components thereof, and a separate vector or construct contains a nucleic acid sequence encoding one or more additional polypeptide(s). In some embodiments, the nucleic acid sequence encoding the recombinant receptor and the nucleic acid sequence encoding the one or more additional polypeptide(s) are operably linked to two different promoters. In some embodiments, the nucleic acid encoding the recombinant receptor is present upstream of the nucleic acid encoding the one or more additional polypeptide(s). In some embodiments, the nucleic acid encoding the recombinant receptor is present downstream of the nucleic acid encoding one or more additional polypeptide(s).

In certain cases, one polynucleotide contains nucleic acid sequences encode two or more different polypeptide chains, e.g., a recombinant receptor and one or more additional polypeptide(s), e.g., a marker and/or an effector molecule. In some embodiments, the nucleic acid sequences encoding two or more different polypeptide chains, e.g., a recombinant receptor and one or more additional polypeptide(s), are present in two separate polynucleotides. For example, two separate polynucleotides are provided, and each can be individually transferred or introduced into the cell for expression in the cell. In some embodiments, the nucleic acid sequences encoding the marker and the nucleic acid sequences encoding the recombinant receptor are present or inserted at different locations within the genome of the cell. In some embodiments, the nucleic acid sequences encoding the marker and the nucleic acid sequences encoding the recombinant receptor are operably linked to two different promoters.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, the nucleic acid sequences encoding the recombinant receptor and the nucleic acid sequences encoding the one or more additional polypeptide(s) are operably linked to the same promoter and are optionally separated by an internal ribosome entry site (IRES), or a nucleic acid encoding a self-cleaving peptide or a peptide that causes ribosome skipping, such as a 2A element. For example, an exemplary marker, and optionally a ribosome skipping sequence, can be any as disclosed in PCT Pub. No. WO2014031687.

In some embodiments, transcription units can be engineered as a bicistronic unit containing an IRES, which allows coexpression of gene products (e.g. encoding the recombinant receptor and the additional polypeptide) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, e.g., de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A), equine rhinitis A virus (E2A), Thosea asigna virus (T2A), and porcine teschovirus-1 (P2A) as described in U.S. Patent Pub. No. 20070116690.

In some embodiments, the polynucleotide encoding the recombinant receptor and/or additional polypeptide is contained in a vector or can be cloned into one or more vector(s). In some embodiments, the one or more vector(s) can be used to transform or transfect a host cell, e.g., a cell for engineering. Exemplary vectors include vectors designed for introduction, propagation and expansion or for expression or both, such as plasmids and viral vectors. In some aspects, the vector is an expression vector, e.g., a recombinant expression vector. In some embodiments, the recombinant expression vectors can be prepared using standard recombinant DNA techniques.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech).

In some embodiments, the vector is a viral vector, such as a retroviral vector. In some embodiments, the polynucleotide encoding the recombinant receptor and/or additional polypeptide(s) are introduced into the cell via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy 18:1748-1757; and Hackett et al. (2010) Molecular Therapy 18:674-683).

In some embodiments, one or more polynucleotide(s) are introduced into cells using recombinant infectious virus particles, such as, e.g., vectors derived from simian virus 40 (SV40), adenoviruses, adeno-associated virus (AAV). In some embodiments, one or more polynucleotide(s) are introduced into T cells using recombinant lentiviral vectors or retroviral vectors, such as gammaretroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the vector is a retroviral vector. In some aspects, a retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In some embodiments, the vector (e.g., a viral vector) is a double stranded DNA vector. In some embodiments, the DNA vector is a closed-end vector. In some embodiments, the closed-end vector is Doggybone™ DNA (dbDNA). Closed-end DNA vectors, including dbDNA, are known in the art and have been described in e.g., Karda et al., Gene Ther (2019) 26:86-92.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, the polynucleotide encoding the recombinant receptor and/or one or more additional polypeptide(s), is introduced into a population containing cultured cells, such as by retroviral transduction, transfection, or transformation.

In some embodiments, one or more polynucleotide(s) are introduced into a T cell using electroporation (see, e.g., Chicaybam et al, (2013) PLoS ONE 8(3): e60298 and Van Tedeloo et al. (2000) Gene Therapy 7(16): 1431-1437). For examples, in some embodiments, one or more RNA molecules is introduce into a T cell using electroporation. Methods and systems for electroporation are known in the art, including e.g. Nucleofection® Technology (Lonza). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material, e.g., polynucleotides and/or vectors, into immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987) and other approaches described in, e.g., International Pat. App. Pub. No. WO 2014055668, and U.S. Pat. No. 7,446,190.

In some embodiments, the one or more polynucleotide(s) or vector(s) encoding a recombinant receptor and/or additional polypeptide(s) may be introduced into cells, e.g., T cells, either during or after expansion. This introduction of the polynucleotide(s) or vector(s) can be carried out with any suitable retroviral vector, for example. Resulting genetically engineered cells can then be liberated from the initial stimulus (e.g., anti-CD3/anti-CD28 stimulus) and subsequently be stimulated with a second type of stimulus (e.g., via a de novo introduced recombinant receptor). This second type of stimulus may include an antigenic stimulus in form of a peptide/MHC molecule, the cognate (cross-linking) ligand of the genetically introduced receptor (e.g. natural antigen and/or ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some embodiments, the T cells are activated prior to introduction of the polynucleotide or vector. In some embodiments, the T cells are incubated with e.g., anti-CD3/anti-CD28 antibodies. In some embodiments, the T cells are incubated with apololipoprotein E (ApoE) prior to, during, and/or subsequent to introduction of the polynucleotide or vector. For example, in some instances, the T cells are incubated with ApoE (e.g. ApoE4) prior to incubation with LNPs containing the polynucleotide or a composition thereof. In some embodiments, the T cells are incubated with about 1 g/mL ApoE4 prior to incubation with LNPs containing the polynucleotide or a composition thereof.

### 2. LNP-Based Delivery

Provided herein are methods of genetically engineering immune cells, comprising introducing one or more nucleic acid molecule(s) into a cell (e.g., a T cell) by incubating the immune cell with LNPs and/or co-LNPs, or a composition thereof, comprising the one or more nucleic acid molecule(s). In some embodiments, the method comprises incubating the immune cell with one or more of any of the LNPs and/or co-LNPs described herein.

In some embodiments, the method comprises incubating the immune cell with one or more of any of the LNPs described herein.

In some embodiments, the method comprises incubating the immune cell with a RNA LNP and a DNA LNP. In some embodiments, such methods are used to achieve insertion of a transgene, including into a targeted genomic locus. In some embodiments, the method comprises incubating the immune cell with a RNA LNP comprising mRNA encoding a transposase, and a DNA LNP comprising a transposon. In some embodiments, the transposon comprises a transgene encoding for a recombinant receptor (e.g., a CAR). In some embodiments, the method comprises incubating the immune cell with a first RNA LNP comprising mRNA encoding a recombinant nuclease capable of inducing a DNA break, a second RNA LNP comprising a guide RNA (gRNA), and a DNA LNP comprising a repair template (e.g., HDR template DNA). In some embodiments, the method comprises incubating the immune cell with a RNA LNP comprising a guide RNA (gRNA) and a DNA LNP comprising a repair template (e.g., HDR template DNA). In some embodiments, the method comprises incubating the immune cell with a RNA LNP gRNA complexed with a recombinant nuclease (e.g., Cas) and a DNA LNP comprising a repair template (e.g., HDR template DNA). In some embodiments, the gRNA is a single guide RNA (sgRNA) comprising a crispr RNA (crRNA) and a tracrRNA. In some embodiments, the recombinant nuclease is Cas. In some embodiments, the Cas is Cas9. In some embodiments, the Cas is Cas12a. In some embodiments, the HDRt DNA encodes a recombinant receptor (e.g., a CAR).

In some embodiments, the method comprises incubating the immune cell with two different RNA LNPs. In some embodiments, such methods are used to achieve knockdown or knockout of a target genomic locus. In some aspects, the method comprises incubating the immune cell with a first RNA LNP comprising mRNA encoding a recombinant nuclease capable of inducing a DNA break and a second RNA LNP comprising a guide RNA (gRNA). In some embodiments, the gRNA is a single guide RNA (sgRNA) comprising a crispr RNA (crRNA) and a tracrRNA. In some embodiments, the recombinant nuclease is Cas. In some embodiments, the Cas is Cas9. In some embodiments, the Cas is Cas12a.

In some embodiments, the method comprises incubating the immune cell with one or more co-formulated LNP(s) (co-LNPs) or compositions thereof. In some embodiments, incubating the immune cell with one or more co-LNPs may increase transduction efficiency, such as by reducing competitive uptake that would result from incubating the immune cell with separately formulated LNPs. In some embodiments, the method comprises incubating the immune cell with a RNA co-LNP. In some embodiments, the methods comprise incubating the immune cell with a RNA co-LNP containing mRNA encoding a recombinant nuclease capable of inducing a DNA break (e.g., Cas), and a gRNA. In some embodiments, such methods are used to achieve knockdown or knockout of a target genomic locus. In some embodiments, the method comprises incubating the immune cell with a RNA co-LNP and a DNA LNP. In some embodiments, the methods comprise incubating the immune cell with a RNA co-LNP comprising mRNA encoding a recombinant nuclease capable of inducing a DNA break (e.g., Cas) and a gRNA and a DNA LNP comprising HDRt DNA. In some embodiments, the methods comprising incubating the immune cell with a co-LNP containing RNA and DNA. In some aspects, the methods comprise incubating the immune cell with a co-LNP containing a gRNA complexed with a recombinant nuclease capable of inducing a DNA break and HDRt DNA. In some aspects, the methods comprise incubating the immune cell with a co-LNP containing a gRNA, mRNA encoding a recombinant nuclease capable of inducing a DNA break, and HDRt DNA. In some embodiments, the gRNA is a single guide RNA (sgRNA) comprising a crispr RNA (crRNA) and a tracrRNA. In some embodiments, such methods are used to achieve insertion of a transgene, including into a targeted genomic locus. In some embodiments, the recombinant nuclease is Cas. In some embodiments, the Cas is Cas9. In some embodiments, the Cas is Cas12a.

In some embodiments, at the time of incubating the immune cell with the LNP, the co-LNP, both, or any composition thereof, the immune cell is activated. In some embodiments, at the time of incubating the immune cell with the LNP or a composition thereof, the immune cell is activated. In some embodiments, at the time of incubating the immune cell with the co-LNP or a composition thereof, the immune cell is activated. In some embodiments, at the time of incubating the immune cell with the LNP or a composition thereof and the co-LNP or a composition thereof, the immune cell is activated. In some embodiments, at the time of incubating the immune cell with the LNP, the co-LNP, both, or any composition thereof, the immune cell expresses CD25, CD26, CD27, CD28, CD30, CD71, CD154, CD40L, CD134, or a combination thereof. In some embodiments, at the time of incubating the immune cell with the LNP or a composition thereof, the immune cell expresses CD25, CD26, CD27, CD28, CD30, CD71, CD154, CD40L, CD134, or a combination thereof. In some embodiments, at the time of incubating the immune cell with the co-LNP or a composition thereof, the immune cell expresses CD25, CD26, CD27, CD28, CD30, CD71, CD154, CD40L, CD134, or a combination thereof. In some embodiments, at the time of incubating the immune cell with the LNP or a composition thereof and the co-LNP or a composition thereof, the immune cell expresses CD25, CD26, CD27, CD28, CD30, CD71, CD154, CD40L, CD134, or a combination thereof. In some embodiments, the immune cell is incubated under stimulating conditions prior to incubating the immune cell with the LNP, the co-LNP, both, or any composition thereof. In some embodiments, the immune cell is incubated under stimulating conditions for between about 24 hours and about 72 hours, or for about 48 hours.

In some embodiments, the stimulating conditions comprise incubation with a stimulatory reagent capable of activating an intracellular signaling domain of a component of a TCR complex and an intracellular signaling domain of a costimulatory molecule. In some embodiments, the stimulatory reagent comprises a primary agent that binds to CD3 and a secondary agent that binds to a T cell costimulatory molecule. In some embodiments, the costimulatory molecule is selected from the group consisting of CD28, 4-1BB, OX40, and ICOS. In some embodiments, the costimulatory molecule is CD28. In some embodiments, the primary agent is an anti-CD3 antibody or antigen-binding fragment, and the secondary agent is an anti-CD28 antibody or antigen-binding fragment.

In some embodiments, the immune cell is incubated with apolipoprotein E (ApoE) prior to, during, and/or subsequent to incubating the immune cell with the LNP, the co-LNP, both, or any composition thereof. In some embodiments, the immune cell is incubated with apolipoprotein E (ApoE) prior to incubating the immune cell with the LNP, the co-LNP, both, or any composition thereof. In some embodiments, the immune cell is incubated with apolipoprotein E (ApoE) during the incubating the immune cell with the LNP, the co-LNP, both, or any composition thereof. In some embodiments, the immune cell is incubated with apolipoprotein E (ApoE) prior to and during the incubating the immune cell with the LNP, the co-LNP, both, or any composition thereof. In some embodiments, the immune cell is incubated with about 1 g/mL of ApoE. In some embodiments, ApoE is ApoE2, ApoE3, or ApoE4. In some embodiments, the ApoE is ApoE4. In some embodiments, the immune cell is incubated with about 1 g/mL of ApoE4.

In some embodiments, the immune cell is a T cell. In some embodiments, the T cell is a primary T cell. In some embodiments, the T cell is a CD4+ T cell or a CD8+ T cell.

Also provided herein are genetically engineered cells produced by any of the methods provided herein.

### 3. Hybrid Delivery: LNPs and Electroporation

Provided herein are methods of genetically engineering immune cells, comprising introducing one or more nucleic acid molecule(s) into a cell (e.g., a T cell) by (1) incubating the immune cell with LNPs and/or co-LNPs, or a composition thereof, comprising the one or more nucleic acid molecule(s); and (2) electroporating the immune cell to introduce one or more nucleic acid molecule(s) into the immune cell. In some embodiments, the methods comprise (1) incubating the immune cell with LNPs and/or co-LNPs, or a composition thereof, comprising a DNA molecule; and (2) electroporating the immune cell to introduce an RNA molecule into the immune cell. In some embodiments, the methods comprise (1) incubating the immune cell with LNPs and/or co-LNPs, or a composition thereof, comprising an RNA molecule; and (2) electroporating the immune cell to introduce an RNA molecule into the immune cell. In some embodiments, electroporating the immune cell is carried out by any method and/or system known in the art, including e.g., Nucleofection® technology (Lonza).

In some aspects, it is contemplated herein that a hybrid non-viral delivery approach to introducing one or more nucleic acid molecule(s) into a cell (e.g., a T cell) can be achieved by introducing a nucleic acid molecule (e.g., DNA) into the immune cell by use of a LNP comprising the nucleic acid molecule and by introducing another nucleic acid molecule (e.g., RNA) into the immune cell by use of electroporation. In some embodiments, it is observed herein that use of the hybrid non-viral delivery approach as described may achieve high levels of transduction efficiency while minimizing toxicity.

In some embodiments, the method comprises incubating the immune cell with a DNA LNP and/or a RNA LNP and electroporating the immune cell with RNA. In some embodiments, such methods are used to achieve insertion of a transgene, including into a targeted genomic locus. In some embodiments, the method comprises incubating the immune cell with mRNA encoding a transposase, and incubating the immune cell with a DNA LNP comprising a transposon. In some embodiments, the transposon comprises a transgene encoding for a recombinant receptor (e.g., a CAR). In some embodiments, the method comprises electroporating the immune cell with mRNA encoding a recombinant nuclease capable of inducing a DNA break, and incubating the immune cell with a RNA LNP comprising a guide RNA (gRNA) and a DNA LNP comprising a repair template (e.g., HDR template DNA). In some embodiments, the method comprises electroporating the immune cell with a gRNA, and incubating the immune cell with a RNA LNP comprising mRNA encoding a recombinant nuclease capable of inducing a DNA break and a DNA LNP comprising a repair template (e.g., HDR template DNA). In some embodiments, the gRNA is a single guide RNA (sgRNA) comprising a crispr RNA (crRNA) and a tracrRNA. In some embodiments, the recombinant nuclease is Cas. In some embodiments, the Cas is Cas9. In some embodiments, the Cas is Cas12a. In some embodiments, the HDRt DNA encodes a recombinant receptor (e.g., a CAR).

In some embodiments, the method comprises incubating the immune cell with a RNA LNP and electroporating the immune cell with RNA. In some embodiments, such methods are used to achieve knockdown or knockout of a target genomic locus. In some aspects, the method comprises electroporating the immune cell with mRNA encoding a recombinant nuclease capable of inducing a DNA break and introducing the immune cell with a RNA LNP comprising a guide RNA (gRNA). In some aspects, the method comprises electroporating the immune cell with a gRNA and introducing the immune cell with a RNA LNP comprising mRNA encoding a recombinant nuclease capable of inducing a DNA break. In some embodiments, the recombinant nuclease is Cas. In some embodiments, the Cas is Cas9. In some embodiments, the Cas is Cas12a.

In some embodiments, the method comprises incubating the immune cell with a co-formulated LNP comprising RNA and/or DNA and electroporating the immune cell with RNA. In some embodiments, the method comprises incubating the immune cell with a co-formulated LNP comprising RNA and DNA and electroporating the immune cell with RNA. In some embodiments, the co-formulated LNP comprises a gRNA and HDRt DNA. Thus, in some aspects, the immune cell is incubated with a co-LNP comprising gRNA and HDRt DNA, and the immune cell is electroporated with mRNA encoding a recombinant nuclease capable of inducing a DNA break. In some embodiments, the co-formulated LNP comprises mRNA encoding a recombinant nuclease capable of inducing a DNA break and HDRt DNA. Thus, in some aspects, the immune cell is incubated with a co-LNP comprising mRNA encoding a recombinant nuclease capable of inducing a DNA break and HDRt DNA, and the immune cell is electroporated with a gRNA. In some embodiments, the gRNA is a single guide RNA (sgRNA) comprising a crispr RNA (crRNA) and a tracrRNA. In some embodiments, the recombinant nuclease is Cas. In some embodiments, the Cas is Cas9. In some embodiments, the Cas is Cas12a. In some embodiments, the HDRt DNA encodes a recombinant receptor (e.g., a CAR).

It is contemplated herein that the introducing and the electroporating may be carried out in any order. For example, in some aspects, the introducing the immune cell with the LNP or co-LNP is carried out prior to electroporating the immune cell with the RNA molecule. In some aspects, the electroporating the immune cell with the RNA molecule is carried out prior to introducing the immune cell with the LNP or co-LNP.

In some embodiments, at the time of incubating the immune cell with the LNP, the co-LNP, both, or any composition thereof, the immune cell is activated. In some embodiments, at the time of electroporating the immune cell with an RNA molecule, the immune cell is activated. In some aspects, the method comprises, in order, activating the immune cell, electroporating the immune cell with the RNA, and incubating the immune cell with the LNP or co-LNP.

In some embodiments, an activated cell expresses CD25, CD26, CD27, CD28, CD30, CD71, CD154, CD40L, CD134, or a combination thereof. In some embodiments, activating the immune cell comprises incubating the immune cell under stimulating conditions. In some embodiments, the immune cell is incubated under stimulating conditions for between about 24 hours and about 72 hours, or for about 48 hours.

In some embodiments, the stimulating conditions comprise incubation with a stimulatory reagent capable of activating an intracellular signaling domain of a component of a TCR complex and an intracellular signaling domain of a costimulatory molecule. In some embodiments, the stimulatory reagent comprises a primary agent that binds to CD3 and a secondary agent that binds to a T cell costimulatory molecule. In some embodiments, the costimulatory molecule is selected from the group consisting of CD28, 4-1BB, OX40, and ICOS. In some embodiments, the costimulatory molecule is CD28. In some embodiments, the primary agent is an anti-CD3 antibody or antigen-binding fragment, and the secondary agent is an anti-CD28 antibody or antigen-binding fragment.

In some embodiments, the immune cell is incubated with apolipoprotein E (ApoE) prior to, during, and/or subsequent to incubating the immune cell with the LNP, the co-LNP, both, or any composition thereof. In some embodiments, the immune cell is incubated with apolipoprotein E (ApoE) prior to incubating the immune cell with the LNP, the co-LNP, both, or any composition thereof. In some embodiments, the immune cell is incubated with apolipoprotein E (ApoE) during the incubating the immune cell with the LNP, the co-LNP, both, or any composition thereof. In some embodiments, the immune cell is incubated with apolipoprotein E (ApoE) prior to and during the incubating the immune cell with the LNP, the co-LNP, both, or any composition thereof. In some embodiments, the immune cell is incubated with apolipoprotein E (ApoE) prior to electroporating the cell with the RNA molecule. In some embodiments, the immune cell is incubated with about 1 μg/mL of ApoE. In some embodiments, ApoE is ApoE2, ApoE3, or ApoE4. In some embodiments, the ApoE is ApoE4. In some embodiments, the immune cell is incubated with about 1 μg/mL of ApoE4.

In some embodiments, the immune cell is a T cell. In some embodiments, the T cell is a primary T cell. In some embodiments, the T cell is a CD4+ T cell or a CD8+ T cell.

Also provided herein are genetically engineered cells produced by any of the methods provided herein.

### D. Exemplary Features of the Cells

Provided herein are genetically engineered cells generated by any of the methods described herein, and compositions thereof.

In some embodiments, a composition of genetically engineered cells produced by any of the methods herein contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable cells. In some embodiments, the composition contains at least at or about 75% viable cells. In certain embodiments, the composition contains at least at or about 85%, at least at or about 90%, or at least at or about 95% viable cells. In some embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD3+ T cells. In particular embodiments, the composition contains at least at or about 75% viable CD3+ T cells. In certain embodiments, the composition contains at least at or about 85%, at least at or about 90%, or at least at or about 95% viable CD3+ T cells. In some embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD4+ T cells. In certain embodiments, the composition contains at least at or about 75% viable CD4+ T cells. In particular embodiments, the composition contains at least at or about 85%, at least at or about 90%, or at least at or about 95% viable CD4+ T cells. In particular embodiments, the composition contains at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% viable CD8+ T cells. In some embodiments, the composition contains at least at or about 75% viable CD8+ T cells. In certain embodiments, the composition contains at least at or about 85%, at least at or about 90%, or at least at or about 95% viable CD8+ T cells.

In particular embodiments, a composition of genetically engineered cells produced by any of the methods herein contains a low portion and/or frequency of cells that are undergoing and/or are prepared, primed, and/or entering apoptosis. In particular embodiments, the composition contains a low portion and/or frequency of cells that are positive for an apoptotic marker. In some embodiments, less than at or about 40%, less than at or about 35%, less than at or about 30%, less than at or about 25%, less than at or about 20%, less than at or about 15%, less than at or about 10%, less than at or about 5%, or less than at or about 1% of the cells of the composition express, contain, and/or are positive for an apoptotic marker. In certain embodiments, less than at or about 25% of the cells of the composition express, contain, and/or are positive for a marker of apoptosis. In certain embodiments, less than at or about less than at or about 10% cells of the composition express, contain, and/or are positive for an apoptotic marker. In certain embodiments, less than at or about less than at or about 5% cells of the composition express, contain, and/or are positive for an apoptotic marker. In certain embodiments, less than at or about less than at or about 1% cells of the composition express, contain, and/or are positive for an apoptotic marker.

In some embodiments, a composition generated or produced in connection with the provided methods contains cells (e.g. T cells) expressing a recombinant receptor, e.g., a TCR or a CAR. In some embodiments, expressing a recombinant receptor may include, but is not limited to, having one or more recombinant receptor proteins localized at the cell membrane and/or cell surface, having a detectable amount of recombinant receptor protein, having a detectable amount of mRNA encoding the recombinant receptor, having or containing a recombinant polynucleotide that encodes the recombinant receptor, and/or having or containing an mRNA or protein that is a surrogate marker for recombinant receptor expression.

In some embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the composition express the recombinant receptor. In certain embodiments, at least or about 50% of the cells of the composition express the recombinant receptor. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD3+ T cells of the composition express the recombinant receptor. In some embodiments, at least or about 50% of the CD3+ T cells of the composition express the recombinant receptor. In certain embodiments, at least or about 5%, at least or about 10%, at least or about 20%, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the cells of the composition are CD3+ T cells that express the recombinant receptor. In some embodiments, at least or about 50% of the cells of the composition are CD3+ T cells that express the recombinant receptor.

In particular embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD4+ T cells of the composition express the recombinant receptor. In particular embodiments, at least or about 50% of the CD4+ T cells of the composition express the recombinant receptor. In some embodiments, at least or about 30%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 97%, at least or about 99%, or more than 99% of the CD8+ T cells of the composition express the recombinant receptor. In certain embodiments, at least or about 50% of the CD8+ T cells of the composition express the recombinant receptor.

In particular embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) cells of the composition are viable cells, e.g., cells negative for an apoptotic marker, such as a caspase (e.g., an activated caspase-3). In certain embodiments, at least at or about 85%, at least at or about 90%, or at least at or about 95% of recombinant receptor-expressing (e.g., CAR+) cells of the composition are viable cells, e.g., cells negative for an apoptotic marker, such as a caspase (e.g., an activated caspase-3). In some embodiments, at least at or about 90% of recombinant receptor-expressing (e.g., CAR+) cells of the composition are viable cells, e.g., cells negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of CD3+ T cells of the composition are viable cells, e.g., cells negative for an apoptotic marker, such as a caspase (e.g., an activated caspase-3). In certain embodiments, at least at or about 85%, at least at or about 90%, or at least at or about 95% of CD3+ T cells of the composition are viable cells, e.g., cells negative for an apoptotic marker, such as a caspase (e.g., an activated caspase-3). In particular embodiments, at least at or about 90% of CD3+ T cells of the composition are viable cells, e.g., cells negative for an apoptotic marker, such as a caspase (e.g., an activated caspase-3). In some embodiments, at least at or about 50%, at least at or about 60%, at least at or about 70%, at least at or about 75%, at least at or about 80%, at least at or about 85%, at least at or about 90%, at least at or about 95%, at least at or about 99%, or at least at or about 99.9% of recombinant receptor-expressing (e.g., CAR+) CD3+ T cells of the composition are viable cells, e.g., cells negative for an apoptotic marker, such as a caspase (e.g., an activated caspase-3). In particular embodiments, at least at or about 85%, at least at or about 90%, or at least at or about 95% of recombinant receptor-expressing (e.g., CAR+) CD3+ T cells of the composition are viable cells, e.g., cells negative for an apoptotic marker, such as a caspase (e.g., an activated caspase-3). In certain embodiments, at least at or about 90% of recombinant receptor-expressing (e.g., CAR+) CD3+ T cells of the composition are viable cells, e.g., cells negative for an apoptotic marker, such as a caspase (e.g., an activated caspase-3).

In particular embodiments, a majority of the cells of the composition are naïve or naïve-like cells, central memory cells, and/or effector memory cells. In particular embodiments, a majority of the cells of the composition are naïve-like or central memory cells. In some embodiments, a majority of the cells of the composition are central memory cells. In some aspects, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24(5):563-571.

In certain embodiments, the cells of the composition have a high portion and/or frequency of naïve-like T cells or T cells that are surface positive for a marker expressed on naïve-like T cells. In certain embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+CCR7+, where the cells are CD45RA+ or CD45RA−. In certain embodiments, naïve-like T cells or the T cells that are surface positive for a marker expressed on naïve-like T cells are CD62L-CCR7+.

### IV. Compositions and Formulations

### A. Engineered Cell Compositions and Formulations

In some embodiments, the engineered cells produced by any of the methods disclosed herein, is provided as a composition or formulation, such as a pharmaceutical composition or formulation. Such compositions can be used in accord with the provided methods, articles of manufacture, and/or with the provided compositions, such as in adoptive cell therapy and/or gene therapy applications.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The formulation or composition may also be administered in combination with another form of treatment useful for the particular indication, disease, or condition being prevented or treated with the cells or agents, where the respective activities do not adversely affect one another. Thus, in some embodiments, the pharmaceutical composition is administered in combination with deep brain stimulation (DBS).

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by stereotactic injection (e.g., using a catheter). In some embodiments, a given dose is administered by a single bolus administration of the cells or agent. In some embodiments, it is administered by multiple bolus administrations of the cells or agent, for example, over a period of months or years. In some embodiments, the agents or cells can be administered by stereotactic injection into the brain, such as in the striatum.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous. For example, non-pluripotent cells (e.g., fibroblasts) can be obtained from a subject, and administered to the same subject following reprogramming and differentiation. When administering a therapeutic composition (e.g., a pharmaceutical composition containing a genetically reprogrammed and/or differentiated cell or an agent that treats or ameliorates symptoms of a disease or disorder, such as a neurodegenerative disorder), it will generally be formulated in a unit dosage injectable form (solution, suspension, emulsion). Formulations include those for stereotactic administration, such as into the brain (e.g. the striatum).

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes

### V. Methods of Administration

In some aspects, the engineered cells produced by the methods described herein, e.g., cells genetically engineered using the LNPs or compositions thereof, optionally in combination with electroporation, can be used in connection with a method of treatment, e.g., including administering any of the engineered cells or compositions containing engineered cells that have been produced using the methods provided herein. In some aspects, also provided are methods of administering any of the engineered cells or compositions containing engineered cells described herein to a subject, such as a subject that has a disease or disorder. In some aspects, also provided are uses of any of the engineered cells or compositions containing engineered cells described herein or produced by the methods described herein, for treatment of a disease or disorder. In some aspects, also provided are uses of any of the engineered cells or compositions containing engineered cells described herein or produced by the methods described herein, for the manufacture of a medicament for the treatment of a disease or disorder. In some aspects, also provided are any of the engineered cells or compositions containing engineered cells described herein or produced by the methods described herein, for use in treatment of a disease or disorder, or for administration to a subject having a disease or disorder.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen _{1B} (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT) vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen, such as a viral antigen (e.g., a viral antigen from HIV, HCV, HBV), bacterial antigens, and/or parasitic antigens.

In some embodiments, the antibody or an antigen-binding fragment (e.g. scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the disease or condition is a B cell malignancy. In some embodiments, the B cell malignancy is a leukemia or a lymphoma. In some aspects, the disease or condition is acute lymphoblastic leukemia (ALL), adult ALL, chronic lymphoblastic leukemia (CLL), non-Hodgkin lymphoma (NHL), or Diffuse Large B-Cell Lymphoma (DLBCL). In some cases, the disease or condition is an NHL, such as or including an NHL that is an aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B). In some aspects, the recombinant receptor, such as a CAR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30, or combinations thereof.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known. See, for instance, Wadwa et al., J. Drug Targeting 3: 1 1 1 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

### A. Dosing

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, 10 million cells, about 15 million cells, about 20 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, the dose of cells is a flat dose of cells or fixed dose of cells such that the dose of cells is not tied to or based on the body surface area or weight of a subject.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the dose of genetically engineered cells comprises from or from about 1×10⁵ to 5×10⁸ total CAR-expressing T cells, 1×10⁵ to 2.5×10⁸ total CAR-expressing T cells, 1×10⁵ to 1×10⁸ total CAR-expressing T cells, 1×10⁵ to 5×10⁷ total CAR-expressing T cells, 1×10⁵ to 2.5×10⁷ total CAR-expressing T cells, 1×10⁵ to 1×10⁷ total CAR-expressing T cells, 1×10⁵ to 5×10⁶ total CAR-expressing T cells, 1×10⁵ to 2.5×10⁶ total CAR-expressing T cells, 1×10⁵ to 1×10⁶ total CAR-expressing T cells, 1×10⁶ to 5×10⁸ total CAR-expressing T cells, 1×10⁶ to 2.5×10⁸ total CAR-expressing T cells, 1×10⁶ to 1×10⁸ total CAR-expressing T cells, 1×10⁶ to 5×10⁷ total CAR-expressing T cells, 1×10⁶ to 2.5×10⁷ total CAR-expressing T cells, 1×10⁶ to 1×10⁷ total CAR-expressing T cells, 1×10⁶ to 5×10⁶ total CAR-expressing T cells, 1×10⁶ to 2.5×10⁶ total CAR-expressing T cells, 2.5×10⁶ to 5×10⁸ total CAR-expressing T cells, 2.5×10⁶ to 2.5×10⁸ total CAR-expressing T cells, 2.5×10⁶ to 1×10⁸ total CAR-expressing T cells, 2.5×10⁶ to 5×10⁷ total CAR-expressing T cells, 2.5×10⁶ to 2.5×10⁷ total CAR-expressing T cells, 2.5×10⁶ to 1×10⁷ total CAR-expressing T cells, 2.5×10⁶ to 5×10⁶ total CAR-expressing T cells, 5×10⁶ to 5×10⁸ total CAR-expressing T cells, 5×10⁶ to 2.5×10⁷ total CAR-expressing T cells, 5×10⁶ to 1×10⁸ total CAR-expressing T cells, 5×10⁶ to 5×10⁷ total CAR-expressing T cells, 5×10⁶ to 2.5×10⁷ total CAR-expressing T cells, 5×10⁶ to 1×10⁷ total CAR-expressing T cells, 1×10⁷ to 5×10⁸ total CAR-expressing T cells, 1×10⁷ to 2.5×10⁸ total CAR-expressing T cells, 1×10⁷ to 1×10⁸ total CAR-expressing T cells, 1×10⁷ to 5×10⁷ total CAR-expressing T cells, 1×10⁷ to 2.5×10⁷ total CAR-expressing T cells, 2.5×10⁷ to 5×10⁸ total CAR-expressing T cells, 2.5×10⁷ to 2.5×10⁸ total CAR-expressing T cells, 2.5×10⁷ to 1×10⁸ total CAR-expressing T cells, 2.5×10⁷ to 5×10⁷ total CAR-expressing T cells, 5×10⁷ to 5×10⁸ total CAR-expressing T cells, 5×10⁷ to 2.5×10⁸ total CAR-expressing T cells, 5×10⁷ to 1×10⁸ total CAR-expressing T cells, 1×10⁸ to 5×10⁸ total CAR-expressing T cells, 1×10⁸ to 2.5×10⁸ total CAR-expressing T cells, or 2.5×10⁸ to 5×10⁸ total CAR-expressing T cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁸ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

### VI. Articles of Manufacture and Kits

Also provided are kits and articles of manufacture, such as those containing reagents for performing the methods provided herein, e.g., reagents for producing LNPs and compositions thereof and/or reagents for introducing a nucleic acid molecule into a T cell using LNPs and/or compositions thereof. In some aspects, the kits or articles of manufacture can contain reagents and/or nucleic acids for use in engineering or manufacturing processes to generate the engineered T cells.

In some embodiments, the kits can contain reagents and/or consumables required for producing LNPs and compositions thereof. In some embodiments, the kits can contain reagents and/or consumables required for delivery of nucleic acid into T cells using such LNPs and/or compositions thereof. In some embodiments, the kits can contain T cells engineered using the LNPs and/or compositions thereof, such as for use in adoptive cell therapy. The various components of the kit may be present in separate containers or certain compatible components may be precombined into a single container. In some embodiments, the kits further contain instructions for using the components of the kit to practice the provided methods.

Also provided are articles of manufacture, which may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

In some embodiments, an article of manufacture contains two or more containers. In some embodiments, the first container comprises a first composition and a second composition, wherein the first composition comprises a first population of the engineered T cells used for the immunotherapy, e.g., CD4+ T cell therapy, and the second composition comprises a second population of the engineered T cells, wherein the second population may be engineered separately from the first population, e.g., CD8+ T cell therapy. In some embodiments, the first and second cell compositions contain a defined ratio of the engineered cells, e.g., CD4+ and CD8+ cells (e.g., 1:1 ratio of CD4+:CD8+ CAR+ T cells).

The article of manufacture may further include a package insert indicating that the compositions can be used to treat a particular condition.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In certain embodiments, “about X” refers to a value of ±25%, ±10%, ±5%, ±2%, ±1%, ±0.1%, or ±0.01% of X.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, for instance, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

As used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone.

### VIII. Exemplary Embodiments

Among the provided embodiments are:
1. A lipid nanoparticle (LNP) comprising:
   (1) an ionizable lipid comprising a diketopiperazine ring core, wherein the ionizable lipid is an ionizable amino lipid; and
   (2) a deoxyribonucleic acid (DNA) molecule.
2. The LNP of embodiment 1, wherein the ionizable lipid is an ionizable amino lipidoid.
3. The LNP of embodiment 1 or embodiment 2, wherein the mass fraction of the ionizable lipid is between about 35% and about 45%.
4. The LNP of any of embodiments 1-3, wherein the ionizable lipid comprises an unsaturated tail, optionally an unsaturated linoleic tail.
5. The LNP of any of embodiments 1-2, wherein the ionizable lipid is: (a) OF-C4-Deg-Lin, or an analog thereof; (b) cKK-E12, or an analog thereof; or (c) DLin-KC2-DMA, or an analog thereof.
6. The LNP of any of embodiments 1-5, wherein the ionizable lipid is OF-C4-Deg-Lin; and the mass fraction of the ionizable lipid is between about 35% and about 45%.
7. The LNP of any of embodiments 1-5, wherein the ionizable lipid is cKK-E12; and the mass fraction of the ionizable lipid is between about 35% and about 45%.
8. A lipid nanoparticle (LNP) comprising:
   (1) an ionizable lipid, or an analog thereof, wherein the ionizable lipid is DLin-KC2-DMA; and
   (2) a deoxyribonucleic acid (DNA) sequence,
   wherein the mass fraction of the ionizable lipid is between about 35% and about 45%.
9. The LNP of any of embodiments 1-8, comprising a helper lipid.
10. The LNP of any of embodiments 1-9, comprising a polyethylene glycol (PEG)-conjugated lipid.
11. The LNP of any of embodiments 1-10, comprising cholesterol.
12. The LNP of any of embodiments 1-11, wherein the mass fraction of the ionizable lipid is about 40%.
13. The LNP of any of embodiments 9-12, wherein the mass fraction of the helper lipid is between about 18% and about 22%.
14. The LNP of any of embodiments 9-13, wherein the mass fraction of the helper lipid is about 19%.
15. The LNP of any of embodiments 10-14, wherein the mass fraction of the PEG-conjugated lipid is between about 2% and about 3%.
16. The LNP of any of embodiments 10-15, wherein the mass fraction of the PEG-conjugated lipid is about 2.5%.
17. The LNP of any of embodiments 11-16, wherein the mass fraction of the cholesterol is between about 30% and about 40%.
18. The LNP of any of embodiments 11-17, wherein the mass fraction of the cholesterol is about 35%.
19. The LNP of any of embodiments 9-18, wherein the helper lipid is 1-stearoyl-2-oleoyl-sn-glycero-3-phosphocholine (SOPC).
20. The LNP of any of embodiments 10-19, wherein the PEG-conjugated lipid is DMG-PEG2000.
21. The LNP of any of embodiments 1-20, wherein the mass fraction of the DNA molecule is between about 3% and about 4%.
22. The LNP of any of embodiments 1-21, wherein the mass fraction of the DNA molecule is about 3.5%.
23. A lipid nanoparticle (LNP) comprising:
   (1) between about 35% and about 45% mass fraction of an ionizable lipid, wherein the ionizable lipid is OF-C4-Deg-Lin;
   (2) between about 3% and about 4% mass fraction of a deoxyribonucleic acid (DNA) molecule;
   (3) between about 18% and about 22% mass fraction of a helper lipid;
   (4) between about 2% and about 3% mass fraction of a polyethylene glycol (PEG)-conjugated lipid; and
   (5) between about 30% and about 40% mass fraction of cholesterol.
24. A lipid nanoparticle (LNP) comprising:
   (1) between about 35% and about 45% mass fraction of an ionizable lipid, wherein the ionizable lipid is cKK-E12;
   (2) between about 3% and about 4% mass fraction of a deoxyribonucleic acid (DNA) molecule;
   (3) between about 18% and about 22% mass fraction of a helper lipid;
   (4) between about 2% and about 3% mass fraction of a polyethylene glycol (PEG)-conjugated lipid; and
   (5) between about 30% and about 40% mass fraction of cholesterol.
25. A lipid nanoparticle (LNP) comprising:
   (1) between about 35% and about 45% mass fraction of an ionizable lipid, wherein the ionizable lipid is DLin-KC2-DMA;
   (2) between about 3% and about 4% mass fraction of a deoxyribonucleic acid (DNA) molecule;
   (3) between about 18% and about 22% mass fraction of a helper lipid;
   (4) between about 2% and about 3% mass fraction of a polyethylene glycol (PEG)-conjugated lipid; and
   (5) between about 30% and about 40% mass fraction of cholesterol.
26. The LNP of any of embodiments 1-25, wherein the DNA molecule comprises a transgene.
27. The LNP of embodiment 26, wherein the transgene encodes a recombinant receptor.
28. The LNP of any of embodiments 1-27, wherein the DNA molecule is a closed end DNA (ceDNA) vector.
29. The LNP of embodiment 28, wherein the transgene is positioned between protelomerase binding sequences.
30. The LNP of embodiment 26 or embodiment 27, wherein the transgene is operably linked to a promoter and positioned between inverted terminal repeats (ITRs).
31. The co-LNP of any of embodiments 27-30, wherein the recombinant receptor is a chimeric antigen receptor (CAR) or a T cell receptor (TCR).
32. The LNP of any of embodiments 27-31, wherein the recombinant receptor is a CAR.
33. The LNP of embodiment 31 or embodiment 32, wherein the CAR comprises an extracellular antigen-binding domain, a transmembrane domain, and an intracellular region.
34. The LNP of embodiment 33, wherein the extracellular antigen-binding domain is an antibody or an antigen-binding fragment thereof that binds to an antigen that is associated with, or expressed on, a cell or tissue of a disease or condition.
35. The LNP of embodiment 34, wherein the antigen is selected from the group consisting of αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), and Wilms Tumor 1 (WT-1).
36. The LNP of any of embodiments 33-35, wherein the intracellular region comprises an intracellular signaling domain that is or comprises an intracellular signaling domain of a CD3 chain, or a signaling portion thereof.
37. The LNP of any of embodiments 33-36, wherein the intracellular region comprises one or more costimulatory signaling domain(s) comprising an intracellular signaling domain selected from the group consisting of: a CD28, a 4-1BB, an ICOS, or a signaling portion thereof.
38. The LNP of any of embodiments 1-25, wherein the DNA molecule comprises a single-stranded DNA oligonucleotide (ssODN) or a double-stranded DNA oligonucleotide (dsODN), the ssODN or the dsODN comprising a nucleotide sequence that is homologous to a target genomic locus.
39. A co-formulated lipid nanoparticle (co-LNP) comprising:
   (1) a deoxyribonucleic acid (DNA) molecule and a ribonucleic acid (RNA) molecule; and
   (2) a first ionizable lipid and a second ionizable lipid.
40. The co-LNP of embodiment 39, further comprising a third ionizable lipid.
40. The co-LNP of embodiment 39 or embodiment 40, wherein (i) the DNA molecule is associated with the first ionizable lipid; and (ii) the RNA molecule is associated with the second ionizable lipid and/or the third ionizable lipid.
41. A co-formulated lipid nanoparticle (co-LNP) comprising a fusion of a first lipid nanoparticle (LNP) and a second lipid nanoparticle (LNP), wherein, prior to fusion:
   (1) the first LNP comprises:
      (i) a deoxyribonucleic acid (DNA) molecule; and
      (ii) a first ionizable lipid; and
   (2) the second LNP comprises:
      (i) a ribonucleic acid (RNA) molecule; and
      (ii) a second ionizable lipid.
42. The co-LNP of embodiment 41, wherein the first ionizable lipid and/or the second ionizable lipid is an ionizable amino lipid.
43. The co-LNP of embodiment 41 or embodiment 42, comprising a volumetric ratio of the first LNP to the second LNP that is between about 3:1 and about 1:3.
44. The co-LNP of embodiment 41 or embodiment 42, further comprising a third LNP, wherein the third LNP comprises, prior to fusion: (i) an RNA molecule; and (ii) a third ionizable lipid.
45. The co-LNP of embodiment 44, wherein the third ionizable lipid is an ionizable amino lipid.
46. The co-LNP of embodiment 44 or embodiment 45, comprising a volumetric ratio of the first LNP to the second and third LNPs that is between about 3:1 and about 1:3.
47. The co-LNP of any of embodiments 39-46, comprising a first helper lipid and a second helper lipid.
48. The co-LNP of any of embodiments 39-47, comprising a polyethylene glycol (PEG)-conjugated lipid.
49. The co-LNP of any of embodiments 39-48, comprising cholesterol.
50. The co-LNP of any of embodiments 39-49, which has an average size of between about 50 nm and 150 nm, or between about 75 nm and about 125 nm, as measured by dynamic light scattering (DLS).
51. The co-LNP of any of embodiments 49-50, wherein the first ionizable lipid comprises a diketopiperazine ring core.
52. The co-LNP of any of embodiments 39-51, wherein the first ionizable lipid comprises an unsaturated tail.
53. The co-LNP of embodiment 52, wherein the unsaturated tail is an unsaturated linoleil tail.
54. The co-LNP of any of embodiments 39-53, wherein the first ionizable lipid is: (a) OF-C4-Deg-Lin, or an analog thereof; (b) cKK-E12, or an analog thereof.
55. The co-LNP of any of embodiments 39-54, wherein the first ionizable lipid is OF-C4-Deg-Lin.
56. The co-LNP of any of embodiments 39-55, wherein the first ionizable lipid and the second ionizable lipid are the same.
57. The co-LNP of any of embodiments 39-55, where the first ionizable lipid and the second ionizable lipids are different.
58. The co-LNP of any of embodiments 39-50 and 57, wherein the first ionizable lipid is DLin-KC2-DMA, or an analog thereof.
59. The co-LNP of any of embodiments 39-50, 57, and 58, wherein the first ionizable lipid is DLin-KC2-DMA.
60. The co-LNP of any of embodiments 39-55 and 57-59, wherein the second ionizable lipid is comprises a diketopiperazine ring core.
61. The co-LNP of any of embodiments 39-55 and 57-60, wherein the second ionizable lipid comprises an unsaturated tail, optionally an unsaturated linoleic tail.
62. The co-LNP of any of embodiments 39-55 and 57-61, wherein the second ionizable lipid is: (a) OF-C4-Deg-Lin, or an analog thereof; or (b) cKK-E12, or an analog thereof.
63. The co-LNP of any of embodiments 39-55 and 57-62, wherein the second ionizable lipid is OF-C4-Deg-Lin, or an analog thereof.
64. The co-LNP any of embodiments 39-54 and 57-63, wherein the second ionizable lipid is OF-C4-Deg-Lin.
65. The co-LNP of any of embodiments 39-55 and 57-64, wherein the second ionizable lipid is cKK-E12, or an analog thereof.
66. The co-LNP of any of embodiments 39-55, 57-62, and 65, wherein the second ionizable lipid is cKK-E12.
67. The co-LNP of any of embodiments 39-55 and 57-59, wherein the second ionizable lipid is DLin-MC3-DMA, or an analog thereof.
68. The co-LNP of any of embodiments 39-55, 57-59, and 67, wherein the second ionizable lipid is DLin-MC3-DMA.
69. The co-LNP of any of embodiments 40, 41, and 44-68, wherein the second ionizable lipid and the third ionizable lipid are the same.
70. The co-LNP of any of embodiments 47-69, wherein the first helper lipid is 1-stearoyl-2-oleoyl-sn-glycero-3-phosphocholine (SOPC).
71. The co-LNP of any of embodiments 47-70, wherein the second helper lipid is 1,2-distearoyl-sn-glycero-3-phosphocholine (DSPC).
72. The co-LNP of any of embodiments 44-71, wherein the third LNP comprises a third helper lipid.
73. The co-LNP of embodiment 72, wherein the second helper lipid and the third helper lipid are the same.
74. The co-LNP of any of embodiments 39-73, wherein the DNA molecule comprises a transgene.
75. The co-LNP of embodiment 74, wherein the transgene encodes a recombinant receptor.
76. The LNP of any of embodiments 39-74, wherein the DNA molecule is a closed end DNA vector.
77. The co-LNP of embodiment 76, wherein the transgene is positioned between protelomerase binding sequences.
78. The co-LNP of embodiment 74 or embodiment 75, wherein the transgene is operably linked to a promoter and positioned between inverted terminal repeats (ITRs).
79. The co-LNP of any of embodiments 39-78, wherein the RNA molecule encodes a transposase.
80. The co-LNP of embodiment 79, wherein the transposase is a piggyBac transposase or a Sleeping Beauty transposase.
81. The co-LNP of any of embodiments 39-73, wherein the DNA molecule comprises a single-stranded DNA oligonucleotide (ssODN) or a double-stranded DNA oligonucleotide (dsODN), the ssODN or the dsODN comprising a nucleotide sequence that is homologous to a target genomic locus.
82. The co-LNP of embodiment 81, wherein the RNA molecule is or comprises a guide RNA (gRNA).
83. The co-LNP of embodiment 82, wherein the gRNA is a single guide RNA (sgRNA).
84. The co-LNP of embodiment 82 or embodiment 83, wherein the gRNA is complexed with a recombinant nuclease capable of inducing a DNA break.
85 The co-LNP of any of embodiments 81-83, wherein the RNA molecule is or comprises a nucleotide sequence encoding a recombinant nuclease capable of inducing a DNA break.
86. The co-LNP of any of embodiments 81-83 and 85, wherein the RNA molecule is: (1) a gRNA; and (2) a nucleotide sequence encoding a recombinant nuclease capable of inducing a DNA break.
87. The co-LNP of any of embodiments 44-75, 81-83, 85, and 86, wherein the RNA molecule of the second LNP is a gRNA, and the RNA molecule of the third LNP is a nucleotide sequence encoding a recombinant nuclease capable of inducing a DNA break.
88. The co-LNP of any of embodiments 84-87, wherein the recombinant nuclease is a zinc finger nuclease (ZFN), a transcription activator-like effector nuclease (TALEN), or a CRISPR-associated nuclease (Cas).
89. The co-LNP of embodiment 88, wherein the Cas is Cas9 or Cas12a.
90. The co-LNP of any of embodiments 75-89, wherein the recombinant receptor is a chimeric antigen receptor (CAR) or a T cell receptor (TCR).
91. The co-LNP of any of embodiments 75-90, wherein the recombinant receptor is a CAR.
92. The co-LNP of embodiment 90 or embodiment 91, wherein the CAR comprises an extracellular antigen-binding domain, a transmembrane domain, and an intracellular region.
93. The co-LNP of embodiment 92, wherein the extracellular antigen-binding domain is an antibody or an antigen-binding fragment thereof that binds to an antigen that is associated with, or expressed on a cell or tissue of a disease or condition.
94. The co-LNP of embodiment 93, wherein the antigen is selected from the group consisting of αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR VIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), and Wilms Tumor 1 (WT-1).
95. The co-LNP of any of embodiments 92-94, wherein the intracellular region comprises an intracellular signaling domain that is or comprises an intracellular signaling domain of a CD3 chain, or a signaling portion thereof.
96. The co-LNP of any of embodiments 92-95, wherein the intracellular region comprises one or more costimulatory signaling domain(s) comprising an intracellular signaling domain selected from the group consisting of: a CD28, a 4-1BB, an ICOS, or a signaling portion thereof.
97. A method of producing a lipid nanoparticle (LNP), the method comprising:
   (1) adding to an organic solvent comprising ethanol:
      (a) an ionizable lipid;
      (b) a helper lipid;
      (c) a polyethylene glycol (PEG)-conjugated lipid; and
      (d) cholesterol,
   thereby generating an organic phase;
   (2) adding to an aqueous solvent having an acidic pH, a deoxyribonucleic acid (DNA) molecule, thereby generating an aqueous phase; and
   (3) combining the organic phase and the aqueous phase by laminar flow mixing in a device, thereby generating an LNP.
98. The method of embodiment 97, wherein the ionizable lipid is an ionizable amino lipid selected from the group consisting of OF-C4-Deg-Lin, an analog thereof, cKK-E12, an analog thereof, DLin-KC2-DMA, and an analog thereof.
99. The method of embodiment 97 or embodiment 98, wherein:
   a flow rate of the aqueous phase in the device is between about 8 mL/min and about 10 mL/min; and/or
   a flow rate of the organic phase in the device is between about 2 mL/min and about 4 mL/min.
100. The method of embodiment 99, wherein the flow rate of the aqueous phase is about 9 mL/min, and the flow rate of the organic phase is about 3 mL/min.
101. The method of any of embodiments 97-100, wherein the aqueous solvent is an acetate buffer.
102. The method of embodiment 101, wherein the pH of the acetate buffer is between about 3.0 and about 4.5, optionally 4.0.
103. The method of embodiment 101 or embodiment 102, further comprising collecting the generated LNP from the device in the acetate buffer.
104. The method of embodiment 103, further comprising washing the collected LNP with an isotonic buffer.
105. The method of embodiment 103 or embodiment 104, further comprising filtering the LNP in the isotonic buffer with a filter to remove an LNP greater than 200 nM in diameter.
106. A LNP produced by the method of any of embodiments 97-105.
107. A co-formulated lipid nanoparticle (co-LNP) comprising:
   (1) a first ribonucleic acid (RNA) molecule and a second ribonucleic acid (RNA) molecule; and
   (2) a first ionizable lipid and a second ionizable lipid,
   wherein one of the first and second RNA molecules encodes a recombinant nuclease capable of inducing a DNA break; and the other of the first and second RNA molecules is a guide RNA (gRNA).
108. A co-formulated lipid nanoparticle (co-LNP) comprising a fusion of a first lipid nanoparticle (LNP) and a second lipid nanoparticle (LNP), wherein:
   (1) the first LNP comprises:
      (i) a ribonucleic acid (RNA) molecule; and
      (ii) a first ionizable lipid; and
   (2) the second LNP comprises:
      (i) a ribonucleic acid (RNA) molecule; and
      (ii) a second ionizable lipid,
   wherein one of the first and second RNA molecules encodes a recombinant nuclease capable of inducing a DNA break; and the other of the first and second RNA molecules is a guide RNA (gRNA).
109. The co-LNP of embodiment 107 or embodiment 108, wherein the gRNA is a single guide RNA (sgRNA).
110. The co-LNP of any of embodiments 107-109, wherein the recombinant nuclease is a zinc finger nuclease (ZFN), a transcription activator-like effector nuclease (TALEN), or a CRISPR-associated nuclease (Cas).
111. The co-LNP of embodiment 110, wherein the Cas is Cas9 or Cas12a.
112. The co-LNP of any of embodiments 107-111, comprising a volumetric ratio of the first LNP to the second LNP that is about 1:1.
113. The co-LNP of any of embodiments 107-112, wherein the first ionizable lipid is an amino lipidoid comprising a diketopiperazine ring core.
114. The co-LNP of any of embodiments 107-113, wherein the first ionizable lipid comprises an unsaturated tail, optionally an unsaturated linoleic tail.
115. The co-LNP of any of embodiments 107-114, wherein the first ionizable lipid is: (a) OF-C4-Deg-Lin, or an analog thereof; or (b) cKK-E12, or an analog thereof.
116. The co-LNP of any of embodiments 107-115, wherein the first ionizable lipid is OF-C4-Deg-Lin, or an analog thereof.
117. The co-LNP of any of embodiments 107-115, wherein the first ionizable lipid is cKK-E12, or an analog thereof.
118. The co-LNP of any of embodiments 107-112, wherein the first ionizable lipid is DLin-MC3-DMA, or an analog thereof.
119. The co-LNP of any of embodiments 107-118, wherein the first ionizable lipid and the second ionizable lipid are the same.
120. The co-LNP of any of embodiments 107-115, wherein the second ionizable lipid is DLin-MC3-DMA, or an analog thereof.
121. The co-LNP of any of embodiments 107-118, wherein the second ionizable lipid is cKK-E12, or an analog thereof.
122. A method of producing a co-formulated lipid nanoparticle (co-LNP), comprising:
   (1) mixing, in an acidic buffer:
      (a) a first lipid nanoparticle (LNP) comprising a first ionizable lipid and a nucleic acid molecule; and
      (b) a second LNP comprising a second ionizable lipid and a ribonucleic acid (RNA) molecule,
   thereby generating a composition comprising the first LNP and the second LNP; and
   (2) neutralizing the composition comprising the first LNP and the second LNP, thereby generating a co-LNP, which is a fusion of the first LNP and the second LNP,
   wherein the nucleic acid molecule in (a) is a deoxyribonucleic acid (DNA) molecule or a ribonucleic acid (RNA) molecule.
123. The method of embodiment 122, wherein the nucleic acid molecule in (a) is a DNA molecule.
124. The method of embodiment 122, wherein the nucleic acid molecule in (a) is an RNA molecule.
125. The method of any of embodiments 122-124, wherein the volumetric ratio of the first LNP to the second LNP in the composition is between about 3:1 and about 1:3.
126. The method of any of embodiments 122, 123, and 125, further comprising mixing, in the acidic buffer, (c) a third LNP comprising a third ionizable lipid and an RNA molecule, thereby generating a composition comprising the first, second, and third LNPs.
127. The method of embodiment 126, wherein the volumetric ratio of the first LNP to the second and third LNPs in the composition is between about 3:1 and about 1:3.
128. The method of any of embodiments 122-127, wherein the acidic buffer is an acetate buffer.
129. The method of any of embodiments 122-128, wherein the acidic buffer has a pH of between about 3.0 and about 4.5, or of 4.0.
130. The method of any of embodiments 122-129, wherein the acidic buffer is neutralized to a pH of between about 6.0 and about 7.5, or between about 6.5 and about 7.0.
131. The method of any of embodiments 122-130, wherein neutralizing the composition comprising the first LNP and the second LNP comprises adding an isotonic buffer.
132. The method of embodiment 131, wherein the isotonic buffer has a pH of about 7.4.
133. The method of embodiment 131 or embodiment 132, wherein neutralizing the composition comprising the first LNP and the second LNP comprises adding at least about 6 parts of the isotonic buffer to 1 part of the acidic buffer.
134. The method of any of embodiments 131-133, wherein neutralizing the composition comprising the first LNP and the second LNP comprises adding between about 6-7 parts of the isotonic buffer to 1 part of the acidic buffer.
135. The method of any of embodiments 131-134, wherein the isotonic buffer is phosphate buffered saline (PBS).
136. A co-LNP produced by the method of any of embodiments 122-135.
137. A lipid nanoparticle (LNP) comprising:
   (a) an ionizable lipid;
   (b) a helper lipid that is 1,2-distearoyl-sn-glycero-3-phosphocholine (DSPC);
   (c) a polyethylene glycol (PEG)-conjugated lipid that is DMG-PEG2000;
   (d) cholesterol; and
   (e) a ribonucleic acid (RNA) molecule.
138. The LNP of embodiment 137, wherein the ionizable lipid is an ionizable amino lipid selected from the group consisting of OF-C4-Deg-Lin, an analog thereof, cKK-E12, an analog thereof, DLin-MC3-DMA, and an analog thereof.
139. The LNP of embodiment 137 or embodiment 138, wherein:
   the ionizable lipid is a lipidoid comprising a diketopiperazine amino core; and/or
   the ionizable lipid comprises an unsaturated tail, optionally an unsaturated linoleic tail.
140. The LNP of any of embodiments 137-139, wherein the mass fraction of the ionizable lipid is between about 50% and about 65%.
141. The LNP of any of embodiments 137-140, wherein the mass fraction of DSPC is between about 10% and about 15%.
142. The LNP of any of embodiments 137-141, wherein the mass fraction of DMG-PEG2000 is between about 5% and about 7.5%.
143. The LNP of any of embodiments 137-142, wherein the mass fraction of cholesterol is between about 15% and about 25%.
144. The LNP of any of embodiments 137-143, wherein the mass fraction of the RNA molecule is between about 3% and about 10%.
145. The LNP of any of embodiments 137-139, wherein the mass fraction of the ionizable lipid is between about 32% and about 36%.
146. The LNP of any of embodiments 137-139 and 145, wherein the mass fraction of DSPC is between about 15% and about 20%.
147. The LNP of any of embodiments 137-139, 145, and 146, wherein the mass fraction of DMG-PEG2000 is between about 3.5% and about 5.5%.
148. The LNP of any of embodiments 137-139, and 145-147, wherein the mass fraction of cholesterol is between about 35% and about 45%.
149. The LNP of any of embodiments 137-139, and 145-148, wherein the mass fraction of the RNA molecule is between about 3% and about 4%.
150. The LNP of any of embodiments 137-149, wherein the RNA molecule encodes a transposase.
151. The LNP embodiment 150, wherein the transposase is a piggyBac transposase or a Sleeping Beauty transposase.
152. The LNP of any of embodiments 137-149, wherein the RNA molecule comprises a guide RNA (gRNA).
153. The co-LNP of embodiment 152, wherein the gRNA is a single guide RNA (sgRNA).
154. The LNP of embodiment 152 or embodiment 153, wherein the gRNA is complexed with a recombinant nuclease capable of inducing a DNA break.
155. The LNP of any of embodiments 137-149, and 152-154, wherein the RNA sequence comprises a nucleotide sequence encoding a recombinant nuclease capable of inducing a DNA break.
156. The LNP of embodiment 154 or embodiment 155, wherein the recombinant nuclease is a zinc finger nuclease (ZFN), a transcription activator-like effector nuclease (TALEN), or a CRISPR-associated nuclease (Cas).
157. The LNP of any of embodiments 154-156, wherein the recombinant nuclease is a Cas nuclease, optionally Cas9 or Cas12a.
158. A composition comprising: (1) a LNP of any of embodiments 1-38; and (2) a lipid nanoparticle comprising a ribonucleic acid (RNA) molecule.
159. A composition comprising: (1) a LNP of any of embodiments 1-38; and (b) a LNP of any of embodiments 137-157.
160. A co-formulated lipid nanoparticle (co-LNP) comprising a fusion of: (1) a LNP of any of embodiments 1-38; and (2) a lipid nanoparticle (LNP) comprising an ribonucleic acid (RNA) molecule.
161. A co-formulated lipid nanoparticle (co-LNP) comprising a fusion of (1) a lipid nanoparticle comprising a deoxyribonucleic acid (DNA) molecule; and (2) a LNP of any of embodiments 137-157.
162. A co-formulated lipid nanoparticle (co-LNP) comprising a fusion of (1) a LNP of any of embodiments 1-38; and (2) a LNP of any of embodiments 137-157.
163. A combination of: (1) a LNP of any of embodiments 1-38; and (2) a ribonucleoprotein (RNP) complex.
164. A composition comprising the LNP of any of embodiments 1-38 and 106.
165. A composition comprising the co-LNP of any of embodiments 39-96 and 136.
166. A composition comprising the LNP of any of embodiments 137-157.
167. A method of genetically engineering an immune cell, the method comprising:
   (1) introducing a ribonucleic acid (RNA) molecule into an immune cell by electroporation; and
   (2) incubating the immune cell with the LNP of any of embodiments 1-38 and 106 or the composition of embodiment 164.
168. A method of genetically engineering an immune cell, the method comprising:
   (1) introducing a ribonucleoprotein complex (RNP) into an immune cell by electroporation; and
   (2) incubating the immune cell with the LNP of any of embodiments 1-38 and 106 or the composition of embodiment 164.
169. A method of genetically engineering an immune cell, the method comprising incubating the immune cell with (1) the LNP of any of embodiments 1-38 and 106 or the composition of embodiment 164; and (2) the LNP of any of embodiments 137-157 or the composition of embodiment 166.
170. A method of genetically engineering an immune cell, the method comprising incubating an immune cell with the co-LNP of any of embodiments 39-96, 107-121, and 136 or the composition of embodiment 165.
171. The method of any of embodiments 167-170, wherein the immune cell is a lymphocyte.
172. The method of any of embodiments 167-171, wherein the immune cell is a T cell.
173. The method of embodiment 172, wherein the T cell is a primary T cell.
174. The method of embodiment 172 or embodiment 173, wherein the T cell is a CD4+ T cell or a CD8+ T cell.
175. The method of any of embodiments 167-174, wherein, at the time of incubating the immune cell with the LNP, the co-LNP, or the composition, the immune cell is activated.
176. The method of any of embodiments 167-175, wherein, at the time of incubating the immune cell with the LNP, the co-LNP, or the composition, the immune cell expresses CD25, CD26, CD27, CD28, CD30, CD71, CD154, CD40L, CD134, or a combination thereof.
177. The method of any of embodiments 167-176, wherein the immune cell is incubated under stimulating conditions prior to incubating the immune cell with the LNP, the co-LNP, or the composition.
178. The method of embodiment 177, wherein the immune cell is incubated under stimulating conditions for between about 24 hours and about 72 hours, or for about 48 hours.
179. The method of embodiment 177 or embodiment 178, wherein the stimulating conditions comprise incubation with a stimulatory reagent capable of activating an intracellular signaling domain of a component of a TCR complex and an intracellular signaling domain of a costimulatory molecule.
180. The method of embodiment 179, wherein the stimulatory reagent comprises a primary agent that binds to CD3 and a secondary agent that binds to a T cell costimulatory molecule.
181. The method of embodiment 180, wherein the costimulatory molecule is selected from the group consisting of CD28, 4-1BB, OX40, and ICOS.
182. The method of embodiment 180 or embodiment 181, wherein the primary agent is an anti-CD3 antibody or antigen-binding fragment, and the secondary agent is an anti-CD28 antibody or antigen-binding fragment.
183. The method of any of embodiments 167-182, wherein the immune cell is incubated with apolipoprotein E (ApoE) prior to incubating the immune cell with the LNP, the co-LNP, or the composition.
184. The method of embodiment 183, wherein the ApoE is ApoE4.
185. An immune cell produced by the method of any of embodiments 167-184.
186. A composition comprising a plurality of the immune cell of embodiment 185.

### EXAMPLES

The following examples are included for illustrative purposes only and are not intended to limit the scope of the invention.

### Example 1: Generation of Lipid Nanoparticle Formulations for Nucleic Acid Delivery

Various novel lipid nanoparticle (LNP) formulations as presently disclosed were assessed for their ability to generate LNPs and achieve LNP-mediated DNA delivery in primary human T cells.

Briefly, LNPs each containing an ionizable lipid, a helper lipid, a polyethylene glycol (PEG)-conjugated lipid (“PEG lipid”), cholesterol, and in some cases, the cellular uptake marker 1,1′-dioctadecyl-3,3,3′,3′-tetramethylindodicarbocyanine (DiD) were formulated by laminar flow mixing and analyzed for their ability to deliver and mediate transient expression of DNA in primary human T cells.

To generate the LNPs, an organic phase was prepared by diluting the PEG lipid, the ionizable lipid, the helper lipid, and cholesterol to their specified concentrations in ethanol, and an aqueous phase was prepared by diluting DNA plasmid (e.g., plasmid DNA encoding GFP) to its specified concentration in a 25 mM acetate buffer of pH 4.0. For all formulations tested, the PEG lipid used was 1,2-dimyristoyl-rac-glycero-3-methoxypolyethylene glycol-2000 (DMG-PEG2000), the ionizable lipid used was 2,2-dilinoleyl-4-dimethylaminoethyl-[1,3]-dioxolane (DLin-KC2-DMA), and the helper lipid used was 1-stearoyl-2-oleoyl-sn-glycero-3-phosphocholine (SOPC). The organic and aqueous phases were pipetted into a Precision Nanosystems NanoAssemblr™ Benchtop (NABT) instrument for LNP formulation. The total flow rates for the aqueous and organic phases were programmed into the instrument, as well as the ratio of the aqueous flow rate to the organic flow rate (Aq:Org). Tables E1 and E2, below, provide the specific formulation components and process parameters of all 24 formulations assessed. The ratio of positive to negative charge, in terms of the ratio of amine (N) to phosphate (P) is designated by (N:P).

Immediately following generation of the LNPs, calcium- and magnesium-free phosphate-buffered saline (PBS) was added to the LNPs for solvent removal and washing. The solutions were centrifuged at 2000×g for 30 minutes at 10° Celsius using a 30 kDa centrifugal filter, and then the bottom half of the volume was removed. This step was repeated two more times, for a total of three washes.

The resulting LNPs were sized by dynamic light scattering (DLS) before being stored overnight at 5 Celsius. Following overnight incubation, the LNPs were again sized by DLS, and then sterile filtered in a biosafety cabinet. LNPs having an Z-average particle size greater than 200 nm in diameter were filtered using a 0.45 □m filter. All other LNPs were filtered using a 0.2 □m filter. Following sterile filtration, the LNPs were sized a final time by DLS. The LNPs were also assessed for DNA loading, as determined by the Quant-iT™ PicoGreen™ dsDNA broad range DNA assay. Percent DNA loading was calculated as 100×(signal of sample with Triton-X−signal of sample in DI water)/(signal of sample with Triton-X).

TABLE E1

| | |
|---|---|
| Factor | Range |

Formulation Component

| | |
|---|---|
| Cationic or Ionizable Lipid (mol %) | 40-60 |
| Helper Lipid (mol %) | 5-15 |
| PEG Lipid (mol %) | 0.5-3.0 |
| Cholesterol (mol %) | 23.5-54.5 |

Process Parameter

| | |
|---|---|
| N:P ratio | 3-9 |
| Aqueous Flow Rate (mL/min) | 6-12 |
| Organic Flow Rate (mL/min) | 1.5-4.5 |
| Acetate Buffer Molarity (mM) | 25-200 |

TABLE E2
LNP Formulations

| | Process Parameters | | | | | | | |
|---|---|---|---|---|---|---|---|---|
| | | | Acetate | | LNP Composition | | | |
| | Aq. Flow | Org. Flow | Buffer | | (Mole fraction) | | | |
| LNP | Rate | Rate | Molarity | | Helper | | PEG | Ion. |
| Formulation | (mL/min) | (mL/min) | (mM) | N:P | Lipid | Chol. | Lipid | Lipid |
| 1 | 6 | 4.5 | 112.5 | 3 | 15 | 42 | 3 | 40 |
| 2 | 9 | 1.5 | 25 | 3 | 5 | 54.5 | 0.5 | 40 |
| 3 | 6 | 1.5 | 25 | 9 | 15 | 42 | 3 | 40 |
| 4 | 6 | 4.5 | 25 | 6 | 15 | 24.5 | 0.5 | 60 |
| 5 | 6 | 1.5 | 200 | 9 | 10 | 49.5 | 0.5 | 40 |
| 6 | 12 | 1.5 | 112.5 | 9 | 5 | 34.5 | 0.5 | 60 |
| 7 | 12 | 4.5 | 200 | 2 | 5 | 34.5 | 0.5 | 60 |
| 8 | 12 | 1.5 | 200 | 6 | 5 | 52 | 3 | 40 |
| 9 | 9 | 4.5 | 200 | 9 | 15 | 22 | 3 | 60 |
| 10 | 9 | 3 | 112.5 | 6 | 10 | 38.25 | 1.75 | 50 |
| 11 | 12 | 4.5 | 25 | 3 | 10 | 27 | 3 | 60 |
| 12 | 6 | 1.5 | 25 | 3 | 5 | 32 | 3 | 60 |
| 13 | 12 | 1.5 | 200 | 3 | 15 | 32 | 3 | 50 |
| 14 | 12 | 4.5 | 200 | 9 | 15 | 44.5 | 0.5 | 40 |
| 15 | 12 | 4.5 | 25 | 9 | 5 | 52 | 3 | 40 |
| 16 | 12 | 3 | 25 | 3 | 15 | 44.5 | 0.5 | 40 |
| 17 | 9 | 3 | 112.5 | 6 | 10 | 38.25 | 1.75 | 50 |
| 18 | 6 | 1.5 | 200 | 3 | 15 | 24.5 | 0.5 | 60 |
| 19 | 12 | 1.5 | 25 | 9 | 15 | 23.25 | 1.75 | 60 |
| 20 | 6 | 3 | 200 | 9 | 5 | 32 | 3 | 60 |
| 21 | 6 | 4.5 | 200 | 3 | 5 | 53.5 | 1.5 | 40 |
| 22 | 6 | 4.5 | 25 | 9 | 5 | 44.5 | 0.5 | 50 |
| 23 | 9 | 3 | 25 | 6 | 10 | 38.5 | 1.5 | 50 |
| 23* | 9 | 3 | 25 | 6 | 10 | 38.5 | 1.5 | 50 |
| 24 | 9 | 3 | 112.5 | 6 | 10 | 38.5 | 1.5 | 50 |

*Generated on NanoAssmblr ® Spark ™ instrument.

### Example 2: Transfection of T Cells Using Lipid Nanoparticles

Primary human T cells were obtained from human donors and engineered to transiently express green fluorescent protein (GFP) by transfection with LNPs generated from the formulations described in Example 1 and containing plasmid DNA encoding the GFP.

Separate compositions of CD4+ and CD8+ cells were selected by immunoaffinity-based enrichment from isolated PBMCs from leukapheresis samples from four healthy human donors. The selected cell compositions were cryopreserved, subsequently thawed and combined in culture at a 1:1 ratio of viable CD4+ to viable CD8+ cells. The combined CD4+ and CD8+ cell composition was then incubated for 48 hours with anti-CD3/CD28 beads (1:1 cell-to-bead ratio) in media containing recombinant IL-2, IL-7, and IL-15 to activate (stimulate) the T cells. Following incubation, the combined CD4+ and CD8+ cell composition was transferred to a non-tissue culture plate and LNPs were added to achieve a final total DNA concentration of 0.75 to 6.0 μg/mL superfolder GFP plasmid DNA under the CAG promoter (pCAG-sfGFP DNA), and optionally, 0.2% DiD. The cells were cultured in media supplemented with 1 μg/mL of apolipoprotein E4 (ApoE4) to support cellular uptake of the LNPs.

Cells were analyzed by flow cytometry 24 to 48 hours after transfection to determine cell viability and transient GFP expression. Cell viability was determined by quantification of dead cells using SYTOX™ blue dead cell stain and GFP expression was determined via flow cytometry.

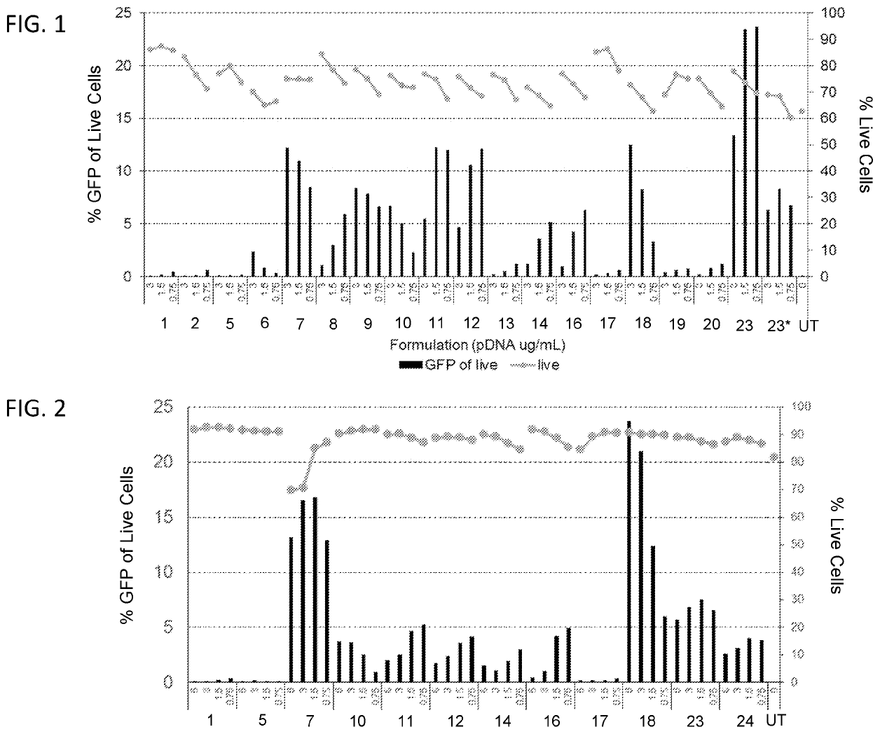

Different batches of generated LNPs were tested, as shown in FIG. 1 and FIG. 2, respectively. Mock-transfected cells (“UT”) served as controls. LNPs generated from certain formulations resulted in a high degree of transient transfection efficiency as observed by the % GFP of live cells (left y-axis and bars). Similar results were seen with similar LNPs generated as a different batch (FIG. 2). In the first batch experiment, LNPs generated from formulation 23 resulted in the greatest percentage of GFP-expressing cells. More than 20% of cells transfected with LNPs of formulation 23 expressed GFP, when either 1.5 or 0.75 g of DNA was used (FIG. 1, bars). In the second batch experiment, LNPs of formulations 7 and 18 resulted in the greatest percentage of GFP-expressing cells, with between 15 and 20% of the transfected cells expressing GFP (FIG. 2, bars).

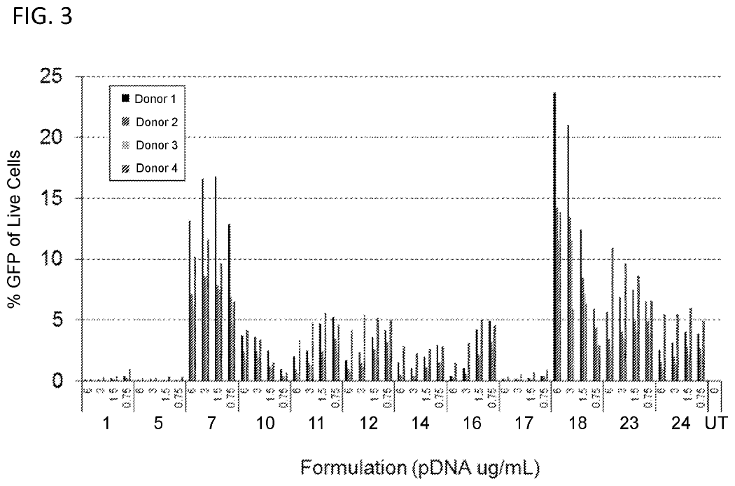

LNPs generated from the formulations shown on the x-axis of FIG. 2 were tested for transient transfection efficiency of T cells from four different healthy human donors. The results are shown in FIG. 3. While interdonor variability in transfection efficiency (% GFP of live cells) was observed following transfection with LNPs generated from different formulations among the four donors, the LNPs observed to have the highest transfection efficiency in FIG. 1 and FIG. 2 resulted in increased expression of GFP across all donors. This finding is consistent with an observation that the effect of LNP formulation outweighed the effect of interdonor variability.

### Example 3: Lipid Nanoparticle Formulations

The effects of LNP formulation components on LNP size, DNA loading into LNPs, and transient transfection efficiency were assessed. In this experiment, LNP formulations were generated to contain a 7.3 kb αCD19 CAR-sfGFP DNA plasmid encoding an exemplary anti-CD19 chimeric antigen receptor (CAR). The CAR contained an anti-CD19 scFv derived from a murine antibody, an immunoglobulin spacer, a transmembrane domain derived from CD28, a costimulatory region derived from 4-1BB, and a CD3-zeta intracellular signaling domain. The vector also encoded a GFP molecule that served as a surrogate marker for CAR expression.

a. Cholesterol Variance

LNPs were generated from different formulations by the methods described in Example 1, in which the mass fraction of the ionizable lipid, helper lipid, and PEG lipid were held at different fixed percentages, while the mass fraction of cholesterol was allowed to vary (“float”). All other aspects of the formulation components and process parameters were maintained at constant values. In total, testing of three different fixed mass fraction percentages for each of the ionizable lipid, the helper lipid, and the PEG-conjugated lipid yielded 18 different formulations. Table E3 and Table E4 set forth the generated LNP formulations (formulations F6 and F7 and formulations F17 and F18 were replicates).

TABLE E3
Formulations with Cholesterol Float

| Class | Variable | Species | Values | | | Units |
|---|---|---|---|---|---|---|
| Component | DNA Plasmid | pCAG- | 3.5 | | | % |
| | Mass Fraction | sfGFP | | | | |
| | Ionizable Lipid | DLin-KC2- | 40 | 50 | 60 | % |
| | Mass Fraction | DMA | | | | |
| | Helper Lipid | SOPC | 5 | 12 | 19 | % |
| | Mass Fraction | | | | | |
| | PEG Lipid Mass | DMG- | 2.5 | 5.0 | 7.5 | % |
| | Fraction | PEG2000 | | | | |
| | Cholesterol | Cholesterol | Float | | | % |
| | Mass Fraction | | | | | |
| Process | Total | Lipids + | 4.29 | | | mg/mL |
| | Concentration | DNA | | | | |
| | Aqueous Flow | DNA in | 6.0 | | | mL/min |
| | Rate | Buffer | | | | |
| | Organic Flow | Lipids in | 4.5 | | | mL/min |
| | Rate | Ethanol | | | | |

| | Temperature | | RT (20) | Degrees |
|---|---|---|---|---|
| | | | | Celsius |
| Buffer | Concentration | Acetate | 25 | mM |
| | pH | | 4.0 | — |

TABLE E4
LNP Formulations with Cholesterol Float

| | PEG | | Ionizable | | Helper |
|---|---|---|---|---|---|
| Formulation | Lipid | DNA | Lipid | Cholesterol | Lipid |
| 1 | 7.5 | 3.5 | 60 | 10 | 19 |
| 2 | 5 | 3.5 | 60 | 12.5 | 19 |
| 3 | 2.5 | 3.5 | 60 | 15 | 19 |
| 4 | 7.5 | 3.5 | 60 | 17 | 12 |
| 5 | 7.5 | 3.5 | 60 | 24 | 5 |
| 6 | 2.5 | 3.5 | 60 | 29 | 5 |
| 7 | 2.5 | 3.5 | 60 | 29 | 5 |
| 8 | 7.5 | 3.5 | 50 | 20 | 19 |
| 9 | 2.5 | 3.5 | 50 | 32 | 12 |
| 10 | 5 | 3.5 | 50 | 36.5 | 5 |
| 11 | 7.5 | 3.5 | 40 | 30 | 19 |
| 12 | 5 | 3.5 | 40 | 32.5 | 19 |
| 13 | 2.5 | 3.5 | 40 | 35 | 19 |
| 14 | 7.5 | 3.5 | 40 | 37 | 12 |
| 15 | 5 | 3.5 | 40 | 39.5 | 12 |
| 16 | 7.5 | 3.5 | 40 | 44 | 5 |
| 17 | 2.5 | 3.5 | 40 | 49 | 5 |
| 18 | 2.5 | 3.5 | 40 | 49 | 5 |
| 19 | 2.5 | 3.5 | 40 | 42 | 12 |
| 20 | 5 | 3.5 | 40 | 46.5 | 5 |

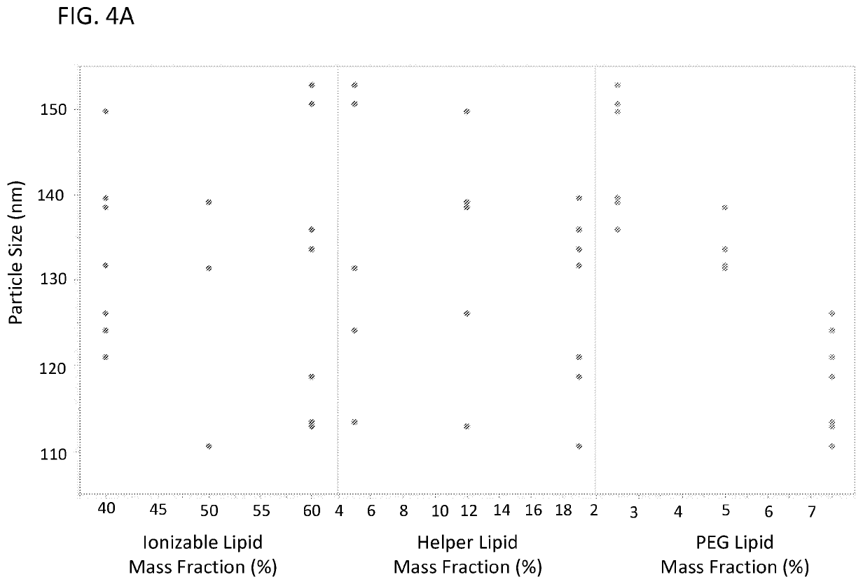

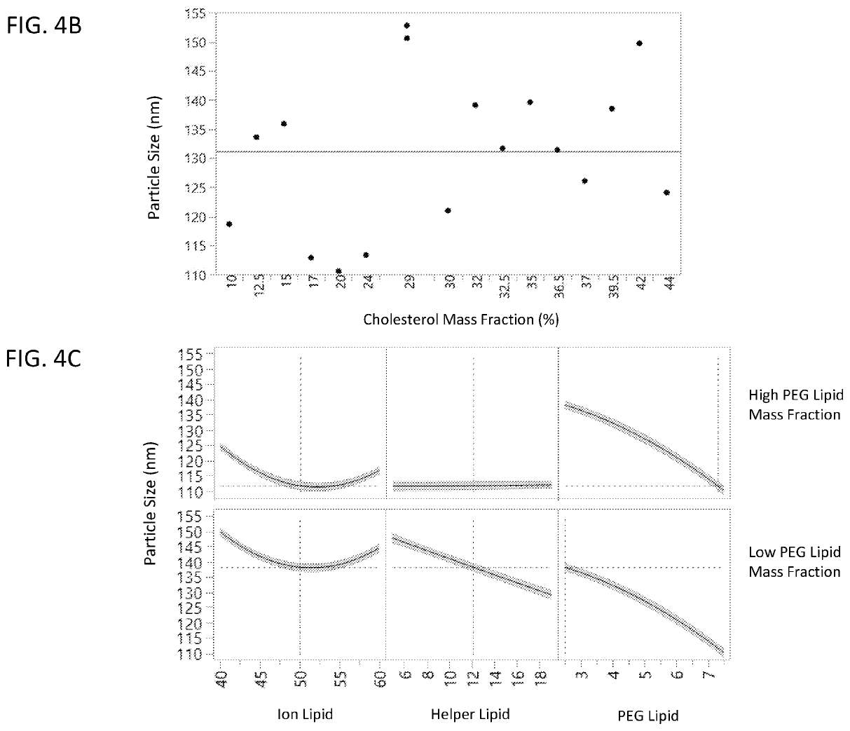

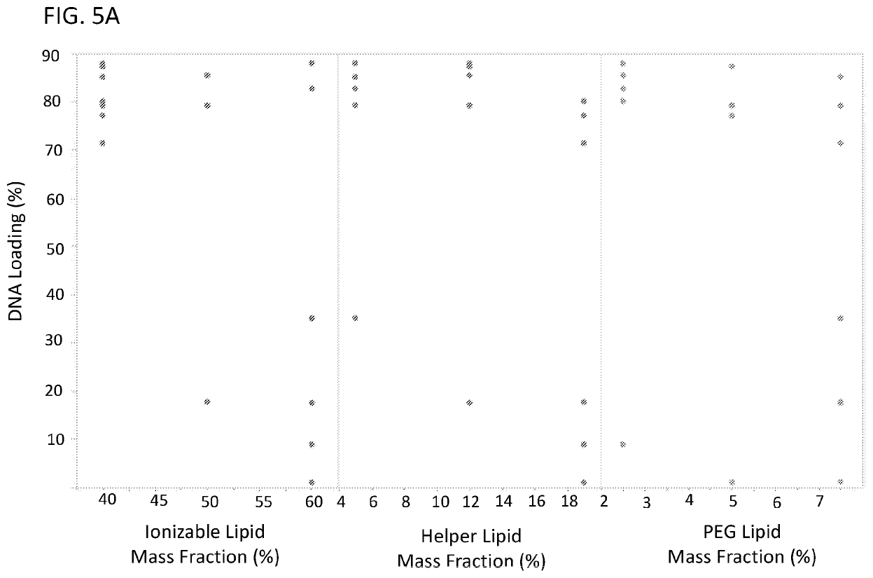

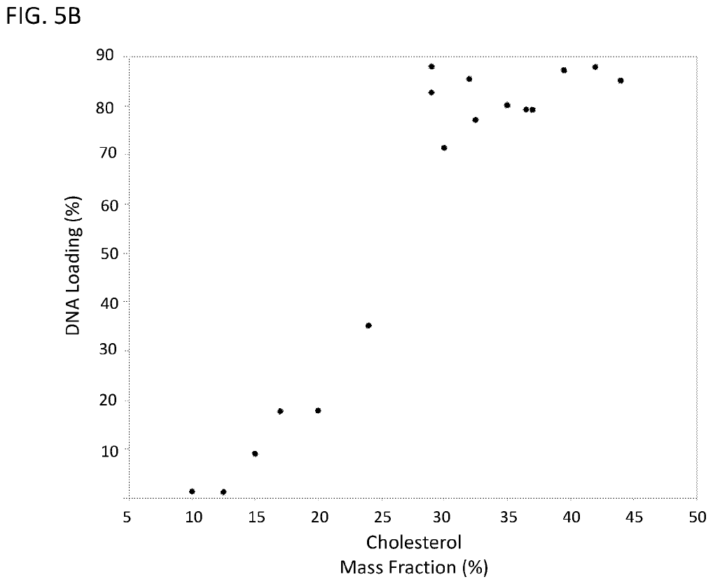

Particle size was assessed by DLS following sterile filtration, as described in Example 1. As shown in FIGS. 4A and 4B, the mass fraction percentage of PEG lipid was observed to affect LNP size (p<0.001; FIG. 4A), whereas there was no statistically significant effect of mass fraction percentages of ionizable lipid (p<0.81; FIG. 4A), helper lipid (p<0.36; FIG. 4A), or cholesterol (p<0.13; FIG. 4B) on particle size. Specifically, increasing the mass fraction of the PEG lipid decreased particle size. A cross-effect between the helper lipid and PEG lipid was observed. At higher PEG lipid mass fraction percentages, the helper lipid was not found to have a significant effect on particle size. However, at lower PEG lipid mass fraction percentages, increasing the helper lipid reduced particle size (FIG. 4C). DNA loading was assessed by Pico Green fluorescence, as described in Example 1. DNA loading, as shown in FIGS. 5A and 5B, was affected by mass fraction percentage of the ionizable lipid (p<0.01; FIG. 5A) and cholesterol (p<0.001; FIG. 5B). Increasing cholesterol mass fraction percentage increased DNA loading, and in particular, high loading (>70%) was achieved when cholesterol mass fraction percentage was greater than 30%. By contrast, increasing ionizable lipid mass fraction percentage decreased DNA loading. There was no statistically significant effect on DNA loading by mass fraction percentage of helper lipid (p<0.06; FIG. 5A) or PEG lipid (p<0.15; FIG. 5A).

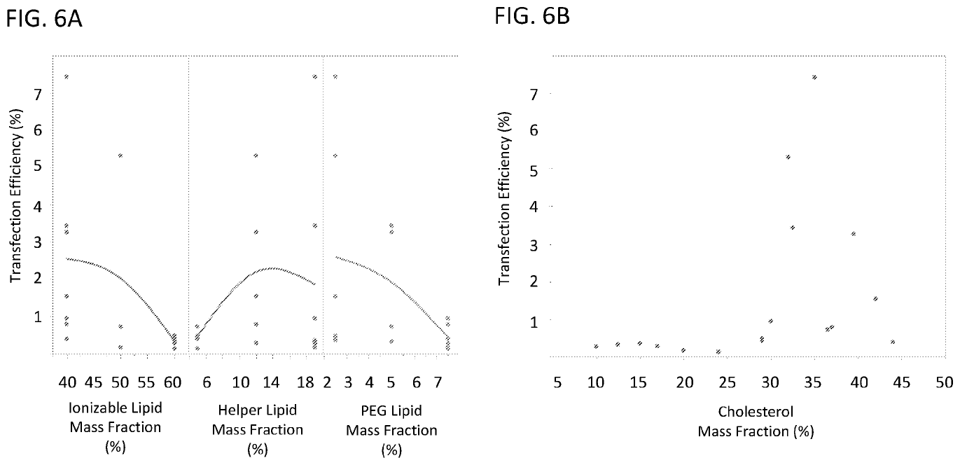

Formulations as described in Table E4 were used to transiently transfect human T cells at a 6.0 μg/mL DNA dosing concentration (final concentration with cells), and the effects of varying the described parameters on cell viability and transfection efficiency of T cells were assessed. A combined CD4+ and CD8+ T cell composition was generated and activated substantially as described in Example 2, and incubated in media with 1 g/mL ApoE4 and LNPs generated from the various formulations for approximately 24 hours at 37° Celsius, 5% CO₂, to allow for transfection. Following incubation, cells were assessed for viability and transfection efficiency as described in Example 2. Increasing ionizable lipid mass fraction percentage decreased transfection efficiency (p<0.05; left panel of FIGS. 6A), while there was no statistically significant effect of helper lipid or PEG lipid mass fraction percentage on transfection efficiency (middle and right panels, respectively, of FIG. 6A). As shown in FIG. 6B, transfection efficiency was very low at a cholesterol mass fraction percentage of less than about 30% and was maximized between 30-40% cholesterol mass fraction percentage.

b. Ionizable Lipid Variance

LNPs were generated from various formulations by the methods described in Example 1, in which the mass fraction of DNA plasmid, helper lipid, and cholesterol were held at different fixed percentages, while the mass fraction percentage of ionizable lipid was allowed to vary (“float”). All other aspects of the formulation components and process parameters were maintained at a constant value. In total, testing of three different mass fraction percentages for each of the DNA plasmid, the helper lipid, and cholesterol yielded 16 different formulations. The LNPs were formulated and assessed substantially as described in the preceding section. Table E5 and Table E6 set forth the different generated LNP formulations (formulations F5 and F6 and formulations F11 and F12 were replicates).

TABLE E5
Formulations with Ionizable Lipid Float

| Class | Variable | Species | Values | | | Units |
|---|---|---|---|---|---|---|
| Component | DNA Plasmid | αCD19 | 3.5 | 6 | 9 | % |
| | Mass Fraction | CAR-sfGFP | | | | |
| | Ionizable Lipid | DLin-KC2- | Float | | | % |
| | Mass Fraction | DMA | | | | |
| | Helper Lipid | SOPC | 7 | 13 | 19 | % |
| | Mass Fraction | | | | | |
| | PEG Lipid Mass | DMG- | 2.5 | | | % |
| | Fraction | PEG2000 | | | | |
| | Cholesterol | Cholesterol | 25 | 30 | 35 | % |
| | Mass Fraction | | | | | |
| Process | Total | Lipids + | 4.29 | | | mg/mL |
| | Concentration | DNA | | | | |
| | Aqueous Flow | DNA in | 6.0 | | | mL/min |
| | Rate | Buffer | | | | |
| | Organic Flow | Lipids in | 4.5 | | | mL/min |
| | Rate | Ethanol | | | | |

| | Temperature | | RT (20) | Degrees |
|---|---|---|---|---|
| | | | | Celsius |
| Buffer | Concentration | Acetate | 25 | mM |
| | pH | | 4.0 | — |

TABLE E6
LNP Formulations with Ionizable Lipid Float

| | PEG | | Ionizable | | Helper |
|---|---|---|---|---|---|
| Formulation | Lipid | DNA | Lipid | Cholesterol | Lipid |
| 1 | 2.5 | 9 | 34.5 | 35 | 19 |
| 2 | 2.5 | 6 | 37.5 | 35 | 19 |
| 3 | 2.5 | 3.5 | 40 | 35 | 19 |
| 4 | 2.5 | 9 | 44.5 | 25 | 19 |
| 5 | 2.5 | 9 | 45.5 | 30 | 13 |
| 6 | 2.5 | 9 | 45.5 | 30 | 13 |
| 7 | 2.5 | 3.5 | 45 | 30 | 19 |
| 8 | 2.5 | 9 | 46.5 | 35 | 7 |
| 9 | 2.5 | 3.5 | 46 | 35 | 13 |
| 10 | 2.5 | 6 | 47.5 | 25 | 19 |
| 11 | 2.5 | 3.5 | 50 | 25 | 19 |
| 12 | 2.5 | 3.5 | 50 | 25 | 19 |
| 13 | 2.5 | 3.5 | 52 | 35 | 7 |
| 14 | 2.5 | 6 | 53.5 | 25 | 13 |
| 15 | 2.5 | 6 | 54.5 | 30 | 7 |
| 16 | 2.5 | 9 | 56.5 | 25 | 7 |
| 17 | 2.5 | 3.5 | 56 | 25 | 13 |
| 18 | 2.5 | 3.5 | 62 | 25 | 7 |

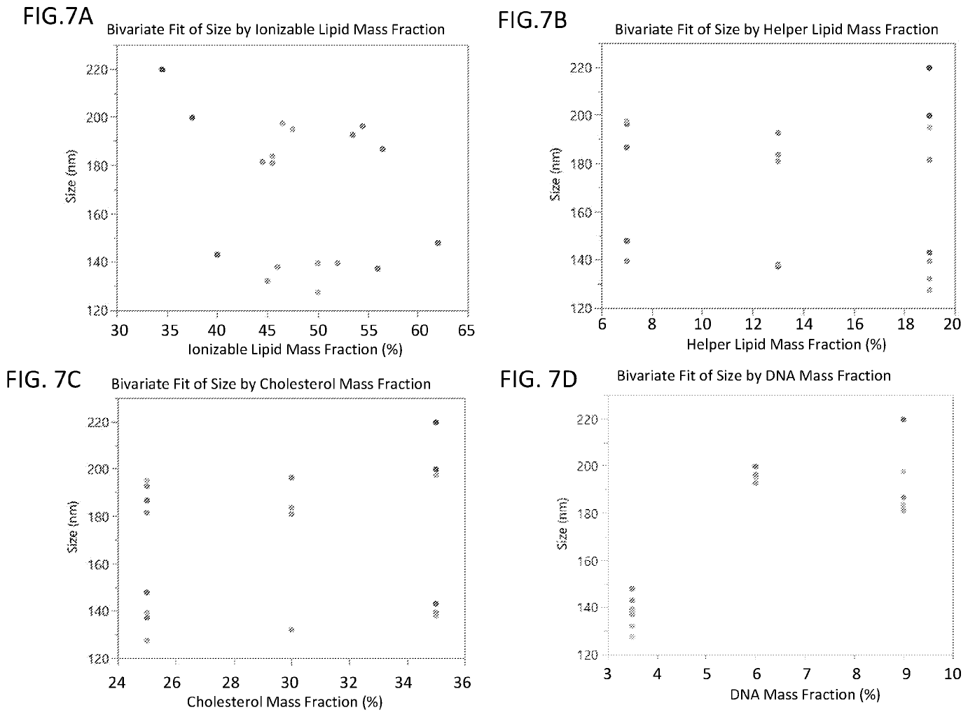

FIGS. 7A-7D. As shown, the mass fraction percentage of DNA plasmid had a statistically significant effect on LNP size (p<0.001; FIG. 7D). Specifically, formulations with high plasmid DNA mass fraction (6% or 9% of the mass fraction) resulted in larger particle size, whereas formulations with 3.5% plasmid DNA mass fraction resulted in smaller particle sizes (125-150 nM in diameter). There was no statistically significant effect of mass fraction percentage of ionizable lipid (p<0.19; FIG. 7A), helper lipid (p<0.74; FIG. 7B), or cholesterol (p<0.56; FIG. 7C) on particle size.

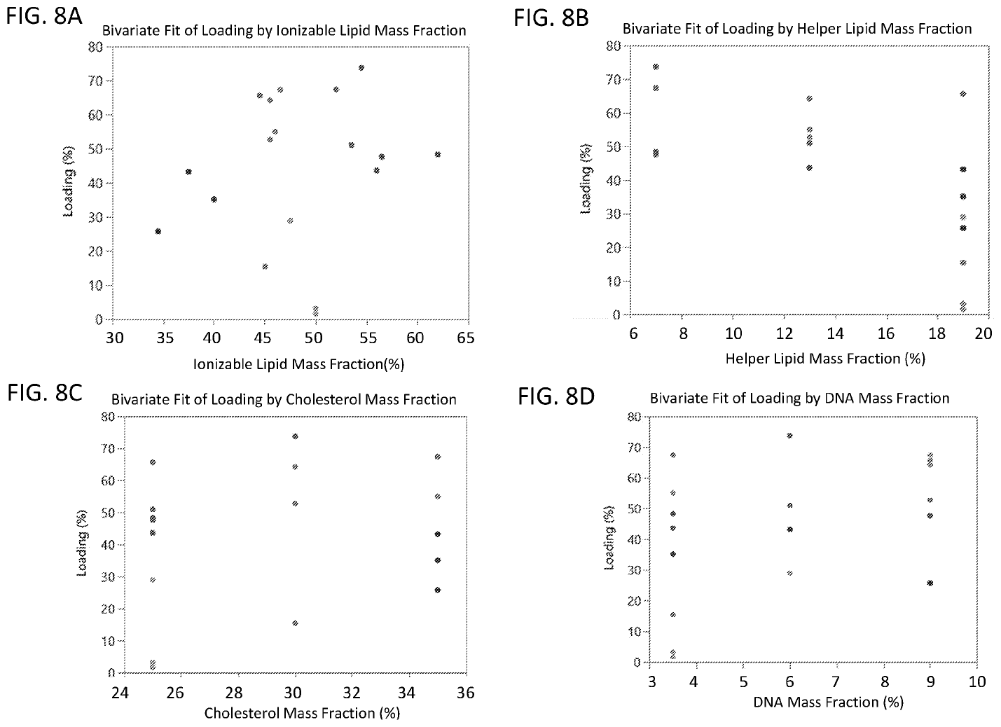

DNA loading into LNPs was assessed by Pico Green fluorescence, as previously described. DNA loading, as shown in FIGS. 8A-8D, was affected by mass fraction percentage of helper lipid (p<0.01; FIG. 8B); increasing helper lipid mass fraction percentage decreased DNA loading. In this experiment, no statistically significant effect of ionizable lipid, cholesterol, or DNA mass fraction percentage on DNA loading was observed (FIGS. 8A, 8C, and 8D, respectively).

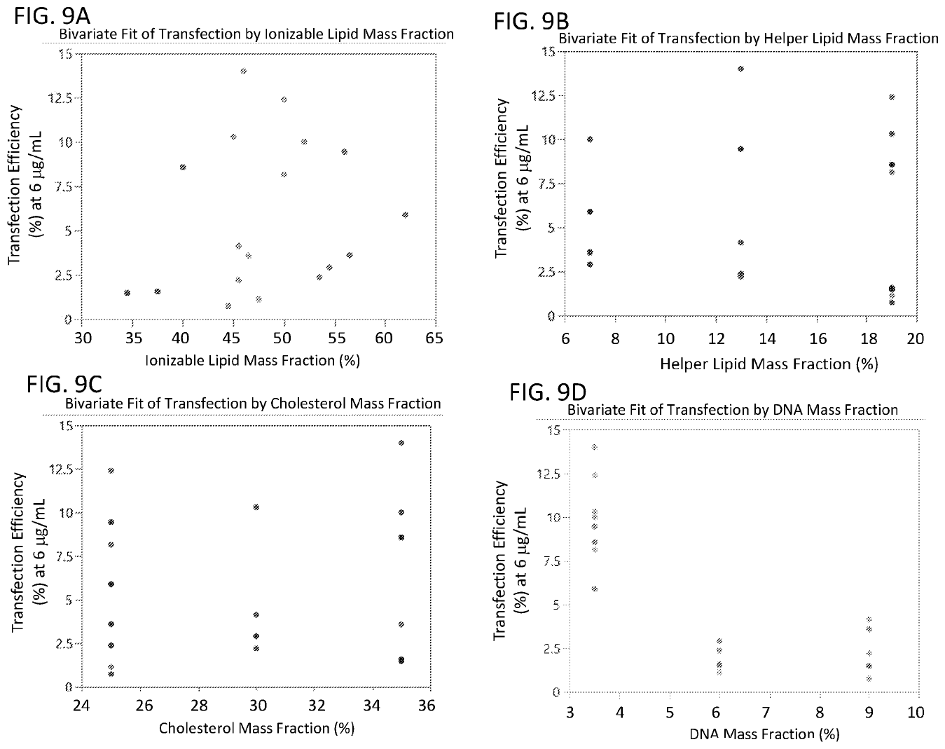

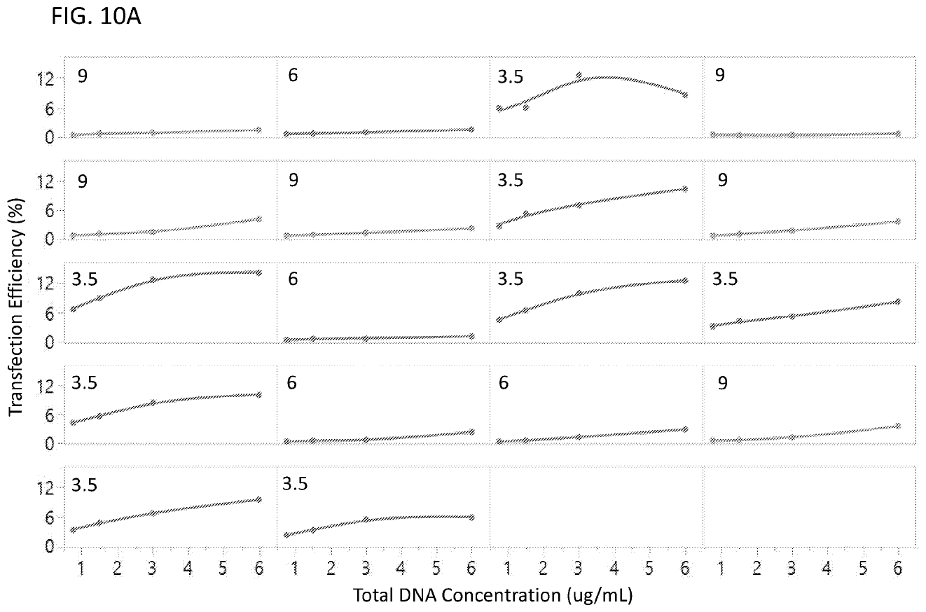

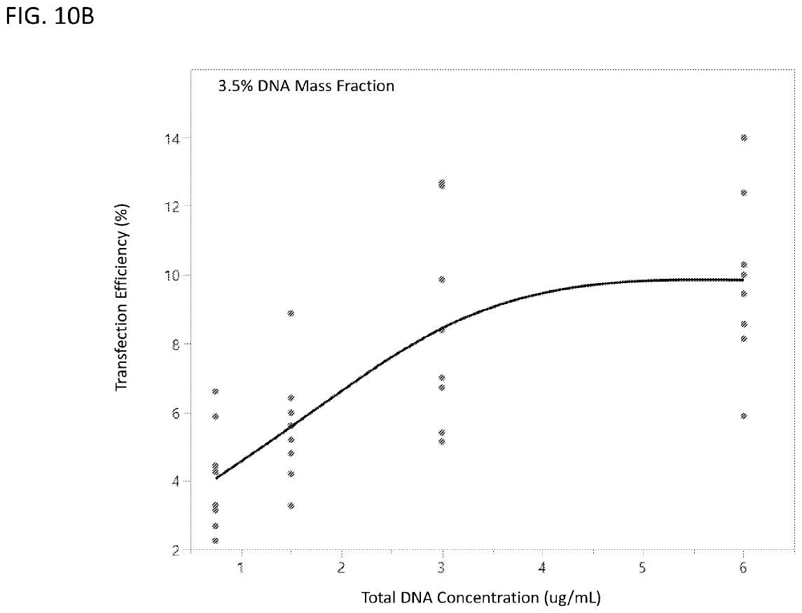

The generated LNPs were assessed for their effects on cell viability and transfection efficiency, as described above. Increasing plasmid DNA mass fraction percentage decreased transfection efficiency (p<0.001; FIG. 9D), whereas there was no statistically significant effect of the ionizable lipid, helper lipid, or cholesterol mass fraction percentage on transfection efficiency at 6.0 μg/mL (FIGS. 9A, 9B, and 9C, respectively). As shown in FIGS. 10A and 10B, when the DNA mass fraction was held constant at 3.5%, transfection efficiency was observed to positively correlate with total DNA concentration (DNA mass fraction percentage given in corner). Thus, while increased DNA mass fraction percentage was observed to negatively affect transfection of cells, increased DNA concentration resulted in higher transfection efficiency.

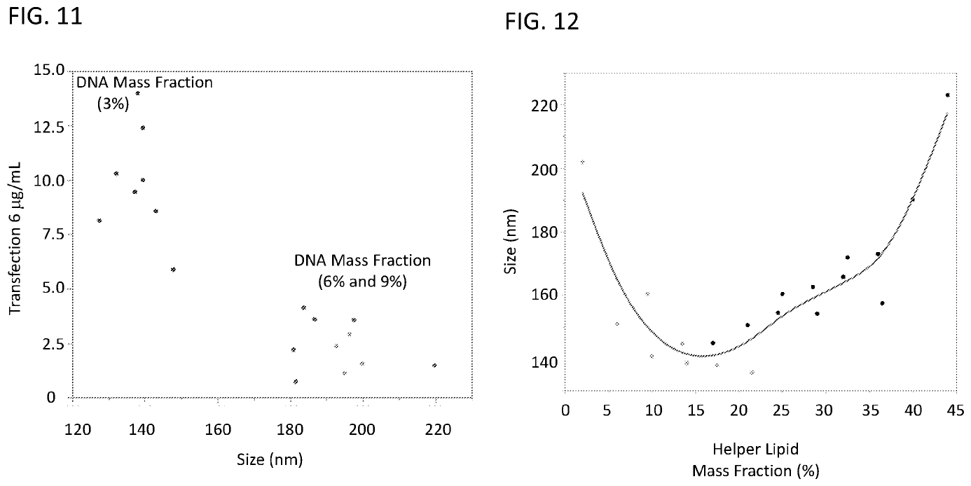

When particle size was compared to transfection efficiency, an inverse correlation was observed, such that transfection efficiency decreased with increased particle size (FIG. 11).

These findings are consistent with an observation that higher DNA concentration at a lower DNA mass fraction (3.5%) may result in higher transfection efficiency. In contrast, formulations with 6% and 9% DNA mass fraction resulted in large particles (>200 nM), which correlated with losses upon sterile filtration (data not shown), and also resulted in low overall transfection efficiencies across all doses. The results also showed that increased helper lipid may decrease DNA loading.

c. Helper Lipid Variance

LNPs were generated from various formulations by the methods described in Example 1, in which the mass fraction of DNA plasmid, PEG lipid, ionizable lipid, and cholesterol were held at different fixed percentages, while the mass fraction of helper lipid was allowed to vary (“float”). All other aspects of the formulation components and process parameters were maintained at a constant value. In all, testing five different ionizable lipid mass fraction percentages and four different cholesterol mass fraction percentages yielded a total of 20 different formulations. The LNPs were formulated and assessed substantially as described in the sections above. Table E7 and Table E8 set forth the different generated LNP formulations.

TABLE E7
Formulations with Helper Lipid Float

| Class | Variable | Species | Values | | | | | | Units |
|---|---|---|---|---|---|---|---|---|---|
| Component | DNA Plasmid | αCD19 | 3.5 | | | | | | % |
| | Mass Fraction | CAR-sfGFP | | | | | | | |
| | Ionizable Lipid | DLin-KC2- | 20 | 27.5 | | 35 | 42.5 | 50 | % |
| | Mass Fraction | DMA | | | | | | | |
| | Helper Lipid | SOPC | Float | | | | | | % |
| | Mass Fraction | | | | | | | | |
| | PEG Lipid Mass | DMG- | 2.5 | | | | | | % |
| | Fraction | PEG2000 | | | | | | | |
| | Cholesterol | Cholesterol | 30 | | 34 | | 35 | 42 | % |
| | Mass Fraction | | | | | | | | |
| Process | Total | Lipids + | 4.29 | | | | | | mg/mL |
| | Concentration | DNA | | | | | | | |
| | Aqueous Flow | DNA in | 6.0 | | | | | | mL/min |
| | Rate | Buffer | | | | | | | |
| | Organic Flow | Lipids in | 4.5 | | | | | | mL/min |
| | Rate | Ethanol | | | | | | | |

| | Temperature | | RT (20) | Degrees |
|---|---|---|---|---|
| | | | | Celsius |
| Buffer | Concentration | Acetate | 25 | mM |
| | pH | | 4.0 | — |

TABLE E8
LNP Formulations with Helper Lipid Float

| | PEG | | Ionizable | | Helper |
|---|---|---|---|---|---|
| Formulation | Lipid | DNA | Lipid | Cholesterol | Lipid |
| 1 | 2.5 | 3.5 | 50 | 42 | 2 |
| 2 | 2.5 | 3.5 | 50 | 38 | 6 |
| 3 | 2.5 | 3.5 | 50 | 34 | 10 |
| 4 | 2.5 | 3.5 | 50 | 30 | 14 |
| 5 | 2.5 | 3.5 | 42.5 | 42 | 9.5 |
| 6 | 2.5 | 3.5 | 42.5 | 38 | 13.5 |
| 7 | 2.5 | 3.5 | 42.5 | 34 | 17.5 |
| 8 | 2.5 | 3.5 | 42.5 | 30 | 21.5 |
| 9 | 2.5 | 3.5 | 35 | 42 | 17 |
| 10 | 2.5 | 3.5 | 35 | 38 | 21 |
| 11 | 2.5 | 3.5 | 35 | 34 | 25 |
| 12 | 2.5 | 3.5 | 35 | 30 | 29 |
| 13 | 2.5 | 3.5 | 27.5 | 42 | 24.5 |
| 14 | 2.5 | 3.5 | 27.5 | 38 | 28.5 |
| 15 | 2.5 | 3.5 | 27.5 | 34 | 32.5 |
| 16 | 2.5 | 3.5 | 27.5 | 30 | 36.5 |
| 17 | 2.5 | 3.5 | 20 | 42 | 32 |
| 18 | 2.5 | 3.5 | 20 | 38 | 36 |
| 19 | 2.5 | 3.5 | 20 | 34 | 40 |
| 20 | 2.5 | 3.5 | 20 | 30 | 44 |

LNP size was assessed by DLS, as previously described. As shown in FIG. 12, when the mass fraction of helper lipid was below 20%, increasing SOPC decreased particle size (p<0.05; left side of graph). By contrast, when the mass fraction of helper lipid was above 20%, increasing helper lipid increased particle size (p<0.01; right side of graph). These findings are consistent with an observation that the effect of helper lipid mass fraction on the size of LNPs is dichotomous, in that the effect is opposite below and above approximately 20% mass fraction.

Formulations with greater than 21% mass fraction helper lipid did not effectively concentrate when centrifuged as part of the solvent removal and washing step, such that the resulting DNA was too dilute. Additionally, formulations with 42% cholesterol mass fraction formed large aggregates and were incapable of being properly sterile filtered. As a result, no DNA loading or transfection analysis were performed in this experiment.

d. PEG Lipid Variance

LNPs were generated from various formulations by the methods described in Example 1. The mass fraction percentage of PEG lipid was allowed to vary (“float”) and compensated with cholesterol, while the mass fraction of DNA plasmid, helper lipid, and ionizable lipid were held at different fixed percentages. All other aspects of the formulation components and process parameters were maintained at a constant value. In all, eight different formulations with varying amounts of DMG-PEG2000 were generated in three batches. Table E9 and Table E10 set forth the different LNP formulations.

TABLE E9
Formulations with PEG Lipid Float

| | | | Mol | Mass | |
|---|---|---|---|---|---|
| Class | Variable | Species | Fraction | Fraction | Units |
| Component | DNA Plasmid | pCAG- | ** | 3.5 | % |
| | | sfGFP | | | |
| | Ionizable Lipid | DLin-KC2- | 50 | 57 | % |
| | | DMA | | | |
| | Helper Lipid | SOPC | 5 | 7 | % |
| | PEG Lipid | DMG- | X | Y | % |
| | | PEG2000 | | | |
| | Cholesterol | Cholesterol | 45-X | 32.5-Y | % |
| Process | Total | Lipids + | 4.285 | | mg/mL |
| | Concentration | DNA | | | |
| | Aqueous Flow | DNA in | 6.0 | | mL/min |
| | Rate | Buffer | | | |
| | Organic Flow | Lipids in | 4.5 | | mL/min |
| | Rate | Ethanol | | | |
| | Temperature | | RT (20) | | Degrees |
| | | | | | Celsius |
| Buffer | Concentration | Acetate | 25 | | mM |
| | pH | | 4.0 | | — |

** Calculated independently from lipid molarity fractions

TABLE E10
LNP Formulations with PEG Lipid Float

| | | | | | |
|---|---|---|---|---|---|
| | PEG | | Ionizable | | Helper |
| Formulation | Lipid | DNA | Lipid | Cholesterol | Lipid |

Batch 1 (Mass Fraction %)

| | | | | | |
|---|---|---|---|---|---|
| 1 | 2 | 3.5 | 57 | 30.5 | 7 |
| 2 | 2.2 | 3.5 | 57 | 30.3 | 7 |
| 3 | 2.5 | 3.5 | 57 | 30 | 7 |
| 4 | 3 | 3.5 | 57 | 29.5 | 7 |
| 5 | 3.5 | 3.5 | 57 | 29 | 7 |
| 6 | 4.6 | 3.5 | 57 | 27.9 | 7 |

Batch 2 (Mass Fraction %)

| | | | | | |
|---|---|---|---|---|---|
| 7 | 2.2 | 3.5 | 57 | 30.3 | 7 |
| 8 | 2.5 | 3.5 | 57 | 30 | 7 |
| 9 | 2.7 | 3.5 | 57 | 29.8 | 7 |
| 10 | 3 | 3.5 | 57 | 29.5 | 7 |

Batch 3 (Mass Fraction %)

| | | | | | |
|---|---|---|---|---|---|
| 11 | 2.5 | 3.5 | 57 | 30 | 7 |
| 12 | 3.5 | 3.5 | 57 | 29 | 7 |
| 13 | 4.3 | 3.5 | 57 | 28.2 | 7 |
| 14 | 4.6 | 3.5 | 57 | 27.9 | 7 |

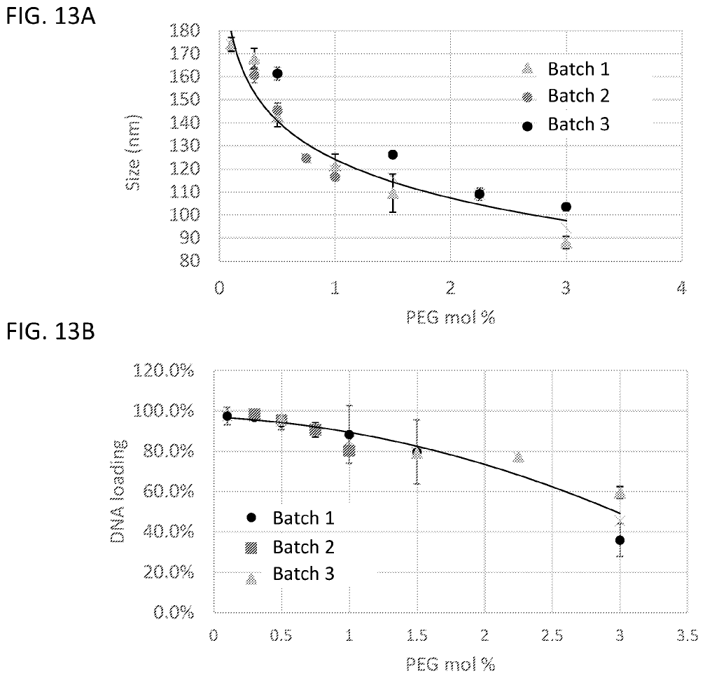

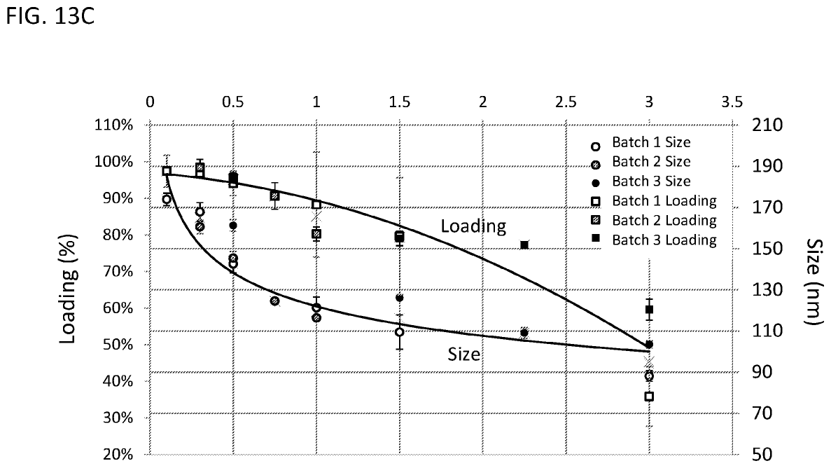

Particle size was assessed by DLS as previously described. The results are shown in FIG. 13A, and demonstrate that molarity fraction of the PEG lipid affected LNP size. Specifically, increasing PEG lipid molarity fraction resulted in smaller particle size. DNA loading into LNPs was assessed by Pico Green fluorescence, as previously described. DNA loading, as shown in FIG. 13B, was affected by PEG lipid molarity fraction; a range of 0.5% to 1.0% PEG lipid molarity fraction (corresponding to 2.5% to 5.0% mass fraction) resulted in DNA loading of greater than 90%. It was found that a range of 0.5% to 1.5% PEG lipid molarity fraction represented a range in which particle size was reduced and DNA loading was increased (FIG. 13C).

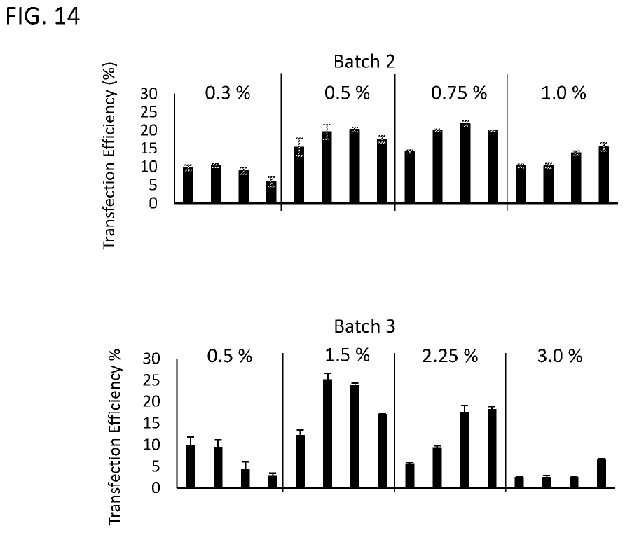

The LNPs were assessed for their effects on transfection efficiency, as described above. As shown in FIG. 14, cell were dosed at either 6.0 g/mL, 3.0 g/mL, 1.5 g/mL, or 0.75 g/mL total DNA (left to right on x-axis, for each PEG condition). Results from batch 2 and batch 3 revealed that formulations with 0.5% to 1.5% molarity fraction of PEG lipid exhibited increased transfection efficiency. In addition, an inverse dose response was observed, wherein the formulations having higher total DNA concentration showed reduced transfection efficiency, especially those formulations having higher PEG lipid molarity fractions.

These results are consistent with a finding that increasing PEG lipid mass fraction may decrease LNP size and DNA loading. However, a molarity fraction of between 0.5% and 1% PEG lipid (corresponding to a mass fraction of between 2.5% and 5%) may represent a range that results in desirable LNP size and DNA loading. Additionally, a PEG lipid molarity fraction of 0.5% to 1.5% may improve transfection efficiency.

e. Simultaneous Variance of All Lipid Nanoparticle Components

In this experiment, each of the components of the LNP formulations was varied simultaneously. The ranges of each component as tested are shown in Table E11. The experiments were performed in two blocks of 20 formulations each, with three replicates across blocks (see Table E12; formulations F4 and F36, F8 and F24, and F18 and F27 were replicates). The LNPs were generated by the methods described in Example 1.

TABLE E11
Mixed Model Formulations

| Class | Variable | Species | Values | Units |
|---|---|---|---|---|
| Component | DNA Plasmid | αCD19 | 3.5-7.5 | % |
| | Mass Fraction | CAR-sfGFP | | |
| | Ionizable Lipid | (DLin-KC2- | 40-55 | % |
| | Mass Fraction | DMA) | | |
| | Helper Lipid | SOPC | 5-20 | % |
| | Mass Fraction | | | |
| | PEG Lipid Mass | DMG- | 2.5-5.0 | % |
| | Fraction | PEG2000 | | |
| | Cholesterol | Cholesterol | 25-35 | % |
| | Mass Fraction | | | |
| Process | Total | Lipids + | 3.0 | mg/mL |
| | Concentration | DNA | | |
| | Aqueous Flow | DNA in | 9.0 | mL/min |
| | Rate | Buffer | | |
| | Organic Flow | Lipids in | 3.0 | mL/min |
| | Rate | Ethanol | | |
| | Temperature | | RT (20) | Degrees |
| | | | | Celsius |
| Buffer | Concentration | Acetate | 25 | mM |
| | pH | | 4.0 | — |

TABLE E12
LNP Formulations with Simultaneous Variance

| | | | | | |
|---|---|---|---|---|---|
| | Ionizable | Helper | PEG | | |
| Formulation | Lipid | Lipid | Lipid | Cholesterol | DNA |

Block 1 (Mass Fraction %)

| | | | | | |
|---|---|---|---|---|---|
| 1 | 40 | 20 | 5 | 29.5 | 5.5 |
| 2 | 47.5 | 13.25 | 2.5 | 33.25 | 3.5 |
| 3 | 55 | 5 | 5 | 30.00 | 5.0 |
| 4 | 42 | 20 | 2.5 | 28 | 7.5 |
| 5 | 47.875 | 5 | 3.75 | 35.875 | 7.5 |
| 6 | 47.25 | 12.25 | 5 | 28 | 7.5 |
| 7 | 55 | 5 | 3.75 | 32.75 | 3.5 |
| 8 | 54.5 | 5 | 5 | 28 | 7.5 |
| 9 | 40 | 20 | 2.5 | 34 | 3.5 |
| 10 | 55 | 11.5 | 5 | 25 | 3.5 |
| 11 | 44.5 | 5 | 5 | 40 | 5.5 |
| 12 | 40 | 12.75 | 3.75 | 40 | 3.5 |
| 13 | 46.5 | 5 | 5 | 40 | 3.5 |
| 14 | 55 | 14 | 2.5 | 25 | 3.5 |
| 15 | 40 | 20 | 5 | 31.5 | 3.5 |
| 16 | 45 | 10.5 | 2.5 | 34.5 | 7.5 |
| 17 | 43 | 5 | 4.5 | 40 | 7.5 |
| 18 | 43 | 7 | 2.5 | 40 | 7.5 |
| 19 | 40 | 19.5 | 5 | 28 | 7.5 |
| 20 | 55 | 5 | 2.5 | 30 | 7.5 |

Block 2 (Mass Fraction %)

| | | | | | |
|---|---|---|---|---|---|
| 21 | 55 | 9.25 | 3.75 | 26.5 | 5.5 |
| 22 | 46.25 | 11.5 | 3.75 | 33 | 5.5 |
| 23 | 40 | 20 | 2.5 | 32 | 5.5 |
| 24 | 54.5 | 5 | 5 | 28 | 7.5 |
| 25 | 43 | 5 | 5 | 39.5 | 7.5 |
| 26 | 47.75 | 20 | 3.75 | 25 | 3.5 |
| 27 | 43 | 7 | 2.5 | 40 | 7.5 |
| 28 | 46.5 | 20 | 5 | 25 | 3.5 |
| 29 | 55 | 5 | 2.5 | 34 | 3.5 |
| 30 | 40 | 11.5 | 5 | 40 | 3.5 |
| 31 | 55 | 5 | 5 | 31.5 | 3.5 |
| 32 | 40 | 14 | 2.5 | 40 | 3.5 |
| 33 | 40 | 20 | 3.9 | 28.6 | 7.5 |
| 34 | 55 | 10.5 | 2.5 | 26.5 | 5.5 |
| 35 | 47 | 5 | 2.5 | 40 | 5.5 |
| 36 | 42 | 20 | 2.5 | 28 | 7.5 |
| 37 | 49 | 5 | 2.5 | 40 | 3.5 |
| 38 | 40 | 12.25 | 5 | 35.25 | 7.5 |
| 39 | 49 | 20 | 2.5 | 25 | 3.5 |
| 40 | 55 | 5 | 2.5 | 30 | 7.5 |

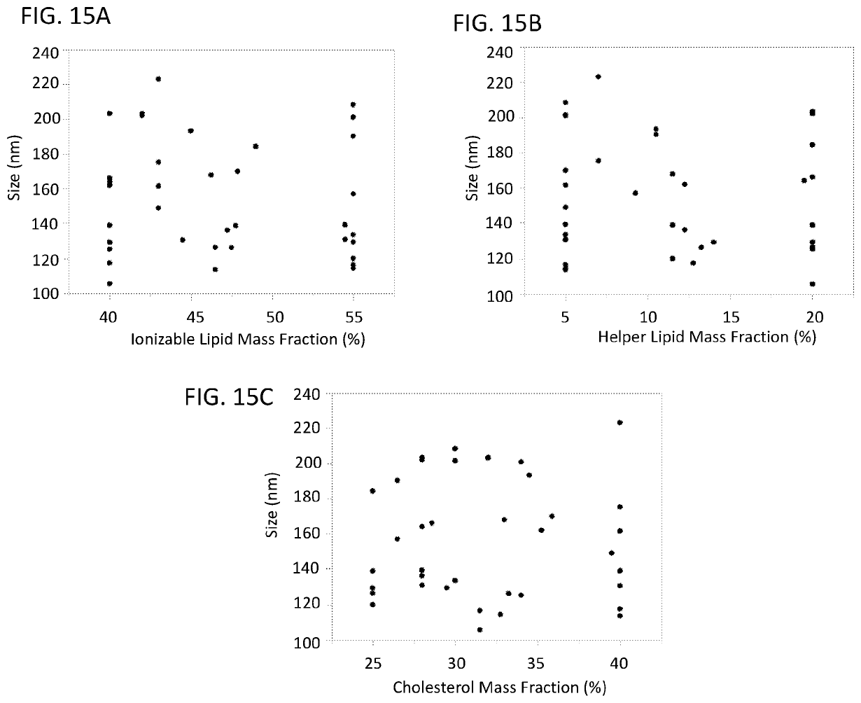

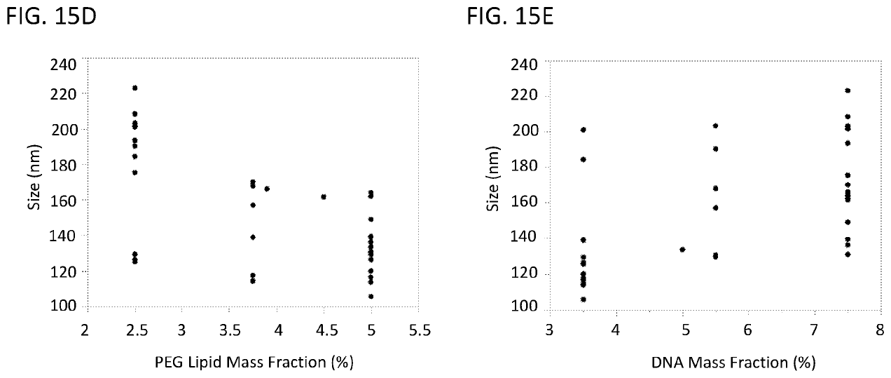

LNP size was assessed by DLS, as shown in FIGS. 15A-15E. Consistent with results above, increasing PEG lipid mass fraction percentage decreased particle size (p<0.001; FIG. 15D), whereas increasing plasmid DNA mass fraction percentage increased particle size (p<0.001; FIG. 15E). No significant effects of ionizable lipid (FIG. 15A), helper lipid (FIG. 15B), or cholesterol (FIG. 15C) mass fraction percentages were observed.

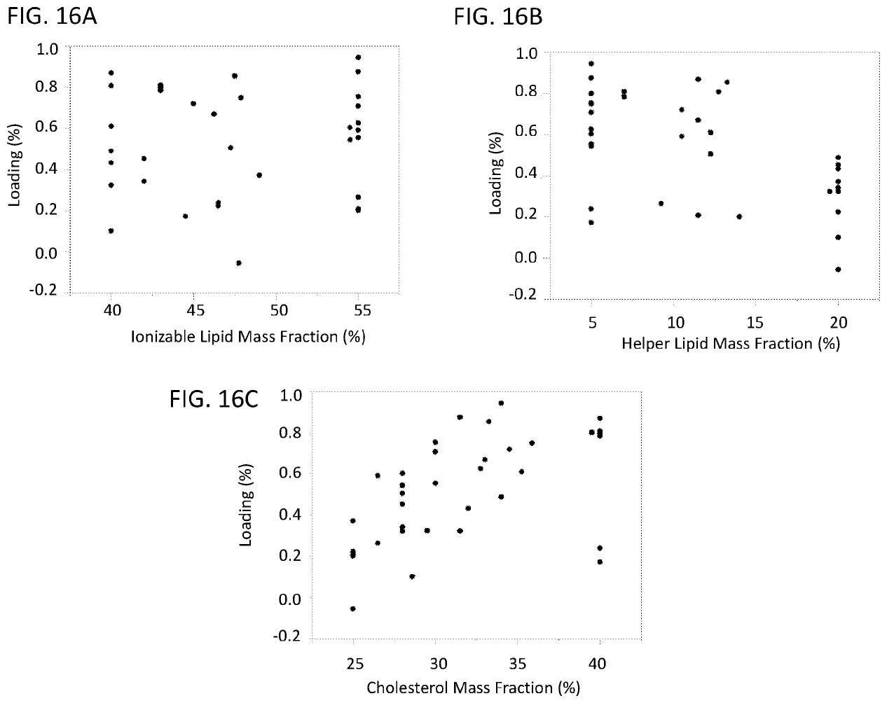

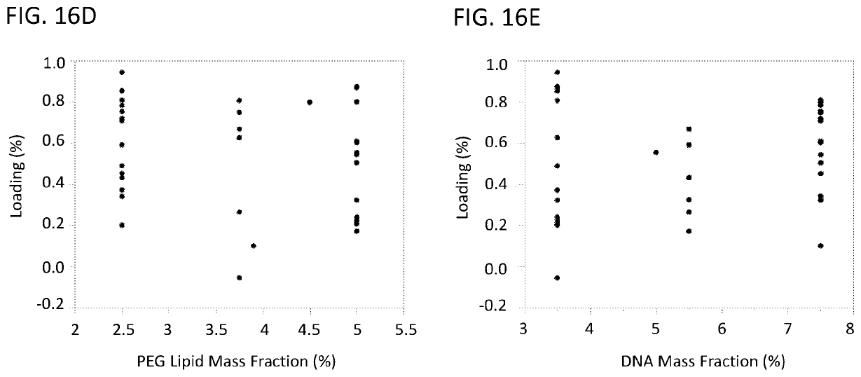

DNA loading was also assessed by PicoGreen, as shown in FIGS. 16A-16E. Consistent with results above, increasing helper lipid mass fraction percentage decreased DNA loading (p<0.001; FIG. 16B), whereas increasing cholesterol mass fraction percentage increased loading (p<0.001; FIG. 16C). No significant effects of ionizable lipid (FIG. 16A), PEG lipid (FIG. 16D), or plasmid DNA (FIG. 16E) mass fraction percentages were observed.

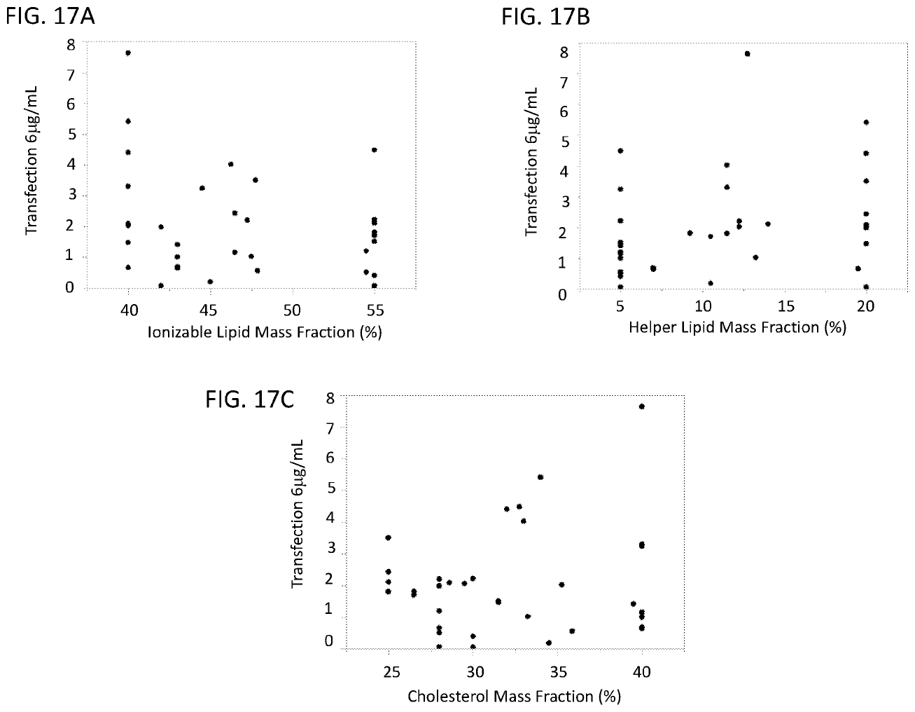

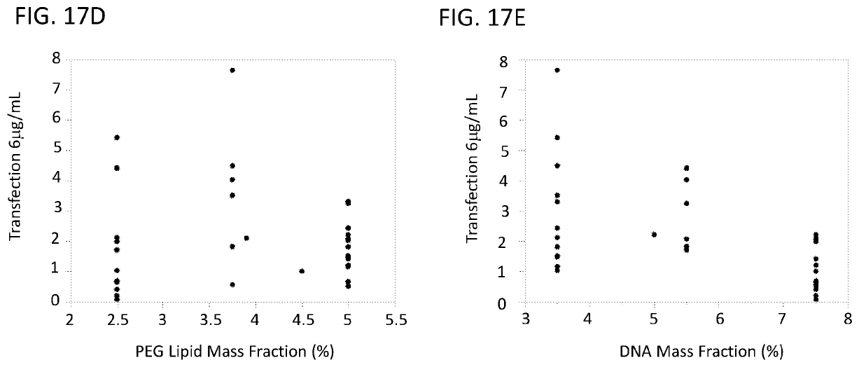

The generated LNPs were assessed for their effects on cell viability and transfection efficiency, as described in the preceding sections (FIGS. 17A-17E). Increasing plasmid DNA mass fraction percentage decreased transfection efficiency, consistent with previous results (p<0.001; FIG. 17E). The other components were not observed to significantly affect transfection efficiency (FIGS. 17A-D).

### Example 4: Effects of Different Ionizable Lipids

Features of the ionizable lipid, such as length of alkyl chain linkers and structure of heterocyclic head group, on LNP particle size, DNA loading, and transient transfection efficiency and viability were assessed.

a. Alkyl Chain Linker Length

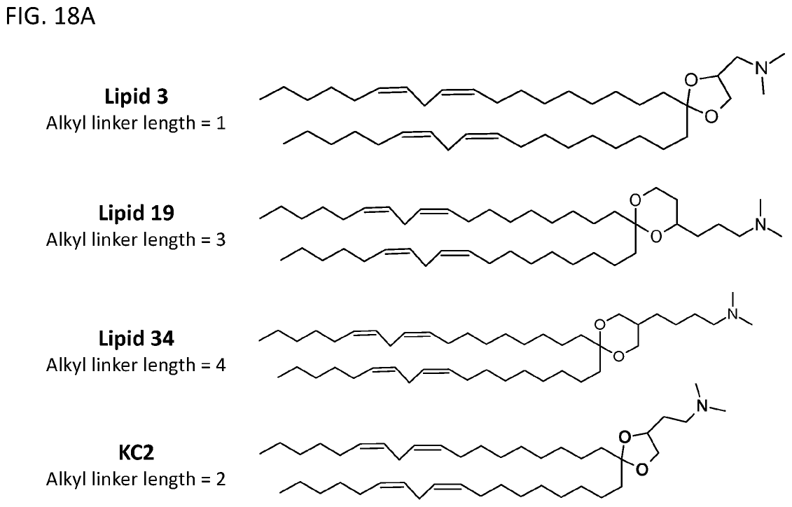

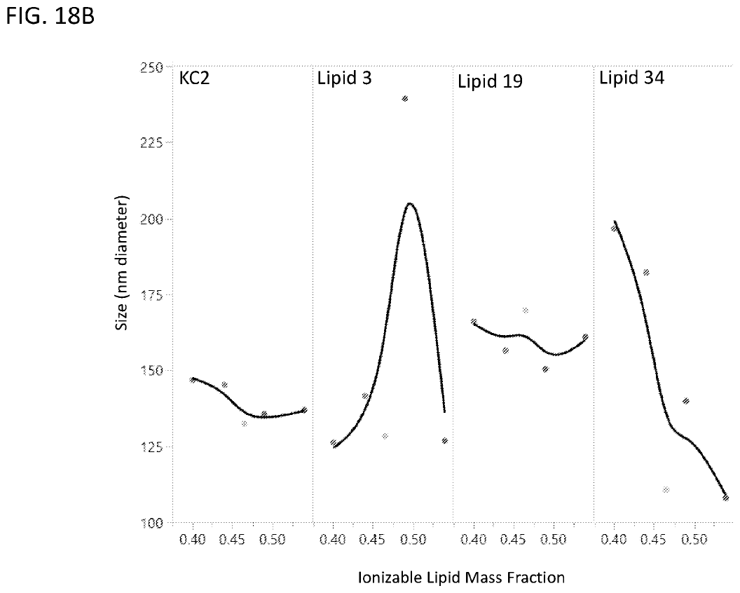

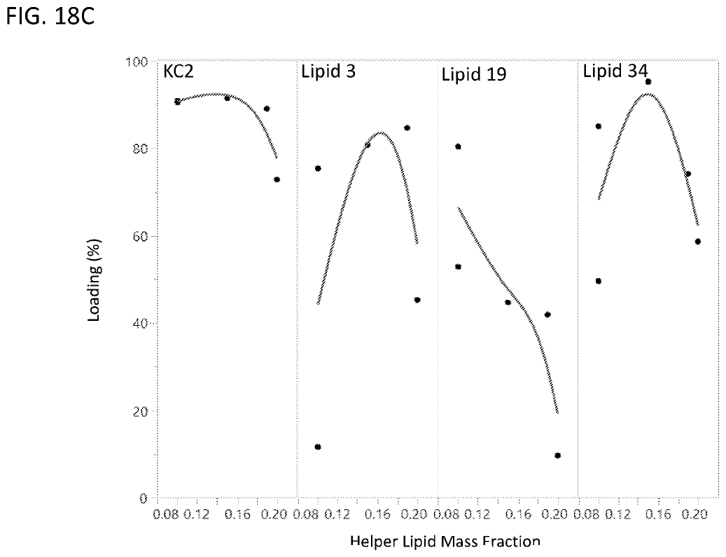

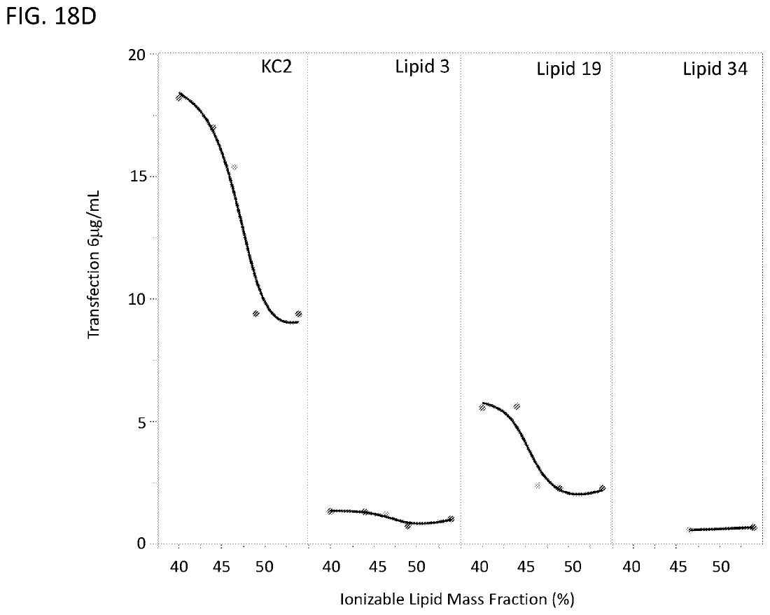

The DLin-KC2-DMA ionizable lipid having an alkyl chain linker length of 2 (reported pKa: 6.68), as described above, was compared to three DLin-KC2-DMA analogs in which the headgroup motif was maintained, but the alkyl chain linker length was 1 (Lipid 3; reported pKa: 5.94), 3 (Lipid 19; reported pKa: 6.95), or 4 (Lipid 34; reported pKa: 7.61) (FIG. 18A).

For each of the four analogs, five different LNP formulations were generated with varying mass fraction percentages of ionizable lipid, helper lipid, and cholesterol, while PEG lipid and DNA mass fraction percentages were held constant (see Table E13). The LNP formulations are shown in Table E14. The formulations were generated by the methods described in Example 1, except as noted.

TABLE E13
LNP Formulations with DLin-KC2-DMA Analogs

| Class | Variable | Species | Values | Units |
|---|---|---|---|---|
| Component | DNA Plasmid | αCD19 | 3.5 | % |
| | Mass Fraction | CAR-sfGFP | | |
| | Ionizable Lipid | (KC2 and | 40-55 | % |
| | Mass Fraction | Analogs) | | |
| | Helper Lipid | SOPC | 10-20 | % |
| | Mass Fraction | | | |
| | PEG Lipid Mass | DMG- | 2.5 | % |
| | Fraction | PEG2000 | | |
| | Cholesterol | Cholesterol | 30-35 | % |
| | Mass Fraction | | | |
| Process | Total | Lipids + | 3.0 | mg/mL |
| | Concentration | DNA | | |
| | Aqueous Flow | DNA in | 9.0 | mL/min |
| | Rate | Buffer | | |
| | Organic Flow | Lipids in | 3.0 | mL/min |
| | Rate | Ethanol | | |
| | Temperature | | RT (~22) | Degrees |
| | | | | Celsius |
| Buffer | Concentration | Acetate | 25 | mM |
| | pH | | 4.0 | — |

TABLE E14
LNP Formulations with DLin-KC2-DMA Analogs

| | Ionizable | Helper | PEG | | |
|---|---|---|---|---|---|
| Formulation | Lipid | Lipid | Lipid | Cholesterol | DNA |
| KC2-1 | 49 | 10 | 2.5 | 35 | 3.5 |
| KC2-2 | 44 | 20 | 2.5 | 30 | 3.5 |
| KC2-3 | 40 | 19 | 2.5 | 35 | 3.5 |
| KC2-4 | 54 | 10 | 2.5 | 30 | 3.5 |
| KC2-5 | 46.5 | 15 | 2.5 | 32.5 | 3.5 |
| Lipid 3-1 | 49 | 10 | 2.5 | 35 | 3.5 |
| Lipid 3-2 | 44 | 20 | 2.5 | 30 | 3.5 |
| Lipid 3-3 | 40 | 19 | 2.5 | 35 | 3.5 |
| Lipid 3-4 | 54 | 10 | 2.5 | 30 | 3.5 |
| Lipid 3-5 | 46.5 | 15 | 2.5 | 32.5 | 3.5 |
| Lipid 19-1 | 49 | 10 | 2.5 | 35 | 3.5 |
| Lipid 19-2 | 44 | 20 | 2.5 | 30 | 3.5 |
| Lipid 19-3 | 40 | 19 | 2.5 | 35 | 3.5 |
| Lipid 19-4 | 54 | 10 | 2.5 | 30 | 3.5 |
| Lipid 19-5 | 46.5 | 15 | 2.5 | 32.5 | 3.5 |
| Lipid 34-1 | 49 | 10 | 2.5 | 35 | 3.5 |
| Lipid 34-2 | 44 | 20 | 2.5 | 30 | 3.5 |
| Lipid 34-3 | 40 | 19 | 2.5 | 35 | 3.5 |
| Lipid 34-4 | 54 | 10 | 2.5 | 30 | 3.5 |
| Lipid 34-5 | 46.5 | 15 | 2.5 | 32.5 | 3.5 |

LNP size and DNA loading were assessed by the methods described in previous Examples. The results showed that LNP and DNA loading were not significantly affected by the alkyl chain linker length (FIG. 18B and FIG. 18C, respectively). LNPs generated with DLin-KC2-DMA were observed to exhibit consistently high DNA loading (FIG. 18C).

A significant effect of lipid identity on transfection efficiency was observed (p<0.001), with linker lengths of 2 (DLin-KC2-DMA) and 3 (Lipid 19) demonstrating higher transfection efficiency compared to the other lipids (FIG. 18D). For both DLin-KC2-DMA and Lipid 19, increasing ionizable lipid mass fraction percentage and decreasing helper lipid mass fraction percentage decreased transfection efficiency.

These findings indicate that DLin-KC2-DMA resulted in the highest transfection efficiency compared to other tested analogs. Among lipids tested, Lipids 3 and 34 with the lowest (5.94) and highest (7.61) reported pKa, as well as respective (n=1, n=4) alkyl chain lengths, exhibited low transfection efficiency.

b. Heterocyclic Headgroup

Various ionizable lipid heterocyclic headgroups were assessed for their effects on LNP size, DNA loading, and transient transfection efficiency.

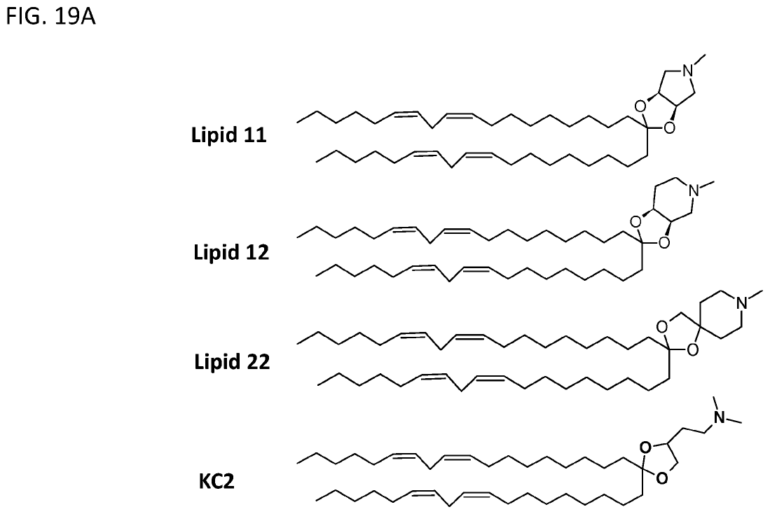

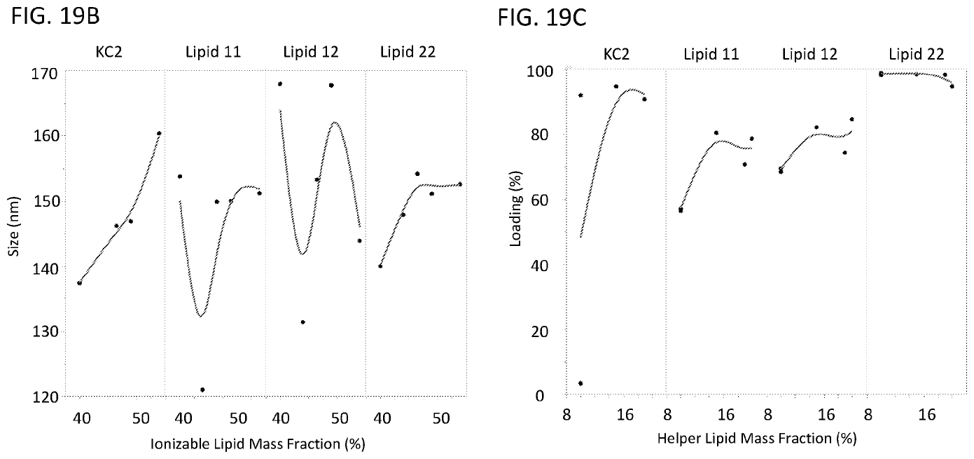

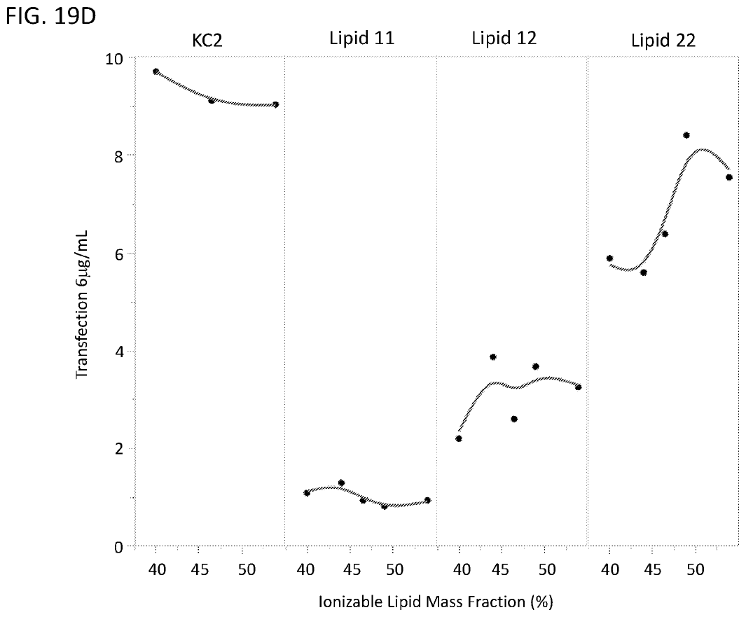

The DLin-KC2-DMA ionizable lipid previously described was compared to three DLin-KC2-DMA analogs in which the heterocyclic headgroup motif differed (Lipids 11, 12, and 22, as shown in FIG. 19A). The reported pKa of Lipids 11, 12, and 22 were 5.65, 5.60, and 6.53, respectively. For each of the four analogs, five different compositions were formulated with varying mass fraction percentages of ionizable lipid, helper lipid, and cholesterol, while the mass fraction percentages of the PEG lipid and DNA were held constant (see Table E15). The LNP formulations are shown in Table E16. The formulations were generated by the methods described in Example 1, except as noted.

TABLE E15
LNP Formulations with DLin-KC2-DMA Analogs

| Class | Variable | Species | Values | Units |
|---|---|---|---|---|
| Component | DNA Plasmid | aCD19 | 3.5 | % |
| | Mass Fraction | CAR-sfGFP | | |
| | Ionizable Lipid | (KC2 and | 40-55 | % |
| | Mass Fraction | Analogs) | | |
| | Helper Lipid | SOPC | 10-20 | % |
| | Mass Fraction | | | |
| | PEG Lipid Mass | DMG- | 2.5 | % |
| | Fraction | PEG2000 | | |
| | Cholesterol | Cholesterol | 30-35 | % |
| | Mass Fraction | | | |
| Process | Total | Lipids + | 3.0 | mg/mL |
| | Concentration | DNA | | |
| | Aqueous Flow | DNA in | 9.0 | mL/min |
| | Rate | Buffer | | |
| | Organic Flow | Lipids in | 3.0 | mL/min |
| | Rate | Ethanol | | |
| | Temperature | | RT (~22) | Degrees |
| | | | | Celsius |
| Buffer | Concentration | Acetate | 25 | mM |
| | pH | | 4.0 | — |

TABLE E16
LNP Formulations with DLin-KC2-DMA Analogs

| | Ionizable | Helper | PEG | | |
|---|---|---|---|---|---|
| Formulation | Lipid | Lipid | Lipid | Cholesterol | DNA |
| KC2-1 | 49 | 10 | 2.5 | 35 | 3.5 |
| KC2-2 | 44 | 20 | 2.5 | 30 | 3.5 |
| KC2-3 | 40 | 19 | 2.5 | 35 | 3.5 |
| KC2-4 | 54 | 10 | 2.5 | 30 | 3.5 |
| KC2-5 | 46.5 | 15 | 2.5 | 32.5 | 3.5 |
| Lipid 11-1 | 49 | 10 | 2.5 | 35 | 3.5 |
| Lipid 11-2 | 44 | 20 | 2.5 | 30 | 3.5 |
| Lipid 11-3 | 40 | 19 | 2.5 | 35 | 3.5 |
| Lipid 11-4 | 54 | 10 | 2.5 | 30 | 3.5 |
| Lipid 11-5 | 46.5 | 15 | 2.5 | 32.5 | 3.5 |
| Lipid 12-1 | 49 | 10 | 2.5 | 35 | 3.5 |
| Lipid 12-2 | 44 | 20 | 2.5 | 30 | 3.5 |
| Lipid 12-3 | 40 | 19 | 2.5 | 35 | 3.5 |
| Lipid 12-4 | 54 | 10 | 2.5 | 30 | 3.5 |
| Lipid 12-5 | 46.5 | 15 | 2.5 | 32.5 | 3.5 |
| Lipid 22-1 | 49 | 10 | 2.5 | 35 | 3.5 |
| Lipid 22-2 | 44 | 20 | 2.5 | 30 | 3.5 |
| Lipid 22-3 | 40 | 19 | 2.5 | 35 | 3.5 |
| Lipid 22-4 | 54 | 10 | 2.5 | 30 | 3.5 |
| Lipid 22-5 | 46.5 | 15 | 2.5 | 32.5 | 3.5 |
| Neg Ctrl | 41.45 | 19.69 | 2.59 | 36.27 | 0.0 |
| PosCtrl | 57 | 7 | 2.5 | 30 | 3.5 |

The results showed that increasing ionizable lipid mass fraction percentage significantly increased the size of LNPs when DLin-KC2-DMA was the ionizable lipid (p<0.0; FIG. 19B). As shown in FIG. 19B, no statistically significant effect of formulation on particle size was observed for other ionizable lipids that were tested. No significant effect of lipid identity or formulation on DNA loading was observed (FIG. 19C).

A significant effect of lipid identity on transfection efficiency was observed, with DLin-KC2-DMA (reported pKa 6.68) and Lipid 22 (reported pKa 6.53) consistently yielding the highest and second-highest transfection efficiency, respectively (p<0.001; FIG. 19D).

### Example 5: Formulation Process Parameters

Various parameters of the LNP formulation process, described in Example 1, were modified to determine the effects on LNP size, DNA loading, and transient transfection efficiency. In this experiment, the mass fraction percentages of individual components of the LNP formulations were held constant, while different fixed values of each process parameter were assessed. The various values tested for each process parameter are shown in Table E17. The particles were generated by the methods described in Example 1.

TABLE E17
Process Parameters for Experimental Formulations

| Class | Variable | Species | Values | | | | Units |
|---|---|---|---|---|---|---|---|
| Component | DNA Plasmid | αCD19 | 3.5 | | | | % |
| | Mass Fraction | CAR-sfGFP | | | | | |
| | Ionizable Lipid | (DLin-KC2- | 40 | | | | % |
| | Mass Fraction | DMA) | | | | | |
| | Helper Lipid | SOPC | 20 | | | | % |
| | Mass Fraction | | | | | | |
| | PEG Lipid Mass | DMG- | 2.5 | | | | % |
| | Fraction | PEG2000 | | | | | |
| | Cholesterol | Cholesterol | 34 | | | | % |
| | Mass Fraction | | | | | | |
| Process | Total | Lipids + | 4.29 | | 3.0 | | mg/mL |
| | Concentration | DNA | | | | | |
| | Aqueous Flow | DNA in | 3.0 | 6.0 | | 9.0 | mL/min |
| | Rate | Buffer | | | | | |
| | Organic Flow | Lipids in | 3.0 | 4.5 | | 6.0 | mL/min |
| | Rate | Ethanol | | | | | |

| | Temperature | | RT (20) | Degrees |
|---|---|---|---|---|
| | | | | Celsius |
| Buffer | Concentration | Acetate | 25 | mM |
| | pH | | 4.0 | — |

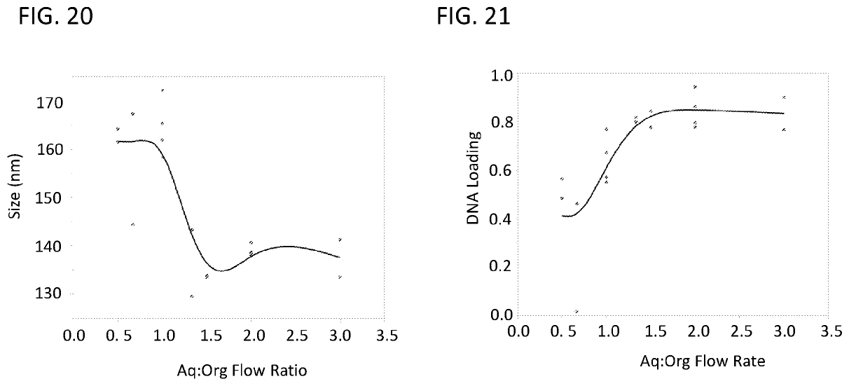

As shown in FIG. 20, increasing the ratio of aqueous flow rate to organic flow rate (Aq:Org) significantly reduced the size of the LNPs (p<0.01). By contrast, no significant effect on LNP size was observed when aqueous flow rate, organic flow rate, or total flow rate was individually changed. Total concentration of lipids and DNA was also not observed to significantly affect LNP size.

Increasing Aq:Org was additionally observed to significantly increase DNA loading (p<0.01), and a DNA loading rate of approximately 80% was observed at all Aq:Org ratios greater than or equal to 1.5 (FIG. 21). However, individually changing aqueous flow rate, organic flow rate, or total flow rate did not significantly affect DNA loading. The total concentration of lipids and DNA was not observed to have a significant effect on DNA loading.

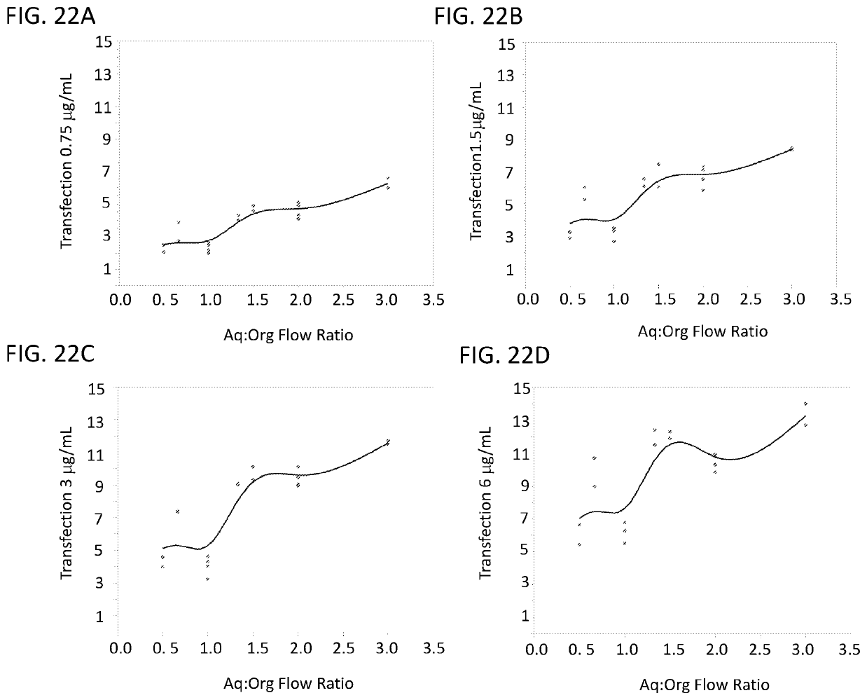

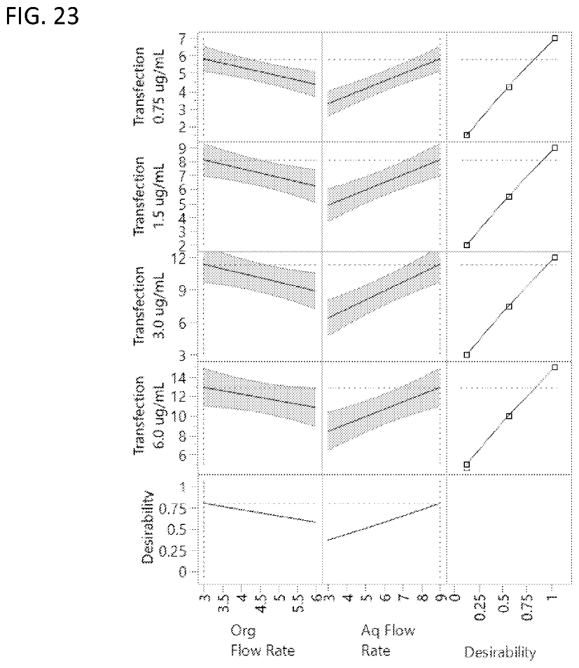

Combined CD4+ and CD8+ T cell compositions were generated, activated and transfected with plasmid DNA substantially as described in preceding Examples, except that cells were transfected with a total DNA concentration of 0.75, 1.5, 3.0, or 6.0 μg/mL. Following incubation of the primary T cells with 1 g/mL ApoE4 and LNPs, cells were assessed for viability and transient transfection efficiency as previously described. The results for Aq:Org flow ratio at the different assessed total DNA concentrations are depicted in FIGS. 22A-22D. As shown, Aq:Org flow rate ratio was observed to have a significant effect on transfection efficiency at all transfection concentrations tested (p<0.001). Optimum transfection efficiency at different aqueous and organic flow rates was predicted using SAS software. It was found that transfection efficiency at all DNA concentrations was maximized at the highest Aq:Org ratio (FIG. 23). However, individually varying aqueous flow rate, organic flow rate, total flow rate, or total concentration of DNA and lipids did not statistically impact transfection efficiency in these studies.

These results demonstrate that increasing the Aq:Org flow ratio reduces LNP particle size, increases DNA loading, and increases transfection efficiency at all doses tested.

### Example 6: Assessment of Lipid Nanoparticle Transfection of Different Donor T Cells

To assess the effects of interdonor variability, combined CD4+ and CD8+ T cell compositions were generated from six different healthy human donors (donors “A” through “F”), activated, and incubated with LNPs produced from three different formulations. Cells were transfected with either 0, 0.75, 1.5, 3, 6, 12, or 24 □g of either pCAG-sfGFP or αCD19 CAR-sfGFP plasmid DNA, substantially as described in preceding examples. The components and formulation process parameters of each formulation are given below in Tables E18 and E19, respectively.

TABLE E18
LNP Formulation Components

| | Mass fraction (%) | | | | | Mol fraction (%) | | | | |
|---|---|---|---|---|---|---|---|---|---|---|
| | Ion | Helper | | | | Ion | Helper | | | |
| Formulation | (KC2) | (SOPC) | PEG | Chol | DNA | (KC2) | (SOPC) | PEG | Chol | N:P |
| 1 | 57 | 7 | 2.5 | 30 | 3.5 | 50.4 | 5 | 0.5 | 44 | 8.6 |
| 2 | 40 | 20 | 2.5 | 34 | 3.5 | 35.3 | 14.4 | 0.5 | 49.8 | 6 |
| 3 | 52.6 | 12.9 | 6.5 | 24.3 | 3.7 | 50 | 10 | 1.5 | 38.5 | 6 |

TABLE E19
LNP Process Parameters

| | | | | | | Total Conc. |
|---|---|---|---|---|---|---|
| | DNA (Aqueous) | | Lipid Mix (Organic) | | | Solids |
| Formulation | mL/min | mg/mL | mL/min | mg/mL | mM | mg/mL |
| 1 | 6 | 0.262 | 4.5 | 9.65 | 7.55 | 4.29 |
| 2 | 9 | 0.14 | 3 | 11.58 | 5.3 | 3 |
| 3 | n/a* | 0.3611 | n/a* | 1.16 | 39.4 | 6.02 |

*Generated on NanoAssemblr ® Spark ™ instrument.

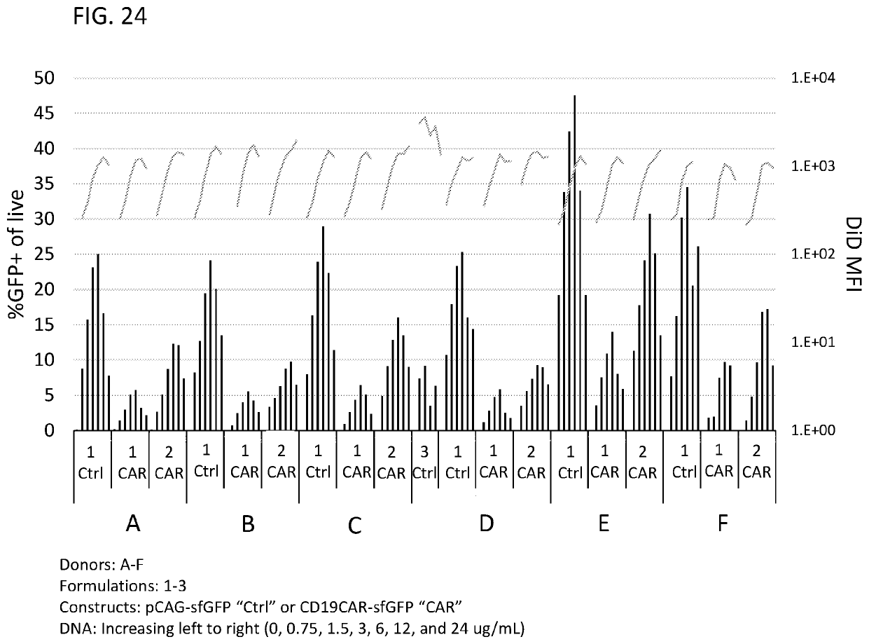

Cellular uptake of LNPs was assessed by inclusion of DiD in the formulations, and transient transfection efficiency was assessed by flow cytometry (FIG. 24; uptake shown by lines and right axis, and GFP expression shown by bars and left axis). Results are shown for each formulation prepared with increasing concentrations of plasmid DNA, shown left to right for each LNP formulation.

Formulation 1, encapsulating the pCAG-sfGFP plasmid, resulted in transfection efficiencies ranging from 24.1% to 47.5% among donors, with an average of 30.9%. Formulation 2, encapsulating the αCD19 CAR-sfGFP plasmid, reached transfection efficiency as high as 30.7% for Donor E. In general, Formulation 2 resulted in higher transfection efficiency of the αCD19 CAR-sfGFP plasmid than Formulation 1.

It was observed that transfection with higher concentrations of DNA resulted in a decrease in LNP uptake. Differences in transfection efficiency (% GFP+) among healthy donors were not explained by differences in LNP uptake.

### Example 7: Lipid Nanoparticle Formulation Stability

LNPs were generated from three different formulations in duplicate using the methods described in Example 1 and analyzed for size and DNA loading following refrigerated storage for one week. The compositions of the three formulations (Control 1, Control 2, and Control 3) are shown in Table E20.

TABLE E20
Formulation Components and Process Parameters

| | | | Values | | | |
|---|---|---|---|---|---|---|
| Class | Variable | Species | Ctrl 1 | Ctrl 2 | Ctrl 3 | Units |
| Component | DNA Plasmid | αCD19 CAR- | 3.5* | 3.5 | 3.5 | % |
| | Mass Fraction | sfGFP | | | | |
| | Ionizable Lipid | (DLin-KC2- | 57 | 57 | 40 | % |
| | Mass Fraction | DMA) | | | | |
| | Helper Lipid | SOPC | 7 | 7 | 20 | % |
| | Mass Fraction | | | | | |
| | PEG Lipid | DMG- | 2.5 | 2.5 | 2.5 | % |
| | Mass Fraction | PEG2000 | | | | |
| | Cholesterol | Cholesterol | 30 | 30 | 34 | % |
| | Mass Fraction | | | | | |
| Process | Total | Lipids + | 4.285 | 4.285 | 3.0 | mg/mL |
| | Concentration | DNA | | | | |
| | Aqueous Flow | DNA in | 6.0 | 6.0 | 9.0 | mL/min |
| | Rate | Buffer | | | | |
| | Organic Flow | Lipids in | 4.5 | 4.5 | 3.0 | mL/min |
| | Rate | Ethanol | | | | |
| | Temperature | | RT (20) | RT (20) | RT (20) | Degrees |
| | | | | | | Celsius |
| Buffer | Concentration | Acetate | 25 | 25 | 25 | mM |
| | pH | | 4.0 | 4.0 | 4.0 | — |

*Ctrl 1 was formulated with pCAG-sfGFP plasmid.

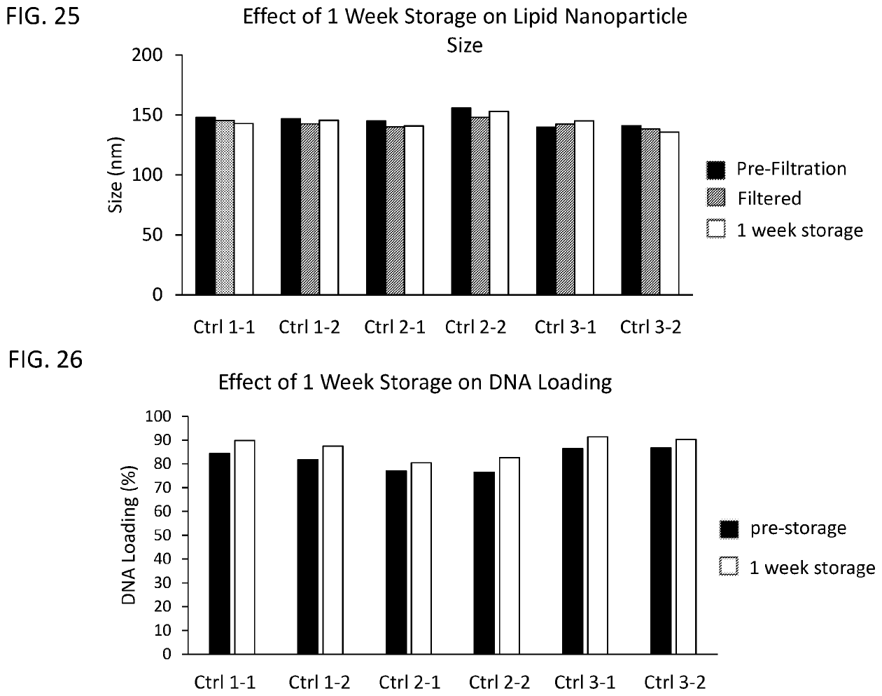

The size of the LNPs was assessed by DLS prior to sterile filtration and storage (“Pre-Filtration”), after sterile filtration but prior to storage (“Filtered”), and after sterile filtration and storage for one week at 4 Celsius (“1 week storage”). As shown in FIG. 25, size of the LNPs for all three formulations was not significantly changed after storage in solution for one week. DNA loading, as assessed by Pico Green fluorescence, was also measured in LNPs generated from the three formulations before (“pre-storage”) and after (“1 week storage”) one week of storage. No significant changes in DNA loading into LNPs were observed when comparing pre- and post-storage formulations (FIG. 26).

Together, these results indicate that the exemplary generated LNPs are stable, both in terms of size and DNA loading, when stored in solution for one week at 4 Celsius.

### Example 8: Lipid Nanoparticle Formulation for DNA Delivery

LNPs from formulations “F9” and “F9A” were generated substantially as described in Example 1 using a Precision Nanosystems NanoAssemblr™ Ignite or Benchtop instrument. F9 and F9A were formulated to contain either an ant-CD19 CAR-sfGFP DNA plasmid (˜7.3 kb) under the EF1a promoter or an anti-CD19 CAR-sfGFP DNA nanoplasmid (˜4.9 kb), where both plasmids encoded the same CAR described in Example 3. In some cases, LNPs generated from the F9 or F9A formulation were subjected to overnight dialysis in PBS prior to washing. Resulting LNPs were found to be stable for up to eight weeks at 4° Celsius. The components and formulation process parameters for the F9 and F9A formulations are given below in Table E21A and Table E21B, respectively.

TABLE E21A
F9 Formulation Components and Process Parameters

| Class | Variable | Species | Value | Units |
|---|---|---|---|---|
| Component | DNA Plasmid Mass | αCD19 CAR-sfGFP | 3.5 | % |
| | Fraction | plasmid or | | |
| | | nanoplasmid | | |
| | Ionizable Lipid Mass | (DLin-KC2-DMA), | 40 | % |
| | Fraction | L37, or L40 | | |
| | Helper Lipid Mass Fraction | SOPC | 19 | % |
| | PEG Lipid Mass Fraction | DMG-PEG2000 | 2.5 | % |
| | Cholesterol Mass Fraction | Cholesterol | 35 | % |
| | Tracking Dye | DID | <1 | % |
| Process | Total Mass | Solids | 6.0 | mg |
| | Total Concentration | Lipids + DNA | 3.0 | mg/mL |
| | Aqueous Flow Rate | DNA in Buffer | 9.0 | mL/min |
| | Organic Flow Rate | Lipids in Ethanol | 3.0 | mL/min |
| | Temperature | | RT (~22) | Degrees Celsius |
| Buffer | Concentration | Acetate | 25 | mM |
| | pH | | 4.0 | — |

TABLE E21B
F9A Formulation Components and Process Parameters

| Class | Variable | Species | Value | Units |
|---|---|---|---|---|
| Component | DNA Plasmid Mass | αCD19 CAR-sfGFP | 3.5 | % |
| | Fraction | plasmid or | | |
| | | nanoplasmid | | |
| | Ionizable Lipid Mass | (OF-C4-Deg-Lin) | 36 | % |
| | Fraction | | | |
| | Helper Lipid Mass Fraction | SOPC | 19 | % |
| | PEG Lipid Mass Fraction | DMG-PEG2000 | 2.5 | % |
| | Cholesterol Mass Fraction | Cholesterol | 35 | % |
| | Non-ionizable Cationic | Lipid T1 | 4 | % |
| | Lipid Mass Fraction | | | |
| | Tracking Dye | DID | <1 | % |
| Process | Total Mass | Solids | 6.0 | mg |
| | Total Concentration | Lipids + DNA | 3.0 | mg/mL |
| | Aqueous Flow Rate | DNA in Buffer | 9.0 | mL/min |
| | Organic Flow Rate | Lipids in Ethanol | 3.0 | mL/min |
| | Temperature | | RT (~22) | Degrees Celsius |
| Buffer | Concentration | Acetate | 25 | mM |
| | pH | | 4.0 | — |

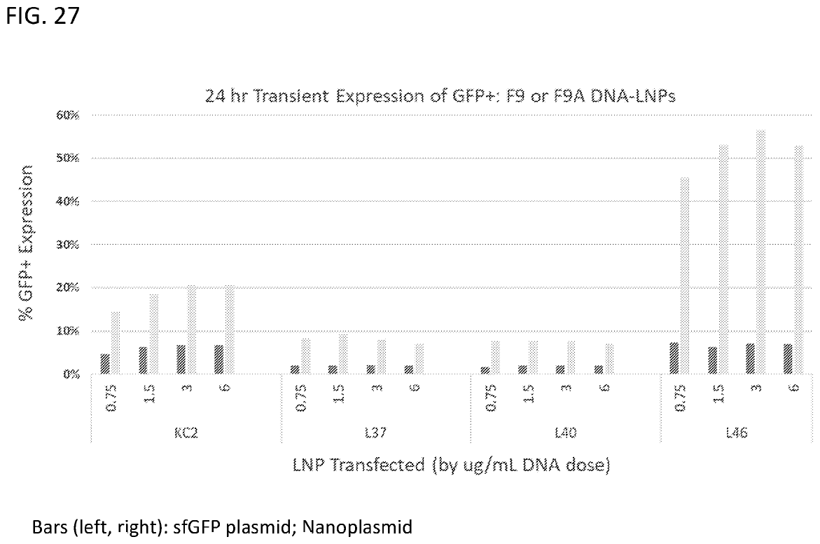

Transient transfection of donor T cells was achieved by incubating T cells from a donor with LNPs generated from the F9 and F9A formulations containing various ionizable lipids, and transfection efficiency was assessed 24 hours later by GFP expression. LNPs containing the OF-C4-Deg-Lin ionizable lipid were observed to result in the highest levels of transient transfection in T cells (FIG. 27).

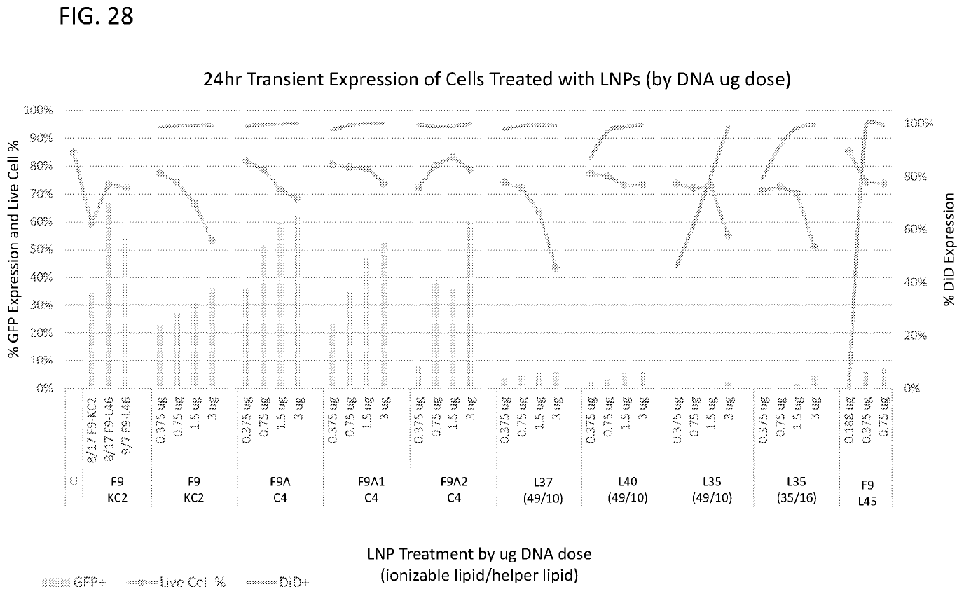

In a related experiment, a variety of ionizable lipids were used in formulations having different mass fractions of ionizable and helper lipids, as compared to F9 and F9A (Table E21C). Specifically, ionizable lipids L40, L37, and L45 were branched, fully saturated lipids, while L35 was a lipidoid having a piperazine ring core, and L45 was a lipidoid having a benzene ring core. Changing the mass fraction percentages of the ionizable and helper lipids did not significantly increase the functionality of the LNPs, as measured by transient GFP expression by cells (FIG. 28).

TABLE E21C
Formulation Components

| Formulations | Ionizable | Cationic | | | | |
|---|---|---|---|---|---|---|
| Made | Lipid | Lipid T1 | SOPC | PEG | Cholesterol | DNA |
| F9-KC2 | 40 | 0 | 19 | 2.5 | 35 | 3.5 |
| F9A C4 | 36 | 4 | 19 | 2.5 | 35 | 3.5 |
| F9A1 C4 | 44.1 | 4.9 | 10 | 2.5 | 35 | 3.5 |
| F9A2 C4 | 31.5 | 3.5 | 16 | 2.5 | 46.5 | 3.5 |
| L40 (49/10) | 49 | 0 | 10 | 2.5 | 35 | 3.5 |
| L37 (49/10) | 49 | 0 | 10 | 2.5 | 35 | 3.5 |
| L35 (49/10) | 49 | 0 | 10 | 2.5 | 35 | 3.5 |
| L35 (35/16) | 35 | 0 | 16 | 2.5 | 46.5 | 3.5 |
| F9 L45 | 40 | 0 | 19 | 2.5 | 35 | 3.5 |

### Example 9: Lipid Nanoparticle Formulation for RNA Delivery

LNP formulations were separately generated for the delivery of RNA, substantially as described in Example 1 using a Precision Nanosystems NanoAssemblr™ Ignite instrument. The components and formulation process parameters for an “M1” RNA formulation, “F19” RNA formulation, and “A-01” RNA formulation are given below in Table E22A, Table E22B, and Table E22C, respectively.

TABLE E22A
M1 Formulation Components and Process Parameters

| Class | Variable | Species | Value | Units |
|---|---|---|---|---|
| Component | RNA Mass Fraction | tdTomato reporter | 3.5 | % |
| | | mRNA | | |
| | Ionizable Lipid Mass | DLin-MC3-DMA | 37.8 | % |
| | Fraction | | | |
| | Helper Lipid Mass Fraction | 1,2-distearoyl-sn- | 15.2 | % |
| | | glycero-3- | | |
| | | phosphocholine | | |
| | | (DSPC) | | |
| | PEG Lipid Mass Fraction | DMG-PEG2000 | 3.5 | % |
| Process | Cholesterol Mass Fraction | Cholesterol | 40 | % |
| | Total Mass | Solids | 1 | mg |
| | Total Concentration | Lipids + mRNA | 1.0 | mg/mL |
| | Aqueous Flow Rate | mRNA in Buffer | 9.0 | mL/min |
| | Organic Flow Rate | Lipids in Ethanol | 3.0 | mL/min |
| Buffer | Temperature | | RT (~22) | Degrees Celsius |
| | Concentration | Acetate | 25 | mM |

TABLE E22B
F19 Formulation Components and Process Parameters

| Class | Variable | Species | Value | Units |
|---|---|---|---|---|
| Component | RNA Mass Fraction | tdTomato reporter | 3.5 | % |
| | | mRNA | | |
| | Ionizable Lipid Mass | DLin-MC3-DMA | 34.4 | % |
| | Fraction | | | |
| | Helper Lipid Mass Fraction | DSPC | 17.6 | % |
| | PEG Lipid Mass Fraction | DMG-PEG2000 | 4.5 | % |
| | Cholesterol Mass Fraction | Cholesterol | 40 | % |
| Process | Total Mass | Solids | 1 | mg |
| | Total Concentration | Lipids + mRNA | 1.0 | mg/mL |
| | Aqueous Flow Rate | mRNA in Buffer | 9.0 | mL/min |
| | Organic Flow Rate | Lipids in Ethanol | 3.0 | mL/min |
| | Temperature | | RT (~22) | Degrees Celsius |
| Buffer | Concentration | Acetate | 25 | mM |
| | pH | | 4.0 | — |

TABLE E22C
A-01 Formulation Components and Process Parameters

| Class | Variable | Species | Value | Units |
|---|---|---|---|---|
| Component | RNA Mass Fraction | tdTomato mRNA | 2.7 | % |
| | Ionizable Lipid Mass | DLin-MC3-DMA | 53.4 | % |
| | Fraction | | | |
| | Helper Lipid Mass Fraction | 1,2-distearoyl-sn- | 13.1 | % |
| | | glycero-3- | | |
| | | phosphocholine | | |
| | | (DSPC) | | |
| | PEG Lipid Mass Fraction | DMG-PEG2000 | 6.3 | % |
| Process | Cholesterol Mass Fraction | Cholesterol | 24.5 | % |
| | Total Mass | Solids | 1 | mg |
| | Total Concentration | Lipids + mRNA | 1.0 | mg/mL |
| | Aqueous Flow Rate | mRNA in Buffer | 9.0 | mL/min |
| | Organic Flow Rate | Lipids in Ethanol | 3.0 | mL/min |
| | Temperature | | RT (~22) | Degrees Celsius |
| Buffer | Concentration | Acetate | 25 | mM |
| | pH | | 4.0 | - |

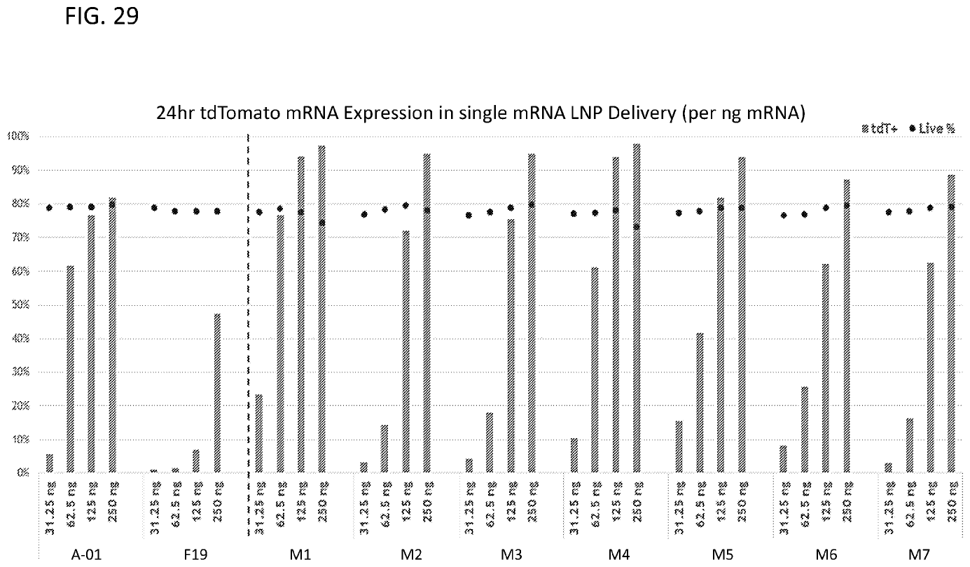

The LNPs were formulated to contain a fluorescent-reporting tdTomato (tdT) mRNA construct. As shown in FIG. 29, LNPs generated from the “A-01” RNA formulation (Table E22C) and the M1 RNA formulation described above (Table E22A) were observed to result in a high frequency of uptake into healthy donor T cells, as determined by the percentage of tdT+ T cells.

### Example 10: Lipid Nanoparticle-Mediated Gene Integration Using Transposon DNA

a. Co-Delivery Using Separate DNA and RNA LNPs

Co-delivery of a piggyBac DNA transposon construct encoding an exemplary anti-CD19 CAR and mRNA encoding the tdTomato (tdT) mRNA construct described in Example 9 was achieved using separate DNA and RNA LNPs generated using a Precision Nanosystems NanoAssemblr™ Ignite or Benchtop instrument.

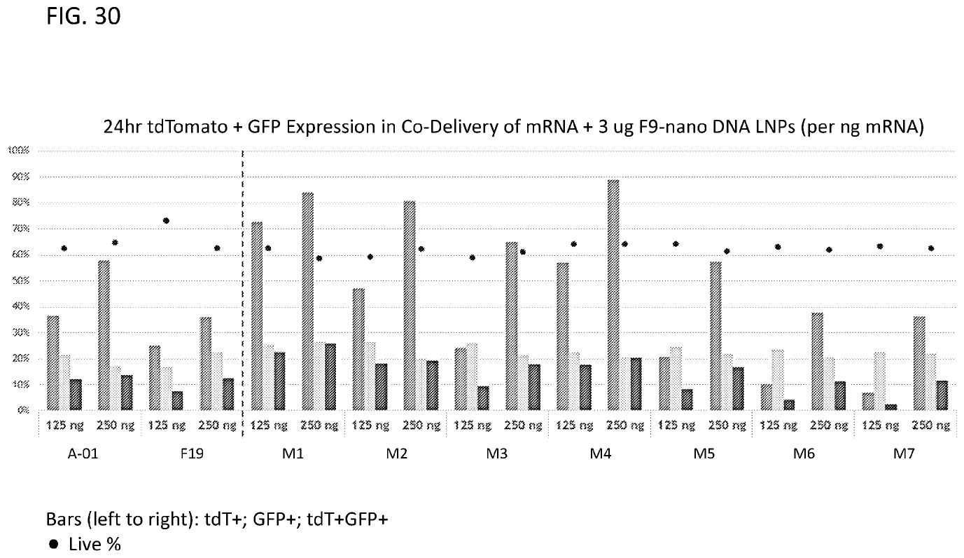

Specifically, LNPs generated from the F9 DNA formulation containing DLin-KC2-DMA were used to deliver 3 μg of a piggyBac DNA transposon construct containing the 4.9 kb CD19 CAR-sfGFP DNA nanoplasmid, in combination with LNPs generated from the same RNA formulations described in Example 9 for the delivery of 125 ng or 250 ng of the tdT reporter mRNA. The number of GFP+, tdT+, and double-positive cells was assessed (FIG. 30).

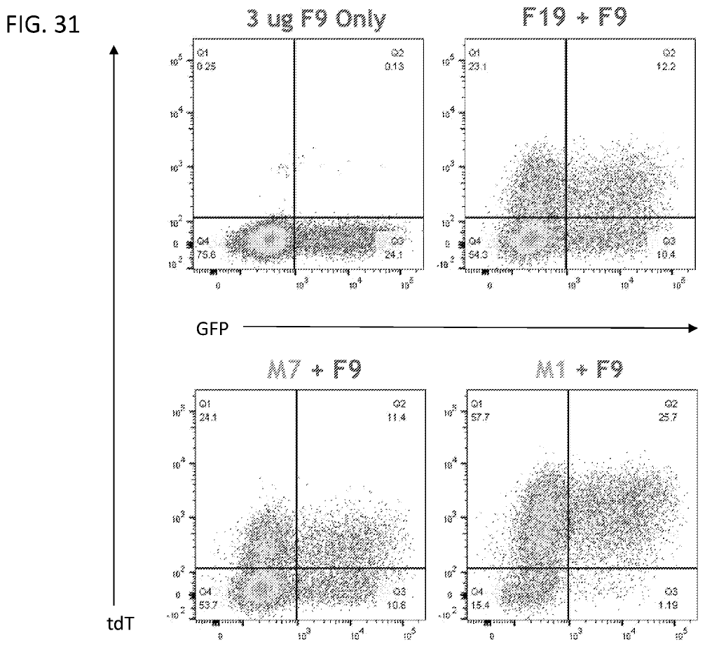

As shown in FIG. 31, the combination of RNA-containing LNPs generated from the M1 RNA formulation and DNA-containing LNPs generated from the F9 DNA formulation resulted in 25.7% of cells expressing both tdT and GFP.

b. Co-Delivery Using Separate DNA LNP and Electroporation of RNA—Hybrid Non-Viral Delivery (NVD) Approach

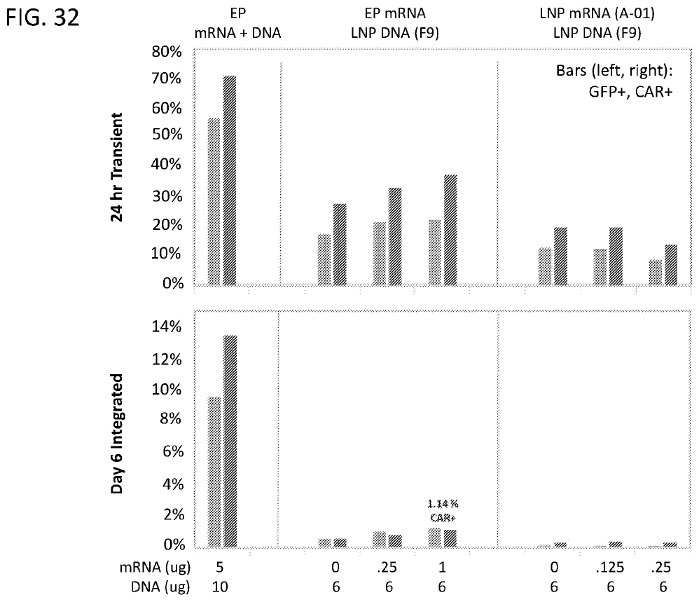

As an alternative to co-delivery of separate DNA and RNA LNPs, healthy donor T cells were electroporated with piggyBac transposase-encoding mRNA prior to transfection with the F9 lipid formulation containing the ionizable lipid DLin-KC2-DMA and loaded with the transposon CD19 CAR-sfGFP DNA plasmid (7.3 kb). Briefly, primary human T cells were thawed and activated substantially as described in Example 2. The T cells were subsequently harvested and washed with an isotonic buffer prior to suspension in a standard electroporation buffer. Electroporation of RNA was achieved with the Lonza Nucleofector® device per standard manufacturer protocol. Immediately following electroporation, cells were transferred to wells of a non-tissue culture treated plate containing fresh media supplemented with 1 μg/mL ApoE4. DNA-containing LNPs were provided in culture and gently mixed with the cells prior to incubation at 37° Celsius. Transient transfection efficiency was evaluated 24 hours after transfection, and gene integration was assessed six days after transfection, as determined by GFP and CAR expression. As a control, both the mRNA and DNA were introduced into the cells by electroporation (“EP mRNA+DNA”) or both were introduced by LNPs. Both transient (FIG. 32, top) and integrated (FIG. 32, bottom) expression of the CD19 CAR was observed to be higher when mRNA was provided by electroporation.

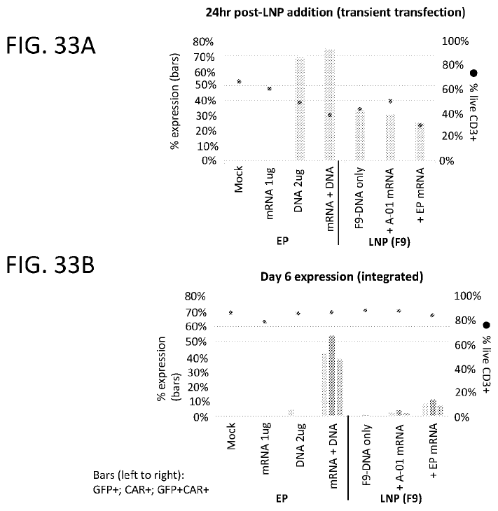

A similar comparative experiment was carried out using the CD19 CAR-sfGFP DNA nanoplasmid (4.9 kb). Approximately 30% of cells were found to transiently express GFP (i.e. at 24-hours post-transfection) when the DNA transposon construct, the transposase mRNA, or both, were introduced by LNPs (FIG. 33A). Approximately 10% of cells were found to have integrated the DNA transposon construct, as determined by GFP expression six days after transfection, when the DNA transposon construct was introduced by LNPs and the transposase mRNA was introduced by electroporation (FIG. 33B).

c. Delivery Using Co-Formulated DNA and RNA LNP

In a different approach, LNPs were co-formulated to contain both a piggyBac DNA transposon containing the CD19 CAR-sfGFP DNA nanoplasmid (4.9 kb) and a piggyBac transposase mRNA construct in the same final LNP formulation using a fusion approach as described herein. To generate the co-LNPs, LNPs generated from the F9 formulation containing DLin-KC2-DMA were used to encapsulate the DNA construct. LNPs generated to encapsulate the RNA construct were made from the “F19” formulation similarly as described in Example 9, but with piggyBac transposase mRNA replacing tdTomato fluorescent reporter mRNA as the RNA cargo. The F19 containing DLin-MC3-DMA was made using the same process, buffer, and component mass fractions of lipids and RNA as described in Table E22B (Table E23).

TABLE E23
F19 Formulation Components and Process Parameters for piggyBac Transposition

| Class | Variable | Species | Value | Units |
|---|---|---|---|---|
| Component | RNA Mass Fraction | piggyBac | 3.5 | % |
| | | transposase mRNA | | |
| | Ionizable Lipid Mass | DLin-MC3-DMA | 34.4 | % |
| | Fraction | | | |
| | Helper Lipid Mass Fraction | DSPC | 17.6 | % |
| | PEG Lipid Mass Fraction | DMG-PEG2000 | 4.5 | % |
| | Cholesterol Mass Fraction | Cholesterol | 40 | % |
| Process | Total Mass | Solids | 1 | mg |
| | Total Concentration | Lipids + mRNA | 1.0 | mg/mL |
| | Aqueous Flow Rate | mRNA in Buffer | 9.0 | mL/min |
| | Organic Flow Rate | Lipids in Ethanol | 3.0 | mL/min |
| | Temperature | | RT (~22) | Degrees Celsius |
| Buffer | Concentration | Acetate | 25 | mM |
| | pH | | 4.0 | — |

The separate DNA- and RNA-containing LNPs were first formulated using the NanoAssemblr™ Ignite instrument and, instead of immediately undergoing PBS neutralization and centrifugal filtration as described in Example 1, were instead allowed to remain in pH 4.0 acetate buffer and then were co-formulated via pH neutralization. Specifically, the acidic F19 mRNA LNPs and the acidic F9 DNA LNPs were mixed together via pipet in a 15 mL conical tube at a 1:3 volume ratio of F19:F9 LNPs, in pH 4.0 acetate buffer, and then neutralized with 7 parts by volume pH 7.4 PBS, allowing for the pH neutralization-mediated fusion of the acidic DNA LNPs and mRNA LNPs into neutral pH co-LNPs containing both the RNA and DNA. The neutralized co-LNPs underwent post-processing steps from centrifugal filtration and onward, as described in Example 1.

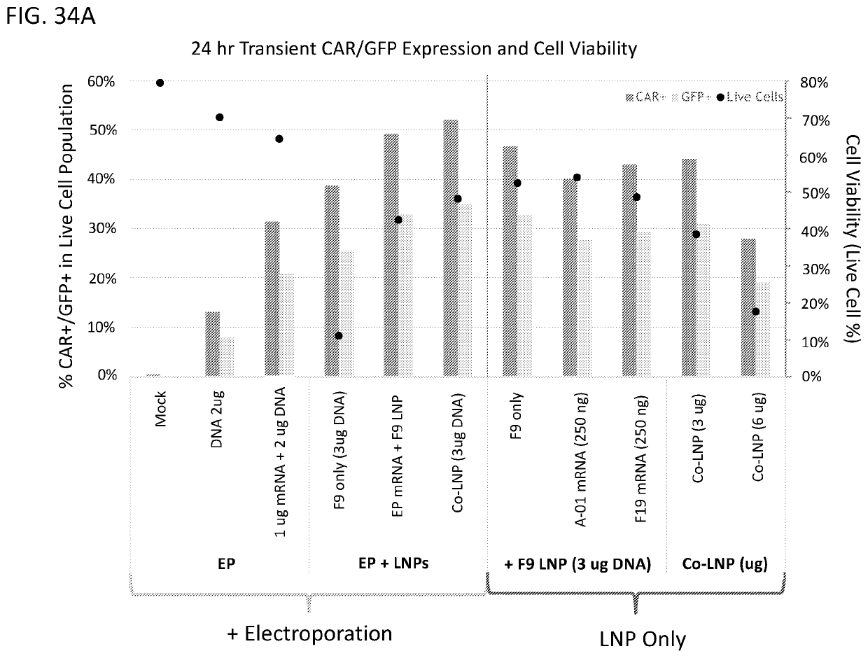

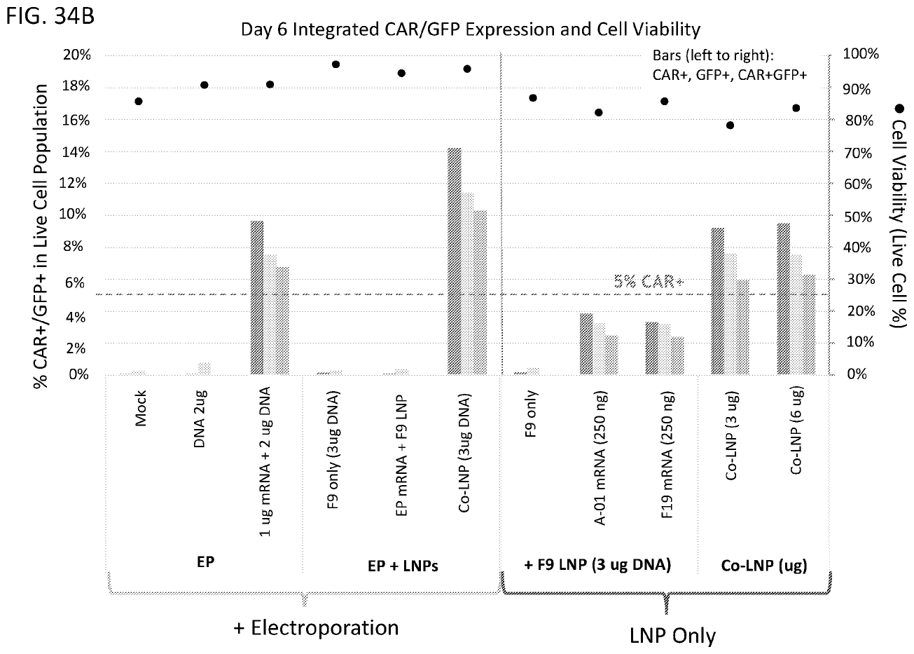

The co-LNPs containing both the transposon and transposase constructs were incubated with healthy donor T cells in media supplemented with 1 g/mL ApoE4, and GFP and CAR expression was assessed after 24 hours (transient expression) or six days (gene integration) (see FIGS. 34A and 34B). Additionally, the expression levels of GFP and CAR following administration of the co-LNPs were compared to expression levels of GFP and CAR following transfection of the T cells using the NVD approach described above. GFP and CAR expression results at 24 hours and six days post-transfection using the NVD approach are also shown in FIGS. 34A and 34B, respectively.

Using the co-LNP approach, greater than 30% of cells were found to transiently express GFP 24 hours after transfection, with greater than 40% of cells expressing the CAR (FIG. 34A). Six days after transfection (without electroporation), approximately 7.5% of co-LNP-treated cells were observed to express GFP, while approximately 9% of cells were observed to express the CAR (FIG. 34B). Among cells that were electroporated in combination with delivery of co-formulated LNPs (i.e., NVD approach), approximately 14% of cells expressed the CAR six days after transfection (FIG. 34B). Co-LNPs were observed to transfect cells at similar or higher rates as compared to using separate DNA- and RNA-containing LNPs. Without wishing to be bound by theory, these data are consistent with a finding that the delivery of co-LNPs containing DNA and RNA in the same LNP formulation may be advantageous by avoiding competition for cellular uptake between separate DNA- and RNA-containing LNPs.

### Example 11: Co-Formulated DNA and RNA LNPs Using OF-C4-Deg-Lin

As in Example 10, co-LNPs were formulated to contain both a piggyBac DNA transposon containing the CD19 CAR-sfGFP DNA nanoplasmid and a piggyBac transposase mRNA construct. To generate the co-LNPs, F9 or F9A DNA LNPs containing either DLin-KC2-DMA (KC2) or OF-C4-Deg-Lin (C4), respectively, were used to encapsulate the DNA construct, and M1 or F19 RNA containing DLin-MC3-DMA (MC3) LNPs were used to encapsulate the mRNA construct. The resulting pH 4.0 DNA LNPs and RNA LNPs were co-formulated (via fusion into co-LNPs) by mixing together at a 1:3 (RNA LNP:DNA LNP) volume ratio in pH 4.0 acetate buffer and then neutralized with 7 parts by volume pH 7.4 PBS, allowing for the fusion of the DNA and mRNA LNPs into co-LNPs containing both RNA and DNA.

The co-LNPs containing both the transposon and transposase constructs were incubated with healthy donor T cells, and GFP and CAR expression was assessed after 24 hours (transient expression) or seven days (integration). As controls, the cells were electroporated (EP), treated with DNA LNPs alone, RNA LNPs alone, or left untreated (UTC).

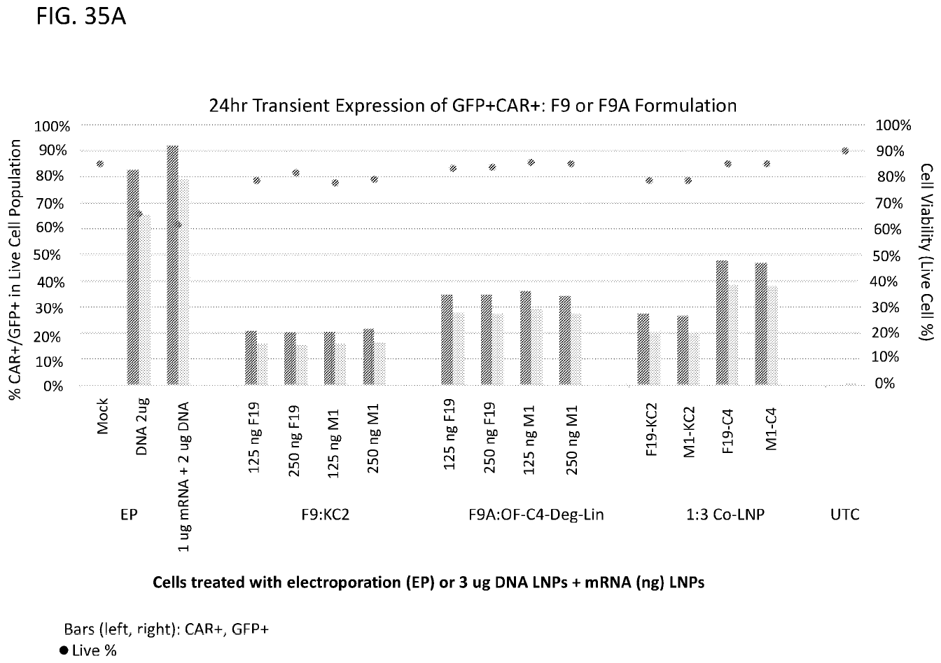

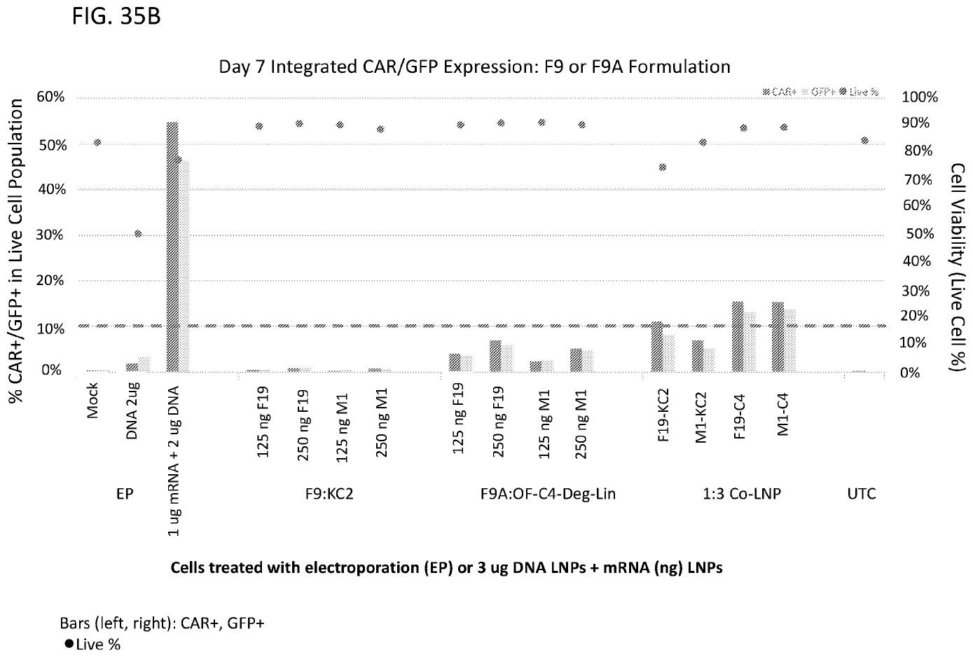

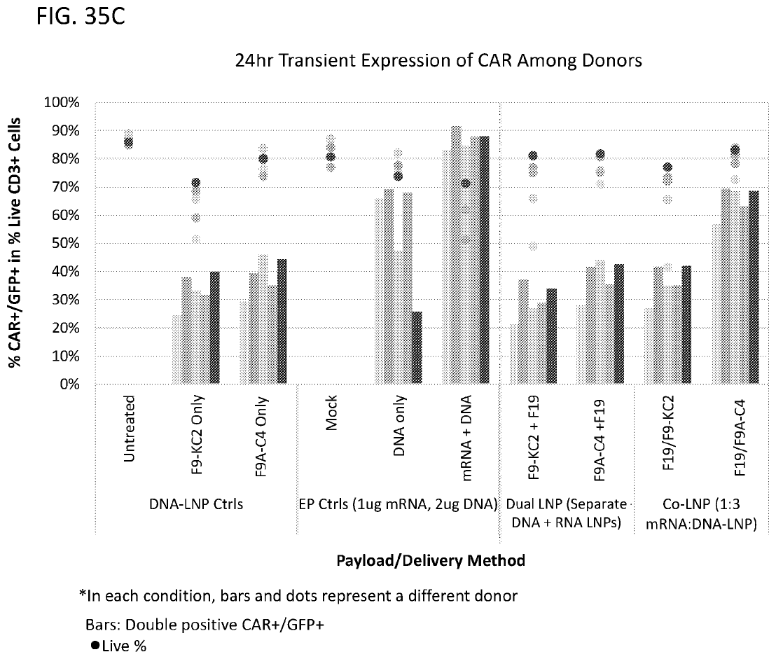

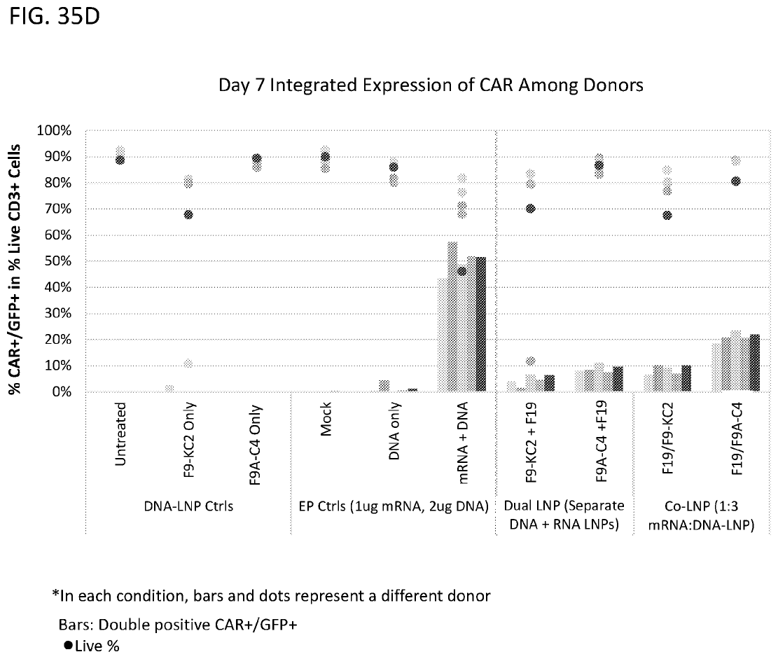

Greater than 30% of cells treated with the co-LNPs generated from the F9A DNA formulation (OF-C4-Deg-Lin) and the M1 or F19 RNA (MC3) formulation (Table E23) were found to transiently express GFP 24 hours after transfection, with greater than 40% of cells expressing the CAR (FIG. 35A). By day 7, among cells incubated with the co-LNPs generated from the F9A DNA formulation (OF-C4-Deg-Lin) and the M1 or F19 RNA (MC3) formulation, approximately 13.6% of cells were observed to express GFP, while approximately 15.5% of cells were observed to express the CAR (FIG. 35B).

The experiments were repeated with the F19 RNA (MC3) formulation (Table E23) across a set of five different healthy donors, using either the co-LNP methods described in this Example, or providing separate, co-delivered DNA and RNA LNPs as described in Example 10. 24 hours after incubation with the co-LNPs, between 30% and 70% of the T cells from the five different donors were observed to express the CAR encoded by the DNA. By contrast, between 20% and 40% of T cells from the donors were observed to express the CAR following incubation with the separate, co-delivered DNA and RNA LNPs (FIG. 35C). As controls, cells were treated with either the DNA LNPs alone, the RNA LNPs alone, or electroporation (EP). By day 7, over 30% of T cells were observed to express the CAR in some donor samples following treatment with co-LNPs (FIG. 35D).

### Example 12: Alternative Co-Formulated LNPs

As in Examples 10 and 11, co-LNPs were generated to contain both a piggyBac DNA transposon containing the CD19 CAR-sfGFP DNA nanoplasmid and a piggyBac transposase mRNA construct. To generate the co-LNPs, the F9A DNA formulation containing OF-C4-Deg-Lin (C4) was used to encapsulate the DNA construct. RNA formulations containing different ionizable lipids (DLin-MC3-DMA (MC3); cKK-E12 (cKK); or OF-C4-Deg-Lin (C4)), were used to encapsulate the mRNA construct. The F19 RNA formulation described in Example 10 (Table E23) contained either DLin-MC3-DMA (MC3) or cKK-E12 (cKK). The F19A formulation (Table E24) contained OF-C4-Deg-Lin (C4). The resulting DNA LNPs and RNA LNPs were co-formulated by mixing together at a 1:3 or 1:2 (RNA LNP:DNA LNP) volume ratio in pH 4.0 acetate buffer and then neutralized in pH 7.4 PBS, allowing for the fusion of the DNA and mRNA LNPs into co-LNPs containing both RNA and DNA.

The co-LNPs were incubated with activated donor T cells, and GFP and CAR expression was assessed after 24 hours (transient expression) or seven days (integration). As controls, the cells were electroporated (EP), treated with DNA LNPs alone, RNA LNPs alone, or left untreated (UTC).

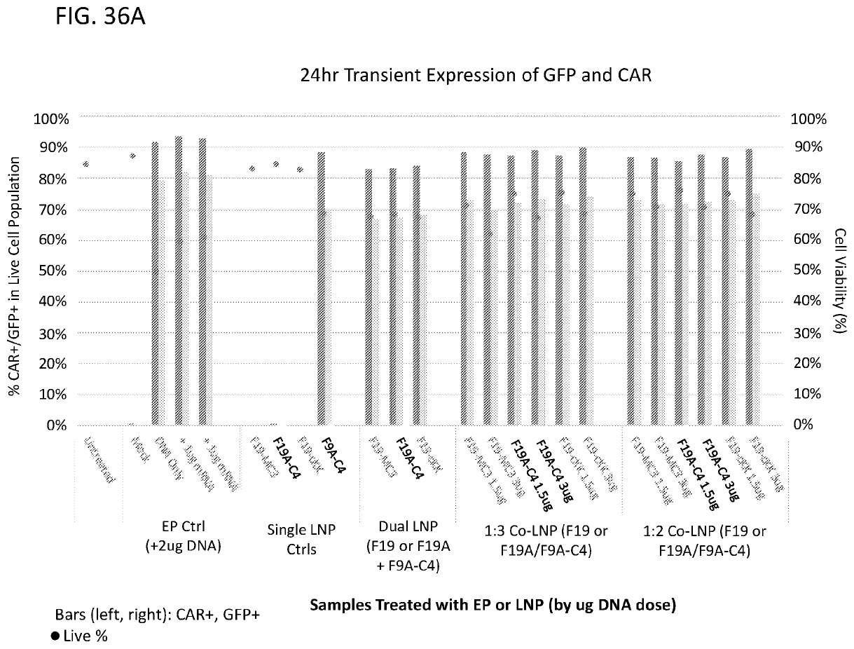

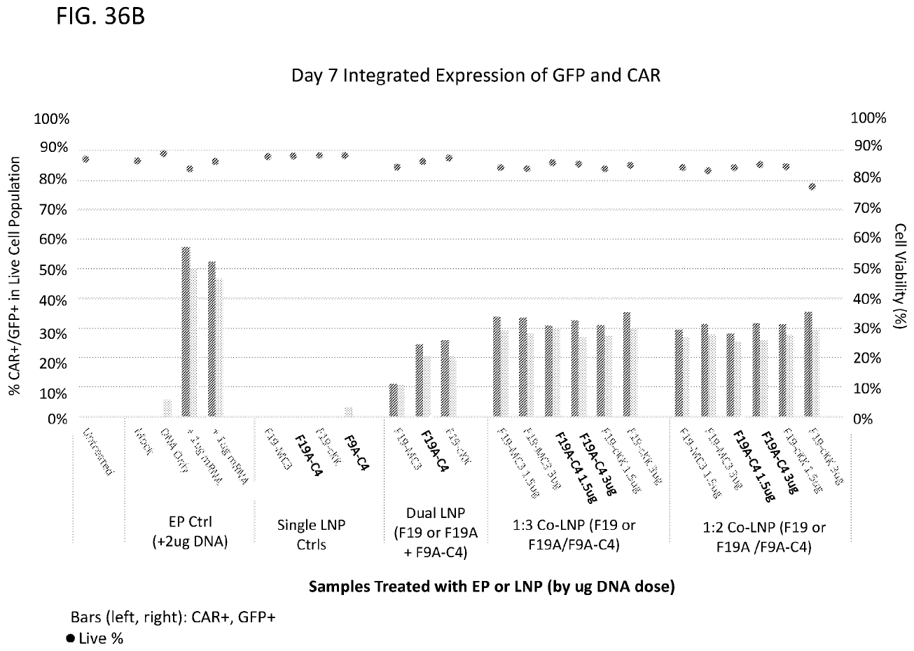

GFP and CAR expression was not found to significantly differ at either 24 hours or 7 days among when F9A (C4) DNA LNPs were co-formulated with F19 (MC3 or cKK) or F19A (C4) RNA LNPs (FIGS. 36A and 36B, respectively). Additionally, no significant differences were observed when cells were activated with either beads or Expamer™ reagent prior to incubation with the co-LNPs.

TABLE E24
F19A Formulation Components and Process Parameters

| Class | Variable | Species | Value | Units |
|---|---|---|---|---|
| Component | RNA Mass Fraction | piggyBac | 3.5 | % |
| | | transposase mRNA | | |
| | Ionizable Lipid Mass | OF-C4-Deg-Lin | 31.0 | % |
| | Fraction | | | |
| | Non-ionizable Cationic | Lipid T1 | 3.44 | % |
| | Lipid Mass Fraction | | | |
| | Helper Lipid Mass Fraction | DSPC | 17.6 | % |
| | PEG Lipid Mass Fraction | DMG-PEG2000 | 4.5 | % |
| | Cholesterol Mass Fraction | Cholesterol | 40 | % |
| Process | Total Mass | Solids | 1 | mg |
| | Total Concentration | Lipids + mRNA | 1.0 | mg/mL |
| | Aqueous Flow Rate | mRNA in Buffer | 9.0 | mL/min |
| | Organic Flow Rate | Lipids in Ethanol | 3.0 | mL/min |
| | Temperature | | RT (~22) | Degrees Celsius |
| Buffer | Concentration | Acetate | 25 | mM |
| | pH | | 4.0 | — |

### Example 13: Lipid Nanoparticle-Mediated Genetic Engineering Using CRISPR/Cas

a. TRAC Knock-Out

The T cell receptor alpha constant (TRAC) locus was knocked out in T cells by delivering LNPs co-encapsulating mRNA encoding a Cas9 nuclease and a guide RNA (gRNA) targeting TRAC.

The F19A RNA LNP formulation (Table E24) described in Example 12 and an alternative RNA LNP formulation “A” (see Table E25), both containing OF-C4-Deg-Lin (C4) as the ionizable lipid, were assessed for their ability to encapsulate and deliver the Cas9 mRNA and TRAC gRNA to T cells. LNPs were generated on the NanoAssemblr Ignite instrument using an aqueous flow rate of 9.0 mL/min and an organic flow rate of 3 mL/min.

TABLE E25
RNA Formulation A: Components and Process Parameters

| Class | Variable | Species | Value | Units |
|---|---|---|---|---|
| Component | Ionizable Lipid Mass | OF-C4-Deg-Lin | 54.2 | % |
| | Fraction | | | |
| | Helper Lipid Mass Fraction | DSPC | 13.64 | % |
| | PEG Lipid Mass Fraction | DMG-PEG2000 | 6.77 | % |
| | Cholesterol Mass Fraction | Cholesterol | 19.39 | % |
| | Non-ionizable Cationic | Lipid T1 | 6 | % |
| | Lipid Mass Fraction | | | |
| | Total RNA Mass Fraction | 1:1 or 1:3 molar | 3.5 or 10 | % |
| | | ratio Cas9:gRNA | | |
| Process | Total Concentration | Lipids + RNA | 1.0 | mg/mL |
| | Aqueous Flow Rate | RNA in Buffer | 9.0 | mL/min |
| | Organic Flow Rate | Lipids in Ethanol | 3.0 | mL/min |
| | Temperature | | RT (~22) | Degrees Celsius |
| Buffer | Concentration | Acetate | 25 | mM |
| | pH | | 4.0 | -— |

The LNPs generated from the F19A and A formulations were formulated with either a 1:1 or 1:3 molar ratio of Cas9 mRNA:TRAC gRNA, and a total RNA mass fraction of either 3.5% (“A”) or 10% (“A10”). The various formulations assessed are shown below in Table E26.

TABLE E26
Mass Fractions of RNA LNP Formulations for Cas9 mRNA and TRAC gRNA Co-delivery

| | | | | | | | | Total RNA |
|---|---|---|---|---|---|---|---|---|
| | | | | | | | | Mass |
| | Ionizable | | DMG- | | Lipid | Cas9 | TRAC | Fraction |
| Formulation | lipid (C4) | DSPC | PEG2000 | Cholesterol | T1 | mRNA | gRNA | (%) |
| F19A | 31.0 | 17.6 | 4.5 | 40 | 3.44 | 0.077 | 3.42 | 3.5 |
| Cas9/TRAC | | | | | | | | |
| 1:1 | | | | | | | | |
| F19A | 31.0 | 17.6 | 4.5 | 40 | 3.44 | 0.026 | 3.47 | 3.5 |
| Cas9/TRAC | | | | | | | | |
| 1:3 | | | | | | | | |
| A | 54.2 | 13.64 | 6.77 | 19.39 | 6.0 | 0.077 | 3.42 | 3.5 |
| Cas9/TRAC | | | | | | | | |
| 1:1 | | | | | | | | |
| A | 54.2 | 13.64 | 6.77 | 19.39 | 6.0 | 0.026 | 3.47 | 3.5 |
| Cas9/TRAC | | | | | | | | |
| 1:2 | | | | | | | | |
| A10 | 48.8 | 12.28 | 6.08 | 17.45 | 5.4 | 0.219 | 9.78 | 10 |
| Cas9/TRAC | | | | | | | | |
| 1:1 | | | | | | | | |

Cells introduced with nucleic acid by electroporation (EP), substantially as described in Example 10b, served as positive controls. In particular, primary T cells from healthy human donors were harvested and washed with isotonic buffer prior to suspension in standard electroporation buffer. Electroporation of Cas9 mRNA and TRAC gRNA was achieved Lonza Nucleofector® device per standard manufacturer protocol. Immediately following electroporation, cells were transferred to wells of a non-tissue culture treated plate containing fresh media and incubated at 37° Celsius.

LNPs generated using the F19A 1:3 formulation exhibited a large amount of aggregation as compared to LNPs generated using the F19A 1:1 formulation and may not have formed stable particles following neutralization with PBS. The total amount of RNA loaded into LNPs, as well as the encapsulation efficiency, are shown in Table E27. Formulation A was observed to result in better loading of RNA into LNPs. In particular, LNPs generated from the F19A formulation could not be dosed at higher than 500 ng of total RNA due to poor loading and low RNA yield.

TABLE E27
Loading of Cas9 mRNA and TRAC gRNA into LNPs

| Formulation | Total RNA concentration | Encapsulation efficiency |
|---|---|---|
| (molar Cas9:gRNA ratio) | (ug/mL) | (%) |
| F19A Cas9/TRAC 1:1 | 4.45 | 41.58 |
| F19A Cas9/TRAC 1:3 | 8.28 | 63.1 |
| A Cas9/TRAC 1:1 | 53 | 92.83 |
| A Cas9/TRAC 1:2 | 60.38 | 93.25 |
| A10 Cas9/TRAC 1:1 | 148.86 | 95.84 |

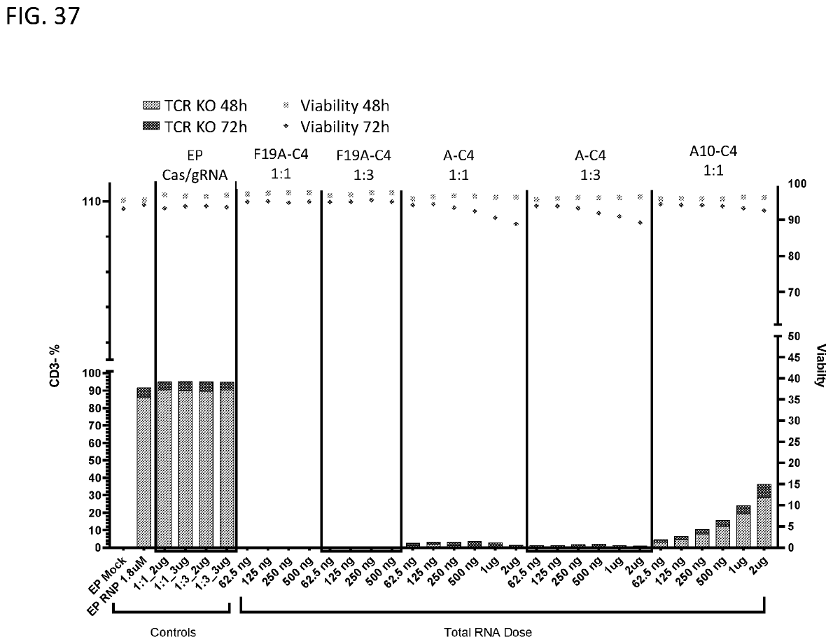

TRAC knockout was assessed in T cells 48 and 72 hours after incubation with LNPs by analyzing the percentage of CD3− cells (CD3 expression is a surrogate for TCR expression). Electroporated cells exhibited greater than 80% and 90% TRAC knockout after 48 and 72 hours, respectively, as assessed by the percentage of CD3− cells. Formulation A10 was the only formulation to exhibit significant TRAC knockout (FIG. 37), indicating that more RNA, a higher lipid:RNA ratio, or both, may be required to achieve significant RNA delivery and gene knockout.

b. TRAC Knock-Out and CAR Knock-In

Knock-in of an HDR template (HDRt) DNA encoding an anti-BCMA CAR into the TRAC locus was assessed using the F9A DNA LNP formulation OF-C4-Deg-Lin as the ionizable lipid. The HDRt was used to deliver either the CAR as encoded by either a 4.0 kB nanoplasmid (NP) or a 4.0 kB closed-end DNA (ceDNA) vector. A RNA LNP formulation “X” was created for generation of RNA LNPs, which contained a 1:1 weight ratio of Cas9 mRNA:gRNA, instead of a 1:1 molar ratio, as in preceding experiments. The components of Formulation X are shown below in Table E28.

TABLE E28
RNA Formulation X: Components and Process Parameters

| Class | Variable | Species | Value | Units |
|---|---|---|---|---|
| Component | Ionizable Lipid Mass | OF-C4-Deg-Lin | 54.2 | % |
| | Fraction | | | |
| | Helper Lipid Mass Fraction | DSPC | 13.64 | % |
| | PEG Lipid Mass Fraction | DMG-PEG2000 | 6.77 | % |
| | Cholesterol Mass Fraction | Cholesterol | 19.39 | % |
| | Non-ionizable Cationic | Lipid T1 | 6 | % |
| | Lipid Mass Fraction | | | |
| | Total RNA | | 5 | % |
| | | | (1:1 weight | |
| | | | Cas9:gRNA) | |

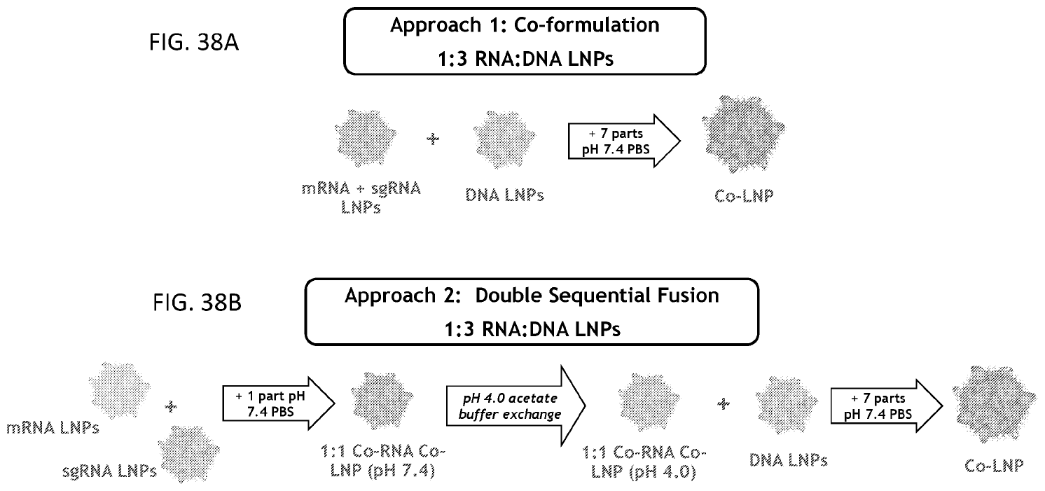

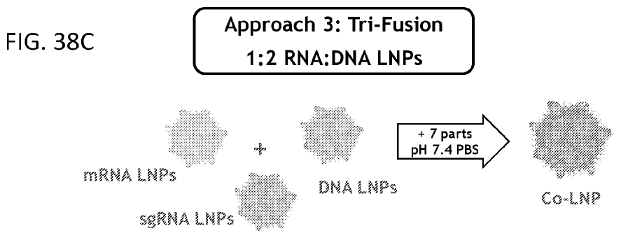

Three different approaches were taken to generate LNPs containing both DNA and RNA. In a first approach, Cas9 mRNA and TRAC gRNA were co-formulated (not fused) together at a 1:1 weight ratio in the aqueous phase into a single LNP formulation containing both RNAs (“RNA LNP”). Prior to neutralization with pH 7.4 PBS, pH 4.0 Cas9 mRNA/gRNA LNPs were mixed together with pH 4.0 DNA LNPs in acetate buffer at a 1:3 volume ratio of RNA LNPs:DNA LNPs, followed by addition of 7 parts pH 7.4 PBS to 1 part LNP solution in acetate buffer (similar to the process as outlined in Example 10c) (FIG. 38A). In a second approach, called “double sequential fusion,” two separate RNA LNPs containing either Cas9 mRNA or TRAC gRNA were generated from Formulation X. The separate Cas9 mRNA- and TRAC gRNA-containing LNPs in pH 4.0 acetate buffer were mixed together at a 1:1 volume ratio of Cas9 mRNA LNP:gRNA LNP, followed by fusion and pH neutralization with the addition of 1 part pH 7.4 PBS to 1 part LNP solution (“co-RNA”). The neutralized 1:1 co-RNA co-LNP, containing Cas9 mRNA and TRAC gRNA, then underwent acidic buffer exchange with 7 parts pH 4.0 acetate buffer added to 1 part LNP solution, and was concentrated using a 30 kDa centrifugal filter. The resulting pH 4.0 1:1 Co-RNA co-LNPs were then mixed with pH 4.0 DNA LNPs containing the HDRt at a 1:3 volume of RNA:DNA LNPs, followed by neutralization with pH 7.4 PBS (mixing 7 parts PBS to 1 part LNP solution) and subsequent centrifugal filtration with a 30 kDa centrifugal filter. (FIG. 38B). In a third approach called “tri-fusion,” a first RNA LNP containing Cas9 mRNA, a second RNA LNP containing TRAC gRNA, and a DNA LNP containing the HDRt were all fused at 1:2 volume of total RNA LNPs:DNA LNPs to generate a single co-formulated LNP containing both RNAs and the HDRt (FIG. 38C). The tri-fusion approach generates a “Tri-LNP”.

As a hybrid non-viral delivery (NVD) approach, primary T cells from healthy human donors were electroporated with either (i) a ribonucleoprotein complex (RNP) containing Cas9 and TRAC gRNA or (ii) a 1:1 weight ratio of Cas9 mRNA and TRAC gRNA prior to transfection with DNA LNPs.

In all approaches, DNA LNPs were generated from the F9A formulation. Following 0.2 m sterile filtration in pH 7.4 PBS, all of the generated LNPs had a similar size distribution of approximately 100-120 nm, as measured by DLS. LNPs containing the ceDNA were observed to have lower encapsulation efficiency but a higher resulting concentration of total DNA, as compared to LNPs containing the NP. A summary of the RNA and DNA formulations used is shown by Table E29.

TABLE E29
Mass Fractions of RNA LNP Formulations for Cas9
mRNA and TRAC gRNA Co-delivery and DNA LNPs

| | | | | | | | | Total |
|---|---|---|---|---|---|---|---|---|
| | | | | | | | | NA Mass |
| RNA | Ionizable | | DMG- | Lipid | | Cas9 | TRAC | Fraction |
| Formulation | lipid (C4) | DSPC | PEG2000 | T1 | Cholesterol | mRNA | gRNA | (%) |
| X (1:1 RNA | 48.8 | 12.28 | 6.08 | 5.4 | 17.45 | 2.5 | 2.5 | 5 |
| weight ratio) | | | | | | | | |
| X (Cas9 | 48.8 | 12.28 | 6.08 | 5.4 | 17.45 | 5.0 | n/a | 5 |
| mRNA only) | | | | | | | | |
| X (gRNA | 48.8 | 12.28 | 6.08 | 5.4 | 17.45 | n/a | 5.0 | 5 |
| only) | | | | | | | | |

| | | | | | | | | Total |
|---|---|---|---|---|---|---|---|---|
| | | | | | | | | NA Mass |
| DNA | Ionizable | | DMG- | Lipid | | NP | | Fraction |
| Formulation | lipid (C4) | SOPC | PEG2000 | T1 | Cholesterol | DNA | ceDNA | (%) |
| F9A | 36 | 19 | 2.5 | 4.0 | 35 | 3.5 | n/a | 3.5 |
| (nanoplasmid) | | | | | | | | |
| F9A | 36 | 19 | 2.5 | 4.0 | 35 | n/a | 3.5 | 3.5 |
| (ceDNA) | | | | | | | | |

Cryopreserved T cells were thawed and activated for 48 hours. Following activation, cells not subjected to the hybrid NVD approach were transfected with the materials as described in media containing 1 μg/mL ApoE4. Following activation, cells subjected to the hybrid NVD approach were harvested and washed with an isotonic buffer prior to suspension in standard electroporation buffer. Electroporation of RNP or Cas9 mRNA and TRAC gRNA was achieved with the Lonza Nucleofector® device per standard manufacturer protocol. Immediately following electroporation, cells were transferred to wells of a non-tissue culture treated plate containing fresh media supplemented with 1 g/mL ApoE4. DNA-containing LNPs were provided in culture and gently mixed with the cells prior to incubation at 37° Celsius.

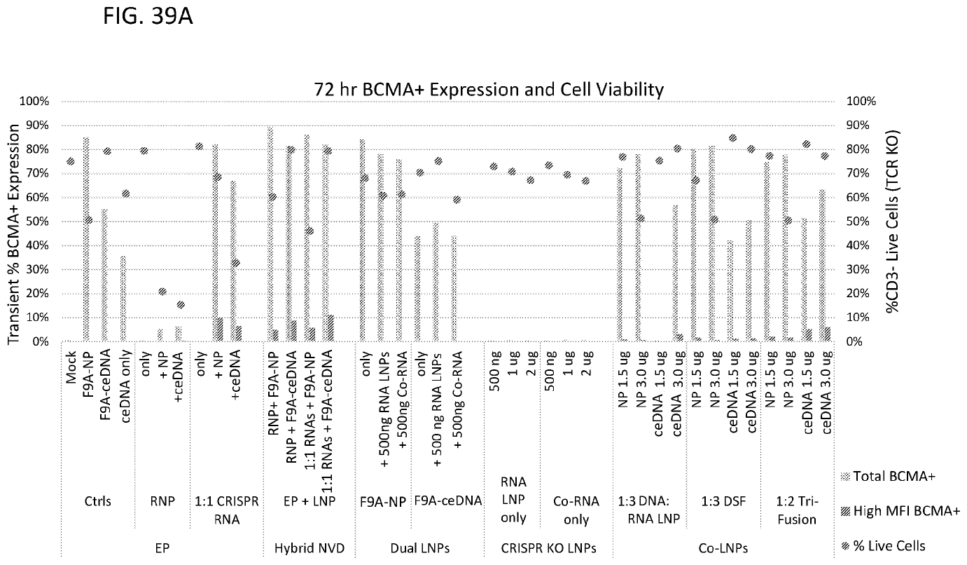

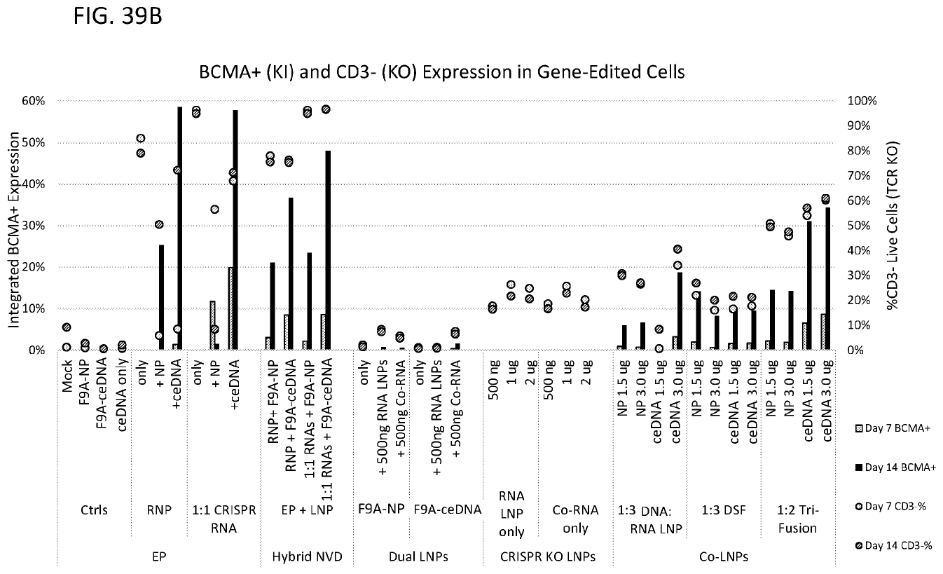

The percent of CD3−/CAR+ cells was assessed 3, 7, and 14 days after transfection. Cell viability was assessed at 2, 3, 5, 7 and 14 days after transfection. At 3 days post-transfection, transient expression of the anti-BCMA CAR was observed in approximately 40-80% of cells incubated with LNPs generated using the double sequential fusion or tri-fusion approach, and in approximately 80-90% of cells subjected to the hybrid NVD approach (FIG. 39A). At 14 days post-transfection, integrated expression of the anti-BCMA CAR was observed in 5-35% of cells incubated with LNPs generated using either the double sequential fusion or tri-fusion approach, and in approximately 20-50% of cells subjected to the hybrid NVD approach (FIG. 39B).

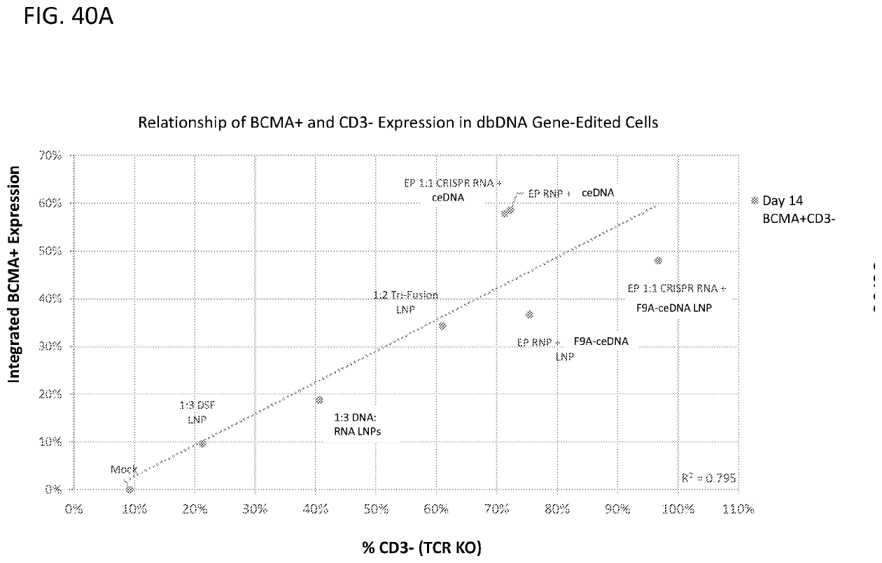

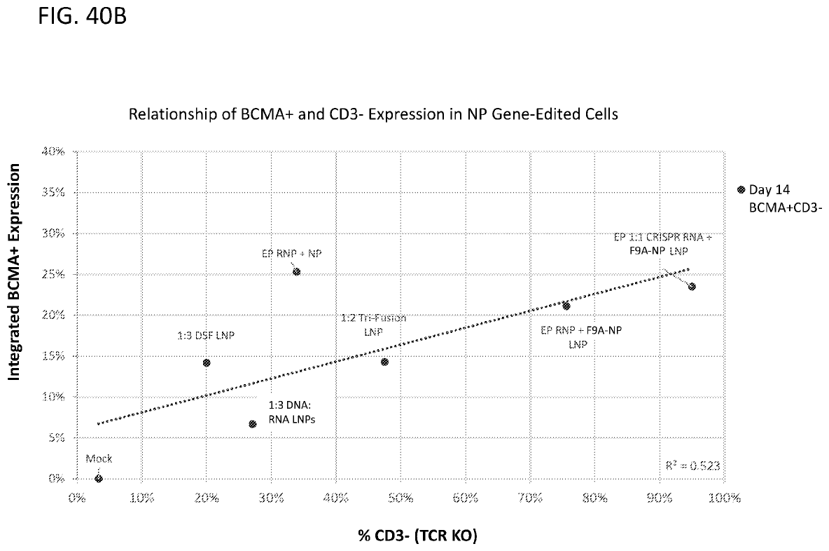

At day 14 post-transfection, a stronger correlation of CD3−/BCMA CAR+ cells was observed with CAR knock-in using ceDNA as compared to the NP (FIGS. 40A and 40B, respectively).

### Example 14: Co-LNP Delivery of Large ceDNA CAR T Payloads

Experiments were conducted to determine whether co-LNPs and tri-LNPs can deliver large ceDNA payloads to cells. Experiments were conducted as described in Example 13b, except as noted below.

a. LNP Formulation

LNPs were formulated as described in Example 13 with Cas9 mRNA, TRAC sgRNA, and CAR closed-end DNA (ceDNA) single-targeted CAR (4 kb in size) same or a bicistronic construct of a tandem dual-targeted CAR (8 kb in size). As described in Example 13b., three different approaches were taken to generate LNPs containing both DNA and RNA (see FIGS. 38A-C). A summary of the RNA and DNA formulations used in these studies is shown in Table E29 (see Example 13b). The DNA LNP formulations contained a 3.5% mass fraction of either the 4 kb single-targeted CAR or the 8 kb tandem dual-targeted CAR The Cas9 mRNA used in this example was of higher purity than the Cas9 used in Example 13b.

Lipid mixtures were mixed together in 200 proof molecular grade ethanol according to mass fractions listed in Table E29, for each type of LNP formulated. Lipid mixes were stored in 2 mL Eppendorf tubes prior to formulation. DNA and RNA aqueous phases for formulation were prepared by dissolving nucleic acids in 25 mM sodium acetate buffer in a 15 mL conical tube.

Lipid mixes and nucleic acid aqueous phases were loaded into BD syringes and attached to a formulation cartridge for the Precision Nanosystems Ignite. The solutions were mixed together to form nanoparticles in pH 4 acetate buffer. Aliquots of these LNP formulations (listed in Table E29) were collected in order to neutralize in pH 7.4 PBS (7 parts PBS:1 part pH 4 particle mixture) to generate single payload LNPs or mix particles with different encapsulated constructs together in a 15 mL conical tube and neutralize with pH 7.4 PBS to fuse and generate “co-LNPs” (co-formulated as described in FIG. 38A) and “tri-LNPs” (co-formulated as described in FIG. 39C).

After PBS neutralization, LNPs underwent buffer exchange via centrifugal filtration in 30 kDa Amicon Ultra centrifugal filters at 2000×G for 2.5 hr. After LNPs were sufficiently concentrated, the formulations were collected in a 3 mL syringe and passed through a 0.2 um filter. LNPs were stored at 4° C. prior to use in primary T cell assays.

b. Primary T Cell Culture Assay

Vials of cryopreserved CD4+ and CD8+ primary T Cells from three different healthy human donors were thawed and activated with Expamer™ cell activation reagent, in a 1:1 CD4+:CD8+ cell ratio (of the same donor) prior to transfection. Cells were incubated and cultured in serum-free media with a mixture of cytokines throughout the duration of the assay.

Activated T cells were harvested and resuspended in fresh media prior to being prepared for transfection, according to either of the below three protocols:

### Electroporation (EP) Controls

Fresh media (without ApoE4) was plated into tissue culture plate and incubated at 37° C.
Cells for EP were harvested and washed with an isotonic buffer prior to suspension in a standard electroporation buffer
For each sample, resuspended cells were mixed with (some combination, depending on the control) gene editing cargo (SpCas9 TRAC ribonucleoprotein (RNP), 4 kb CAR ceDNA, or tandem 8 kb CAR ceDNA) and loaded into electroporation cassettes. Electroporation of was achieved with the Lonza Nucleofector® device per standard manufacturer protocol and then seeded into pre-warmed wells of equilibrated media.
   “Hybrid” NVD [“hNVD”]
Fresh media (supplemented with ApoE4) was plated into tissue culture plate
DNA-LNPs (“F9A” with either 4 kb CAR ceDNA or tandem 8 kb CAR ceDNA) were added to sample wells with media, and the plate was incubated at 37° C. to equilibrate media
Cells for EP were harvested and washed with an isotonic buffer prior to suspension in a standard electroporation buffer. For each sample, resuspended cells were mixed with SpCas9 TRAC ribonucleoprotein (RNP) and loaded into electroporation cassettes. Cells were electroporated according to manufacturing protocol and then seeded into pre-warmed wells containing equilibrated media and DNA-LNPs.

### LNP-Only Delivery

Cells were plated into fresh media supplemented with ApoE4 to support cellular uptake of LNPs. LNPs were added via pipetting into plated cell wells.

Transfected cells were incubated at 37° C., 5% CO₂, sampled for intermittent analysis for gene editing efficiency, cell count, and cell viability, and sub-cultured into fresh media to promote cellular expansion when cells reached confluence. Cells were harvested at a terminal endpoint to evaluate integrated gene-edited expression.

c. Results

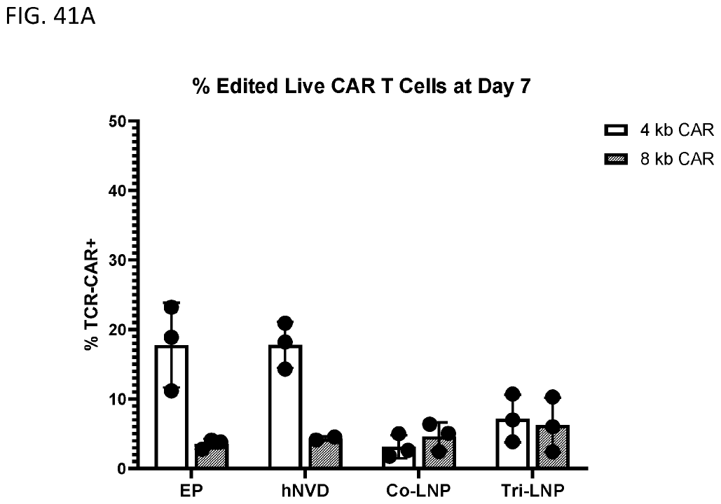

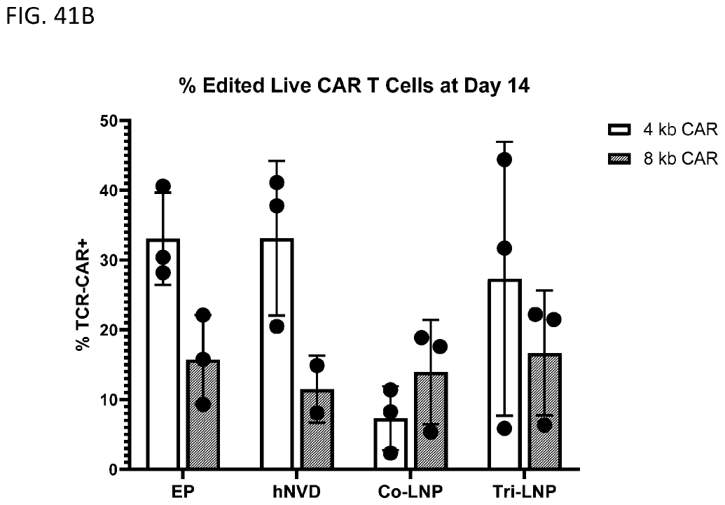

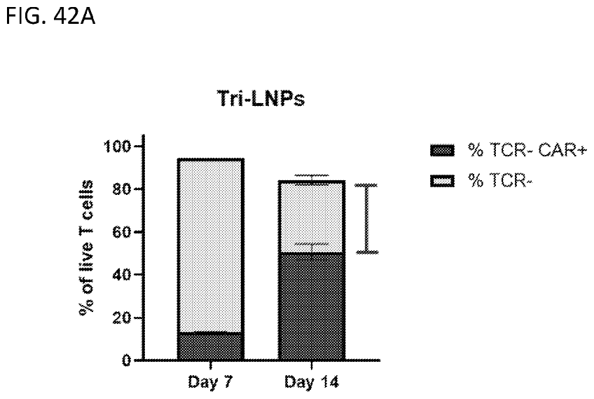

FIG. 41A and FIG. 41B show the percentage of fully edited cells 7 days and 14 days post-transfection, respectively, as an average of transfection results in three different healthy human donors. Fully edited cells display both TCR knockout (TCR−) and expression of CAR T. The percentage of fully edited cells expressing the 8 kb tandem CAR were comparable across the different methods (electroporation, hybrid method, co-LNP and tri-LNP). Notably, at 14 days post-transfection, the tri-LNP gave similar or slightly greater expression of the 8 kb CAR compared to the electroporation and hybrid methods. Hence, the experiment demonstrates that large ceDNA sequences can be effectively delivered to T cells using the fused LNPs of the disclosure.

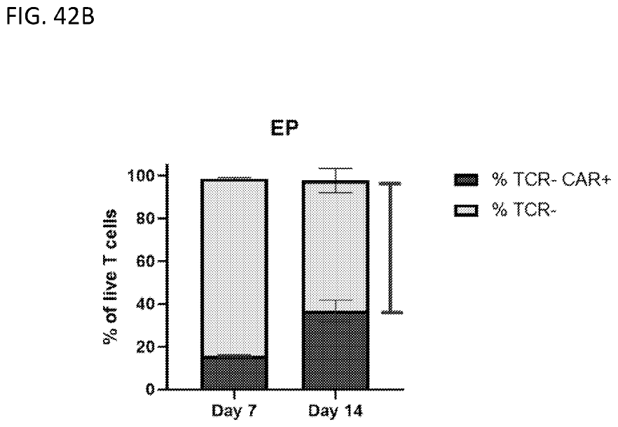

As shown (with delivery of the 4 kb CAR) in FIG. 42A, fully edited (TCR− CAR+) T cell populations enrich over time. The same phenomenon is observed for the electroporated cells, albeit to a much less extent (FIG. 42B).

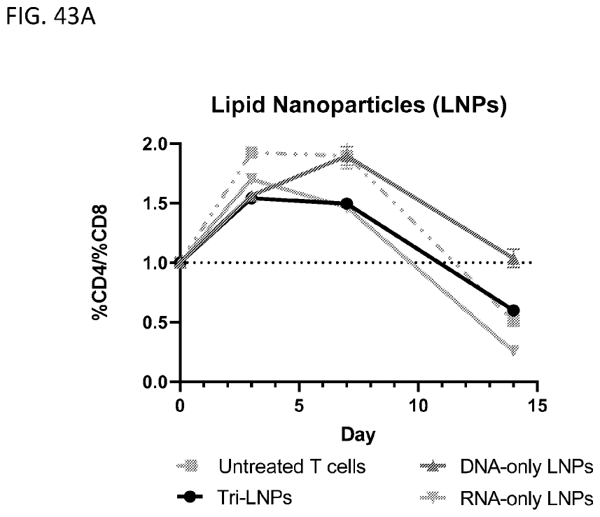

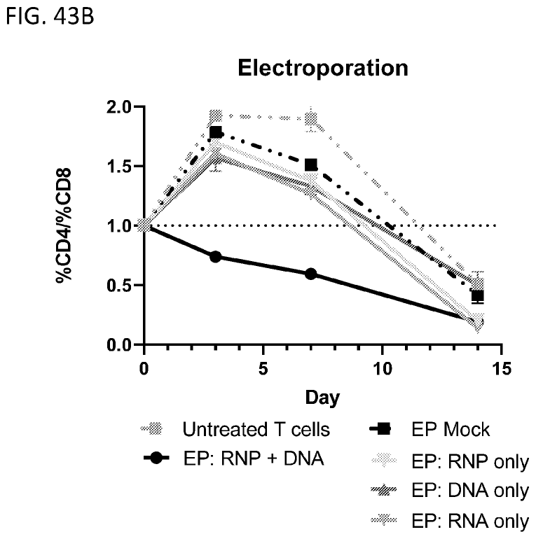

Additionally, transfection with LNPs, particularly tri-LNPs, results in preservation of % CD4/% CD8 ratios for longer periods of time. FIG. 43A show CD4/CD8 ratios at various timepoints following transfection of LNPs. Initially, the CD4 population is enhanced relative to the CD8 population. However, over time higher ratios of CD8 T cells relative to CD4 T cells are observed. A similar phenomenon is observed for electroporating RNP and DNA in T cells. (FIG. 43B). However, the decline in the CD4 T cell population is significantly greater with the electroporated T cells relative to T cells transduced with the LNPs.

### Example 15: Gene Editing Comparison Across Culturing Methods

Experiments were designed to compare gene editing using electroporation and the tri-LNPs of the disclosure across three different culture methods. LNPs were formulated as described in Examples 13 and 14 with the 4 kb CAR ceDNA. However, in these experiments slightly more of the ionizable lipid (C4) and slightly less of the Lipid T1 were used relative to mass fractions represented in Table E29. Specifically, for the 4 kb CAR ceDNA LNP, 39.2% of ionizable lipid (C4) and 0.8% of Lipid T1 were used to prepare the LNPs prior to fusion. For the RNA LNPs, 53.1% of ionizable lipid (C4) and 1.1% of Lipid T1 were used to prepare the LNPs prior to fusion.

a. Cell Culture Methods

Vials of cryopreserved CD4+ and CD8+ primary T Cells from one healthy human donor were thawed and activated as described in Example 14b prior to transfection. Cells were incubated and cultured in serum-free media with cytokines throughout the duration of the assay. Activated T cells were harvested, and resuspended in fresh media prior to being prepared for transfection, according to either of the three protocols described in Table E30 below.

Electroporation controls with SpCas RNP and/or 4 kb CAR ceDNA were prepared as described in Example 14b. Cells were electroporated according to the manufacturer's protocol and then seeded into pre-warmed wells of equilibrated media. LNPs were transfected in cells according to the same protocol described in Example 14b.

Cells cultured in three different expansion methods at 48 hr post-transfection were evaluated and compared for gene-editing efficiency. The three different expansion methods are shown in Table E30.

TABLE E30
Cell culture methods

| | |
|---|---|
| Method A - No | Transfected cells were incubated at 37° C., 5% CO₂ for 72 hr prior to be |
| culture step | sampled for early timepoint flow analysis |
| through 72 hr | |
| incubation | |
| Method B - | Transfected cells were harvested and plated into 24-deep-well plates |
| Complete Media | (DWPs) prior to centrifuging at 500 xG, for 5 min at RT. Supernatant |
| Exchange at 48 hr | media was completely removed via aspiration, and replaced with 1 mL of |
| post-transfection | fresh media. Cells were replated in 24-well static culture plates and |
| | incubated at 37° C., 5% CO₂ prior to early timepoint flow analysis |
| Method C - 1:1 | Transfected cells were mixed and resuspended via pipet prior to being |
| Cell Split at 48 hr | split in a 1:1 ratio of cells:fresh media; specifically, 500 uL volume of |
| post-transfection | transfected cells were removed from the sample wells, and were replaced |
| | with 500 uL fresh media. Cells were incubated at 37° C., 5% CO₂ prior to |
| | early timepoint flow analysis |

Transfected cells were incubated at 37° C., 5% CO₂, sampled for intermittent analysis for gene editing efficiency, cell count, and cell viability, and sub-cultured into fresh media to promote cellular expansion when cells reached confluence, as described in Example 14. Cells were harvested at a terminal endpoint to evaluate integrated gene-edited expression.

b. Results

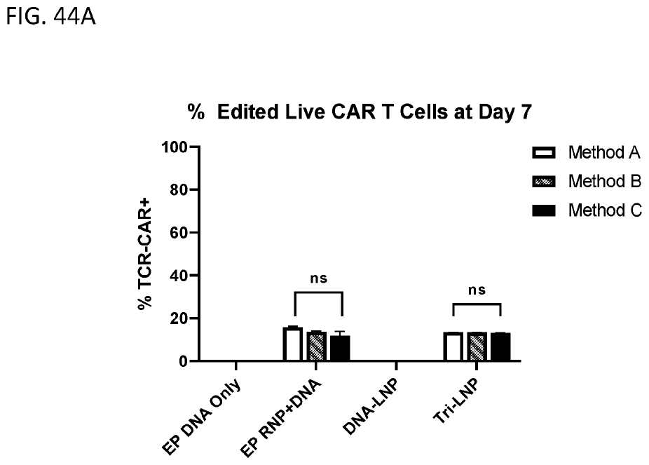

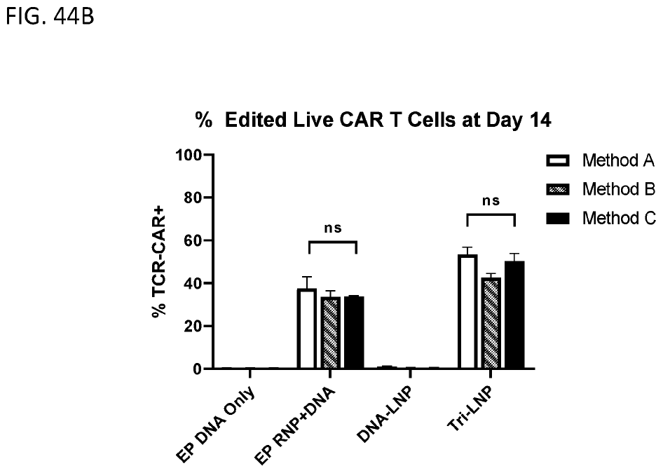

FIG. 44A and FIG. 44B show the percentage of fully edited cells 7 days and 14 days post-transfection, respectively, for the three different culture methods (Methods A-C) described above. Fully edited cells display TCR knockout (TCR−) and expression of CAR. By day 7 post-transfection (FIG. 44A), approximately 13% of cells were fully edited in cells transfected via electroporation or with the tri-LNPs. Cells transfected with only DNA (EP DNA only or DNA-LNP controls) did not display any gene editing. At day 14 post-transfection (FIG. 44B), both electroporated cells and cells transduced with tri-LNPs showed marked increases in gene editing. Notably, the tri-LNP showed improved results across all cell culture methods. Using Method A, gene editing was observed in approximately 55.8% of cells transduced with the tri-LNPs.

### Example 16: Gene Editing Across Donors

An experiment was designed to evaluate performance of tri-LNPs across multiple donors. The T cells of three individual healthy human donors were transduced with tri-LNPs or were electroporated with RNP and ceDNA. Experiments were performed as described in Examples 13b and Example 14.

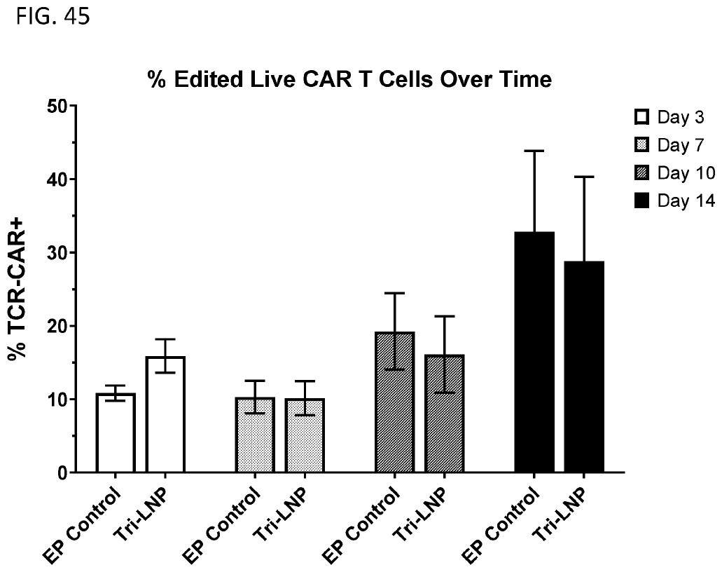

FIG. 45 depicts average percentages of fully edited cells from the three donors on various days post-transfection. In these experiments, the percentage of fully edited cells were comparable for the electroporation method and tri-LNP delivery method.

### Example 17: Fluorescence Resonance Energy Transfer (FRET) Experiments to Assess Co-LNPs

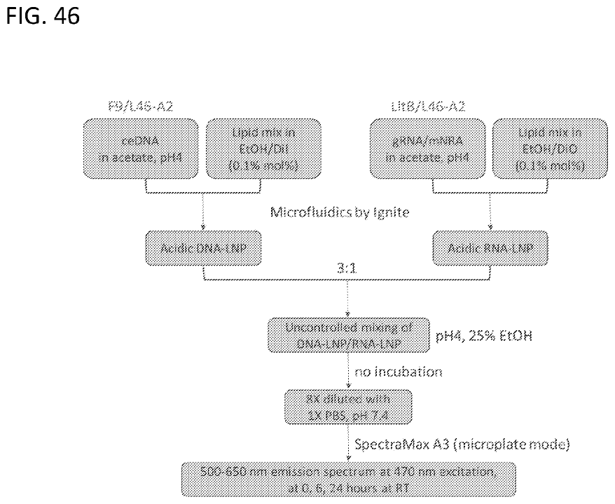

In order to investigate the degree of lipid mixing as DNA and RNA LNPs fuse to form co-LNPs, we applied a FRET (fluorescence resonance energy transfer) assay. FRET dye pairs were used to investigate the fusion between two separate LNPs. In this technique, two populations of LNPs were labeled with a donor and an acceptor fret dye pair, respectively. The labeled LNPs were then mixed and incubated, allowing them to fuse with each other. When the donor and acceptor dyes are in close proximity to one another, a fluorescence resonance energy transfer occurs from donor dye to acceptor dye, resulting in a peak in signal at the donor dye wavelength. For these experiments, DNA LNPs were prepared with 0.1 mol % DiI dye (Invitrogen) and RNA LNPs with 0.1 mol % DiO dye (Invitrogen) following LNP formulation as described in Examples above. To determine the impact of the pH 4 to pH 7 neutralization process in the fusion of DNA LNPs and RNA LNPs to form co-LNPs, samples were prepared according to Table E31. Readings were taken at 0, 6 & 24 hrs post-formulation at room temperature. An experimental overview is provided in FIG. 46. Samples were diluted in a 96 well plate and analyzed via fluorescence plate reader.

TABLE E31
Sample Preparation for FRET analysis

| Sample | Preparation method |
|---|---|
| Co-LNP (pH4) | 3:1 DNA-LNP(pH 4):RNA-LNP(pH 4) mixture, then diluted 8X with Buffer A |
| | (final pH of 4) |
| Co-LNP (pH7) | 3:1 DNA-LNP(pH 4):RNA-LNP(pH 4) mixture, then diluted 8X with PBS |
| | (final pH of 7) |
| Dual-LNP | Dilute acidic DNA- and RNA-LNPs 8X in PBS, then mix them 3:1 v/v (final |
| | pH of 7) |
| DNA-LNP | Dilute acidic LNP 8X in PBS, then mix 3 parts of DNA-LNP with 1 part of |
| | “buffer B” (final pH of 7) |
| RNA-LNP | Dilute acidic LNP 8X in PBS, then mix 1 part of RNA-LNP with 3 parts of |
| | “buffer B” (final pH of 7) |

*buffer A = 3/1 Acetate (pH 4)/ethanol; buffer B = 3/1 Acetate (pH 4)/ethanol:PBS (1:7)

Determination of the lipid fusion level by subsequent FRET signal was calculated as:

$$Em 565 from {FRET}_{( time )} = {RFU}_{co - LNP ( time )} - {RFU}_{DNA - LNP ( time )} - {RFU}_{RNA - LNP ( time )}$$

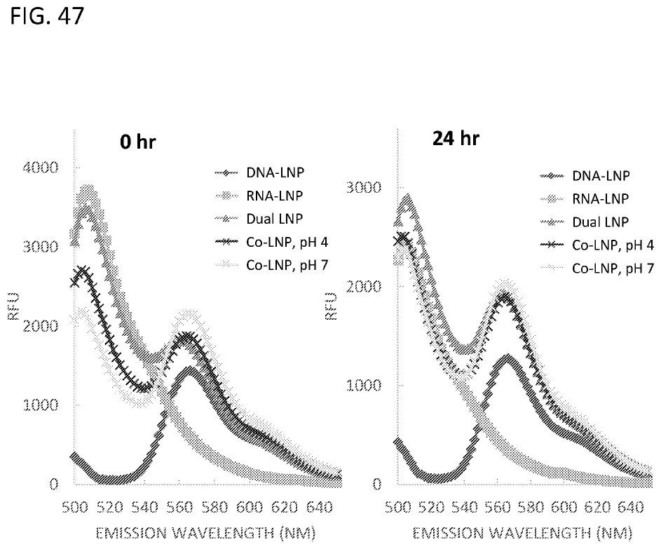

A compilation of four independent experiments (FIG. 47) demonstrated that significant lipid fusion occurs immediately when LNPs are mixed (0 hr timepoint) at pH 4 (Sample: co-LNP, pH 4), and that maximal fusion occurs when LNPs are mixed at pH 4 and then neutralized to pH 7 (Sample: co-LNP, pH7).

FRET efficiency (a proxy for lipid fusion) was calculated as:

$${FRET}_{Time}^{Sample} = {Em}_{Time}^{565} / ( {Em}_{Time}^{565} + {Em}_{Time}^{510} ) ,$$

$$where {Em}^{565} = Fluorescence reading of emission at 565 nm , and$$

$${Em}^{510} = Fluorescence reading of emission at 510 nm$$

$$( Time = incubation time ) .$$

Normalization of the FRET efficiency was calculated as:

$${FRET}_{Time}^{LNP} - {FRET}_{Time}^{m i n} ) / ( {FRET}_{Time}^{M ax} - {FRET}_{Time}^{m i n} ) ,$$

$$where {FRET}^{m i n} = FRET efficiency by the RNA - LNP ( DiO ) , and$$

$${FRET}^{M ax} = FRET efficiency by the DNA - LNP ( DiI )$$

$$( Time = incubation time ) .$$

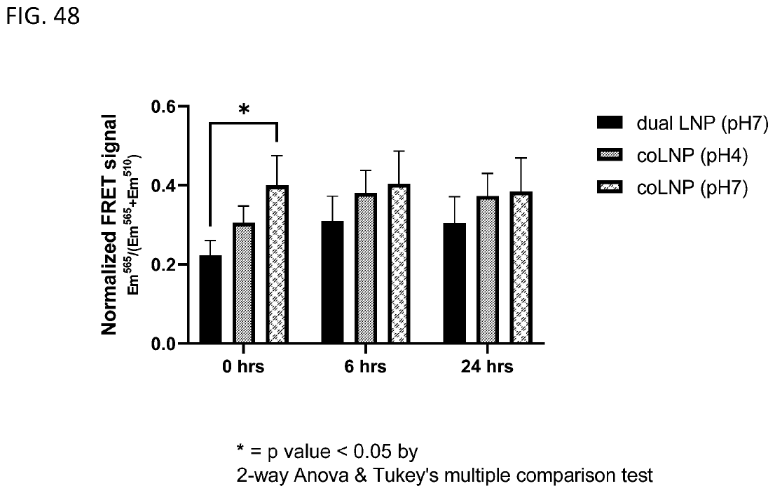

Based on these calculations, a normalized FRET signal was calculated across the four independent signals, which is graphically depicted in FIG. 48. Notably, although DNA and RNA LNPs fuse to some extent when mixed either in a pH 4 or pH 7 buffer, the maximum amount of lipid fusion (and co-LNP formation) is generated when RNA and DNA LNPs are mixed at pH 4 and then neutralized at pH 7. A statistical analysis (2 way anova and subsequent Tukey's multiple comparison test) was used to demonstrate that LNPs prepared by the co-LNP methodology described herein has significantly greater fusion than simply mixing LNPs at pH 7 (dual LNPs).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A co-formulated lipid nanoparticle (co-LNP) comprising a fusion of a first lipid nanoparticle (LNP) and a second lipid nanoparticle (LNP), wherein, prior to fusion:
(1) the first LNP comprises:
(i) a deoxyribonucleic acid (DNA) molecule; and
(ii) a first ionizable lipid; and
(2) the second LNP comprises:
(i) a first ribonucleic acid (RNA) molecule; and
(ii) a second ionizable lipid.

2. The co-LNP of claim 1, wherein the first ionizable lipid and/or the second ionizable lipid is an ionizable amino lipid.

3. The co-LNP of claim 1 or claim 2, comprising a volumetric ratio of the first LNP to the second LNP that is between about 3:1 and about 1:3.

4. The co-LNP of any of claims 1-3, comprising a first helper lipid and a second helper lipid.

5. The co-LNP of any of claims 1-4, further comprising a non-ionizable cationic lipid.

6. The co-LNP of claim 5, wherein the non-ionizable cationic lipid has the following structure:

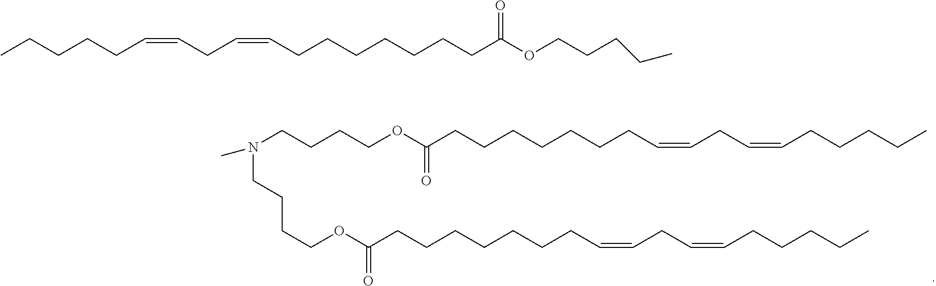

7. The co-LNP of claim 5 or claim 6, wherein the mass fraction of the non-ionizable lipid is between about 0.2% and about 20%.

8. The co-LNP of any of claims 1-7, which has an average size of between about 50 nm and 150 nm, or between about 75 nm and about 125 nm, as measured by dynamic light scattering (DLS).

9. The co-LNP of any of claims 1-8, wherein the first ionizable lipid comprises a diketopiperazine ring core.

10. The co-LNP of any of claims 1-9, wherein the first ionizable lipid comprises an unsaturated tail.

11. The co-LNP of claim 10, wherein the unsaturated tail is an unsaturated linoleil tail.

12. The co-LNP of any of claims 1-11, wherein the first ionizable lipid is: (a) OF-C4-Deg-Lin, or an analog thereof; or (b) cKK-E12, or an analog thereof.

13. The co-LNP of claim 12, wherein the first ionizable lipid is OF-C4-Deg-Lin.

14. The co-LNP of any of claims 1-13, wherein the first ionizable lipid and the second ionizable lipid are the same.

15. The co-LNP of any of claims 1-14, where the first ionizable lipid and the second ionizable lipids are different.

16. The co-LNP of any of claims 1-8, wherein the first ionizable lipid is DLin-KC2-DMA, or an analog thereof.

17. The co-LNP of claim 16, wherein the first ionizable lipid is DLin-KC2-DMA.

18. The co-LNP of any of claims 1-17, wherein the second ionizable lipid is comprises a diketopiperazine ring core.

19. The co-LNP of claim 18, wherein the second ionizable lipid is: (a) OF-C4-Deg-Lin, or an analog thereof; or (b) cKK-E12, or an analog thereof.

20. The co-LNP of claim 19, wherein the second ionizable lipid is OF-C4-Deg-Lin, or an analog thereof.

21. The co-LNP of claim 18, wherein the second ionizable lipid is OF-C4-Deg-Lin.

22. The co-LNP of claim 18, wherein the second ionizable lipid is cKK-E12, or an analog thereof.

23. The co-LNP of claim 22, wherein the second ionizable lipid is cKK-E12.

24. The co-LNP of any of claims 1-17, wherein the second ionizable lipid is DLin-MC3-DMA, or an analog thereof.

25. The co-LNP of any of claim 24, wherein the second ionizable lipid is DLin-MC3-DMA.

26. The co-LNP of any of claims 4-25, wherein the first helper lipid is 1-stearoyl-2-oleoyl-sn-glycero-3-phosphocholine (SOPC).

27. The co-LNP of any of claims 4-26, wherein the second helper lipid is 1,2-distearoyl-sn-glycero-3-phosphocholine (DSPC).

28. The co-LNP of any of claims 1-27, wherein the DNA molecule comprises a transgene.

29. The co-LNP of claim 28, wherein the transgene encodes a recombinant receptor.

30. The co-LNP of claim 29, wherein the transgene is positioned between protelomerase binding sequences.

31. The co-LNP of any one of claims 28-30, wherein the transgene is operably linked to a promoter and positioned between inverted terminal repeats (ITRs).

32. The co-LNP of any of claims 1-31, wherein the DNA molecule is a closed end DNA vector or a nanoplasmid.

33. The co-LNP of any of claims 29-32, wherein the recombinant receptor is a chimeric antigen receptor (CAR) or a T cell receptor (TCR).

34. The co-LNP of any of claim 33, wherein the recombinant receptor is a CAR.

35. The co-LNP of claim 34, wherein the CAR is a bispecific CAR.

36. The co-LNP of claim 35, wherein the bispecific CAR is between about 6 kilobases and 8 kilobases or wherein the bispecific CAR is about 8 kilobases.

37. The co-LNP of any one of claims 34-36, wherein the CAR comprises an extracellular antigen-binding domain, a transmembrane domain, and an intracellular region.

38. The co-LNP of claim 37, wherein the extracellular antigen-binding domain is an antibody or an antigen-binding fragment thereof that binds to an antigen that is associated with, or expressed on a cell or tissue of a disease or condition.

39. The co-LNP of claim 38, wherein the antigen is selected from the group consisting of αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR VIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), and Wilms Tumor 1 (WT-1).

40. The co-LNP of any one of claims 1-39, wherein the first RNA molecule is or comprises a guide RNA (gRNA).

41. The co-LNP of claim 40, wherein the gRNA is a single guide RNA (sgRNA).

42. The co-LNP of claim 40 or claim 41, wherein the gRNA is complexed with a recombinant nuclease capable of inducing a DNA break.

43. The co-LNP of claim 40 or claim 41, wherein the second LNP further comprises a nucleotide sequence encoding a recombinant nuclease capable of inducing a DNA break.

44. The co-LNP of claim 43, wherein the recombinant nuclease is a zinc finger nuclease (ZFN), a transcription activator-like effector nuclease (TALEN), or a CRISPR-associated nuclease (Cas).

45. The co-LNP of claim 44, wherein the Cas is Cas9 or Cas12a.

46. The co-LNP of claim 43, wherein the nucleotide sequence encodes a transposase.

47. The co-LNP of claim 46, wherein the transposase is a piggyBac transposase or a Sleeping Beauty transposase.

48. The co-LNP of any one of claims 1-47, further comprising a third LNP, wherein the third LNP comprises, prior to fusion: (i) a second RNA molecule; and (ii) a third ionizable lipid.

49. The co-LNP of claim 48, wherein the third ionizable lipid is an ionizable amino lipid.

50. The co-LNP of claim 48 or claim 49, comprising a volumetric ratio of the first LNP to the second and third LNPs that is between about 3:1 and about 1:3.

51. The co-LNP of any of claims 48-50, wherein the first RNA molecule of the second LNP is a gRNA, and the second RNA molecule of the third LNP is a nucleotide sequence encoding a recombinant nuclease capable of inducing a DNA break.

52. The co-LNP of any one of claims 1-51, wherein the first LNP and the second LNP are precursor LNPs.

53. The co-LNP of claim 52, wherein the precursor LNPS are prepared in an acidic environment.

54. The co-LNP of claim 53, wherein the pH of the acidic environment is between about 4 and about 5.

55. The co-LNP of any one of claims 1-54, wherein the co-LNP shows a fluorescence energy transfer (FRET).

56. The co-LNP of claim 55, wherein the normalized FRET signal is greater than 0.3

57. The co-LNP of claim 55, wherein the normalized FRET signal is greater than 0.35.

58. The co-LNP of claim 55, wherein the normalized FRET signal is greater than 0.4.

59. The co-LNP of any one of claims 1-58, wherein the mass fraction of the first and second ionizable lipids in the co-LNP is between about 40% and about 55%.

60. The co-LNP of claim 59, wherein the mass fraction of the first and second ionizable lipids in the co-LNP is between about 40% and about 50%.

61. A co-formulated lipid nanoparticle (co-LNP) comprising:
(1) a deoxyribonucleic acid (DNA) molecule and a ribonucleic acid (RNA) molecule; and
(2) a first ionizable lipid and a second ionizable lipid.

62. The co-LNP of claim 61, wherein (i) the DNA molecule is associated with the first ionizable lipid; and (ii) the RNA molecule is associated with the second ionizable lipid.

63. The co-LNP of claim 61, wherein more than 75% of the first ionizable lipid is associated with the DNA molecule and more than 75% of the second ionizable lipid is associated with the first RNA molecule.

64. The co-LNP of claim 61, wherein more than 85% of the first ionizable lipid is associated with the DNA molecule and more than 85% of the second ionizable lipid is associated with the first RNA molecule.

65. The co-LNP of claim 61, wherein more than 95% of the first ionizable lipid is associated with the DNA molecule and more than 95% of the second ionizable lipid is associated with the first RNA molecule.

66. The co-LNP of any one of claims 61-65, further comprising a third ionizable lipid.

67. The co-LNP of claim 66, further comprising a second ribonucleic acid (RNA) molecule.

68. The co-LNP of claim 67, wherein more than 85% of the third ionizable lipid is associated with the second RNA molecule or wherein more than 95% of the third ionizable lipid is associated with the second RNA molecule.

69. The co-LNP of any of claims 61-68, further comprising a non-ionizable cationic lipid.

70. The co-LNP of claim 69, wherein the non-ionizable cationic lipid has the following structure:

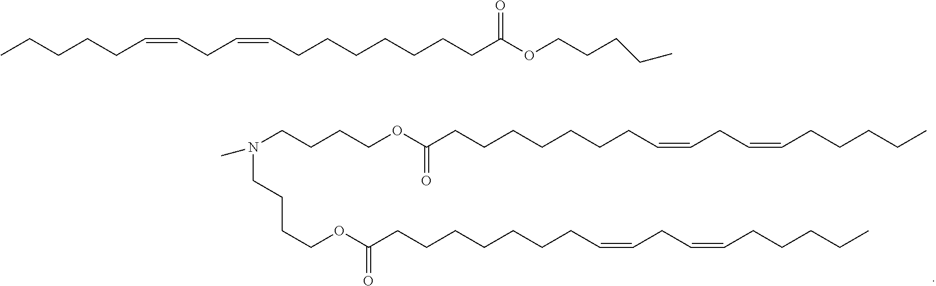

71. The co-LNP of claim 69 or claim 70, wherein the mass fraction of the non-ionizable lipid is between about 0.2% and about 20%.

72. The co-LNP of any of claims 61-71, wherein the first ionizable lipid comprises a diketopiperazine ring core.

73. The co-LNP of claim 72, wherein the first ionizable lipid is: (a) OF-C4-Deg-Lin, or an analog thereof; or (b) cKK-E12, or an analog thereof.

74. The co-LNP of any of claims 61-73, wherein the DNA molecule is a closed end DNA vector or a nanoplasmid.

75. The co-LNP of any of claims 61-74, wherein the DNA molecule comprises a transgene.

76. The co-LNP of claim 75, wherein the transgene encodes a recombinant receptor.

77. The co-LNP of any of claim 76, wherein the recombinant receptor is a chimeric antigen receptor (CAR) or a T cell receptor (TCR).

78. The co-LNP of claim 77, wherein the recombinant receptor is a CAR.

79. The co-LNP of claim 78, wherein the CAR is a bispecific CAR.

80. The co-LNP of any one of claims 67-79, wherein the first RNA is a guide RNA and the second RNA comprises a nucleotide sequence encoding a recombinant nuclease capable of inducing a DNA break.

81. The co-LNP of claim 80, wherein the recombinant nuclease is Cas9 or Cas12a.

82. A co-formulated lipid nanoparticle (co-LNP) comprising:
(1) a first ribonucleic acid (RNA) molecule and a second ribonucleic acid (RNA) molecule; and
(2) a first ionizable lipid and a second ionizable lipid,
wherein one of the first and second RNA molecules encodes a recombinant nuclease capable of inducing a DNA break; and the other of the first and second RNA molecules is a guide RNA (gRNA).

83. A co-formulated lipid nanoparticle (co-LNP) comprising a fusion of a first lipid nanoparticle (LNP) and a second lipid nanoparticle (LNP), wherein:
(1) the first LNP comprises:
(i) a ribonucleic acid (RNA) molecule; and
(ii) a first ionizable lipid; and
(2) the second LNP comprises:
(i) a ribonucleic acid (RNA) molecule; and
(ii) a second ionizable lipid,
wherein one of the first and second RNA molecules encodes a recombinant nuclease capable of inducing a DNA break; and the other of the first and second RNA molecules is a guide RNA (gRNA).

84. The co-LNP of claim 82 or claim 83, wherein the gRNA is a single guide RNA (sgRNA).

85. The co-LNP of any of claims 82-84, wherein the recombinant nuclease is a zinc finger nuclease (ZFN), a transcription activator-like effector nuclease (TALEN), or a CRISPR-associated nuclease (Cas).

86. The co-LNP of claim 85, wherein the Cas is Cas9 or Cas12a.

87. The co-LNP of any of claims 83-86, comprising a volumetric ratio of the first LNP to the second LNP that is about 1:1.

88. A method of producing a co-formulated lipid nanoparticle (co-LNP), comprising:
(1) mixing, in an acidic buffer:
(a) a first lipid nanoparticle (LNP) comprising a first ionizable lipid and a nucleic acid molecule; and
(b) a second LNP comprising a second ionizable lipid and a ribonucleic acid (RNA) molecule,
thereby generating a composition comprising the first LNP and the second LNP; and
(2) neutralizing the composition comprising the first LNP and the second LNP, thereby generating a co-LNP, which is a fusion of the first LNP and the second LNP,
wherein the nucleic acid molecule in (a) is a deoxyribonucleic acid (DNA) molecule or a ribonucleic acid (RNA) molecule.

89. The method of claim 88, wherein the nucleic acid molecule in (a) is a DNA molecule.

90. The method of claim 88, wherein the nucleic acid molecule in (a) is an RNA molecule.

91. The method of any of claims 88-90, wherein the volumetric ratio of the first LNP to the second LNP in the composition is between about 3:1 and about 1:3.

92. The method of any of claims 88, 89, and 91, further comprising mixing, in the acidic buffer, (c) a third LNP comprising a third ionizable lipid and an RNA molecule, thereby generating a composition comprising the first, second, and third LNPs.

93. The method of claim 92, wherein the volumetric ratio of the first LNP to the second and third LNPs in the composition is between about 3:1 and about 1:3.

94. The method of any of claims 88-93, wherein the acidic buffer is an acetate buffer.

95. The method of any of claims 88-94, wherein the acidic buffer has a pH of between about 3.0 and about 4.5, or of 4.0.

96. The method of any of claims 88-95, wherein the acidic buffer is neutralized to a pH of between about 6.0 and about 7.5, or between about 6.5 and about 7.0.

97. The method of any of claims 88-96, wherein neutralizing the composition comprising the first LNP and the second LNP comprises adding an isotonic buffer.

98. The method of claim 97, wherein the isotonic buffer has a pH of about 7.4.

99. The method of claim 97 or claim 98, wherein neutralizing the composition comprising the first LNP and the second LNP comprises adding at least about 6 parts of the isotonic buffer to 1 part of the acidic buffer.

100. The method of any of claims 97-99, wherein neutralizing the composition comprising the first LNP and the second LNP comprises adding between about 6-7 parts of the isotonic buffer to 1 part of the acidic buffer.

101. The method of any of claims 97-100, wherein the isotonic buffer is phosphate buffered saline (PBS).

102. A co-LNP produced by the method of any of claims 88-101.

103. A lipid nanoparticle (LNP) comprising:
(1) an ionizable lipid comprising a diketopiperazine ring core, or wherein the ionizable lipid is an ionizable amino lipid; and
(2) a deoxyribonucleic acid (DNA) molecule.

104. The LNP of claim 103, wherein the ionizable lipid is an ionizable amino lipidoid.

105. The LNP of claim 103 or claim 104, wherein the mass fraction of the ionizable lipid is between about 35% and about 45%.

106. The LNP of any of claims 103-105, wherein the ionizable lipid comprises an unsaturated tail, optionally an unsaturated linoleic tail.

107. The LNP of any of claim 103 or claim 104, wherein the ionizable lipid is: (a) OF-C4-Deg-Lin, or an analog thereof; (b) cKK-E12, or an analog thereof; or (c) DLin-KC2-DMA, or an analog thereof.

108. The LNP of any of claims 103-107, wherein the ionizable lipid is OF-C4-Deg-Lin; and the mass fraction of the ionizable lipid is between about 35% and about 45%.

109. The LNP of any of claims 103-108, wherein the ionizable lipid is cKK-E12; and the mass fraction of the ionizable lipid is between about 35% and about 45%.

110. A lipid nanoparticle (LNP) comprising:
(1) an ionizable lipid, or an analog thereof, wherein the ionizable lipid is DLin-KC2-DMA; and
(2) a deoxyribonucleic acid (DNA) sequence,
wherein the mass fraction of the ionizable lipid is between about 35% and about 45%.

111. The LNP of any of claims 103-110, comprising a helper lipid.

112. The LNP of any of claims 103-111, comprising a polyethylene glycol (PEG)-conjugated lipid.

113. The LNP of any of claims 103-112, comprising cholesterol.

114. The LNP of any of claims 103-113, wherein the mass fraction of the ionizable lipid is about 40%.

115. The LNP of any of claims 111-114, wherein the mass fraction of the helper lipid is between about 18% and about 22%.

116. The LNP of any of claims 111-115, wherein the mass fraction of the helper lipid is about 19%.

117. The LNP of any of claims 112-116, wherein the mass fraction of the PEG-conjugated lipid is between about 2% and about 3%.

118. The LNP of any of claims 103-117, wherein the mass fraction of the PEG-conjugated lipid is about 2.5%.

119. The LNP of any of claims 113-118, wherein the mass fraction of the cholesterol is between about 30% and about 40%.

120. The LNP of any of claims 113-119, wherein the mass fraction of the cholesterol is about 35%.

121. The LNP of any of claims 111-120, wherein the helper lipid is 1-stearoyl-2-oleoyl-sn-glycero-3-phosphocholine (SOPC).

122. The LNP of any of claims 112-121, wherein the PEG-conjugated lipid is DMG-PEG2000.

123. The LNP of any of claims 103-122, wherein the mass fraction of the DNA molecule is between about 3% and about 4%.

124. The LNP of any of claims 103-123, wherein the mass fraction of the DNA molecule is about 3.5%.

125. A lipid nanoparticle (LNP) comprising:
(1) between about 35% and about 45% mass fraction of an ionizable lipid, wherein the ionizable lipid is OF-C4-Deg-Lin;
(2) between about 3% and about 4% mass fraction of a deoxyribonucleic acid (DNA) molecule;
(3) between about 18% and about 22% mass fraction of a helper lipid;
(4) between about 2% and about 3% mass fraction of a polyethylene glycol (PEG)-conjugated lipid; and
(5) between about 30% and about 40% mass fraction of cholesterol.

126. A lipid nanoparticle (LNP) comprising:
(1) between about 35% and about 45% mass fraction of an ionizable lipid, wherein the ionizable lipid is cKK-E12;
(2) between about 3% and about 4% mass fraction of a deoxyribonucleic acid (DNA) molecule;
(3) between about 18% and about 22% mass fraction of a helper lipid;
(4) between about 2% and about 3% mass fraction of a polyethylene glycol (PEG)-conjugated lipid; and
(5) between about 30% and about 40% mass fraction of cholesterol.

127. A lipid nanoparticle (LNP) comprising:
(1) between about 35% and about 45% mass fraction of an ionizable lipid, wherein the ionizable lipid is DLin-KC2-DMA;
(2) between about 3% and about 4% mass fraction of a deoxyribonucleic acid (DNA) molecule;
(3) between about 18% and about 22% mass fraction of a helper lipid;
(4) between about 2% and about 3% mass fraction of a polyethylene glycol (PEG)-conjugated lipid; and
(5) between about 30% and about 40% mass fraction of cholesterol.

128. The LNP of any of claims 123-127, wherein the DNA molecule comprises a transgene.

129. The LNP of claim 128, wherein the transgene encodes a recombinant receptor.

130. The LNP of any of claims 103-129, wherein the DNA molecule is a closed end DNA (ceDNA) vector or a nanoplasmid.

131. The LNP of claim 130, wherein the transgene is positioned between protelomerase binding sequences.

132. The LNP of claim 128 or claim 129, wherein the transgene is operably linked to a promoter and positioned between inverted terminal repeats (ITRs).

133. The co-LNP of any of claims 129-132, wherein the recombinant receptor is a chimeric antigen receptor (CAR) or a T cell receptor (TCR).

134. The LNP of any of claims 129-133, wherein the recombinant receptor is a CAR.

135. The LNP of claim 133 or claim 134, wherein the CAR comprises an extracellular antigen-binding domain, a transmembrane domain, and an intracellular region.

136. The LNP of claim 135, wherein the extracellular antigen-binding domain is an antibody or an antigen-binding fragment thereof that binds to an antigen that is associated with, or expressed on, a cell or tissue of a disease or condition.

137. The LNP of claim 136, wherein the antigen is selected from the group consisting of αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C—C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), and Wilms Tumor 1 (WT-1).

138. The LNP of any of claims 135-137, wherein the intracellular region comprises an intracellular signaling domain that is or comprises an intracellular signaling domain of a CD3 chain, or a signaling portion thereof.

139. The LNP of any of claims 135-138, wherein the intracellular region comprises one or more costimulatory signaling domain(s) comprising an intracellular signaling domain selected from the group consisting of: a CD28, a 4-1BB, an ICOS, or a signaling portion thereof.

140. The LNP of any of claims 103-126, wherein the DNA molecule comprises a single-stranded DNA oligonucleotide (ssODN) or a double-stranded DNA oligonucleotide (dsODN), the ssODN or the dsODN comprising a nucleotide sequence that is homologous to a target genomic locus,

141. A lipid nanoparticle (LNP) comprising:
(a) an ionizable lipid;
(b) a helper lipid that is 1,2-distearoyl-sn-glycero-3-phosphocholine (DSPC);
(c) a polyethylene glycol (PEG)-conjugated lipid that is DMG-PEG2000;
(d) cholesterol; and
(e) a ribonucleic acid (RNA) molecule.

142. The LNP of claim 141, wherein the ionizable lipid is an ionizable amino lipid selected from the group consisting of OF-C4-Deg-Lin, an analog thereof, cKK-E12, an analog thereof, DLin-MC3-DMA, and an analog thereof.

143. The LNP of claim 141 or claim 142, wherein:
the ionizable lipid is a lipidoid comprising a diketopiperazine amino core; and/or
the ionizable lipid comprises an unsaturated tail, optionally an unsaturated linoleic tail.

144. The LNP of any of claims 141-143, wherein the mass fraction of the ionizable lipid is between about 50% and about 65%.

145. The LNP of any of claims 141-144, wherein the mass fraction of DSPC is between about 10% and about 15%.

146. The LNP of any of claims 141-145, wherein the mass fraction of DMG-PEG2000 is between about 5% and about 7.5%.

147. The LNP of any of claims 141-146, wherein the mass fraction of cholesterol is between about 15% and about 25%.

148. The LNP of any of claims 141-147, wherein the mass fraction of the RNA molecule is between about 3% and about 10%.

149. The LNP of any of claims 141-143, wherein the mass fraction of the ionizable lipid is between about 32% and about 36%.

150. The LNP of any of claims 141-143 and 149, wherein the mass fraction of DSPC is between about 15% and about 20%.

151. The LNP of any of claims 141-143, 149, and 150, wherein the mass fraction of DMG-PEG2000 is between about 3.5% and about 5.5%.

152. The LNP of any of claims 141-143, and 149-151, wherein the mass fraction of cholesterol is between about 35% and about 45%.

153. The LNP of any of claims 141-143, and 149-152, wherein the mass fraction of the RNA molecule is between about 2.5% and about 3%.

154. The LNP of any of claims 141-143, and 149-152, wherein the mass fraction of the RNA molecule is between about 3% and about 4%.

155. The LNP of any of claims 141-154, wherein the RNA molecule encodes a transposase.

156. The LNP claim 155, wherein the transposase is a piggyBac transposase or a Sleeping Beauty transposase.

157. The LNP of any of claims 141-154, wherein the RNA molecule comprises a guide RNA (gRNA).

158. The LNP of claim 157, wherein the gRNA is a single guide RNA (sgRNA).

159. The LNP of claim 157 or claim 158, wherein the gRNA is complexed with a recombinant nuclease capable of inducing a DNA break.

160. The LNP of any of claims 141-154, wherein the RNA sequence comprises a nucleotide sequence encoding a recombinant nuclease capable of inducing a DNA break.

161. The LNP of claim 159 or claim 160, wherein the recombinant nuclease is a zinc finger nuclease (ZFN), a transcription activator-like effector nuclease (TALEN), or a CRISPR-associated nuclease (Cas).

162. The LNP of any of claims 159-161, wherein the recombinant nuclease is a Cas nuclease, optionally Cas9 or Cas12a.

163. A composition comprising: (1) a LNP of any of claims 103-140; and (2) a lipid nanoparticle comprising a ribonucleic acid (RNA) molecule.

164. A composition comprising: (1) a LNP of any of claims 103-140; and (b) a LNP of any of claims 141-162.

165. A co-formulated lipid nanoparticle (co-LNP) comprising a fusion of: (1) a LNP of any of claims 103-140; and (2) a lipid nanoparticle (LNP) comprising a ribonucleic acid (RNA) molecule.

166. A co-formulated lipid nanoparticle (co-LNP) comprising a fusion of (1) a lipid nanoparticle comprising a deoxyribonucleic acid (DNA) molecule; and (2) a LNP of any of claims 141-161.

167. A co-formulated lipid nanoparticle (co-LNP) comprising a fusion of (1) a LNP of any of claims 103-140; and (2) a LNP of any of claims 141-162.

168. A combination of: (1) a LNP of any of claims 103-140; and (2) a ribonucleoprotein (RNP) complex.

169. A composition comprising the LNP of any of claims 103-168.

170. A method of genetically engineering an immune cell, the method comprising:
(1) introducing a ribonucleic acid (RNA) molecule into an immune cell by electroporation; and
(2) incubating the immune cell with the LNP of any of claims 102-140 or co-LNP of any of claims 1-87 or 165-168.

171. A method of genetically engineering an immune cell, the method comprising:
(1) introducing a ribonucleoprotein complex (RNP) into an immune cell by electroporation; and
(2) incubating the immune cell with the LNP of any of claims 102-140 or co-LNP of any of claims 1-87 or 165-168.

172. A method of genetically engineering an immune cell, the method comprising incubating the immune cell with (1) the LNP of any of claims 102-140; and (2) the LNP of any of claims 141-162.

173. A method of genetically engineering an immune cell, the method comprising incubating an immune cell with the co-LNP of any of claims 1-87 or 165-168.

174. The method of any of claims 170-173, wherein the immune cell is a lymphocyte.

175. The method of any of claims 170-174, wherein the immune cell is a T cell.

176. The method of claim 175, wherein the T cell is a primary T cell.

177. The method of claim 175 or claim 176, wherein the T cell is a CD4+ T cell or a CD8+ T cell.

178. The method of any of claims 170-177, wherein, at the time of incubating the immune cell with the LNP, the co-LNP, or the composition, the immune cell is activated.

179. The method of any of claims 170-178, wherein, at the time of incubating the immune cell with the LNP, the co-LNP, or the composition, the immune cell expresses CD25, CD26, CD27, CD28, CD30, CD71, CD154, CD40L, CD134, or a combination thereof.

180. The method of any of claims 170-179, wherein the immune cell is incubated under stimulating conditions prior to incubating the immune cell with the LNP, the co-LNP, or the composition.

181. The method of claim 180, wherein the immune cell is incubated under stimulating conditions for between about 24 hours and about 72 hours, or for about 48 hours.

182. The method of claim 180 or claim 181, wherein the stimulating conditions comprise incubation with a stimulatory reagent capable of activating an intracellular signaling domain of a component of a TCR complex and an intracellular signaling domain of a costimulatory molecule.

183. The method of claim 182, wherein the stimulatory reagent comprises a primary agent that binds to CD3 and a secondary agent that binds to a T cell costimulatory molecule.

184. The method of claim 183, wherein the costimulatory molecule is selected from the group consisting of CD28, 4-1BB, OX40, and ICOS.

185. The method of claim 183 or claim 184, wherein the primary agent is an anti-CD3 antibody or antigen-binding fragment, and the secondary agent is an anti-CD28 antibody or antigen-binding fragment.

186. The method of any of claims 170-185, wherein the immune cell is incubated with apolipoprotein E (ApoE) prior to incubating the immune cell with the LNP, the co-LNP, or the composition.

187. The method of claim 186, wherein the ApoE is ApoE4.

188. An immune cell produced by the method of any of claims 170-187.

189. A composition comprising a plurality of the immune cell of claim 188.

190. A method of producing a lipid nanoparticle (LNP), the method comprising:
(1) adding to an organic solvent comprising ethanol:
(a) an ionizable lipid;
(b) a helper lipid;
(c) a polyethylene glycol (PEG)-conjugated lipid; and
(d) cholesterol,
thereby generating an organic phase;
(2) adding to an aqueous solvent having an acidic pH, a deoxyribonucleic acid (DNA) molecule, thereby generating an aqueous phase; and
(3) combining the organic phase and the aqueous phase by laminar flow mixing in a device, thereby generating an LNP.

191. The method of claim 190, wherein the ionizable lipid is an ionizable amino lipid selected from the group consisting of OF-C4-Deg-Lin, an analog thereof, cKK-E12, an analog thereof, DLin-KC2-DMA, and an analog thereof.

192. The method of claim 190 or claim 191, wherein:
a flow rate of the aqueous phase in the device is between about 8 mL/min and about 10 mL/min; and/or
a flow rate of the organic phase in the device is between about 2 mL/min and about 4 mL/min.
